(19) United States
(12) Patent Application Publication
(10) Pub. No.: US 2006/0141600 A1
(43) Pub. Date: Jun. 29, 2006

(54) Methods and compositions related to argonaute proteins

(76) Inventors: Leemor Joshua-Tor, Huntington, NY (US); Ji-Joon Song, Arlington, MA (US); Gregory J. Hannon, Huntington, NY (US); Jidong Liu, Cold Spring Harbor, NY (US); Michelle A. Carmell, Nesconset, NY (US); Fabiola Rivas, Cold Spring Harbor, NY (US)

Correspondence Address: FISH & NEAVE IP GROUP;ROPES & GRAY LLP, ONE INTERNATIONAL PLACE, BOSTON, MA, 02110-2624, US

(21) Appl. No.: 11/192,437

(22) Filed: Jul. 28, 2005

Related U.S. Application Data
(60) Provisional application No. 60/592,269, filed on Jul. 29, 2004.
(60) Provisional application No. 60/592,297, filed on Jul. 28, 2004.

(51) Int. Cl. C12N 9/22; G06F 19/00

(52) U.S. Cl. 435/199; 702/19

(57) ABSTRACT

This invention provides methods and compositions related to Argonaute proteins and, in certain embodiments, the applications of these methods and compositions to treatment and therapeutics based on RNAi.

### RELATED APPLICATIONS

This application claims the benefit of priority to U.S. Provisional Patent Application Nos. 60/592,269, filed on Jul. 29, 2004, and 60/592,297, filed on Jul. 28, 2004, which applications are hereby incorporated by reference in their entireties.

### BACKGROUND OF THE APPLICATION

The presence of double-stranded RNA (dsRNA) in most eukaryotic cells provokes a sequence-specific silencing response known as RNA interference (RNAi) (G. J. Hannon, Nature 418, 244 (2002); A. Fire et al., Nature 391, 806 (1998)). The dsRNA trigger of this process can be derived from exogenous sources or transcribed from endogenous non-coding RNA genes that produce microRNAs (mRNAs) (Hannon, supra; G. Hutvagner et al., Curr. Opin. Genet. Dev. 12, 225 (2002)). RNAi begins with the conversion of dsRNA silencing triggers into small RNAs of ˜21-26 nt in length (A. Hamilton et al., Embo J. 21, 4671 (2002)). This is accomplished by processing of triggers by specialized RNaseIII family nucleases, Dicer and Drosha (E. Bernstein et al., Nature 409, 363 (2001); Y. Lee et al., Nature 425, 415 (2003)). Resulting small RNAs join an effector complex, known as RISC(RNA-Induced Silencing Complex) (S. M. Hammond et al., Nature 404, 293 (2000)). Silencing by RISC can occur via several mechanisms. In flies, plants and fungi, dsRNAs can trigger chromatin remodeling and transcriptional gene silencing (M. F. Mette et al., Embo J. 19, 5194 (2000); I. M. Hall et al., Science 297, 2232 (2002); T. Volpe et al., Science 22, 22 (2002); M. Pal-Bhadra et al., Mol. Cell 9, 315 (2002)). RISC can also interfere with protein synthesis, and this is the predominant mechanism used by miRNAs in mammals (P. H. Olsen et al., Dev. Biol. 216, 671 (1999); D. P. Bartel, Cell 116, 281 (2004)). However, the best-studied mode of RISC action is mRNA cleavage (T. Tuschl et al., Genes Dev. 13, 3191 (1999); P. D. Zamore, Cell 101, 25 (2000)). When programmed with a small RNA that is fully complementary to the substrate RNA, RISC cleaves that RNA at a discrete position, an activity that has been attributed to an unknown RISC component, “Slicer” (S. M. Elbashir et al., Embo J. 20, 6877 (2001); J. Martinez et al., Cell 110, 563 (2002)). Whether or not RISC cleaves a substrate can be determined by the degree of complementarity between the siRNA and mRNA, as mismatched duplexes are often not processed (Elbashir et al., supra). However, even for mammalian miRNAs, which normally repress at the level of protein synthesis, cleavage activity can be detected with a substrate that perfectly matches the miRNA sequence (G. Hutvagner et al., Science 1, 1 (2002)). This prompted the hypothesis that all RISCs are equal with the outcome of the RISC-substrate interaction being determined largely by the character of the interaction between the small RNA and its substrate.

RISC contains two signature components. The first is the small RNA, which co-fractionated with RISC activity in Drosophila S2 cell extracts (Hammond et al., supra) and whose presence correlated with dsRNA-programmed mRNA cleavage in Drosophila embryo lysates (Tuschl et al., supra; Zamore et al., supra). The second is an Argonaute protein, which was identified as a component of purified RISC in Drosophila (S. M. Hammond et al., Science 293, 1146 (2001)). Subsequent studies have suggested that Argonautes are also key components of RISC in mammals, fungi, worms, protozoans and plants (Martinez et al., supra; M. A. Carmell et al., Nat. Struct. Mol. Biol. 11, 214 (2004)). To date, the identity of “Slicer” and the function of Argonaute proteins are unknown.

### BRIEF SUMMARY OF THE APPLICATION

This application provides methods and compositions related to Argonaute proteins.

A first aspect of application provides a crystalline Argonaute. Certain embodiments provide an isolated and purified Argonaute protein having a three-dimensional structure defined by the atomic coordinates such as for example as shown in Table 3. The crystalline Argonaute may comprise an archae Argonaute protein. Alternatively, the crystalline Argonaute may comprise a mammalian Argonaute protein, e.g., a human Argonaute protein such as human Ago-2. Examples of mammalian Argonaute proteins may be Ago-1, Ago-2, Ago-3, or Ago-4.

In certain embodiments, a crystalline Argonaute may comprise an Argonaute protein having an amino acid sequence that is 95% identical to SEQ ID NO: 2 (or human Ago-2) or a homologue, fragment, variant, or derivative thereof. Alternatively, a crystalline Argonaute may comprise an Argonaute protein having an amino acid sequence that is 95% identical to SEQ ID NO: 2 (or human Ago-2) or a homologue, fragment, variant, or derivative thereof.

Certain embodiments provide a crystalline Argonaute comprising a three-dimensional structure defined by all or a portion of the atomic co-ordinates such as for example as set forth in Table 3.

The application also provides native crystals, derivative crystals or co-crystals, that have a root mean square deviation (“r.m.s.d.”) of less than or equal to about 1.5 Angstrom when superimposed, using backbone atoms (N, Cα, C and O), on the structure coordinates listed in Table 3.

A crystalline Argonaute of the application may comprise at least two domains, e.g., a PAZ domain and a PIWI domain. A PIWI domain comprises a carboxylate triad formed by the motif “DDX” (X refers to a third amino acid, e.g., E). A crystalline Argonaute of the application may comprise a PIWI domain having a carboxylate triad formed by D597, D669, and a third amino acid.

A crystalline Argonaute of the application may comprise the following overall architecture: the N-terminus, middle, and PIWI domains form a crescent-shaped base; and the PAZ domain is positioned above the crescent shaped base; resulting in a cleft between said crescent-shaped base and the PAZ domain.

In certain embodiments, a crystalline Argonaute permits an X-ray crystallography resolution better than 2.25 Angstrom.

In certain embodiments, a crystalline Argonaute is soaked with one or more agents to form co-complex structures.

A crystalline Argonaute may comprise a PIWI domain having an active site defined by two or more amino acids, such as for example the “DDX” (X representing a third amino acid, e.g., E) triad. A crystalline Argonaute may comprise a PAZ domain having an active site defined by two or more amino acids. In certain embodiments, an active site is capable of accommodating an agent, e.g., a ligand or an inhibitor. A ligand or an inhibitor may be a nucleic acid molecule, a peptidomimetic, or a small organic molecule. A ligand or an inhibitor may be soaked in to form a co-complex. A nucleic acid molecule that is a ligand or an inhibitor can be a single stranded RNA molecule, e.g., a single stranded RNA molecule comprising between 15-50 nucleotides.

The application further provides an isolated complex comprising an Argonaute protein and a single stranded RNA molecule hybridized to its target nucleic acid. In certain embodiments, the single stranded RNA molecule is bound to the PAZ domain of the Argonaute protein. In certain embodiments, the target nucleic acid further interacts with the crescent-shaped base of the Argonaute protein.

A further aspect of the application provides a method of determining the three-dimensional structure of an Argonaute protein or a mutant, derivative, variant, analogue, homologue, sub-domain or fragment thereof. The method may comprise aligning the amino acid sequence of the Argonaute mutant, derivative, variant, analogue, homologue, sub-domain or fragment with the amino acid sequence of PfAgo or as set forth in SEQ ID NO: 5 to match homologous regions of the amino acid sequences. The method may further comprise modeling the structure of the matched homologous regions of said target Argonaute protein of unknown structure on the corresponding regions of the Argonaute protein structure as defined by the atomic co-ordinates as set forth in Table 3. The method may also comprise determining a conformation for the Argonaute mutant, derivative, variant, analogue, homologue, sub-domain or fragment which substantially preserves the structure of said matched homologous regions.

A further aspect of the application provides a method of identifying an agent that binds an Argonaute protein. The method may comprise applying a 3-dimensional molecular modeling algorithm to the atomic coordinates of an Argonaute protein shown in Table 3 to determine the spatial coordinates of the binding pocket of the Argonaute protein. The method may further comprise electronically screening the stored spatial coordinates of a set of candidate agents against the spatial coordinates of the Argonaute protein binding pocket to identify agents that can bind to the Argonaute protein.

The application also provides a computer-based method for the analysis of the interaction of a molecular structure with an Argonaute protein. The method may comprise providing a structure comprising a three-dimensional representation of said Argonaute protein or a portion thereof, which representation comprises all or a portion of the coordinates set forth in Table 3. The method may further comprise providing a molecular structure to be fitted to said Argonaute protein structure. The method may also comprise fitting the molecular structure to the Argonaute protein structure, e.g., as set forth in the three-dimensional representation.

The application also provides a computer-readable storage medium encoded with the atomic coordinates or an Argonaute protein as shown in Table 3. Other embodiments also provide a data array comprising the atomic coordinates of an Argonaute protein as set forth in Table 3.

The application further provides an electronic representation of a crystal structure of an Argonaute protein. In certain embodiments, the electronic representation may contain atomic coordinate set forth in Table 3. Certain embodiments also provide an electronic representation of a binding site of the Argonaute protein. The binding site may locate in or be defined by the PAZ and/or PIWI domain or a portion thereof. Certain embodiments also provide an electronic representation of a domain of the Argonaute protein, e.g., a PIWI domain and/or a PAZ domain. Certain embodiments also provide an electronic representation of an agent in a binding site of an Argonaute protein, e.g., an active site of the Argonaute protein.

The crystal structure, the electronic representation, as well as other aspects of the application also relate to a method for identifying, designing, and/or optimizing an RNAi construct or RNAi therapeutic of the invention, e.g., to improve an RNAi therapeutic's pharmacokinetic and/or pharmacodynamic profile.

Another aspect of the application relates to a method of obtaining a crystal formed by an Argonaute protein. The crystal may be grown using a precipitant. The crystal may be grown in a buffer, the pH of which buffer may be varied. The crystal may also be grown in the presence of a ligand or an inhibitor that interacts with the Argonaute protein, e.g., a domain of the Argonaute protein. The quality of the crystal can be improved by microseeding.

A further aspect of the application relates to a method of identifying an agent that modulates the activity of an RNAi construct. The method may comprise identifying an agent that modulates the expression and/or activity of an Argonaute protein. The method may involve an Argonaute protein expressed in a cell. The expressed Argonaute protein may be endogenous or exogenous to the cell. In certain embodiments, the agent can modulate (e.g., increase) the RNase activity of the Argonaute protein. The agent may alternatively or further modulate (e.g., increase) the expression of said Argonaute gene. In certain embodiments, an agent modulates the RNase activity and/or expression of an Argonaute protein in a tissue or cell type-specific manner.

In certain embodiments, the application relates to a method of identifying an agent that modulates the activity of an RNAi therapeutic. The method may comprise identifying an agent that modulates the expression and/or activity of an Argonaute protein. The method may involve an Argonaute protein expressed in a cell. The expressed Argonaute protein may be endogenous or exogenous to the cell. In certain embodiments, the agent can modulate (e.g., increase) the RNase activity of the Argonaute protein. The agent may alternatively or further modulate (e.g., increase) the expression of said Argonaute gene. In certain embodiments, an agent modulates the RNase activity and/or expression of an Argonaute protein in a tissue or cell type-specific manner.

In certain embodiments, an RNAi construct or an RNAi therapeutic attenuates the expression of a target nucleic acid molecule. The attenuation may be by 2, 3, 5, 10, or higher fold. The target nucleic acid molecule may comprise an endogenous nucleic acid molecule. Alternatively, the target nucleic acid molecule is a heterologous to the genome of the cell. The heterologous nucleic acid molecule may be a nucleic acid from a pathogen.

An RNAi construct or an RNAi therapeutic of the application may comprise a nucleotide sequence at least 15 nucleotides in length that hybridizes to a target nucleic acid molecule. In certain embodiments, an RNAi construct or an RNAi therapeutic may comprise a hairpin nucleic acid. An RNAi construct or an RNAi therapeutic of the application may also comprise a promoter operably linked to a nucleotuide sequence that hybridizes to a target nucleic acid molecule. The promoter may be tissue or cell type-specific.

A further aspect of the application relates to a method of identifying an agent that potentiates the activity of an RNAi construct. The method may comprise identifying an agent that increases the expression and/or activity of an Argonaute protein. The agent may increase the expression and/or activity of an Argonaute protein in a tissue or cell type-specific manner.

Certain embodiments provides a method of identifying an agent that potentiates the activity of an RNAi therapeutic. The method may comprise identifying an agent that increases the expression and/or activity of an Argonaute protein. The agent may increase the expression and/or activity of an Argonaute protein in a tissue or cell type-specific manner.

Another aspect of the application provides a method of identifying an agent that modulates the activity of an RNAi construct. The method may comprise providing an isolated or recombinant Argonaute protein and assaying the RNase activity of the Argonaute protein in the presence of a candidate agent. A change in the RNase activity of the Argonaute protein in the presence of a candidate agent is indicative of the candidate agent capable of modulating the activity of the RNAi construct. The change may be relative to the RNase activity of the Argonaute protein in the absence of the candidate agent or a baseline or control level of the RNase activity of Argonaute protein. The method may involve an Argonaute protein expressed in a cell. Alternatively, the method may involve an isolated or purified Argonaute protein. The method may further comprise determining the RNase activity of said Argonaute protein in the absence of a candidate agent. The identified agent may modulate the activity of an RNAi construct in a tissue or cell type-specific manner.

Certain embodiments provide a method of identifying an agent that modulates the activity of an RNAi therapeutic. The method may comprise providing an isolated or recombinant Argonaute protein and assaying the RNase activity of the Argonaute protein in the presence of a candidate agent. A change in the RNase activity of the Argonaute protein in the presence of a candidate agent is indicative of the candidate agent capable of modulating the activity of the RNAi therapeutic. The change may be relative to the RNase activity of the Argonaute protein in the absence of the candidate agent or a baseline or control level of the RNase activity of Argonaute protein. The method may involve an Argonaute protein expressed in a cell. Alternatively, the method may involve an isolated or purified Argonaute protein. The method may further comprise determining the RNase activity of said Argonaute protein in the absence of a candidate agent. The identified agent may modulate the activity of an RNAi construct in a tissue or cell type-specific manner.

A further aspect of the application provides a composition for targeted gene inhibition comprising an agent that modulates the RNase activity of an Argonaute protein. The composition may further comprise an RNAi construct or an RNAi therapeutic targeting a gene. In certain embodiments, an agent may potentiate the RNase activity of the Argonaute protein. Alternatively, an agent may inhibit the RNase activity of the Argonaute protein. In certain embodiments, the RNAi construct or therapeutic may target a gene in a first tissue or cell type; the identified agent may potentiate the RNase activity of the Argonaute protein in said first tissue or cell type. In certain embodiments, the identified agent may inhibit the RNase activity of the Argonaute protein in a second tissue or cell type.

The application also provides a pharmaceutical preparation comprising the compositions described herein and a physiologically acceptable carrier.

A further aspect of the invention relates to a cell line that overexpresses an Argonaute protein. The cell line of claim may overexpress a mammalian Argonaute protein, e.g., a human Agonaute protein. A mammalian Agonaute protein may be Ago-1, Ago-2, Ago-3, or Ago-4. The cell line may alternatively overexpress an Argonaute protein having an amino acid sequence that is 95% identical to an amino acid sequence as set forth in SEQ ID NOs.: 1-4, or a homologue, fragment, variant, or derivative thereof. The cell line may alternatively overexpress an Argonaute protein encoded by a nucleic acid molecule having a sequence that is 95% identical to a nucleic acid sequence as set forth in any one of SEQ ID NOs.: 1-4. The cell line may alternatively overexpress an Argonaute protein encoded by a nucleic acid molecule that hybridizes under high stringency conditions to a nucleic acid sequence as set forth in any one of SEQ ID NOs.: 1-4. The cell line may alternatively overexpress an Argonaute protein having an amino acid sequence set forth in any one of SEQ ID NOs.: 1-4.

Another aspect of the application relates to a cell line that expresses a mutant Argonaute protein comprising an amino acid sequence that is different from a naturally-occurring Argonaute protein.

A further aspect of the application relates to a host (e.g., a cell or an animal) wherein the expression of an endogenous Argonaute protein is controlled by, e.g., a transgene (or a nucleic acid construct such as for example the construct based on the Puro PGK vector described herein).

The application also provides an assay for identifying nucleic acid sequences for conferring a particular phenotype in a cell, comprising constructing a library of nucleic acid sequences oriented to produce double stranded RNA. The assay may further comprise ntroducing a dsRNA library into a culture of target cells. The assay may also comprise identifying members of the library which confer a particular phenotype on the cell, and identifying the sequence from the cell which is identical or homologous to the library member.

Another aspect of the invention provides a nucleic acid composition comprising a first nucleic acid comprising an RNAi construct and a second nucleic acid encoding an Argonaute protein. The RNAi construct may comprise a nucleotide sequence encoding a single-strand siRNA; the nucleotide sequence may be operably linked to a promoter. In certain embodiments, the second nucleic acid encodes a human Argonaute protein and may be operably linked to a promoter. Alternatively, the second nucleic acid may encode a non-naturally-occurring Argonaute protein. In certain embodiments, the RNAi construct may be tissue or cell type-specific. The promoters may be tissue or cell type-specific.

A further aspect of the application provides a cell expressing any of the nucleic acid compositions described herein.

### BRIEF DESCRIPTION OF THE DRAWINGS

FIG. 1 shows the crystal structure of Pyrococcus furiosus Argonaute. Stereo ribbon representation of Argonaute with the N-terminal domain shown in blue, the “stalk” in light blue, the PAZ domain in red, the middle domain in green, the PIWI domain in purple and the interdomain connector in yellow. The active site residues are drawn in stick representation. Disordered loops are drawn as dotted lines. The N-terminal, middle and PIWI domains form a crescent base. The “stalk” holds the PAZ domain above the crescent base and the interdomain connector cradles the molecule. This figure as well as FIGS. 2A, 3A,B, 5B were prepared with BobScript (60), MolScript (61) and Raster3D (62, 63).

FIGS. 2A-2B show that the PAZ domains of PfAgo and hAgo1 have very similar structures. (FIG. 2A) Stereo diagram of the superposition of Ca atoms from the PAZ domain of PfAgo in shown in red and the PAZ domain of hAgo1 shown in gray. Dotted lines represent disordered regions. (FIG. 2B) Sequence alignment of the PAZ domains of PfAgo, hAgo1 and DmAgo2 based on the structural superposition of the three domains. The sequence of PfAgo-PAZ domain could not be readily aligned with PAZ domains from other species without knowledge of the structure. The secondary structure elements for PfAgo are shown above the sequence.

FIGS. 3A-3C show that PIWI is an RNase H domain. (FIG. 3A) Ribbon diagrams of the PIWI domain, E. coli RNase HI and M. jannaschii RNase HII. The three structures were superimposed and shown in a similar view with the secondary structure elements of the canonical RNase H fold in color. The active site residues are shown in stick representation. (FIG. 3B) A close-up view of the active sites. This view is rotated ˜180° compared to the view in A. One active site aspartate is always located on β1 of the fold (the red strand) in this family of proteins and another aspartate is always located on β4 of the fold (the green strand). The third active site carboxylate, a glutamic acid, varies in its position. The Mg ²⁺ ion in RNase HI is shown as a pink sphere. A strong difference electron density found in the active site of PIWI that was assigned as a water molecule is shown as a green sphere. (FIG. 3C) Sequence alignment of the PIWI domains from Pf Argonaute and the four human Argonaute proteins. Invariant residues are highlighted in purple and conserved residues are highlighted in blue. The secondary structure elements are shown above the structure. The conserved active site carboxylate residues are marked by a red asterisk.

FIGS. 4A-4B show siRNA binding. (FIG. 4A) A 5′-phosphorylated ss-siRNA (4 nM) was radiolabled by phosphorylation with γ-³²P-ATP and hybridized with an unlabeled complementary strand to yield a ds-siRNA and was gel purified. The ss- and ds-siRNAs were UV-crosslinked to PfAgo and the adducts were resolved by SDS-PAGE. PfAgo binds preferentially to the ss-siRNA compared to the ds-siRNA. (FIG. 4B) Competition experiments were performed with the same labeled ss-siRNA and UV-crosslinking to PfAgo in the presence of increasing amounts of the indicated competitors (from 0 to 400 nM), showing preferential binding to a 5′-phosphorylated ss-siRNA compared to unphosphorylated ss-siRNA.

FIGS. 5A-5C illustrate a model for siRNA-guided mRNA cleavage by Argonaute. (FIG. 5A) Two views of the electrostatic surface potential of PfAgo indicating a positively charged groove suitable for interaction with nucleic acids. The locations of the domains are labeled and the approximate location of the active site in PIWI is marked by a yellow asterisk. The view on the left is slightly tilted on the horizontal axis compared to the view in FIG. 1. Two of the loops were removed for a better view of the groove. The binding groove runs horizontally across the protein bending upwards between the PAZ and N-terminal domains on the right and bending around between the PAZ and middle domains on the left. The view on the right is from the proposed exit groove of the mRNA and looking into the active site area (rotated ˜90° compared to the view on the left). The PIWI domain is behind the middle domain in this view. The coloring scheme depicts potentials <−10 k_{B}T in red and >10 k_{B}T in blue, where k_{B} is the Boltzman constant and T is the absolute temperature. This figure was prepared with GRASP (64).

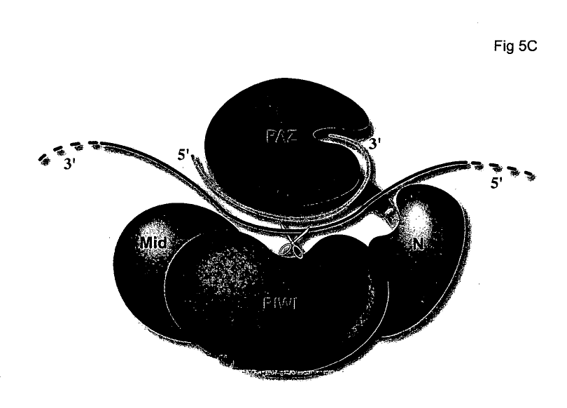

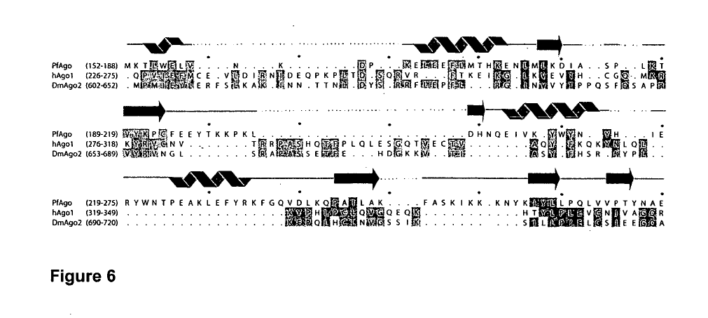

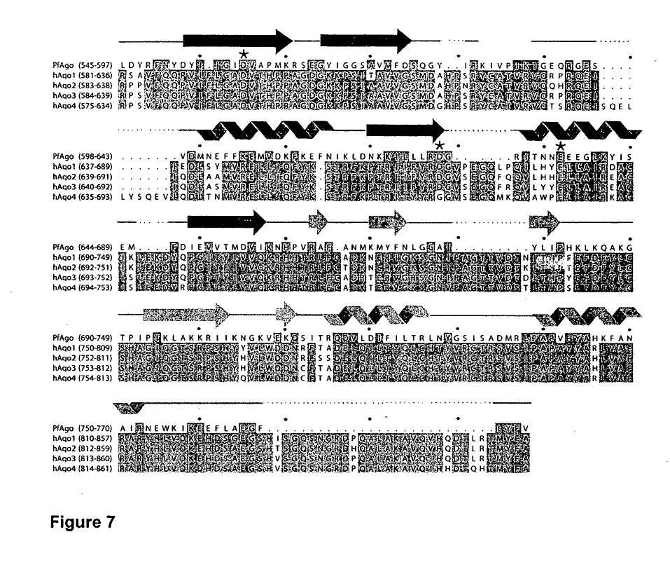

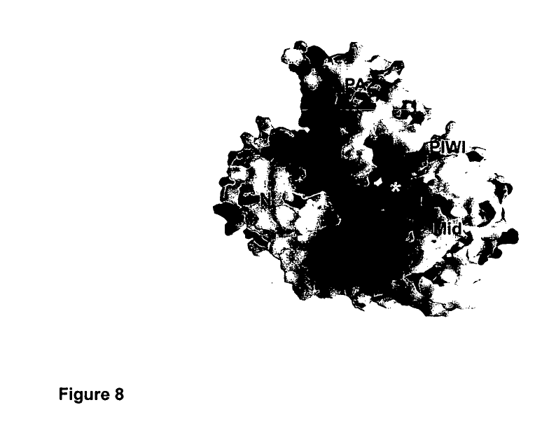

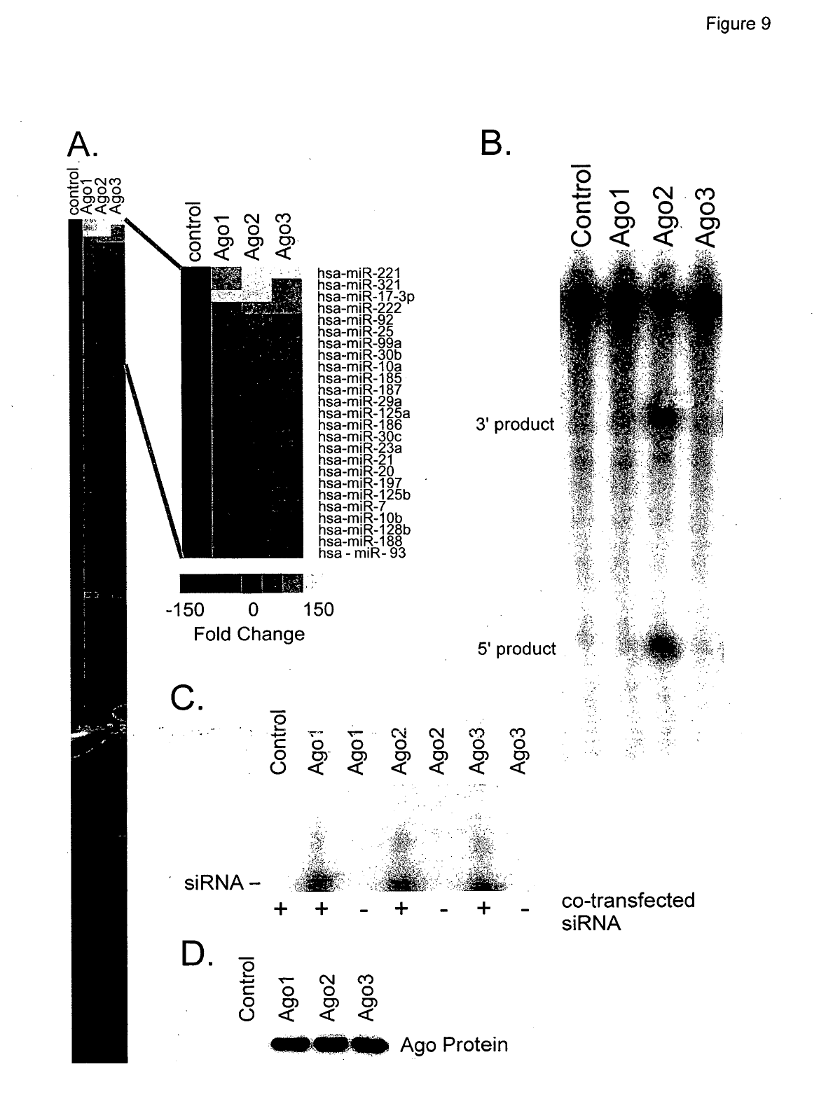

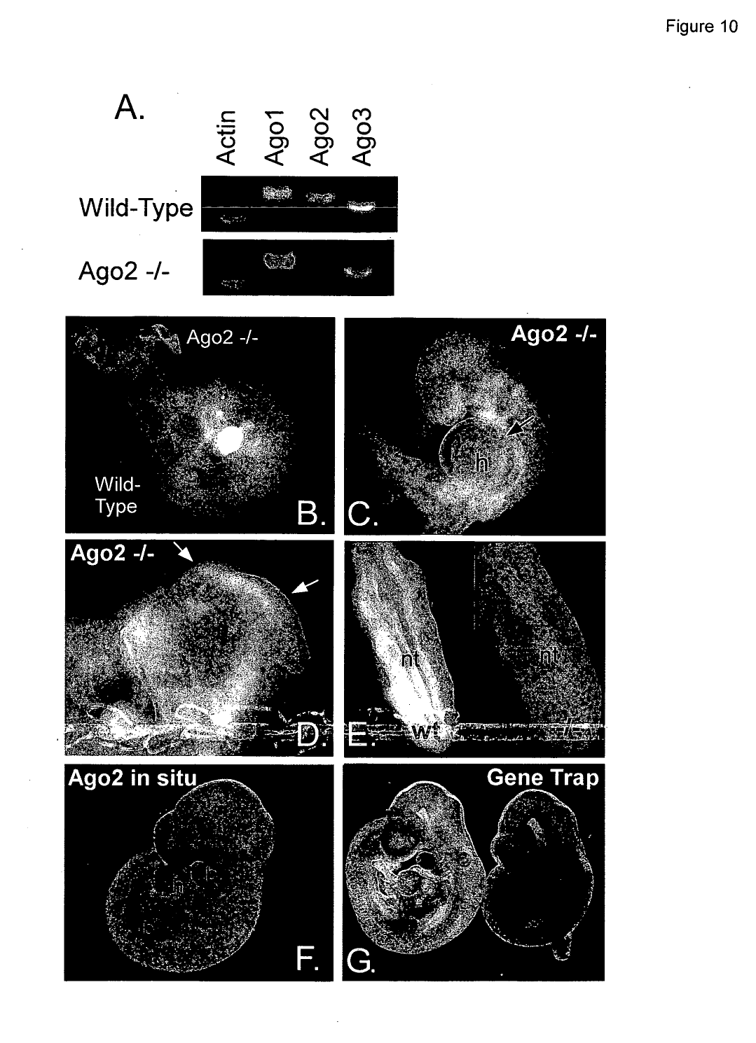

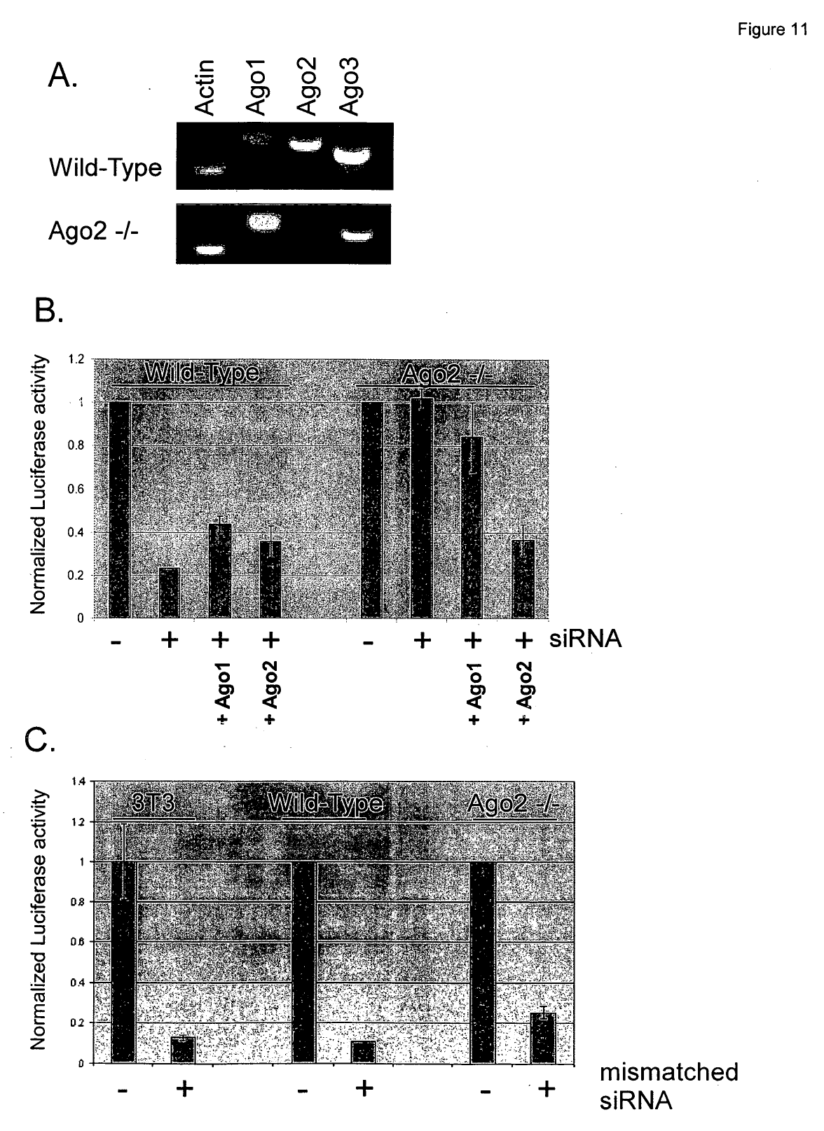

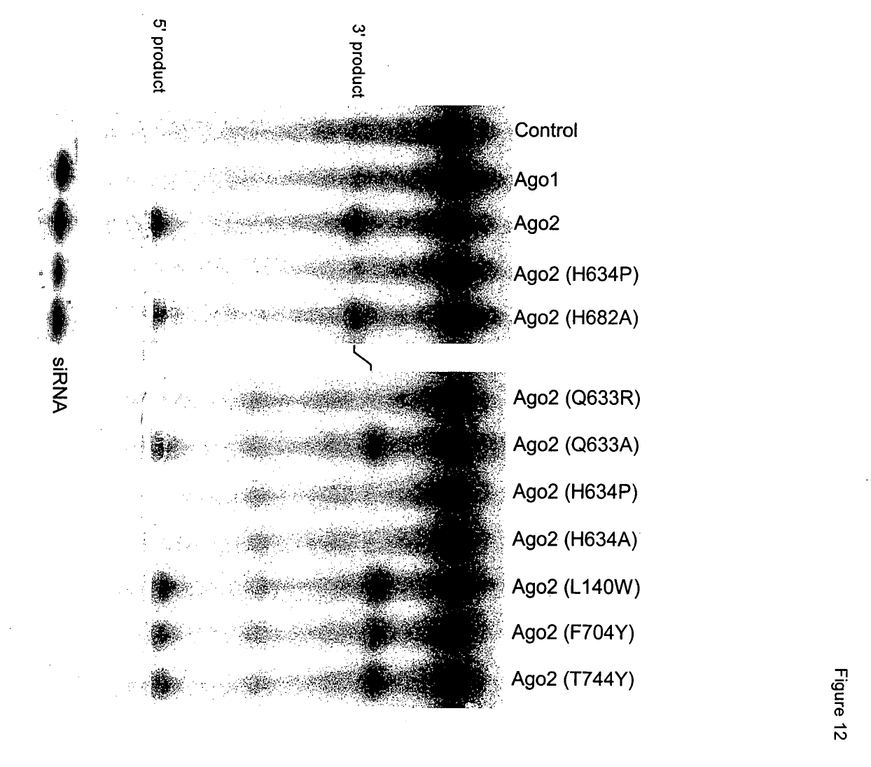

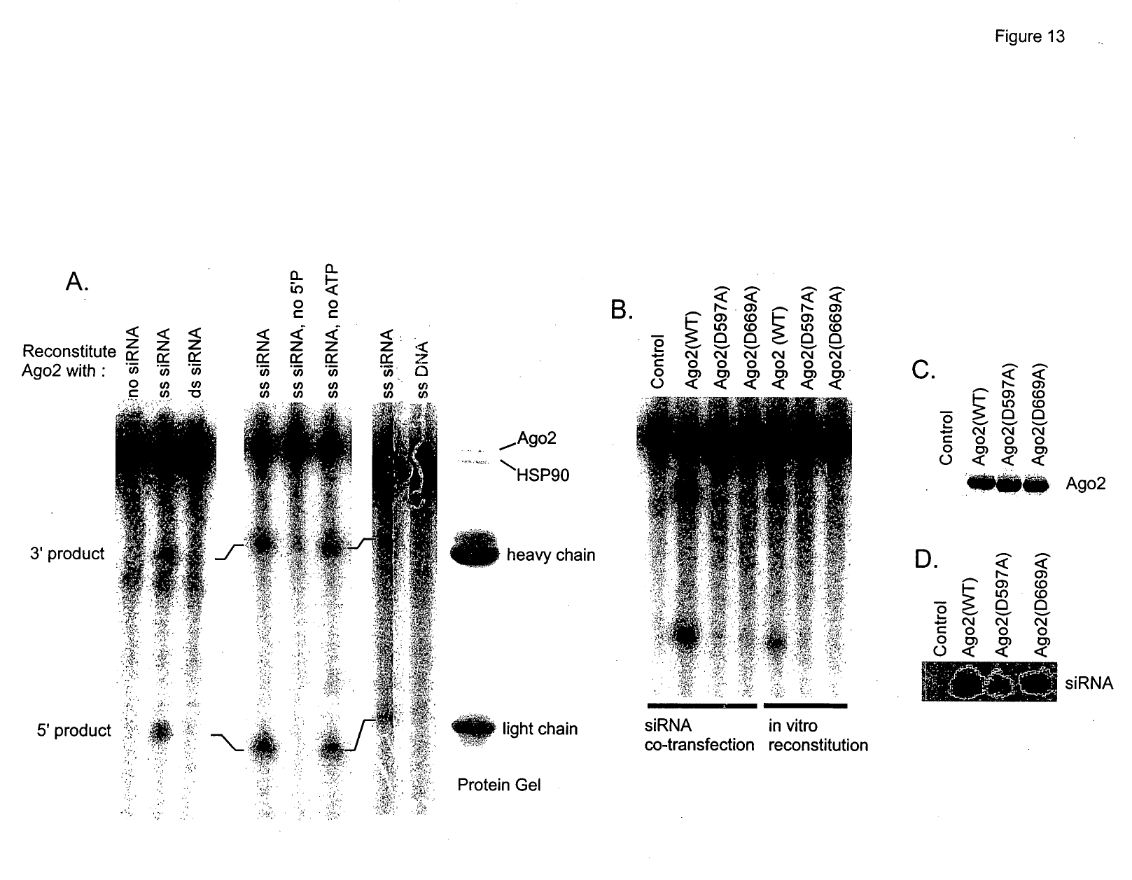

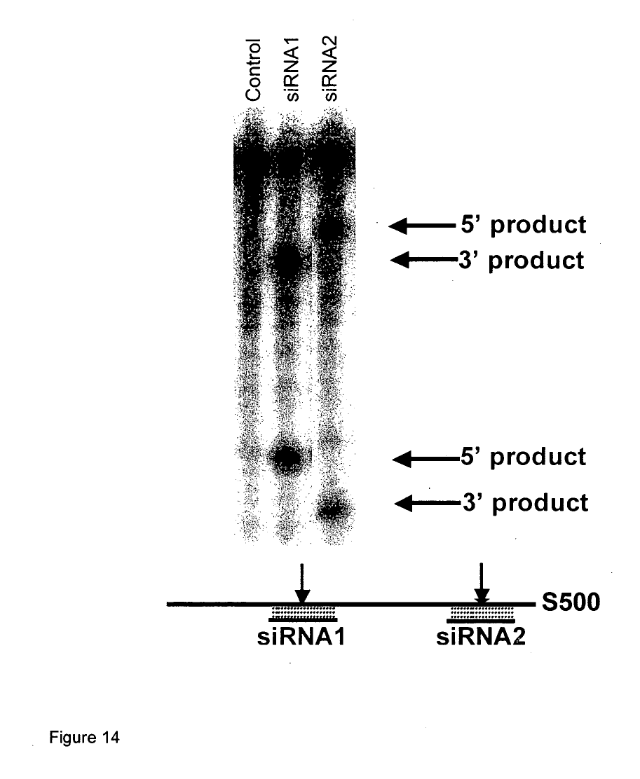

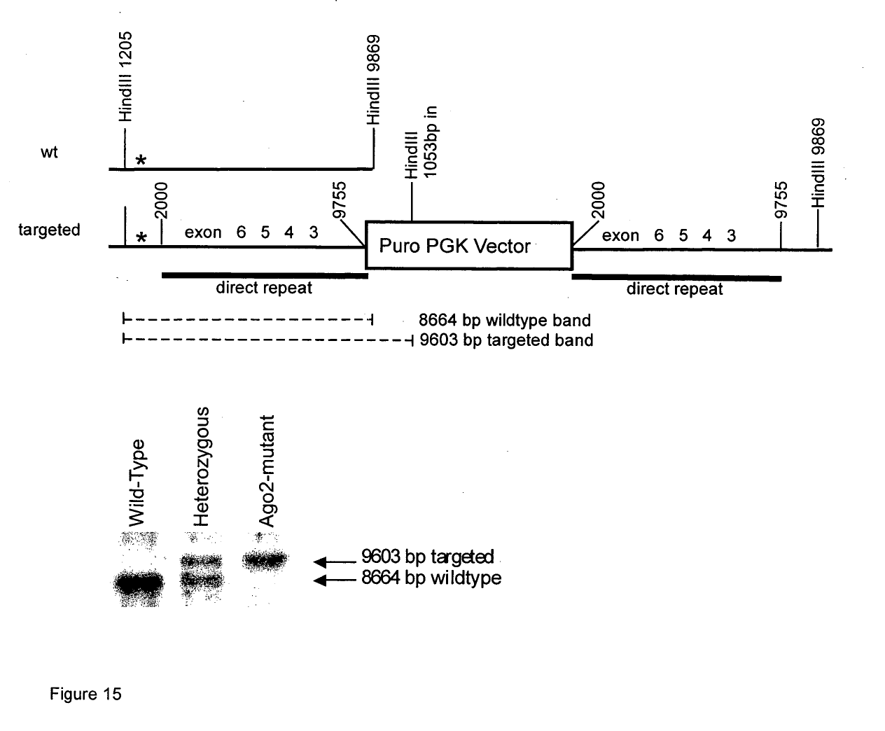

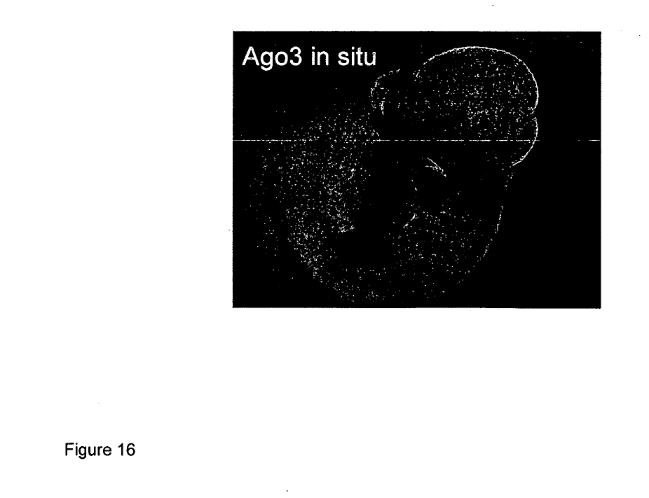

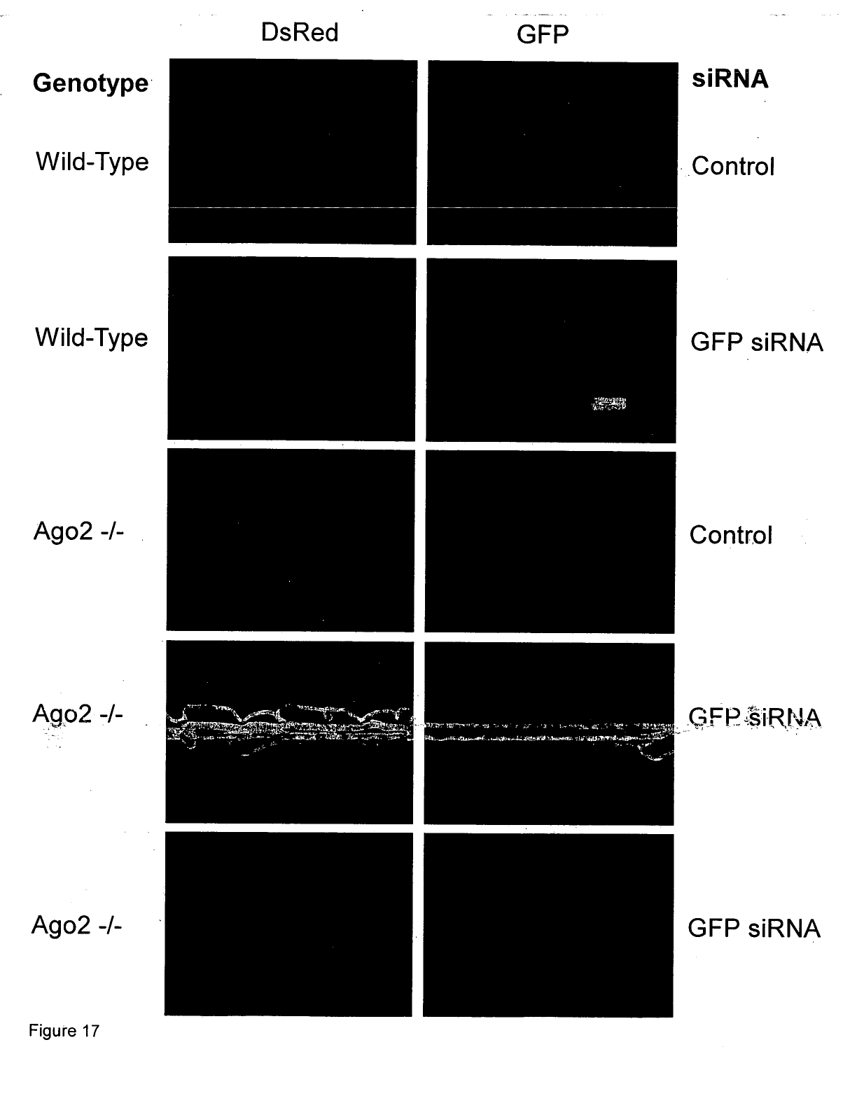

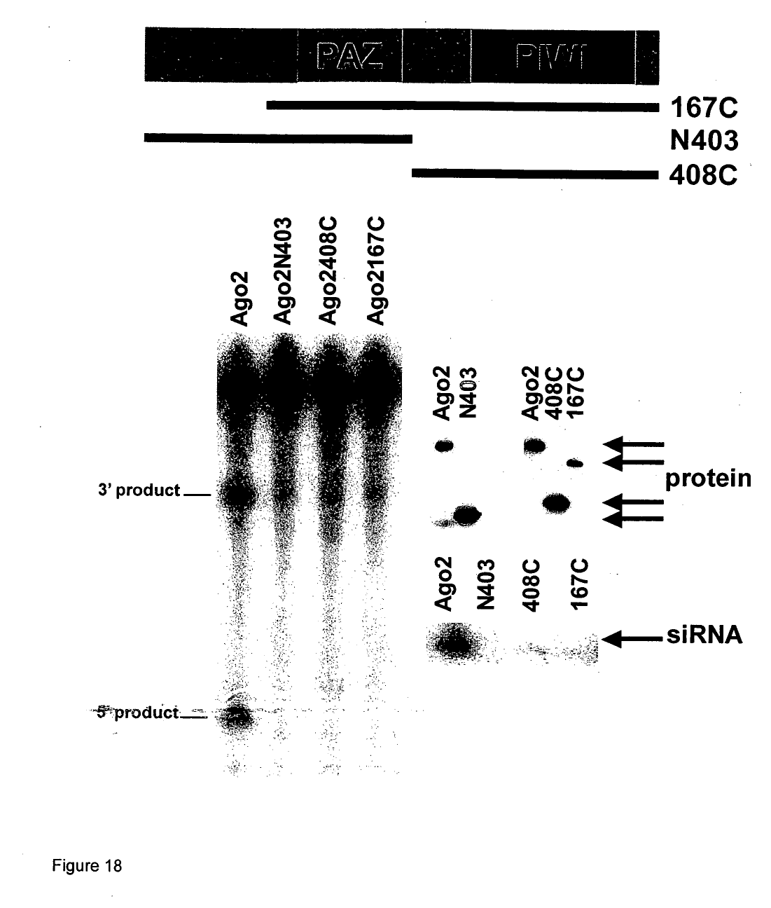

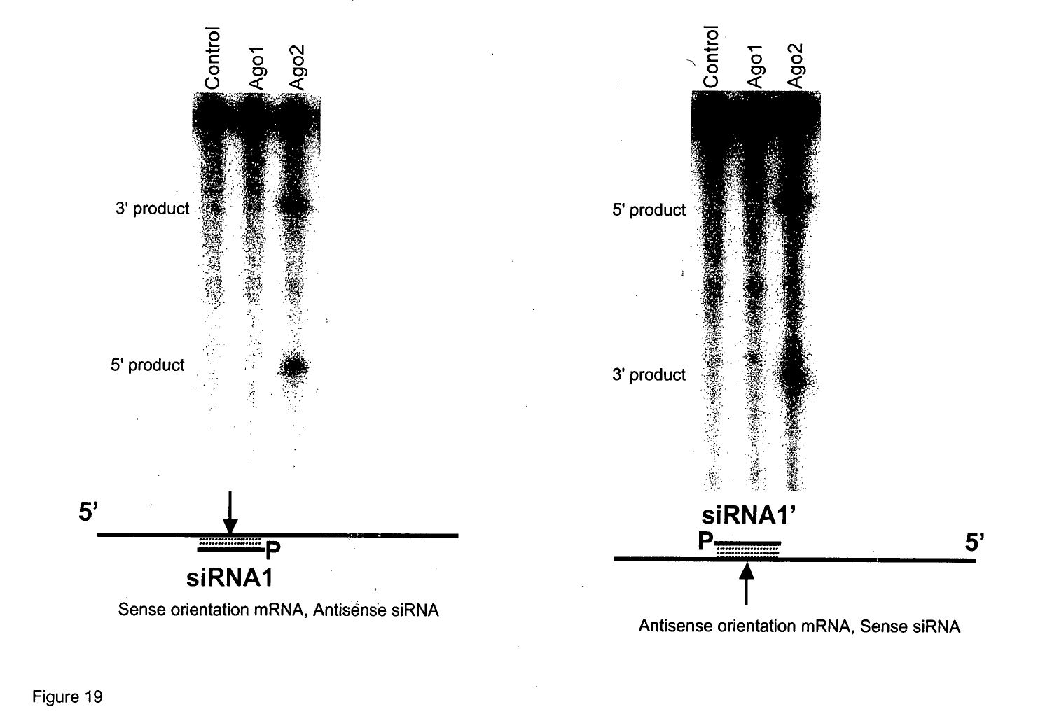

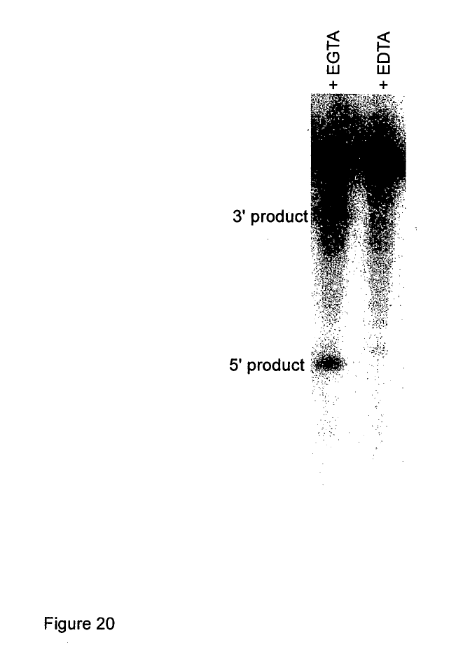

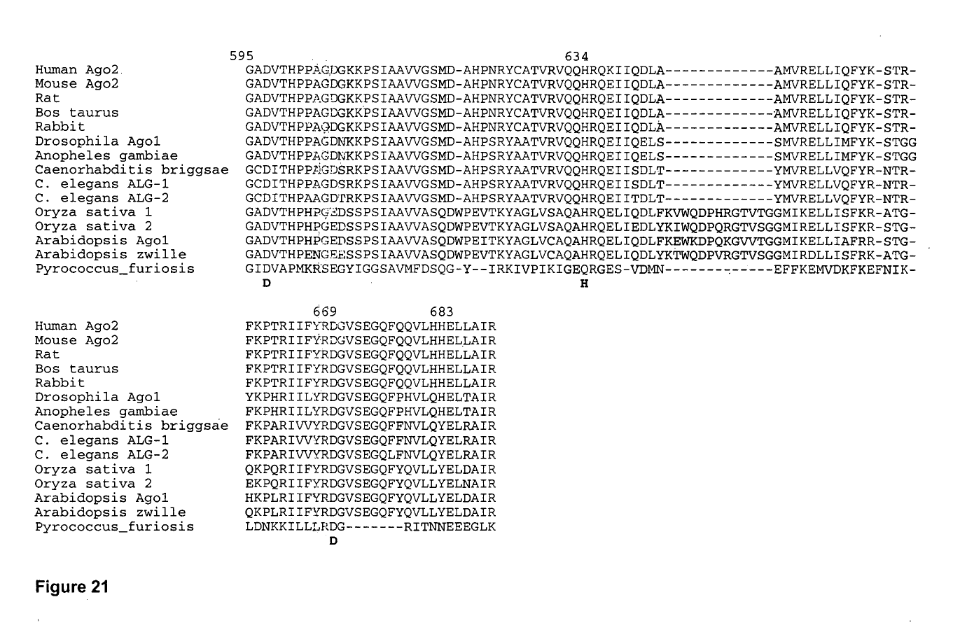

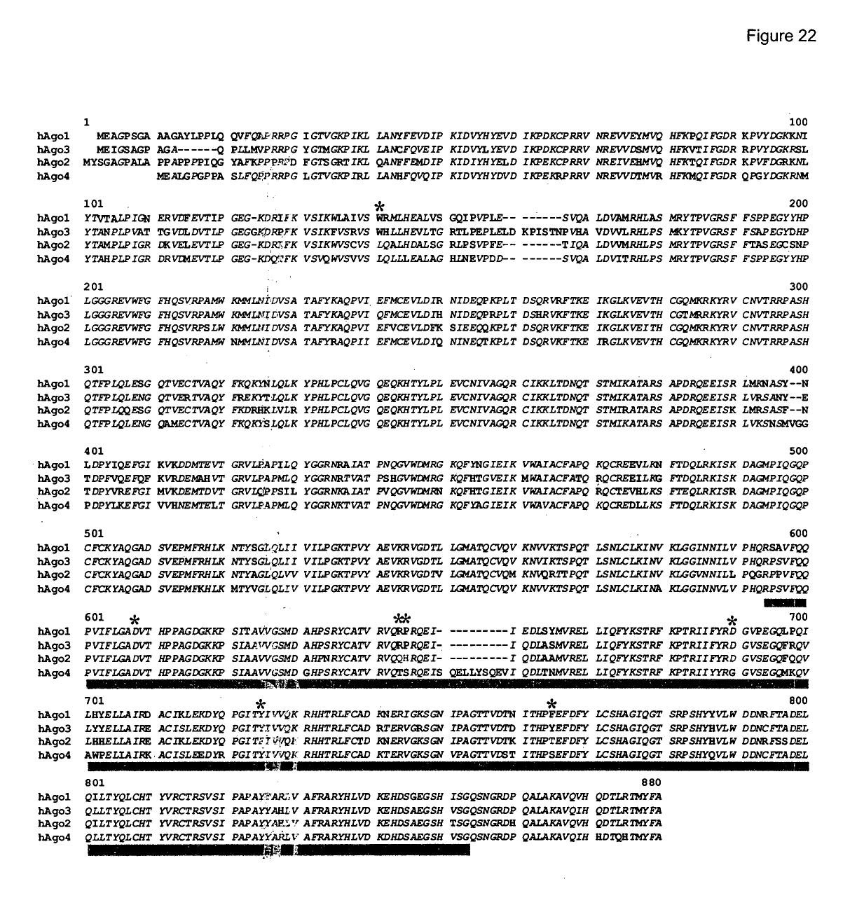

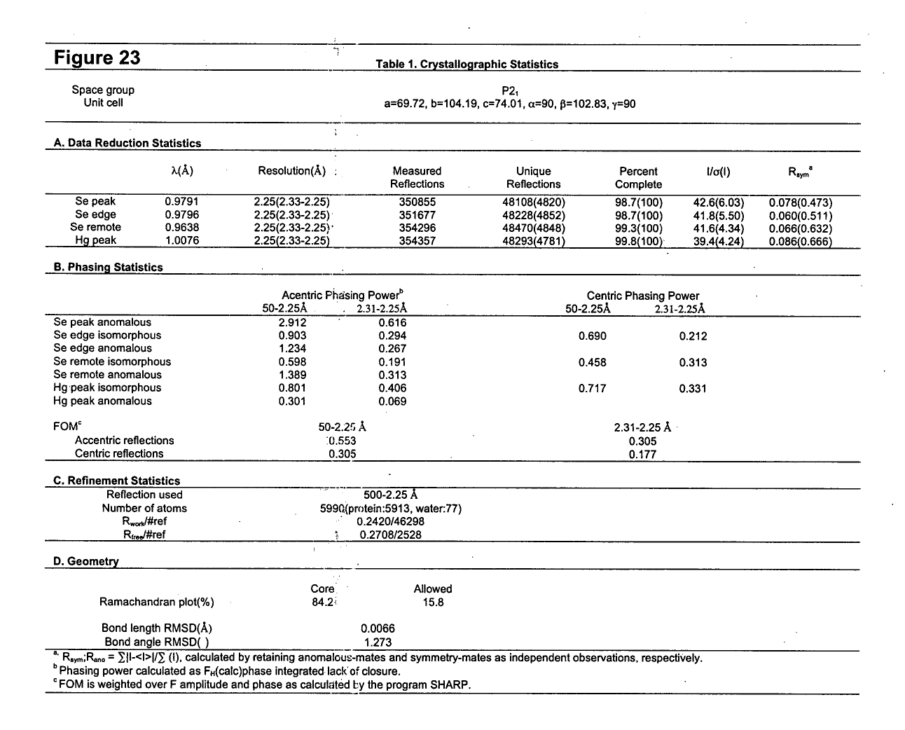

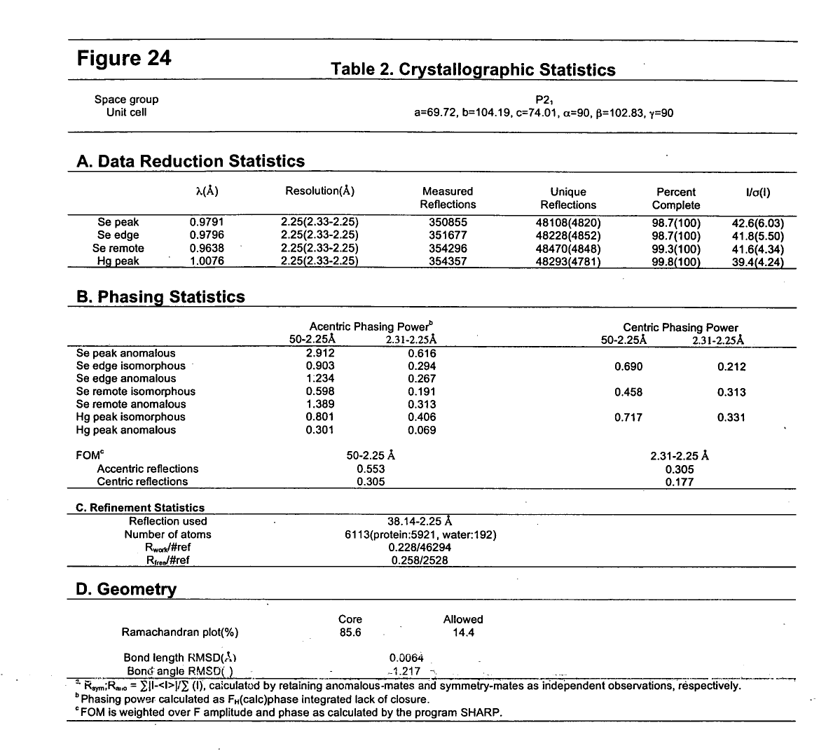

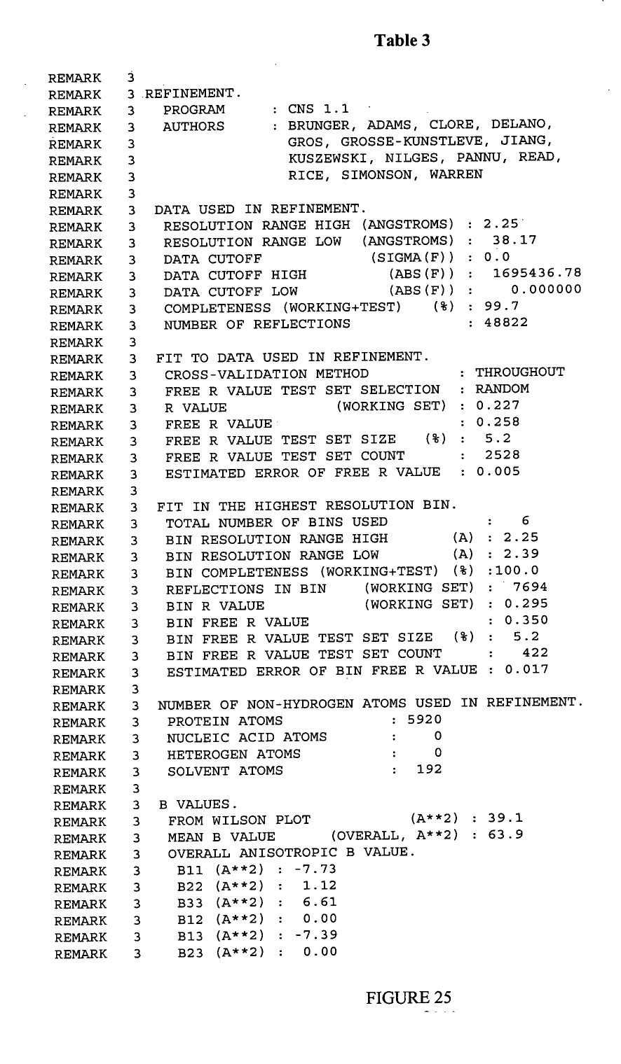

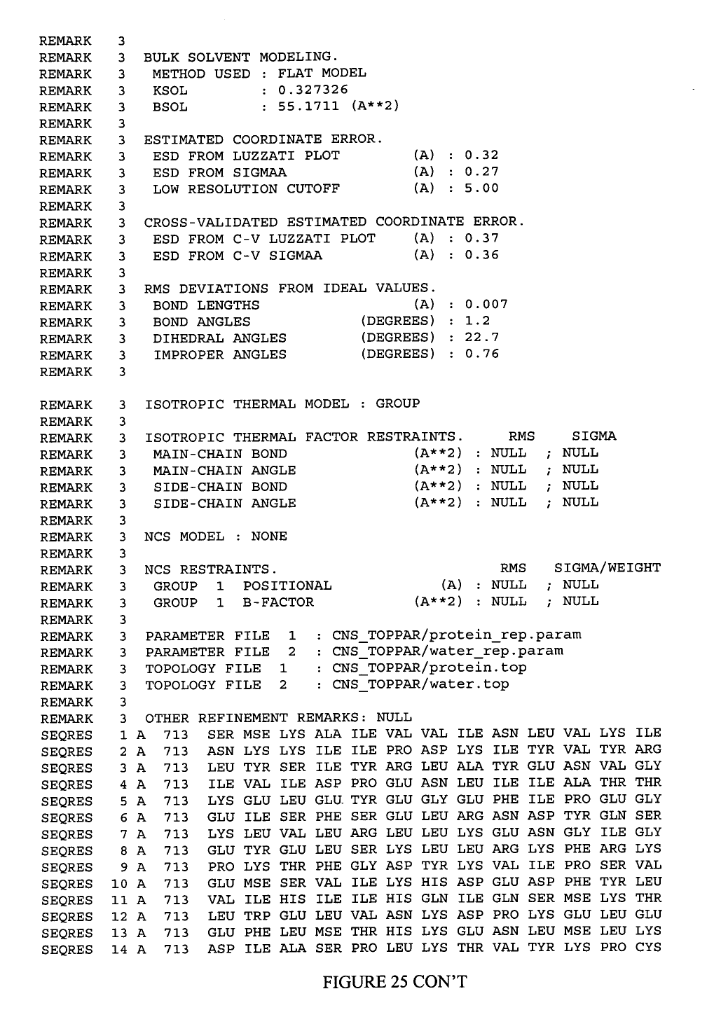

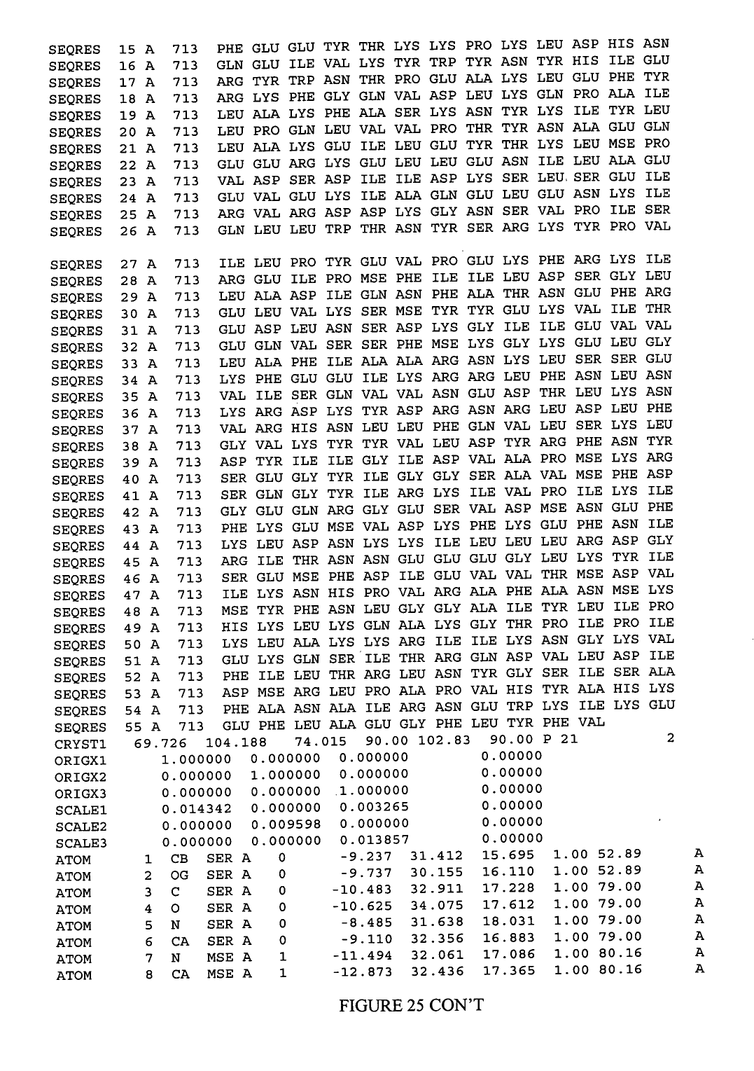

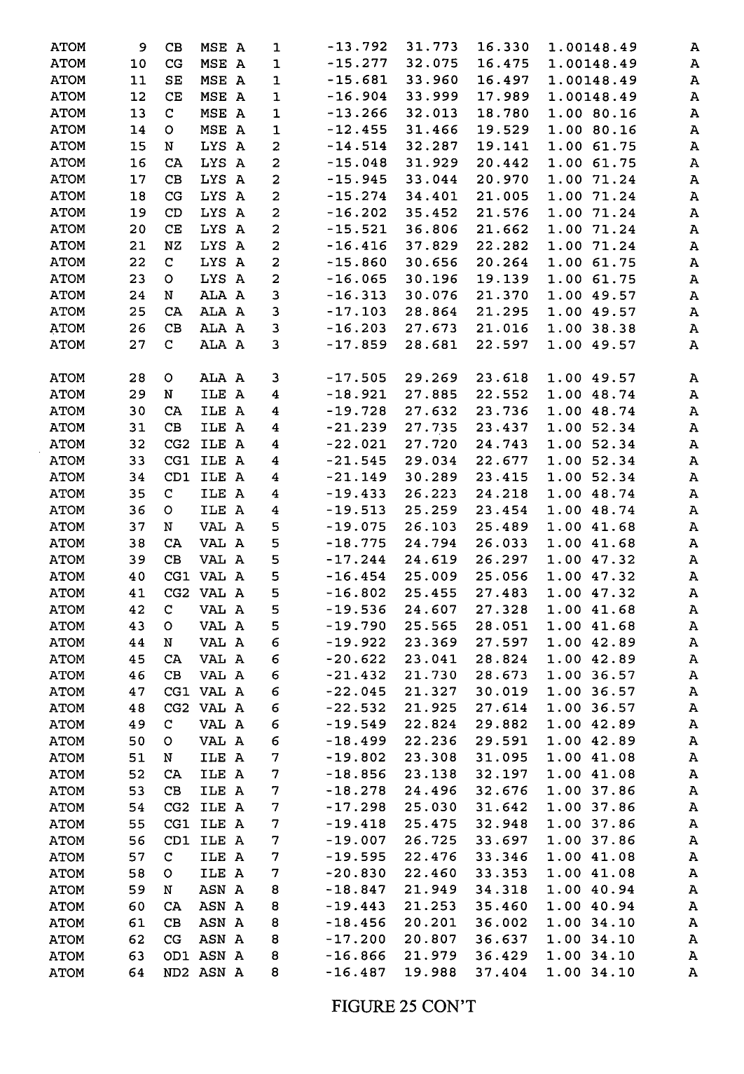

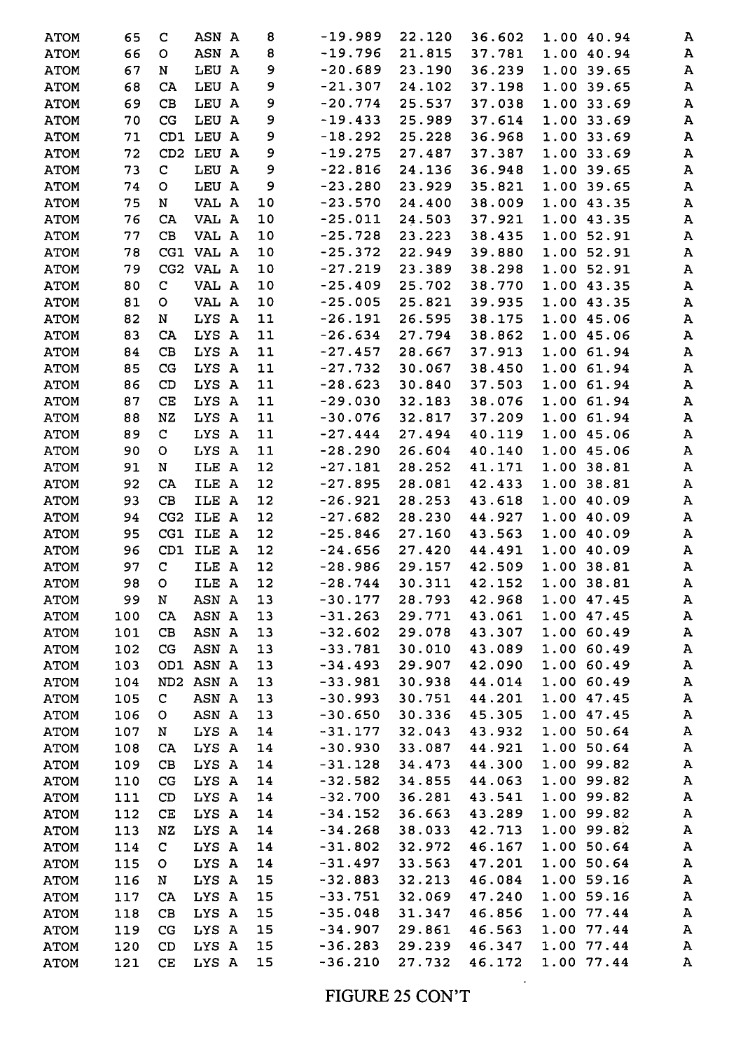

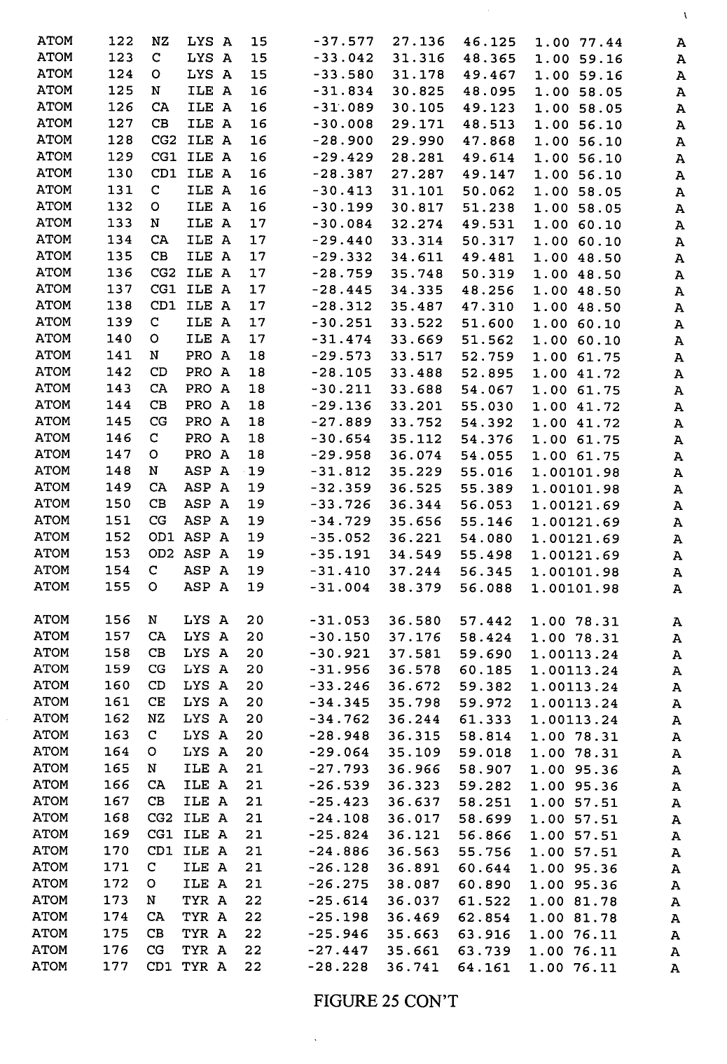

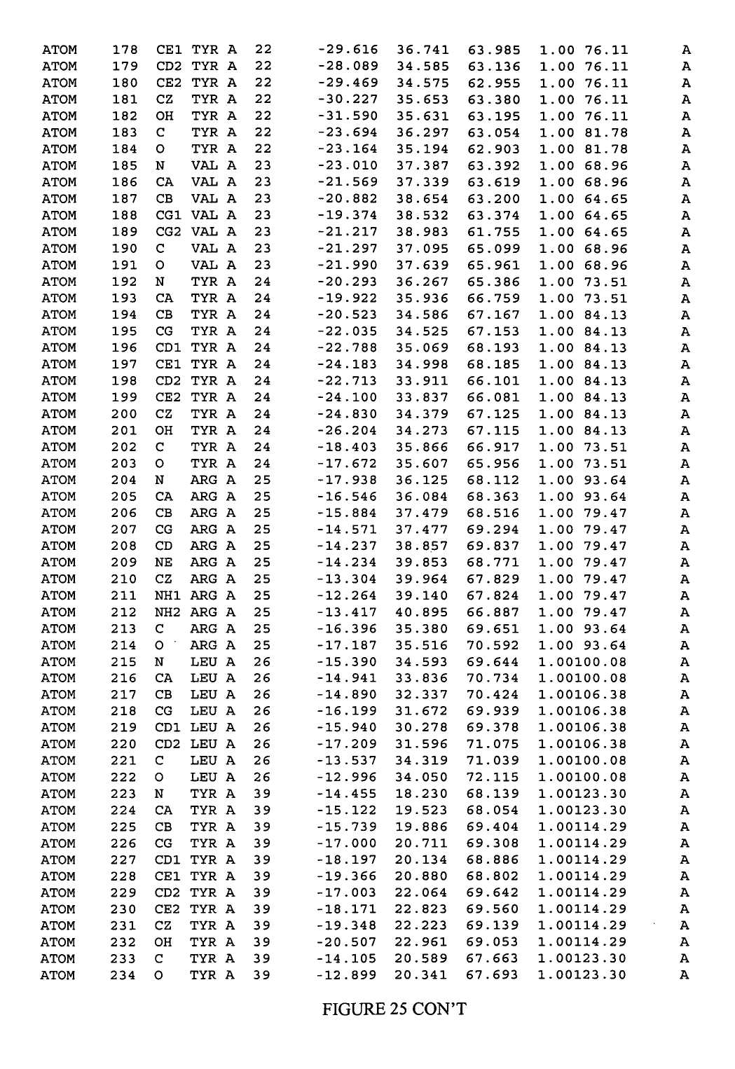

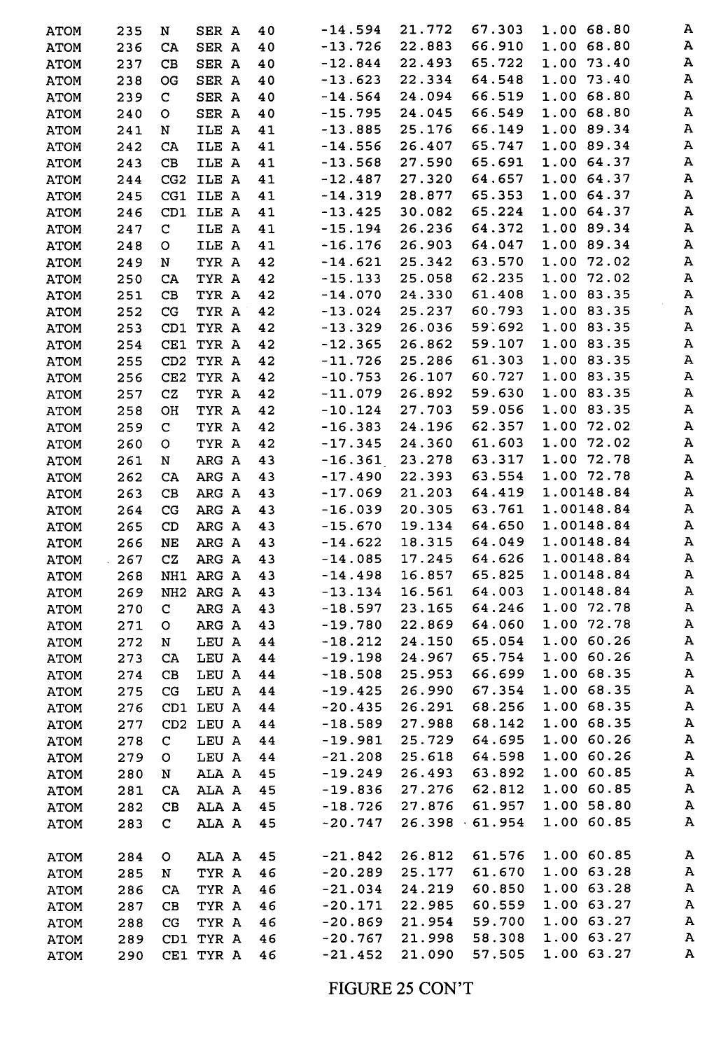

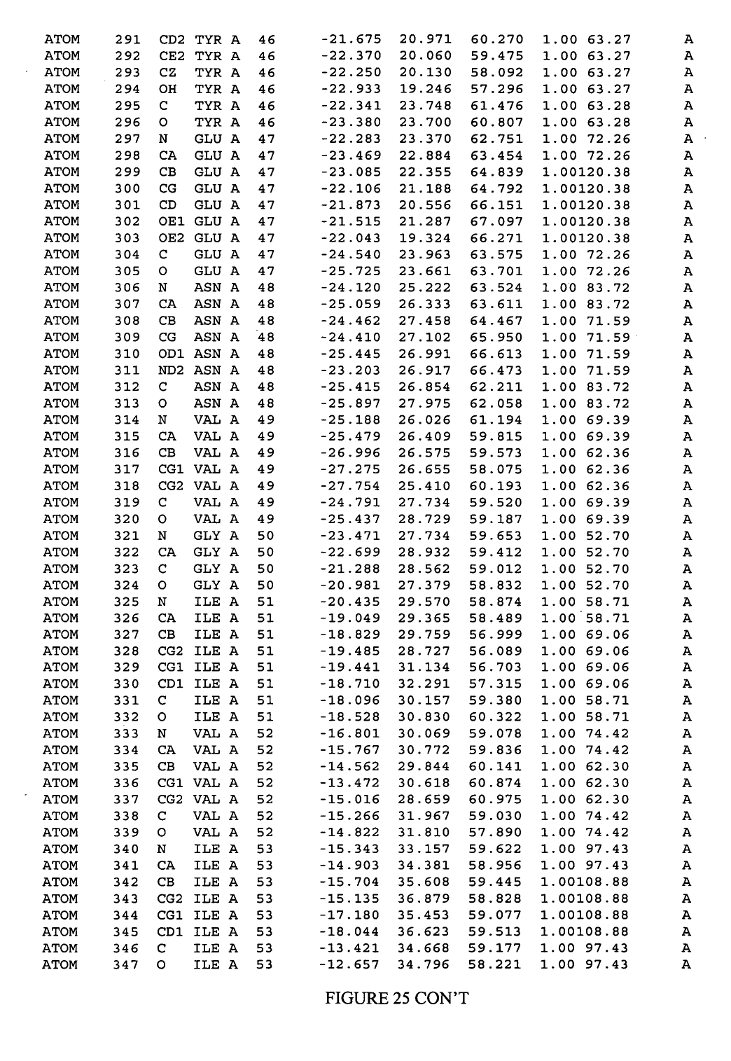

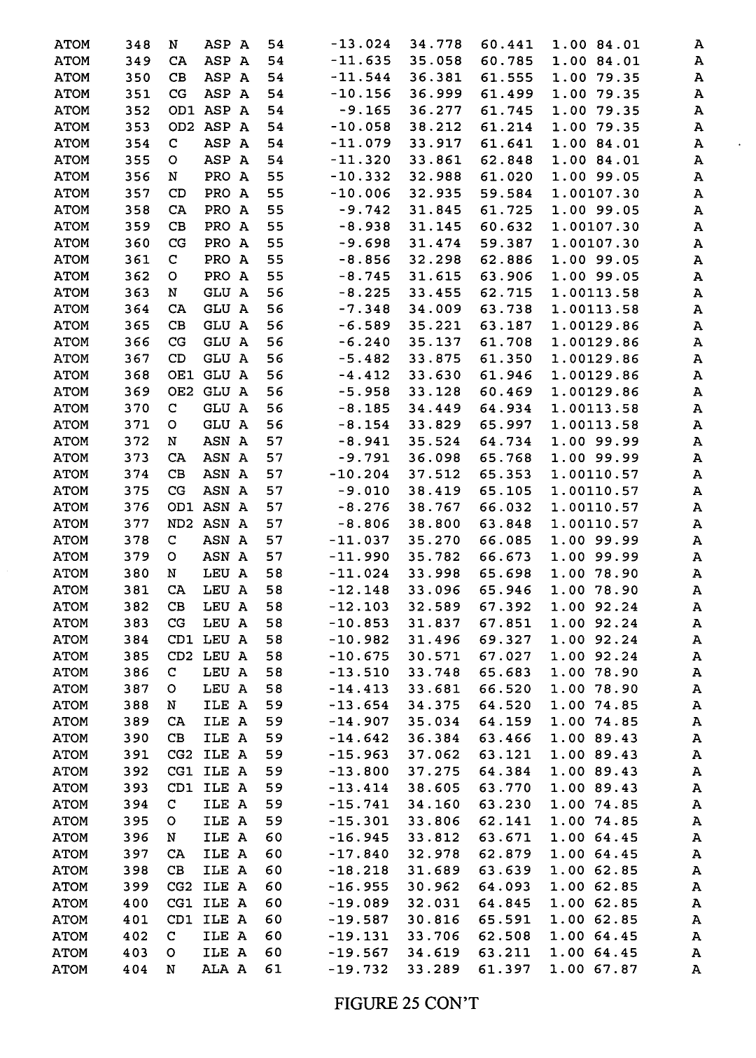

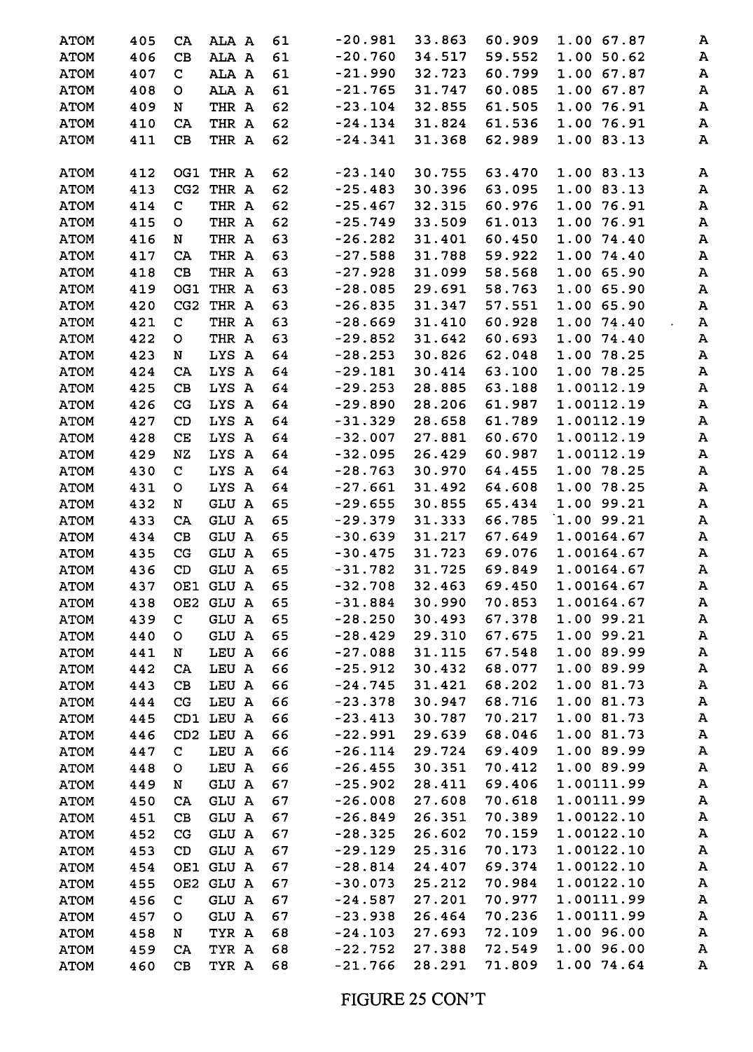

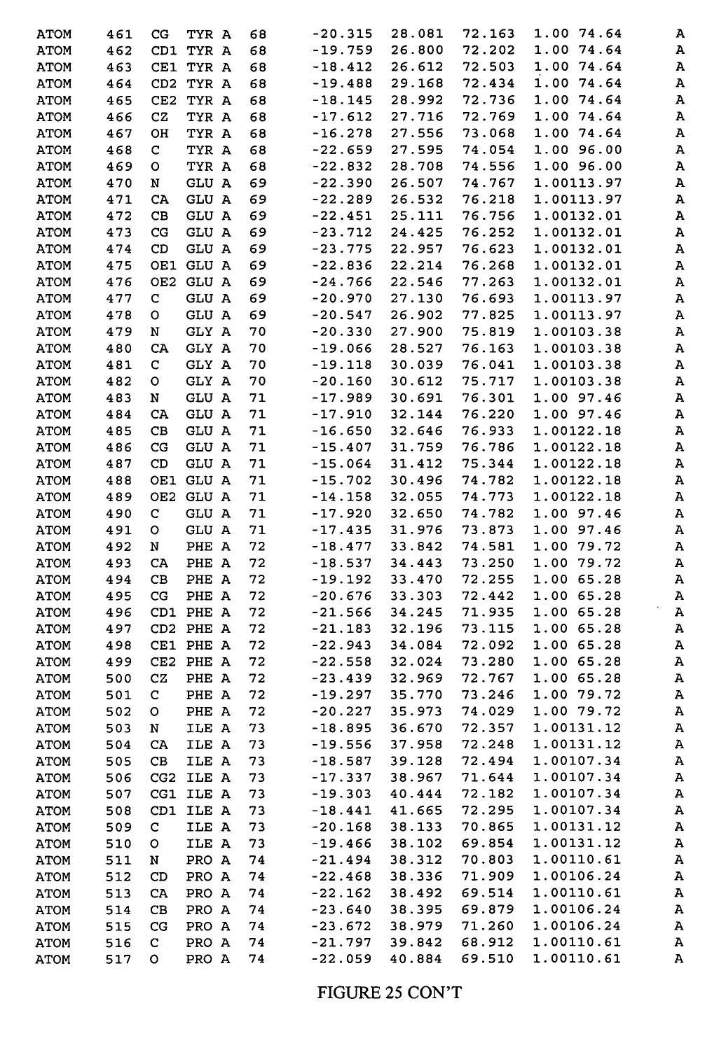

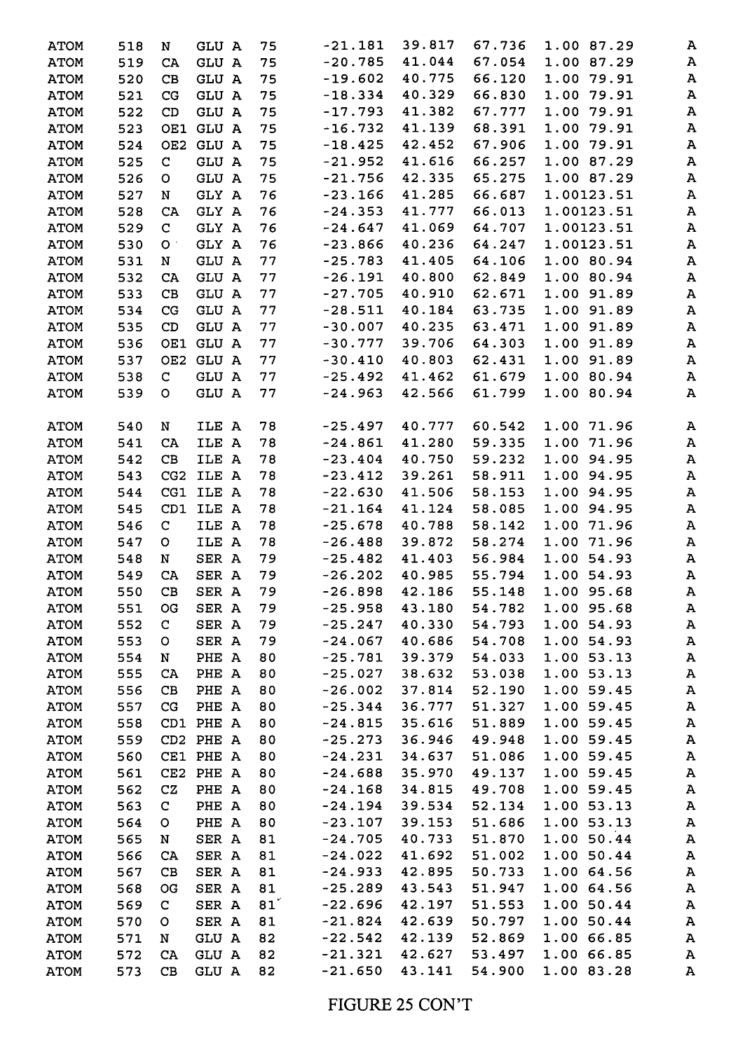

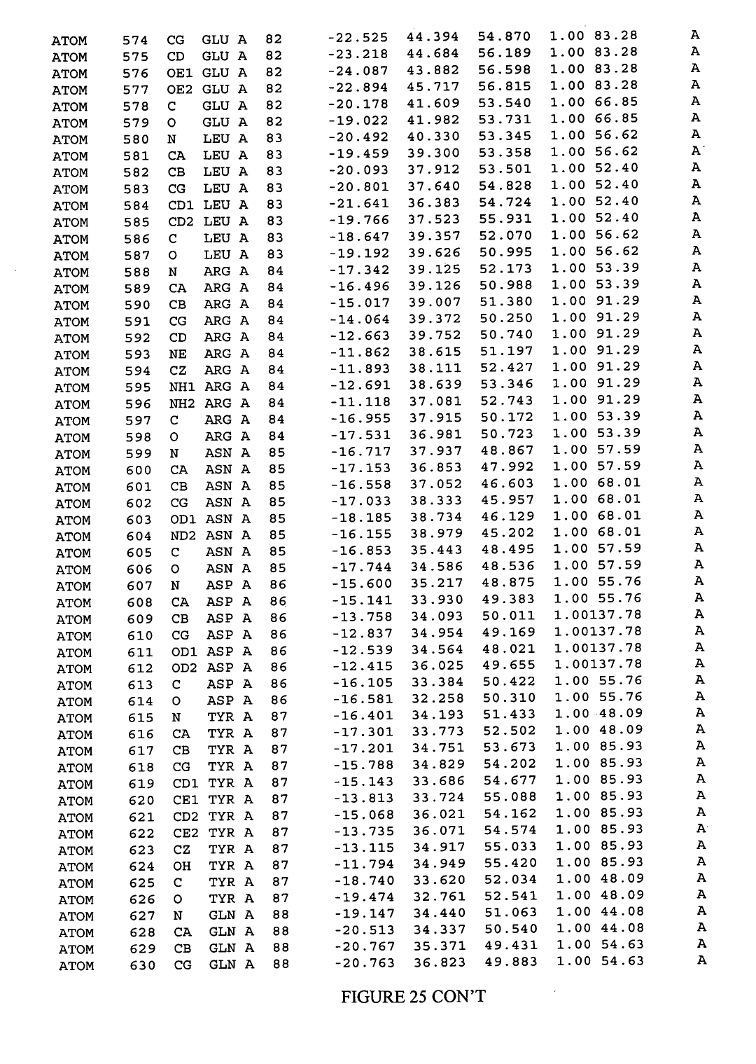

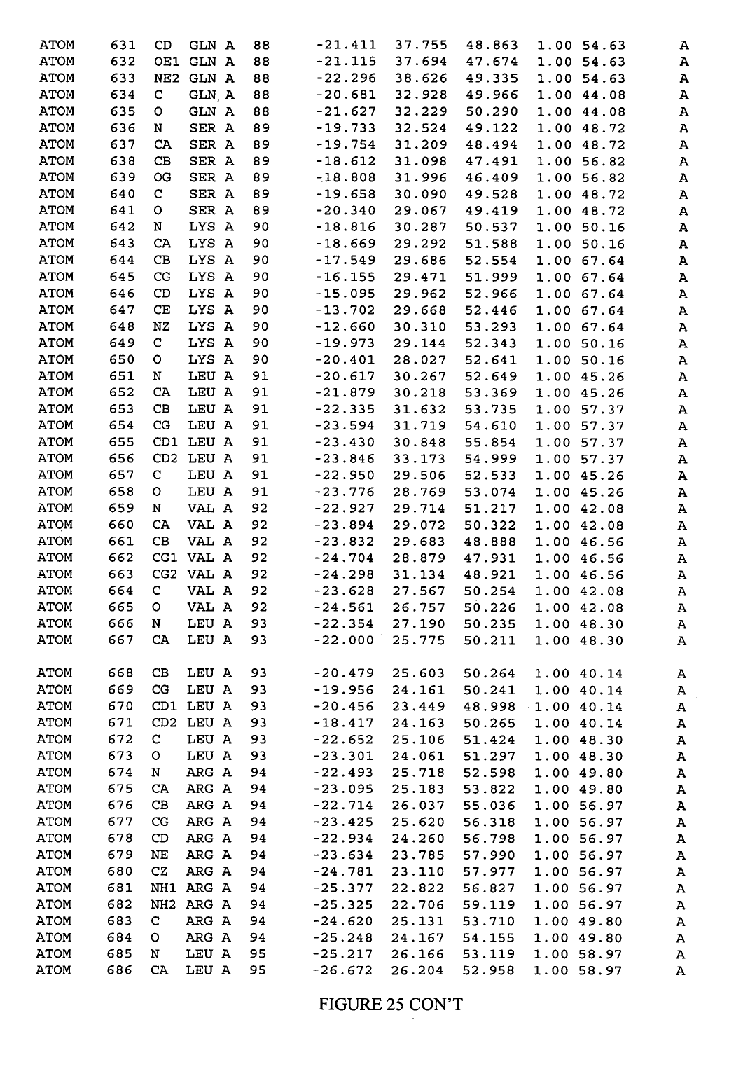

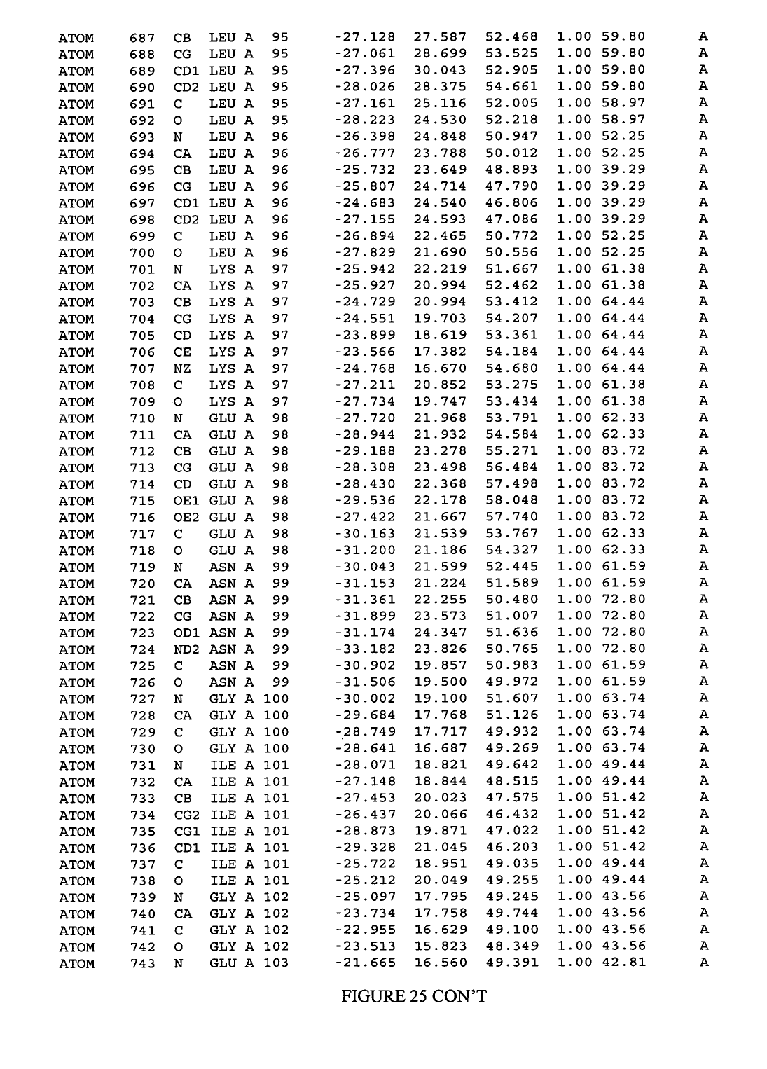

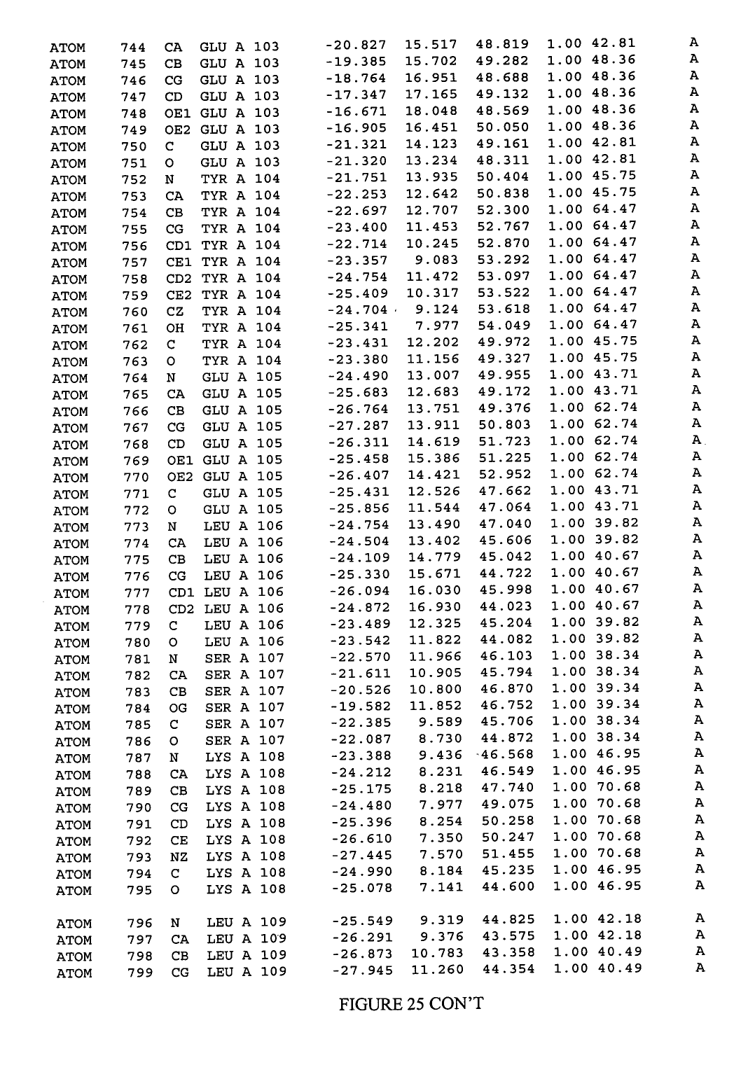

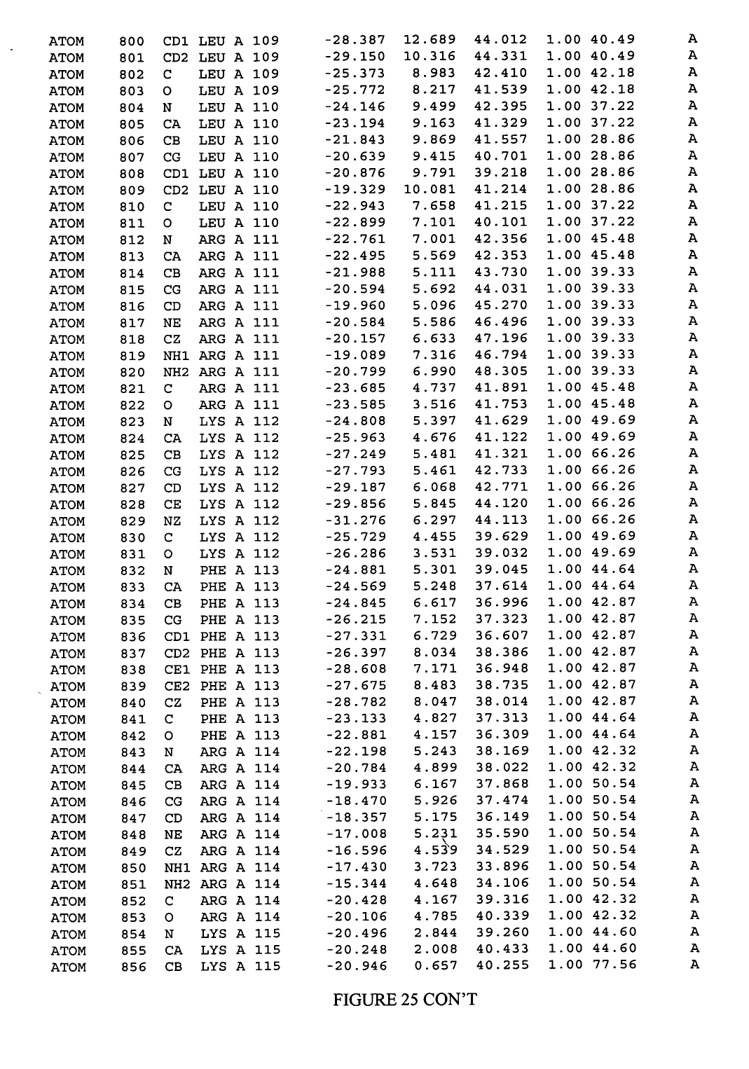

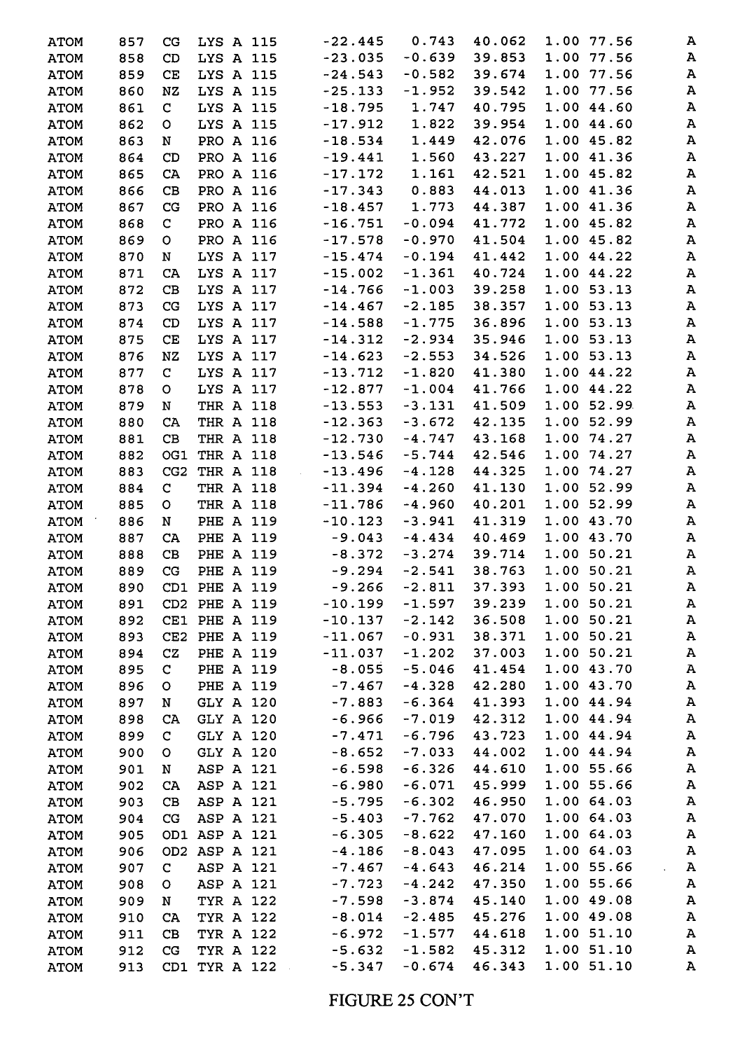

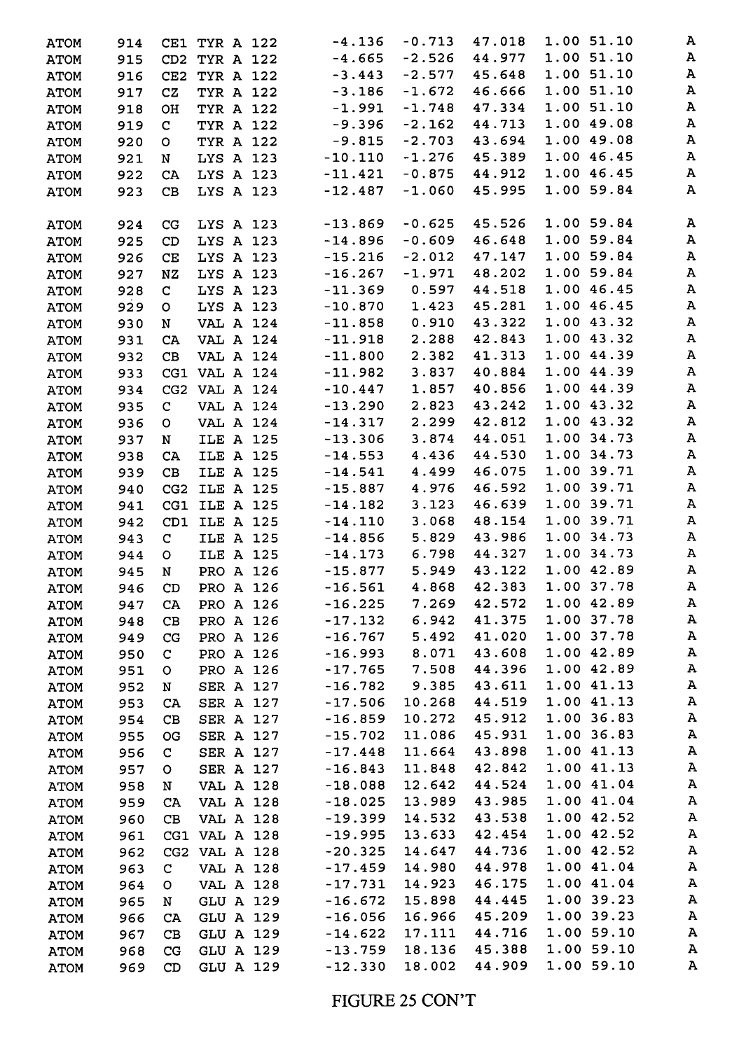

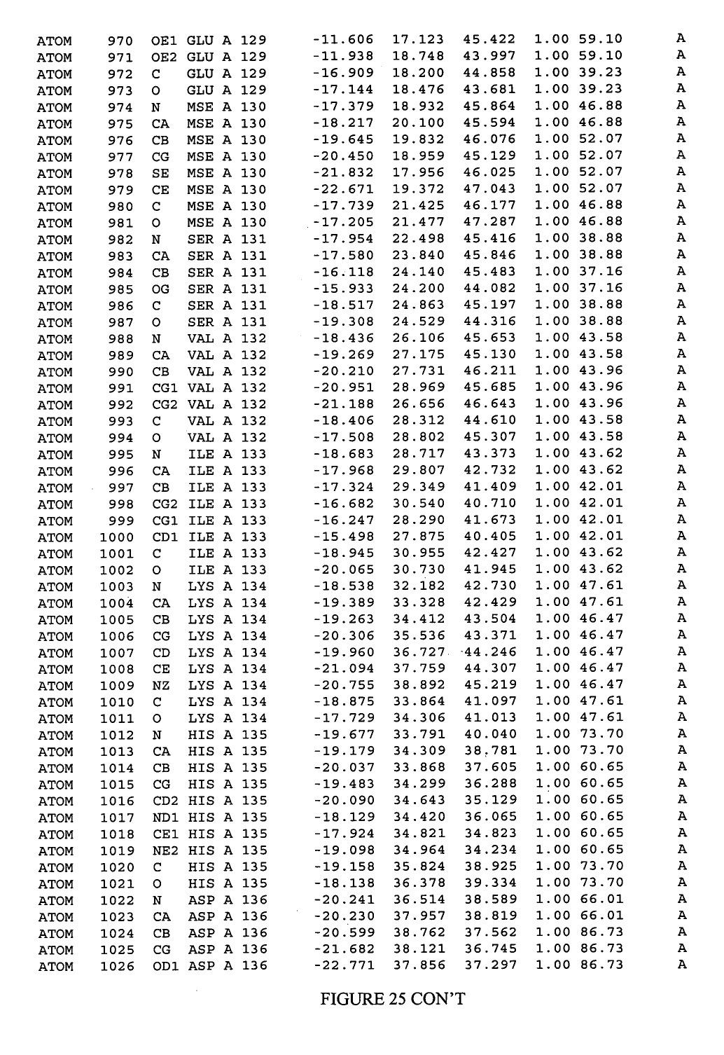

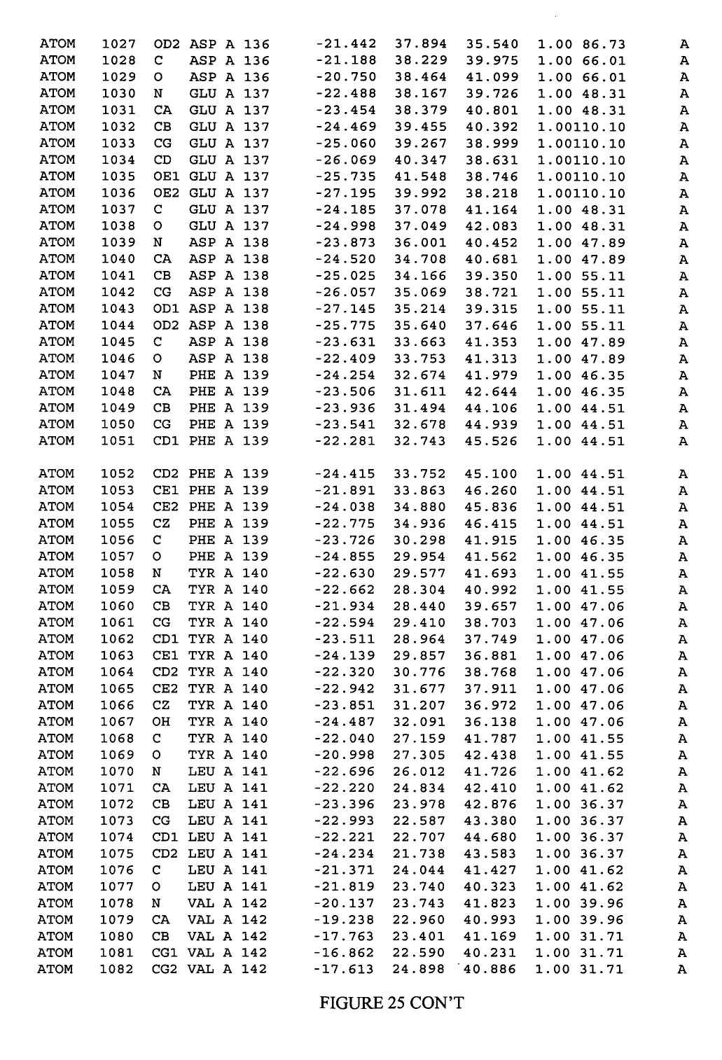

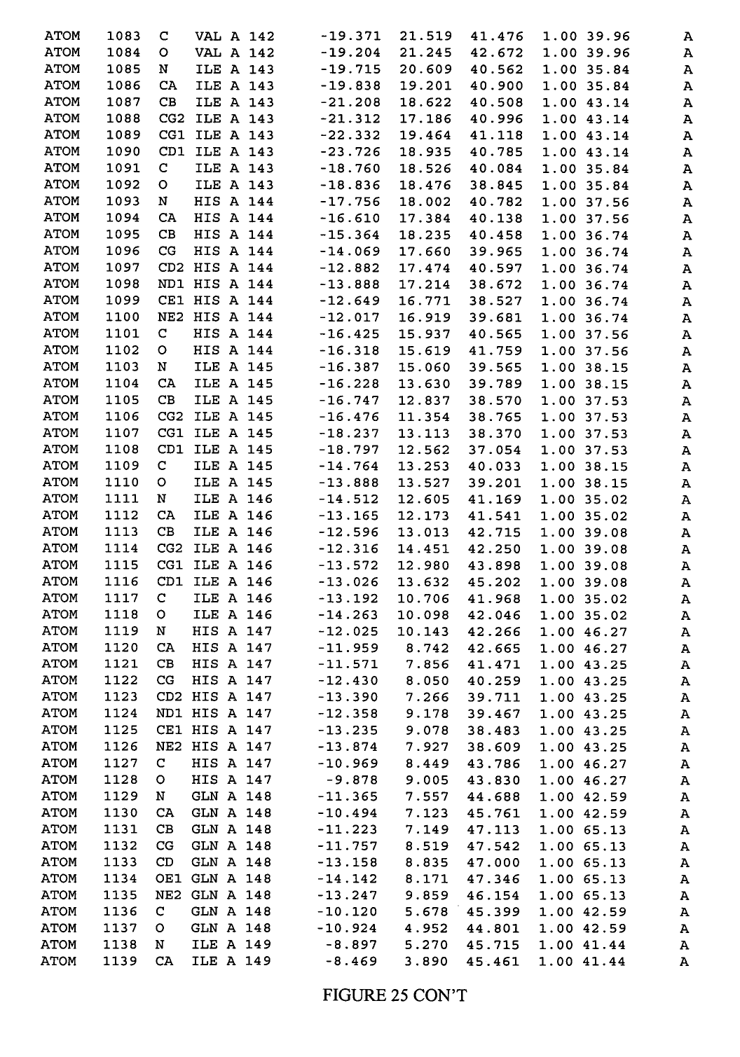

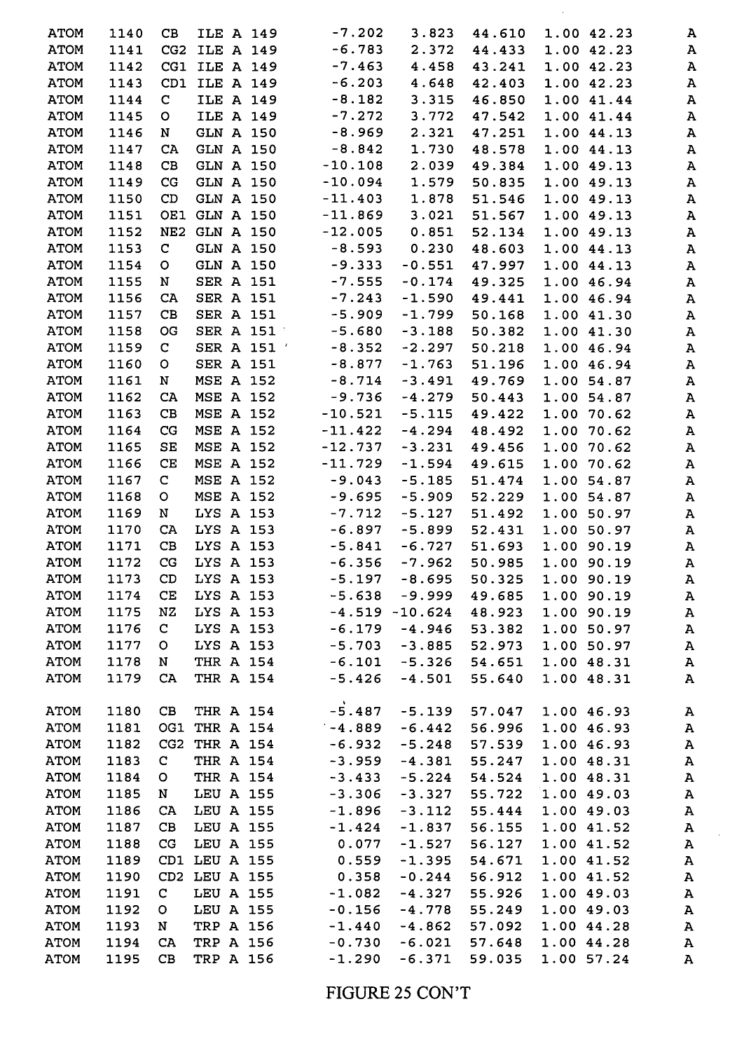

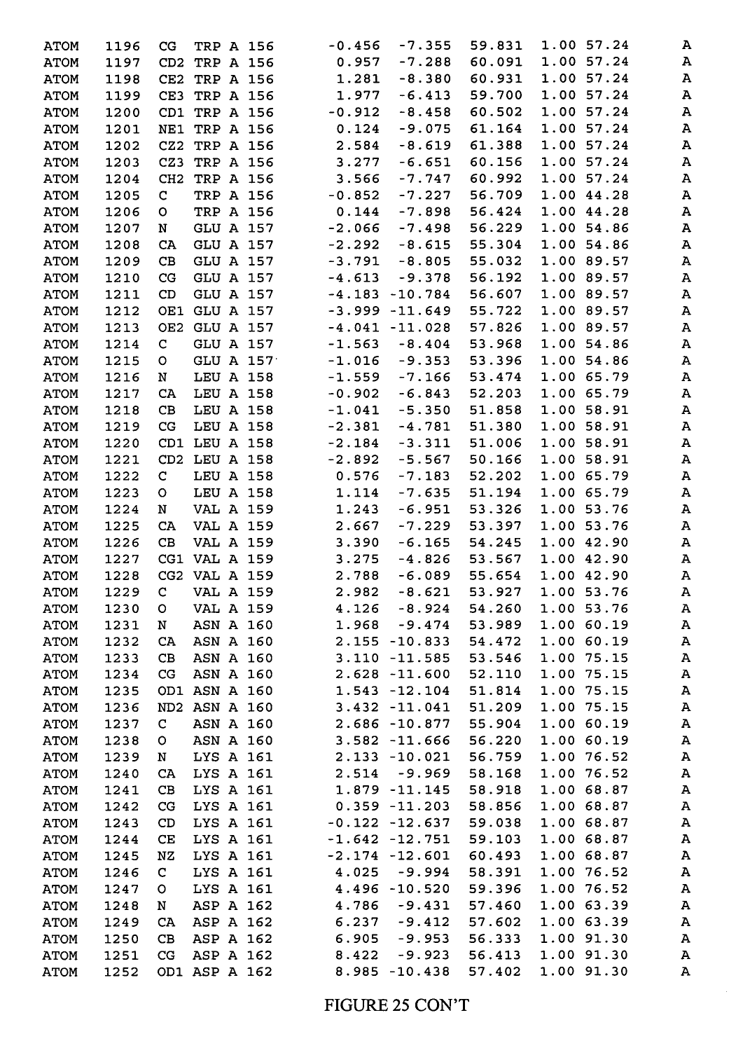

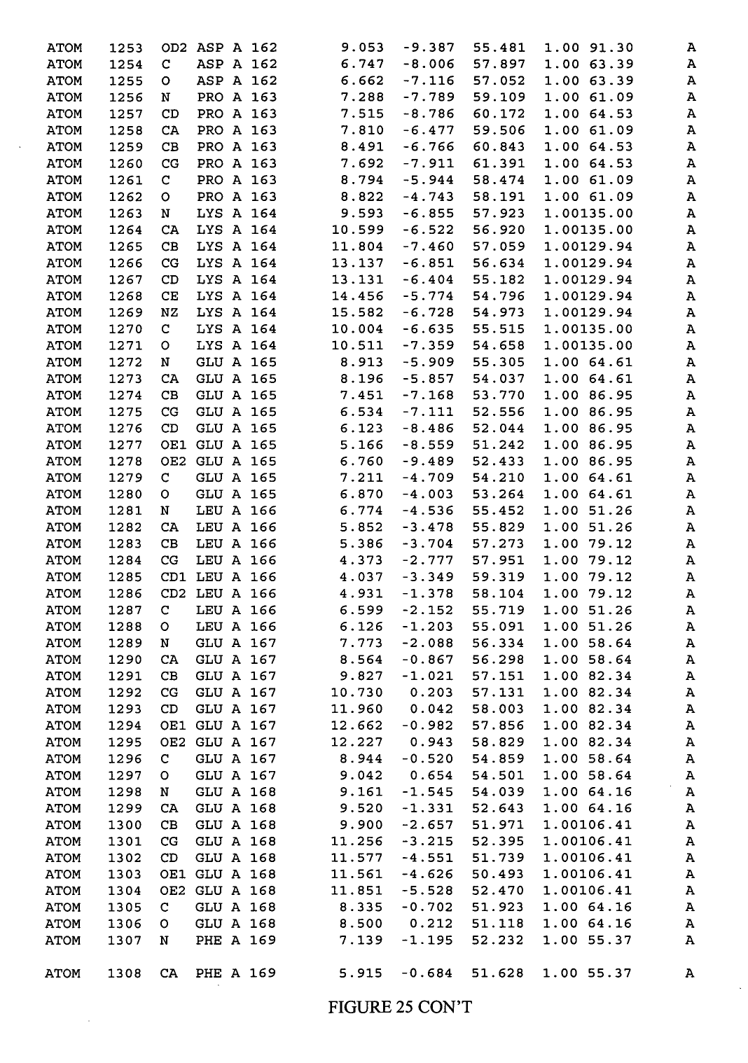

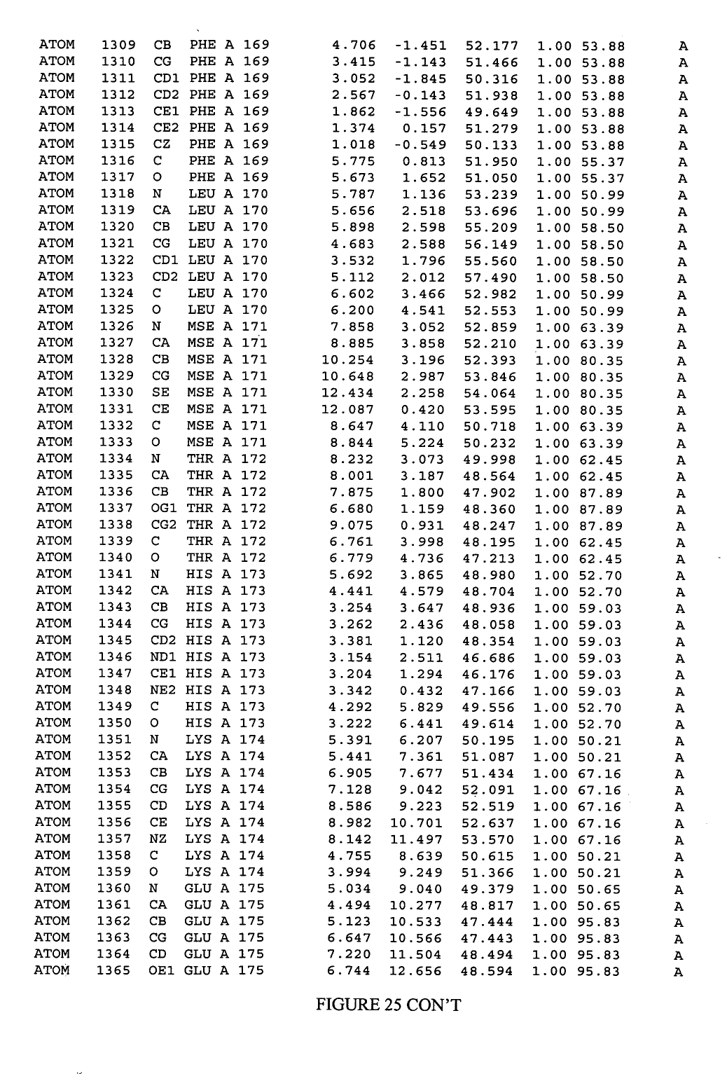

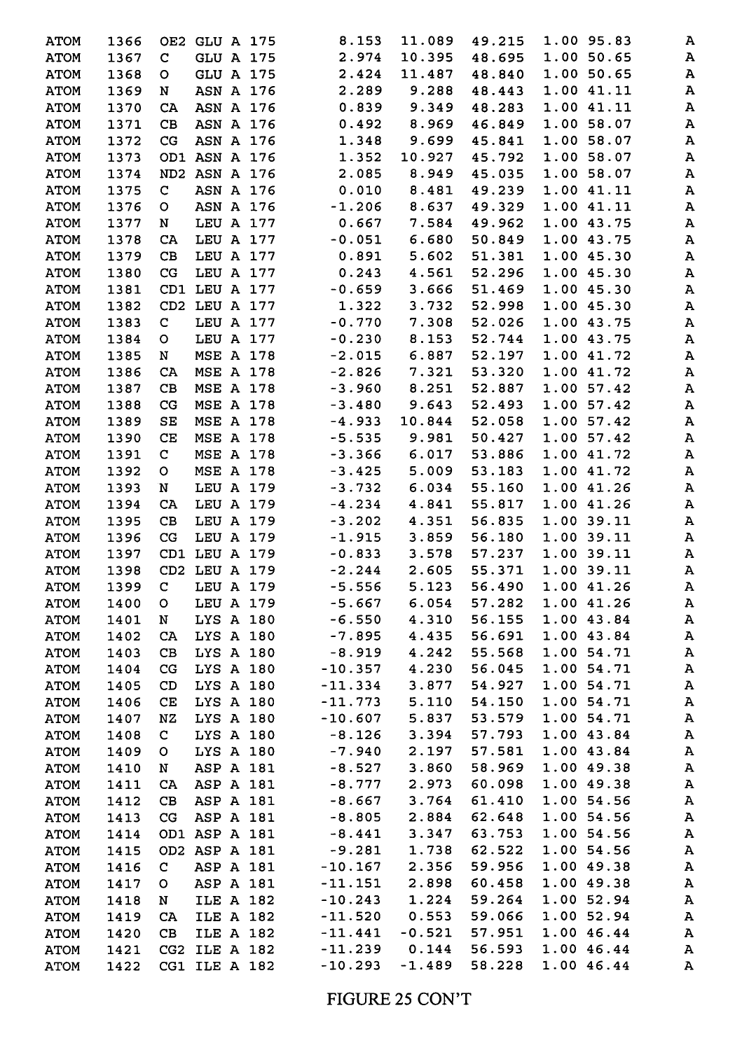

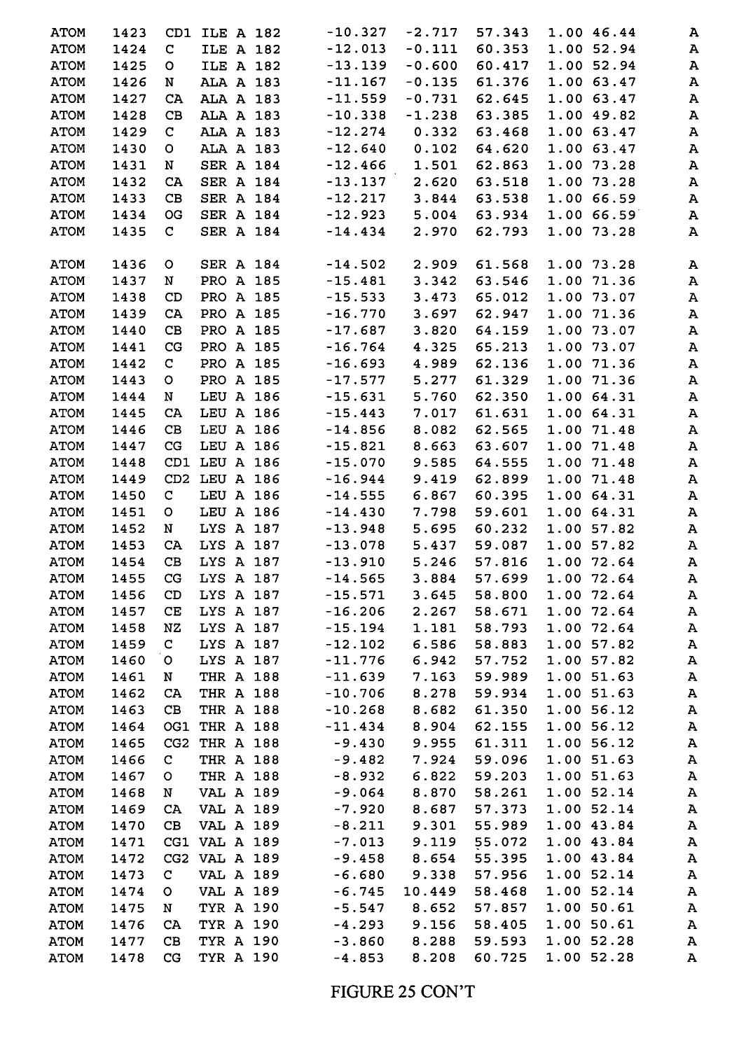

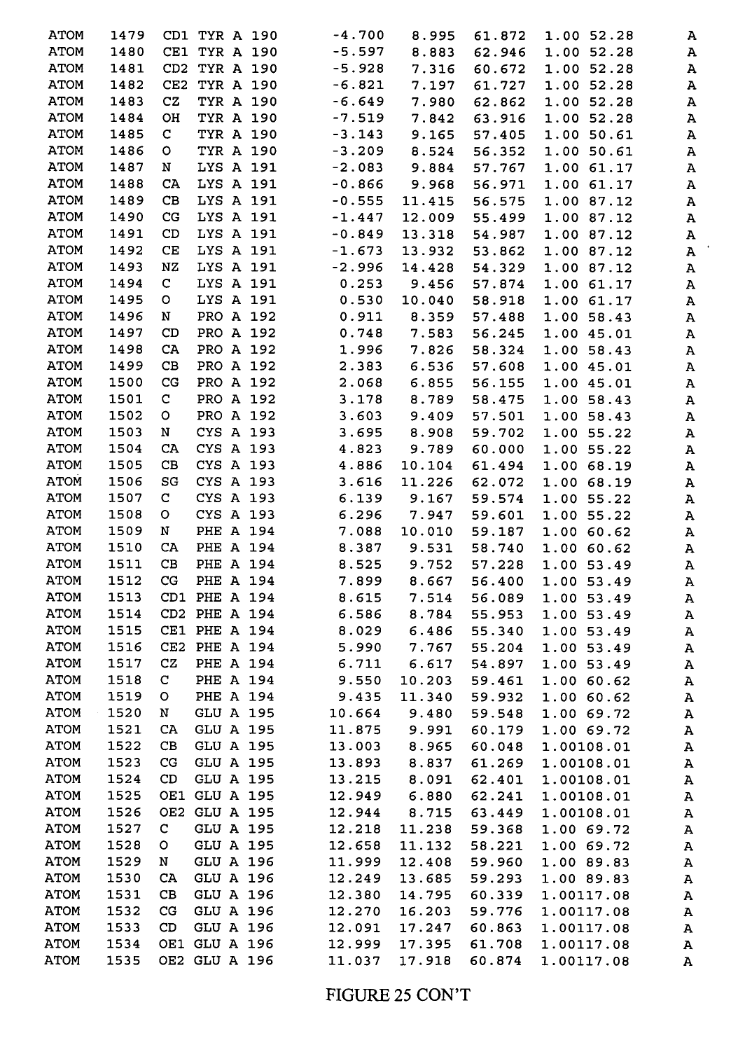

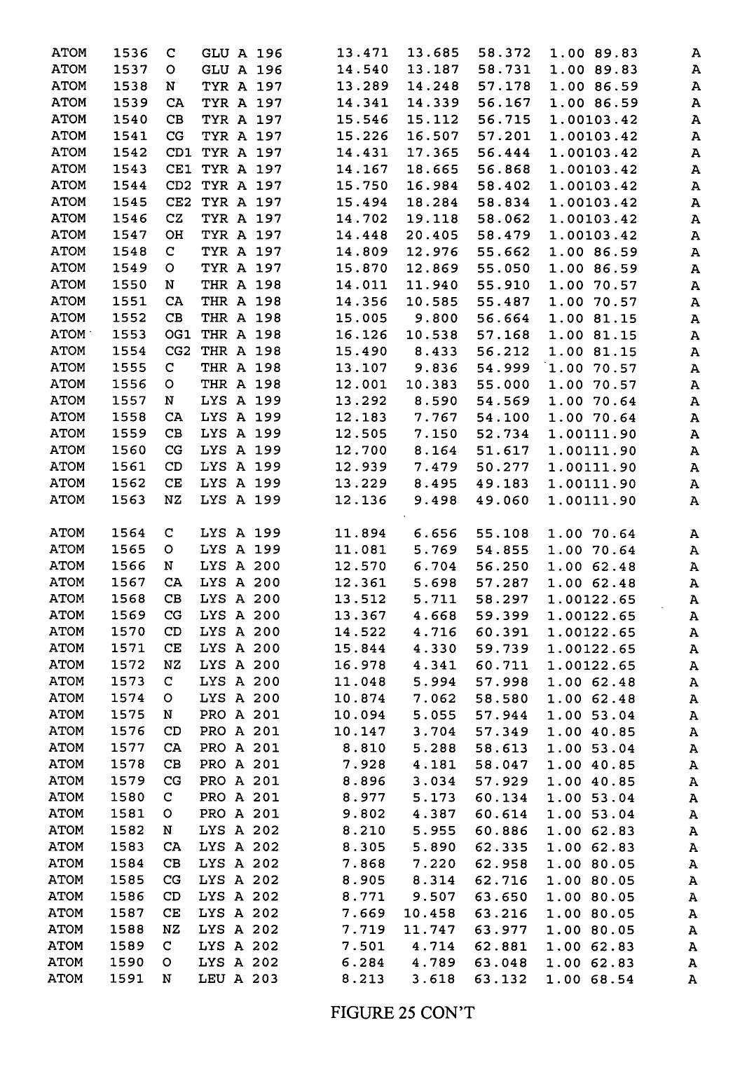

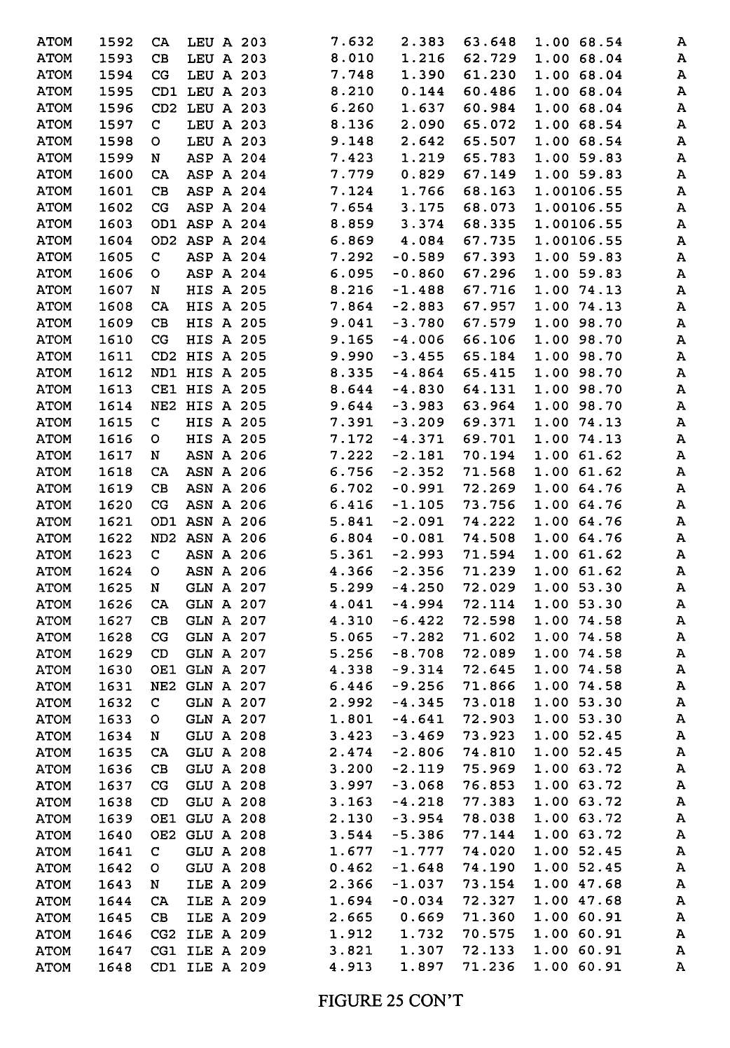

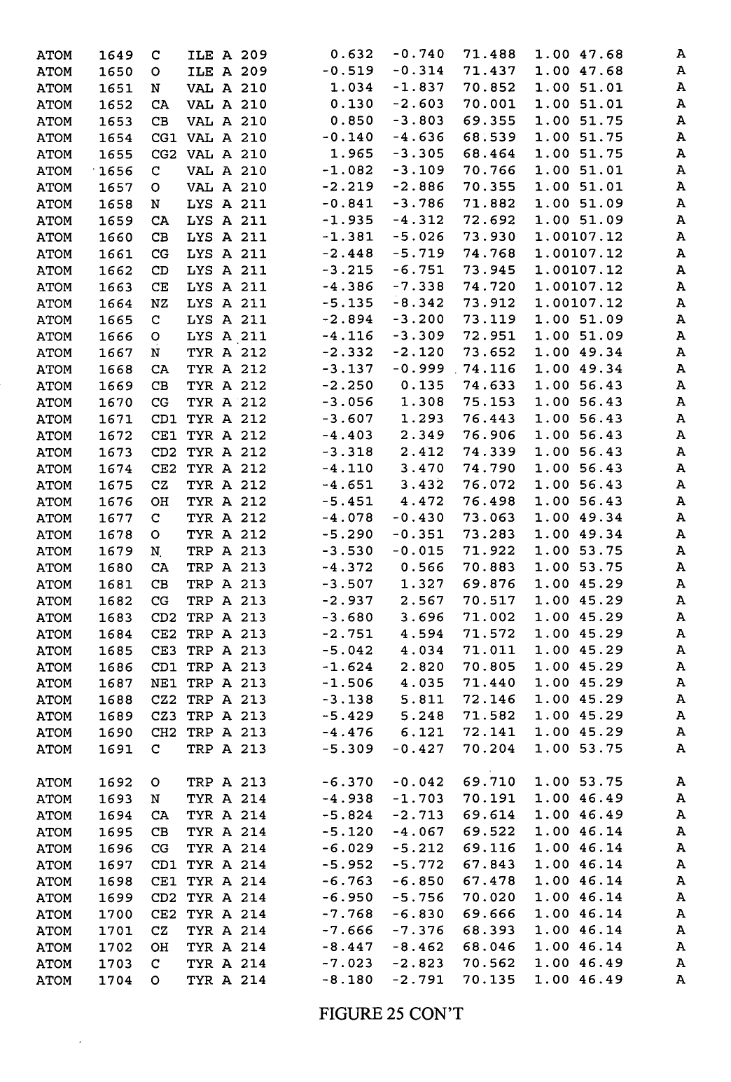

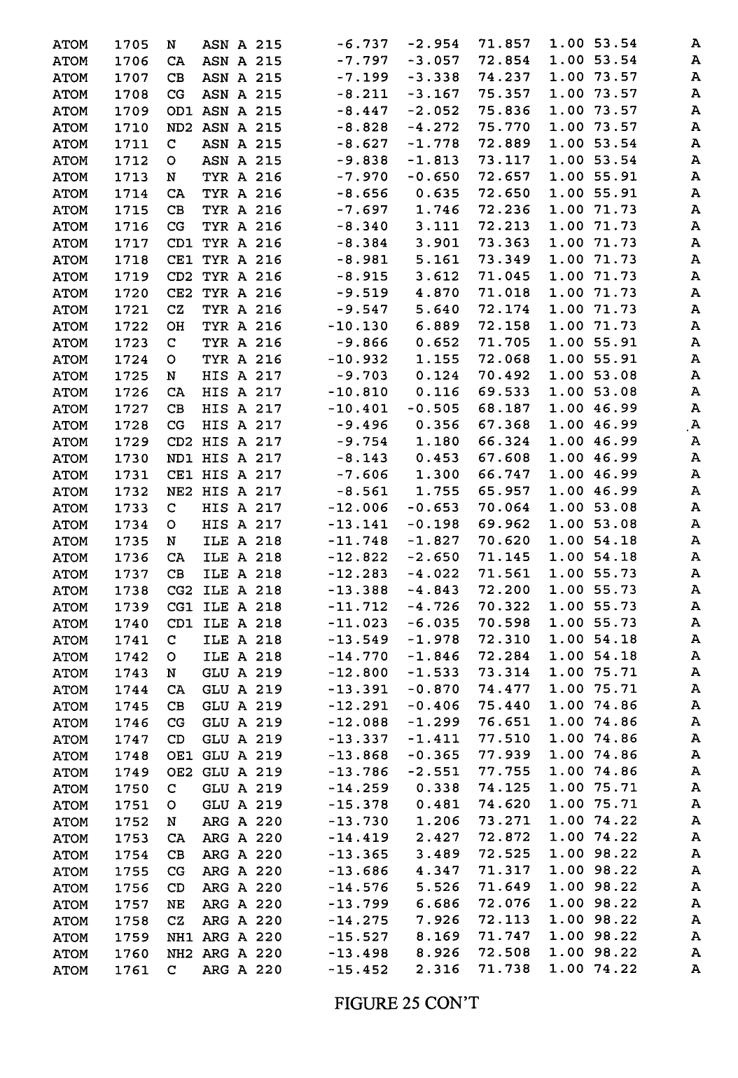

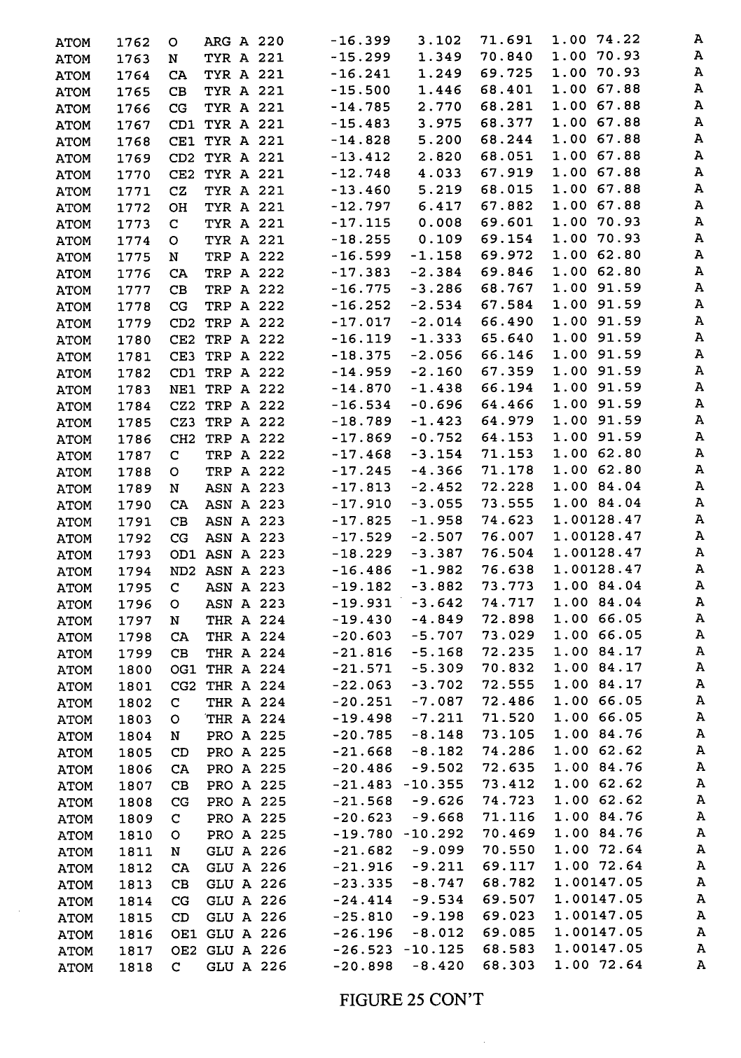

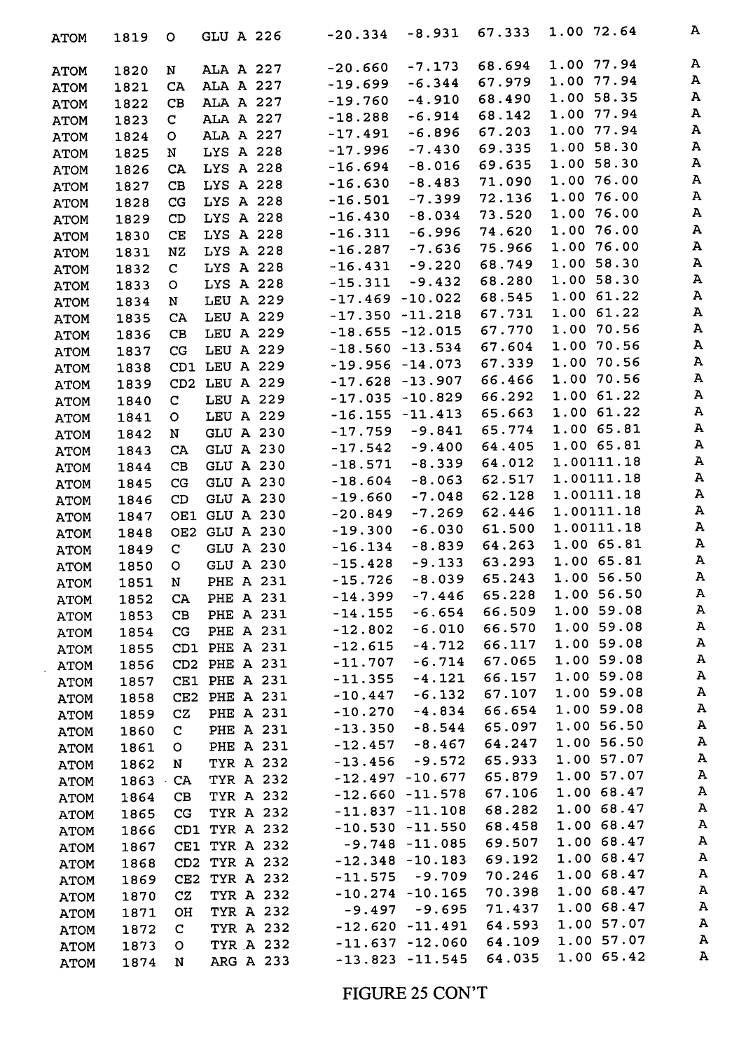

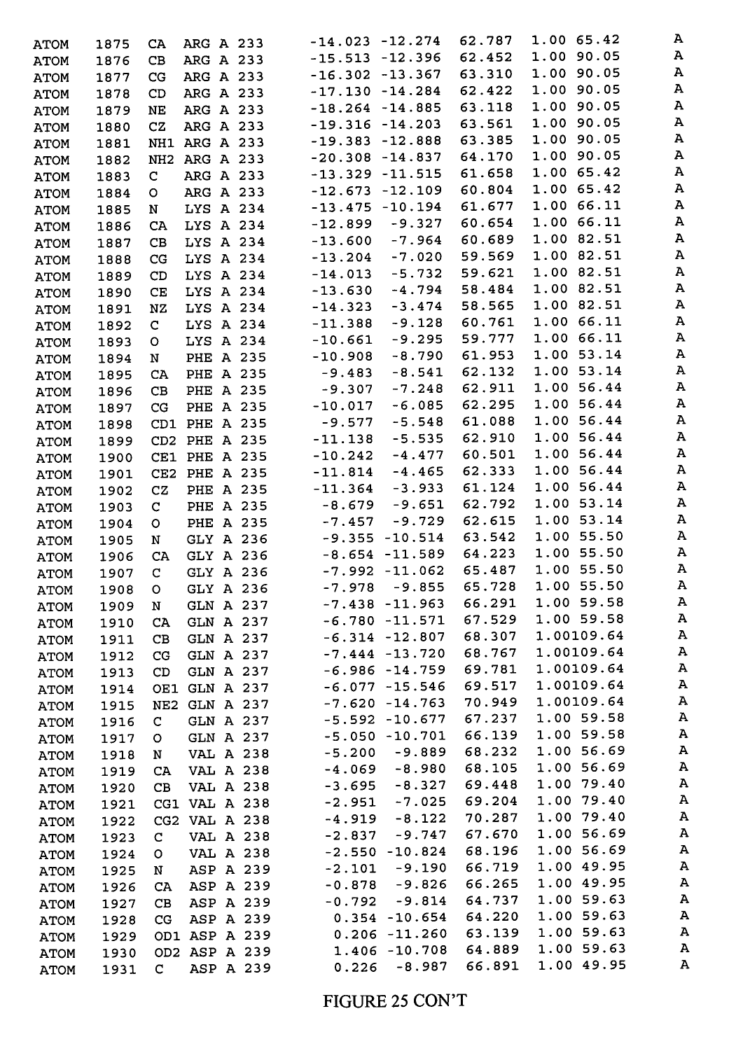

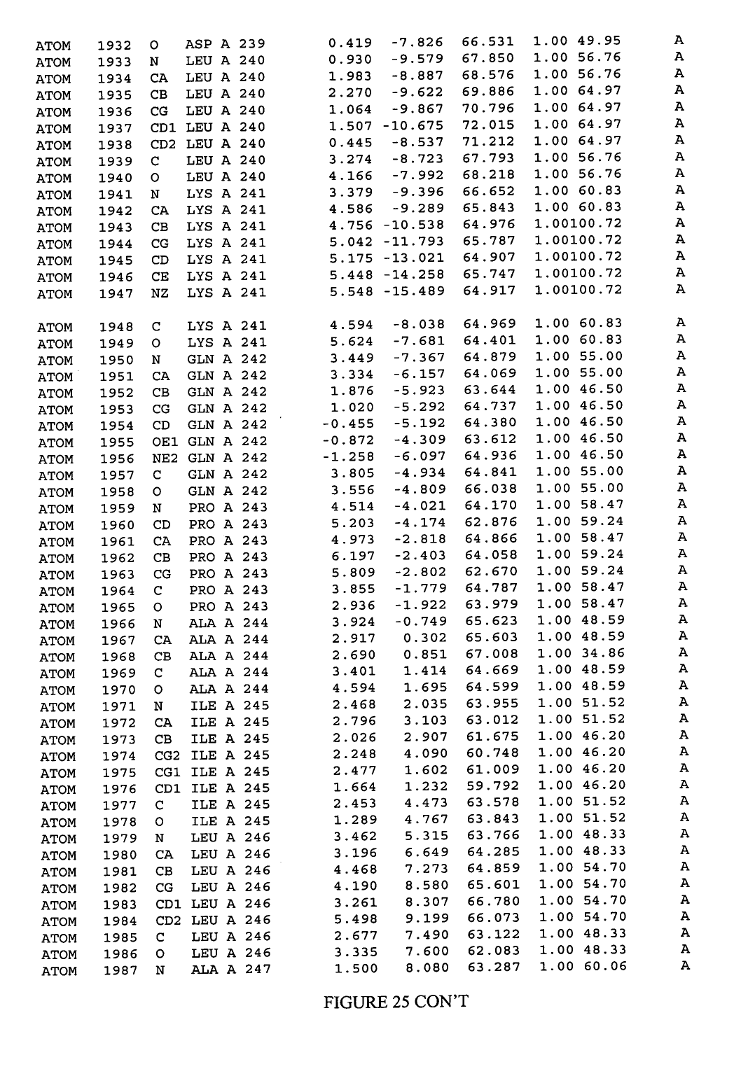

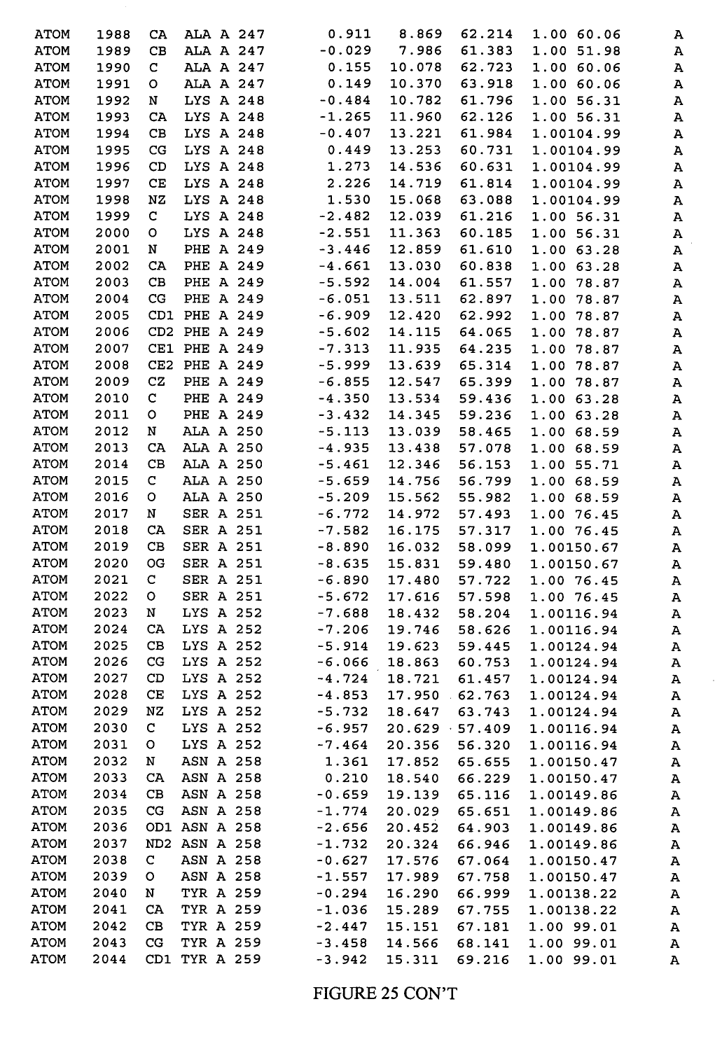

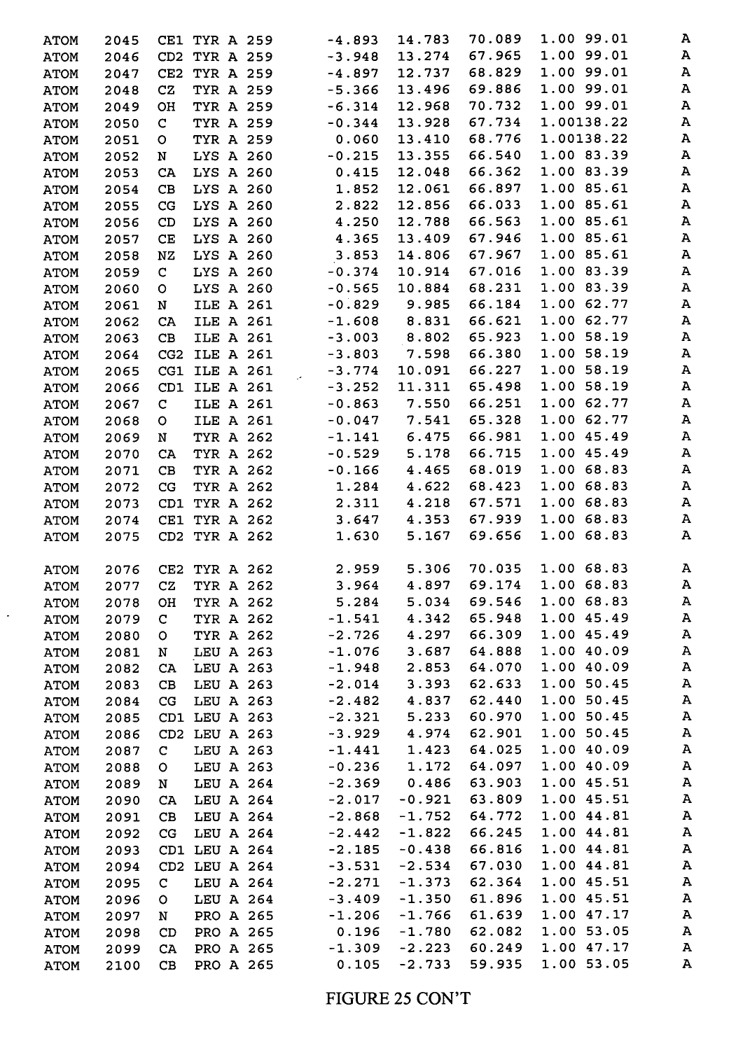

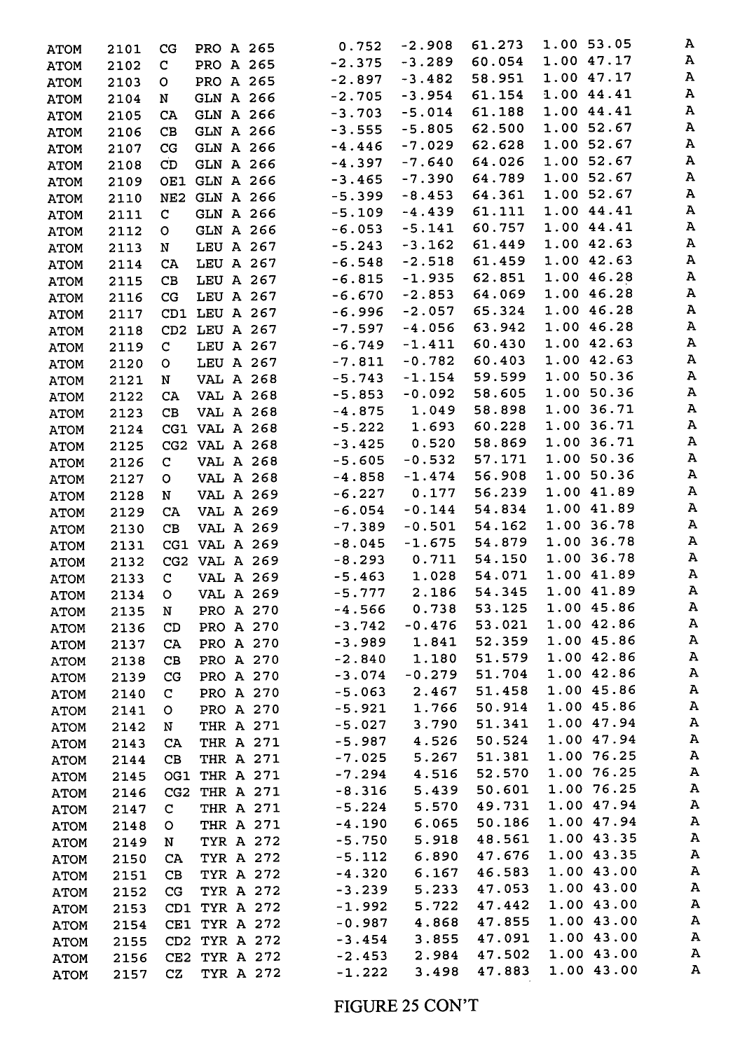

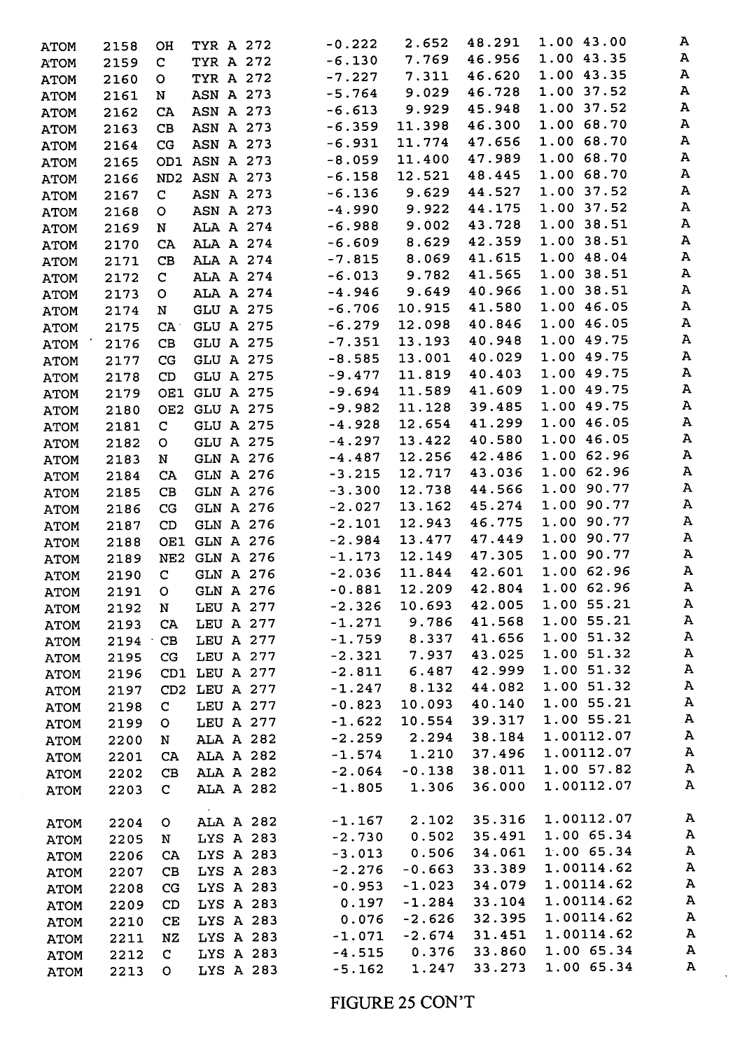

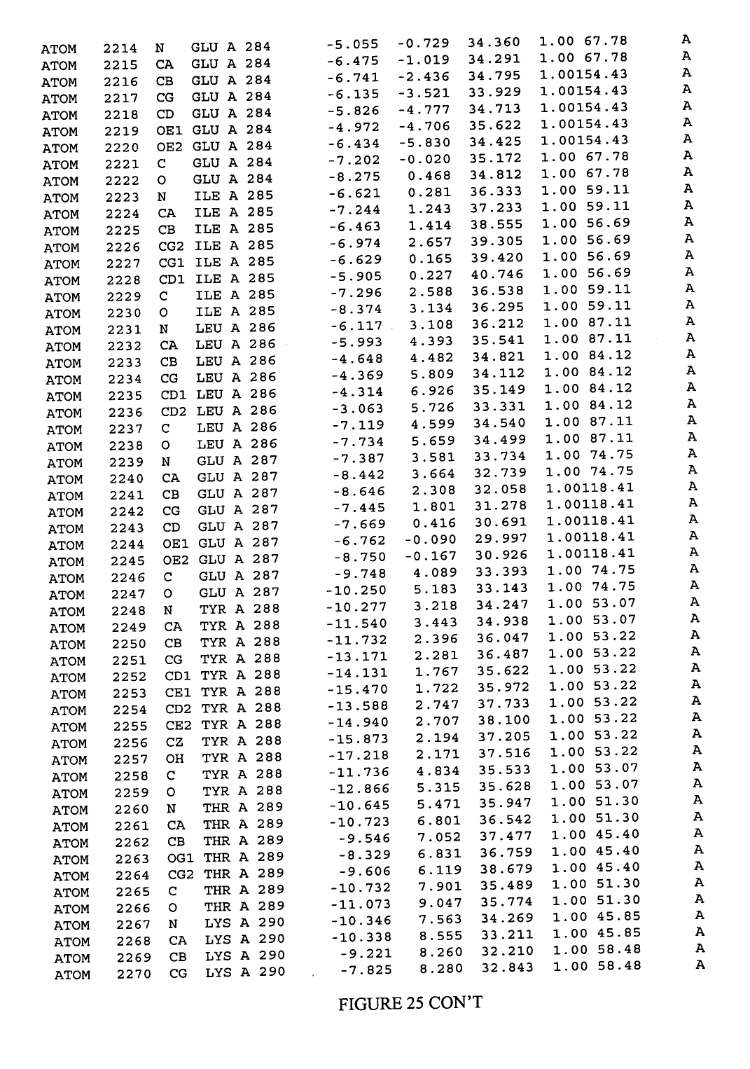

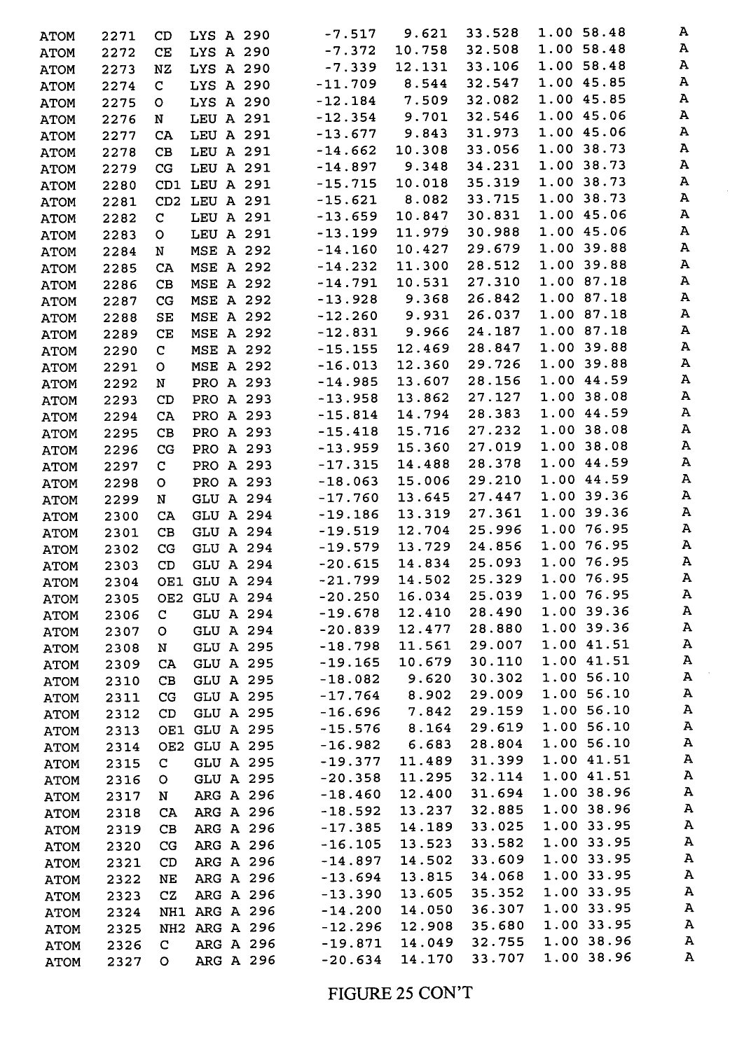

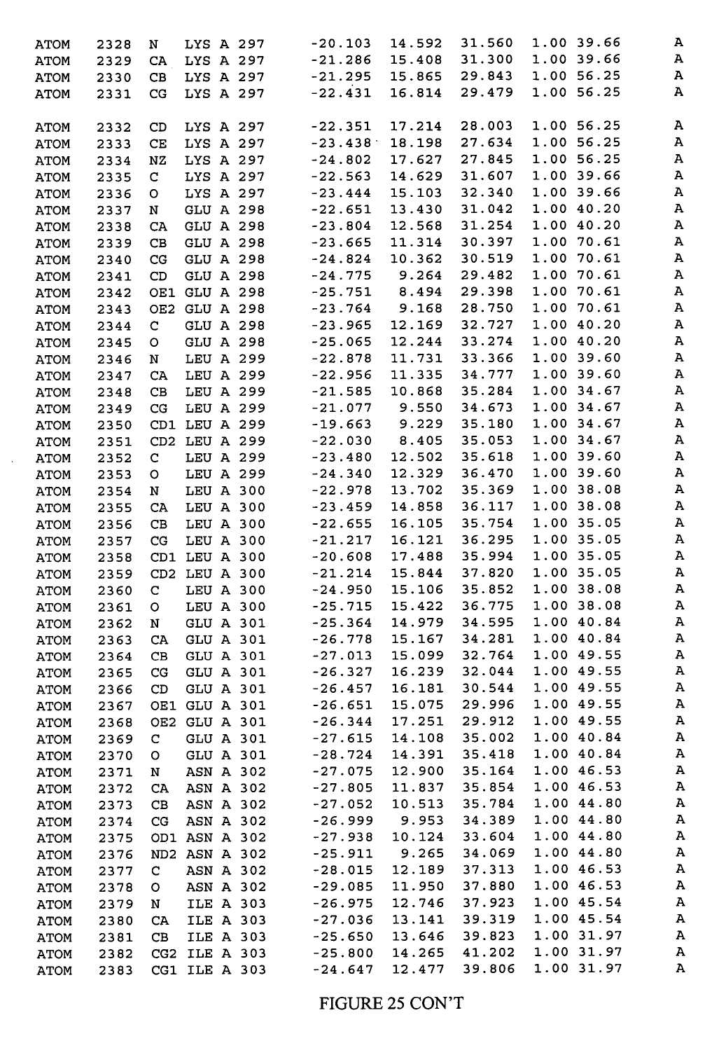

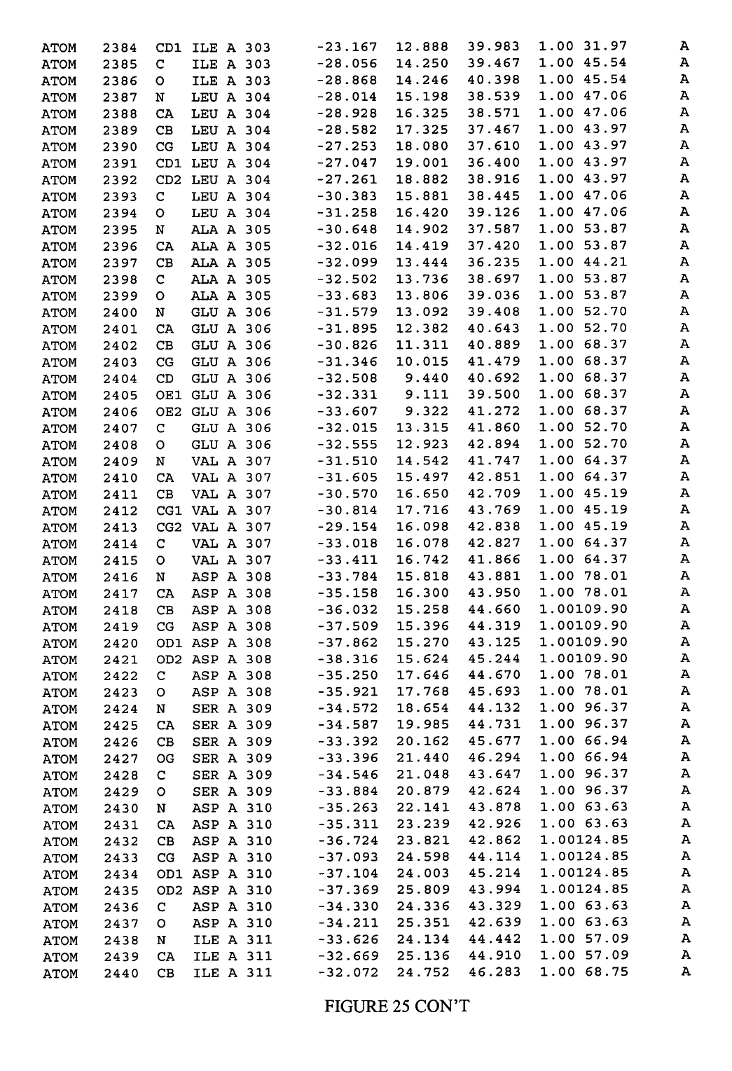

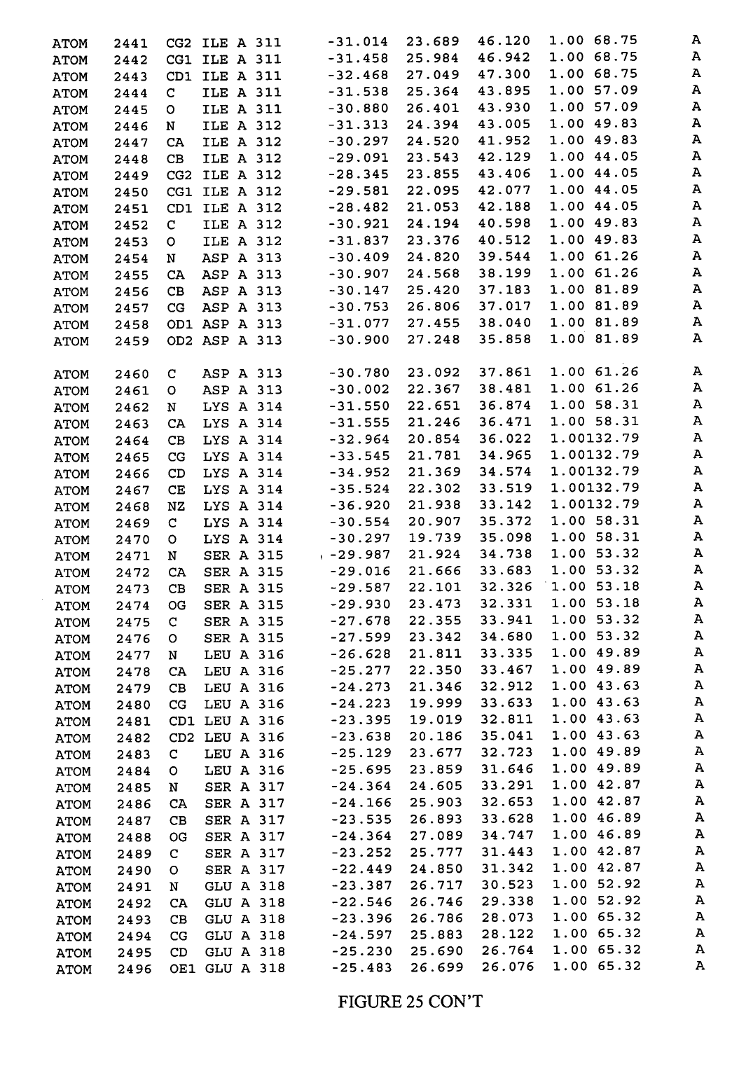

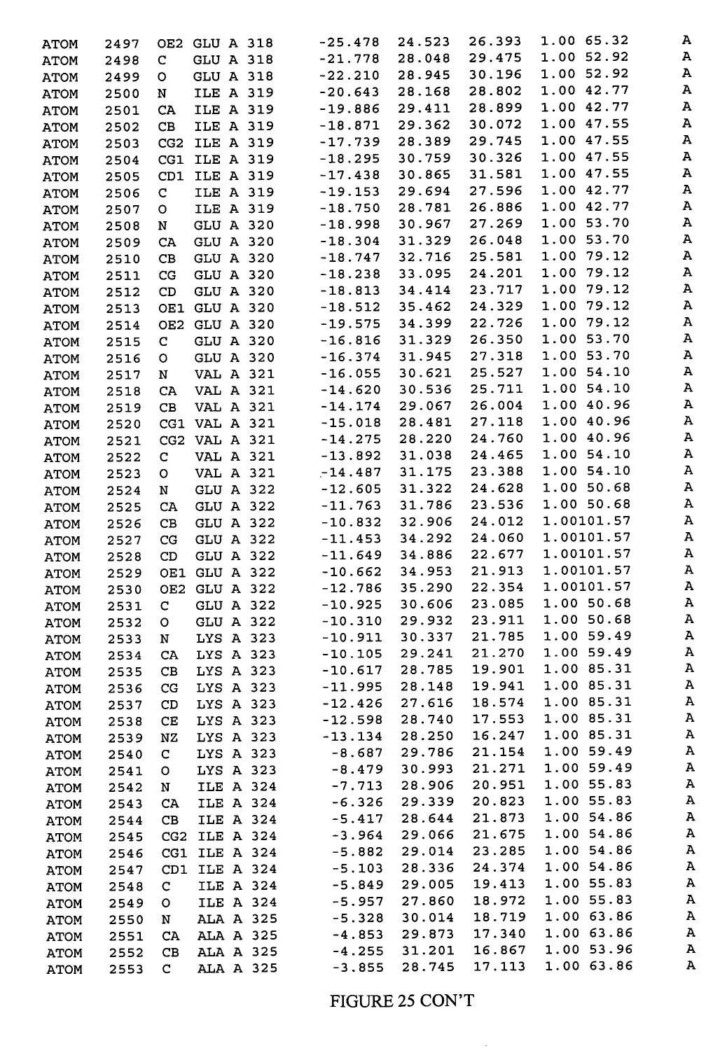

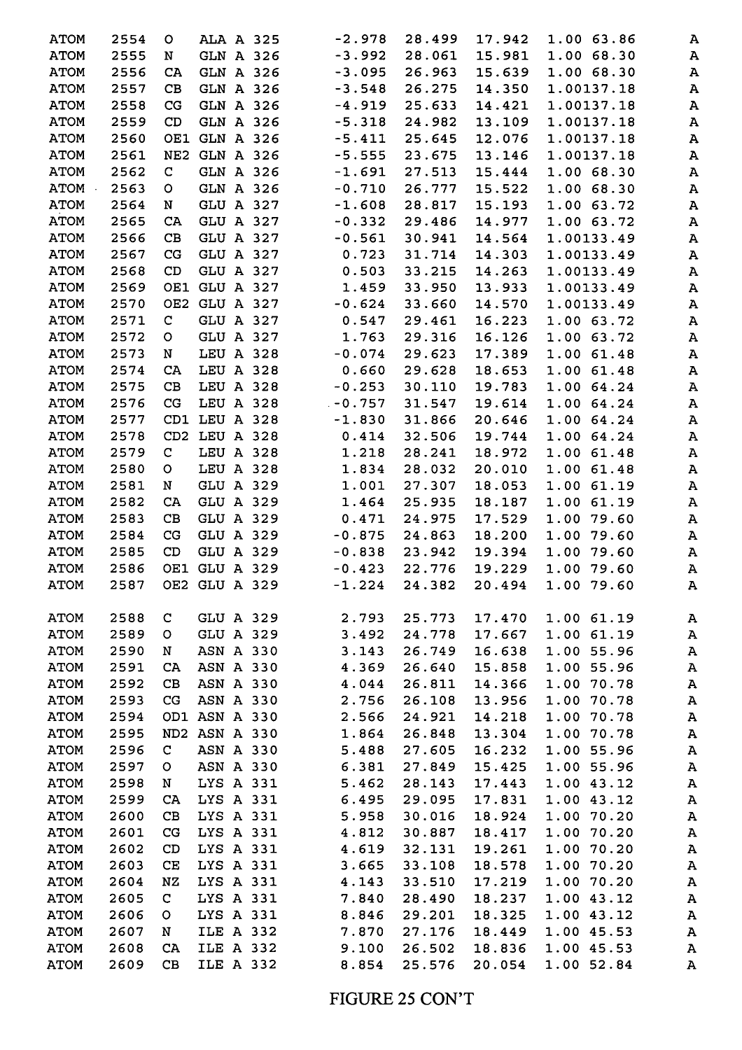

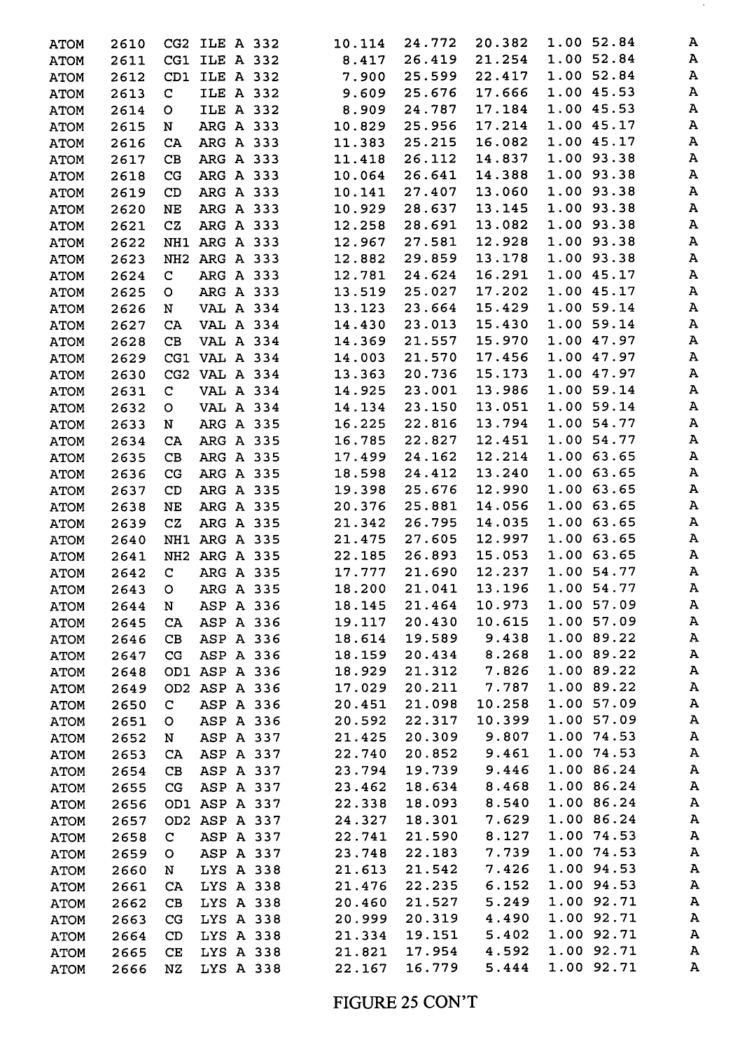

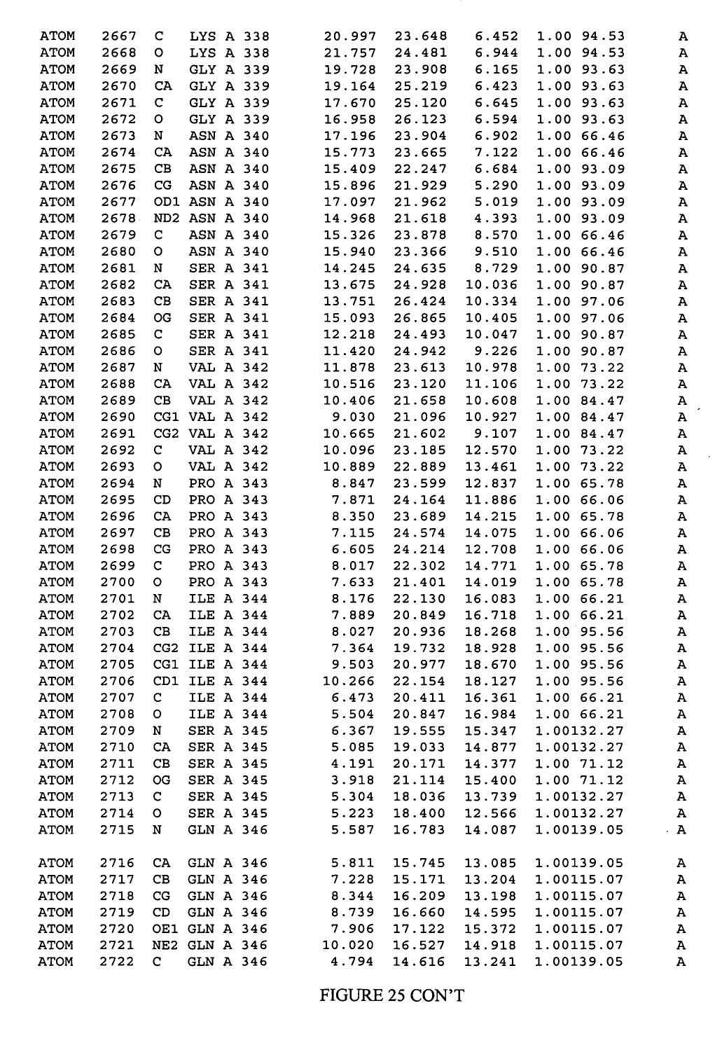

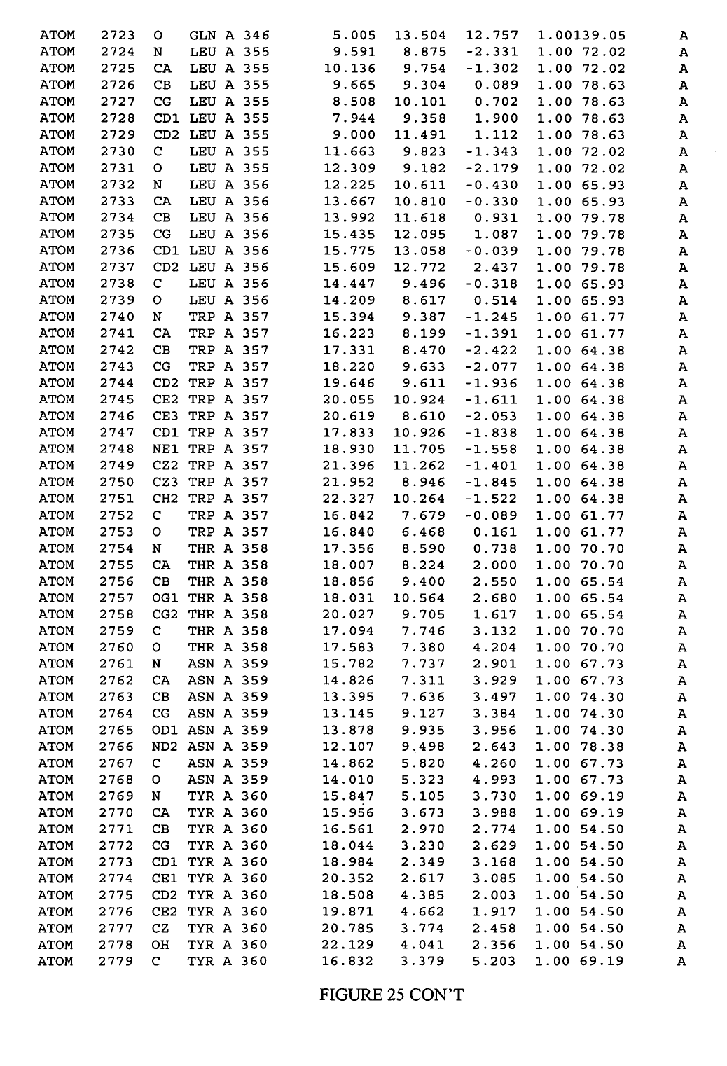

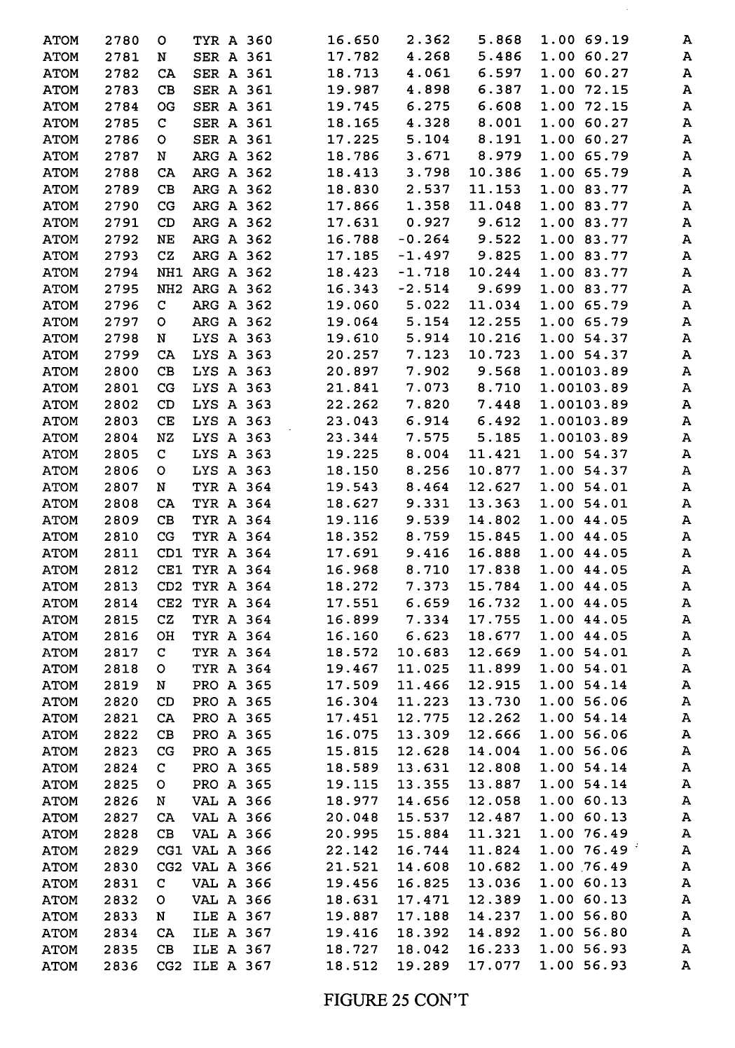

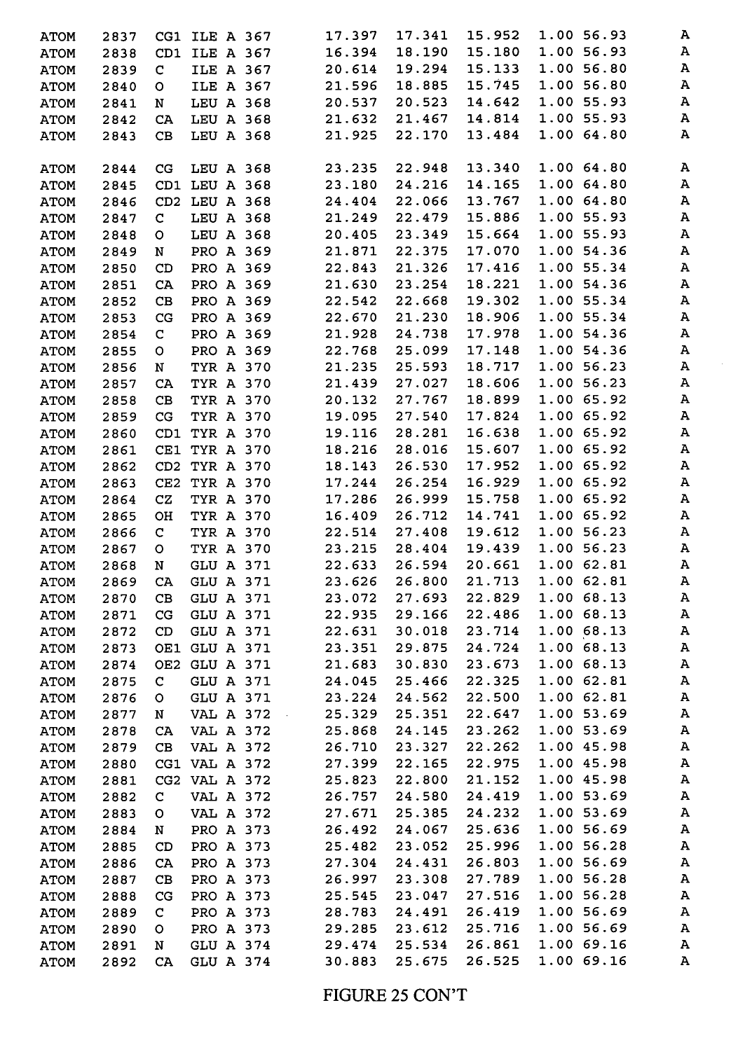

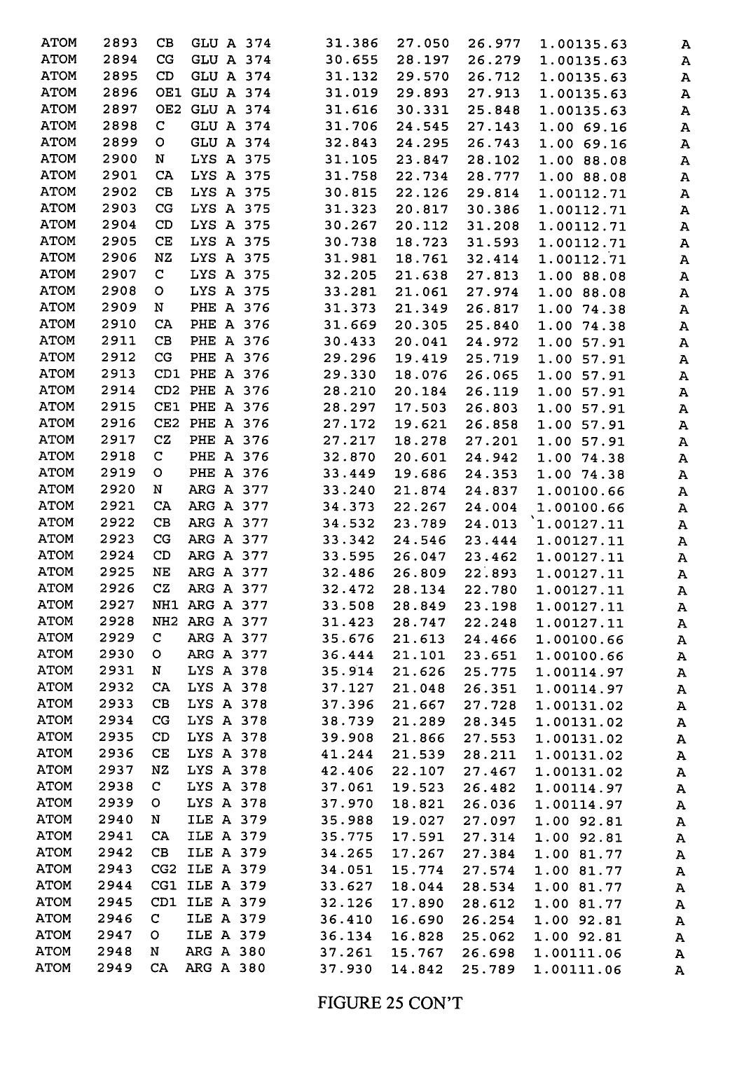

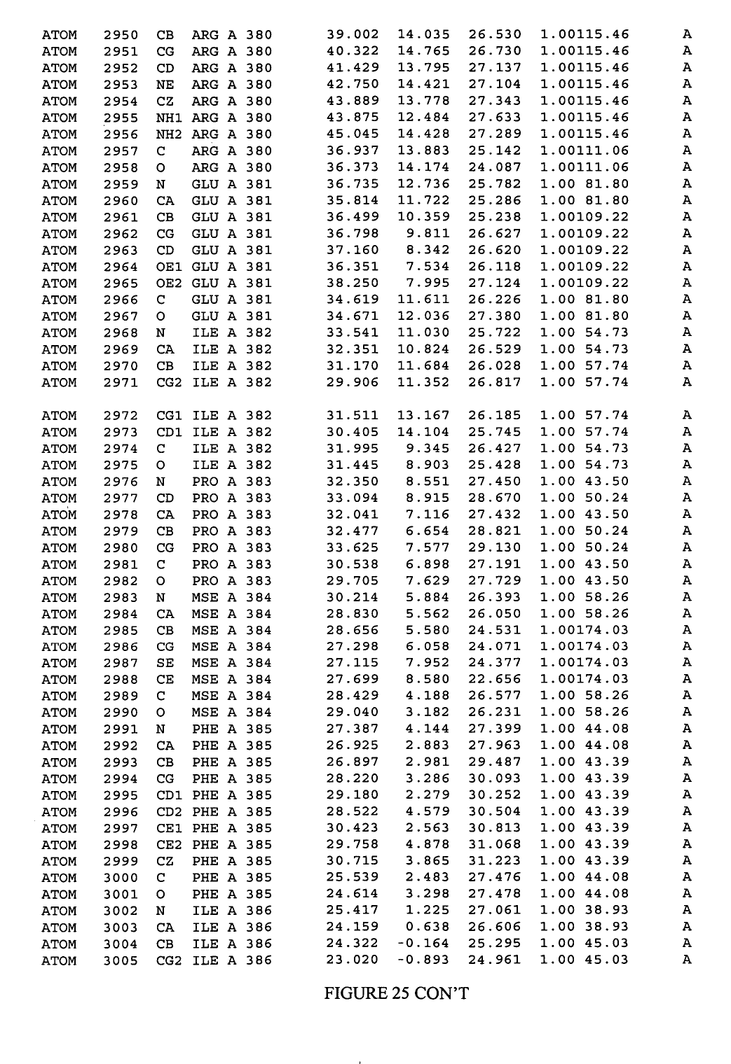

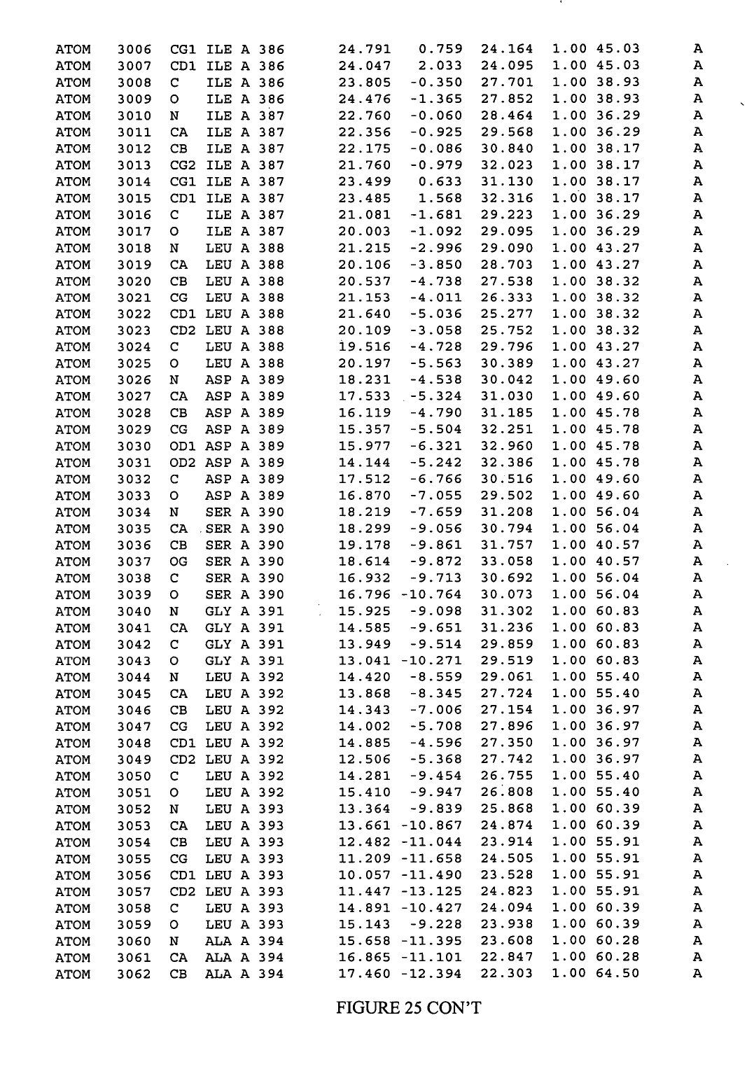

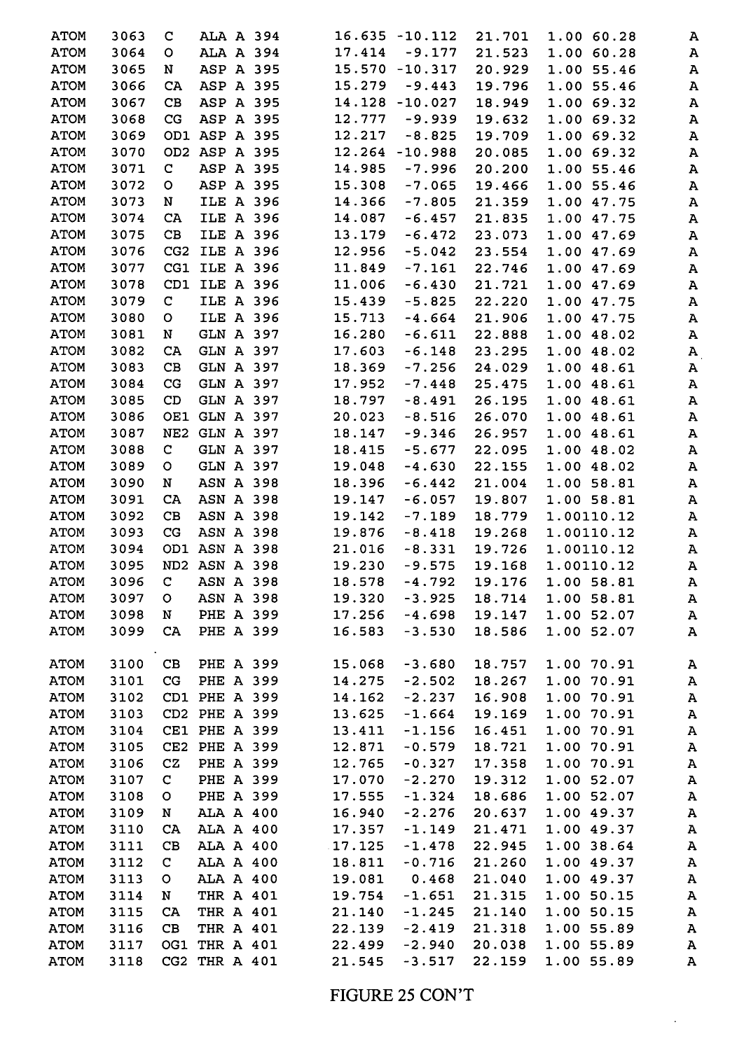

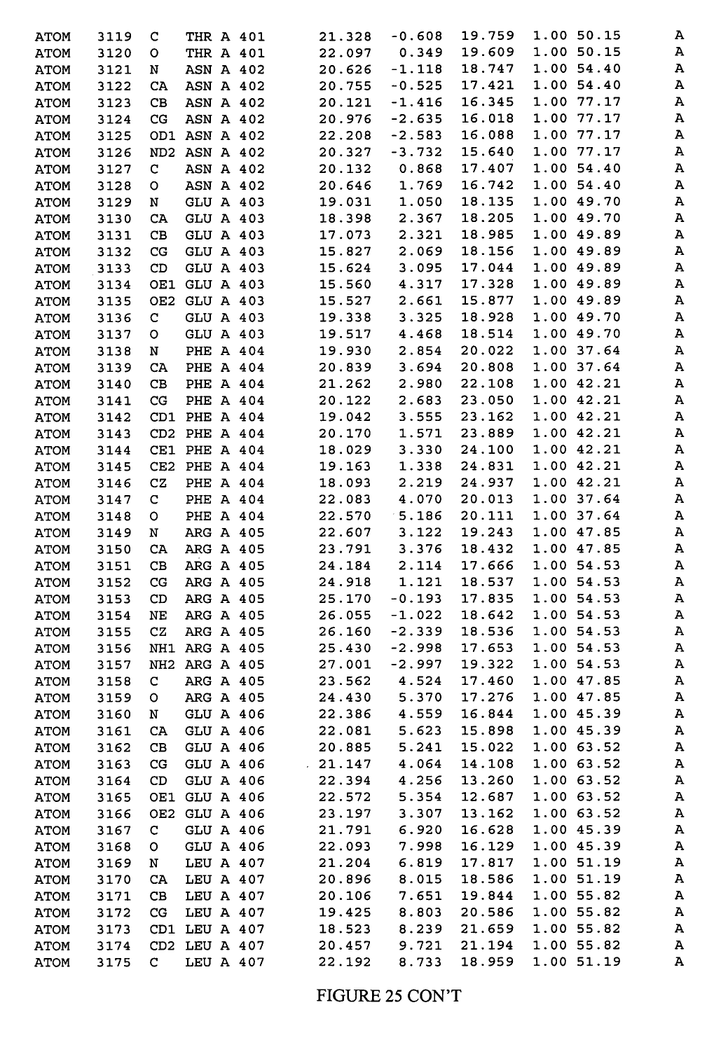

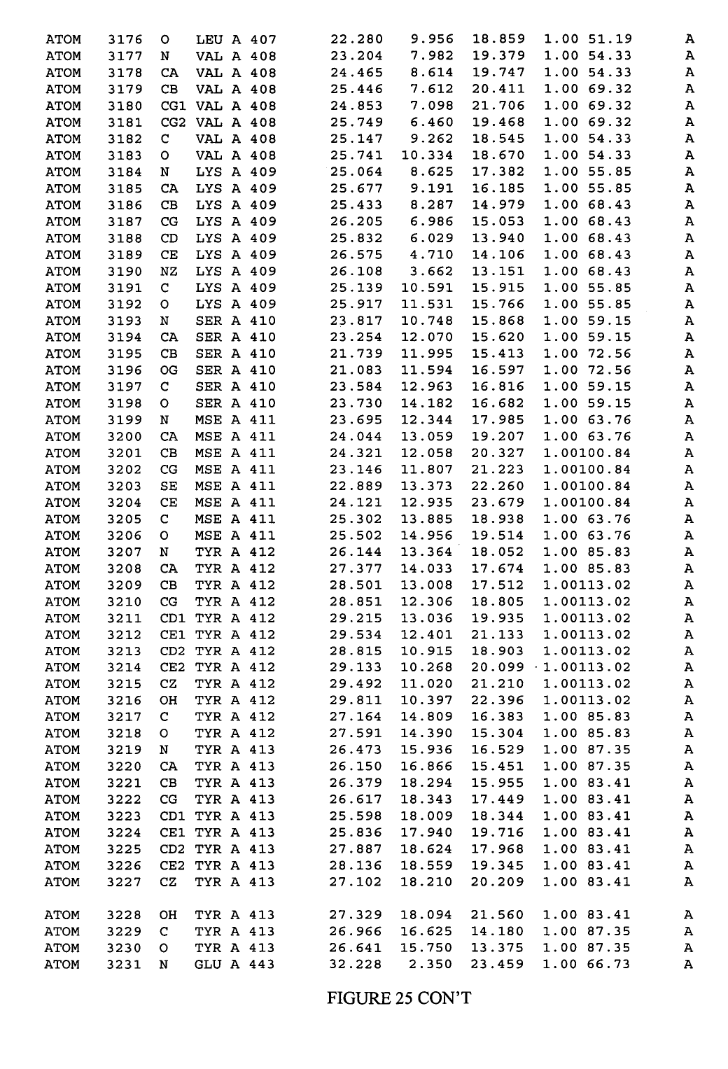

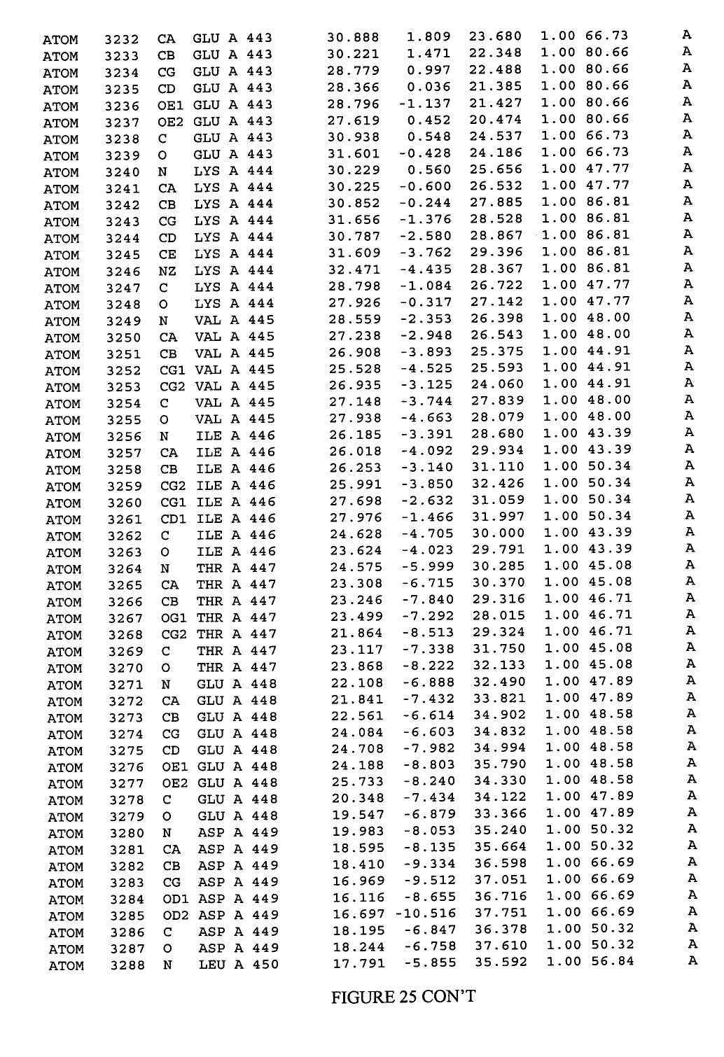

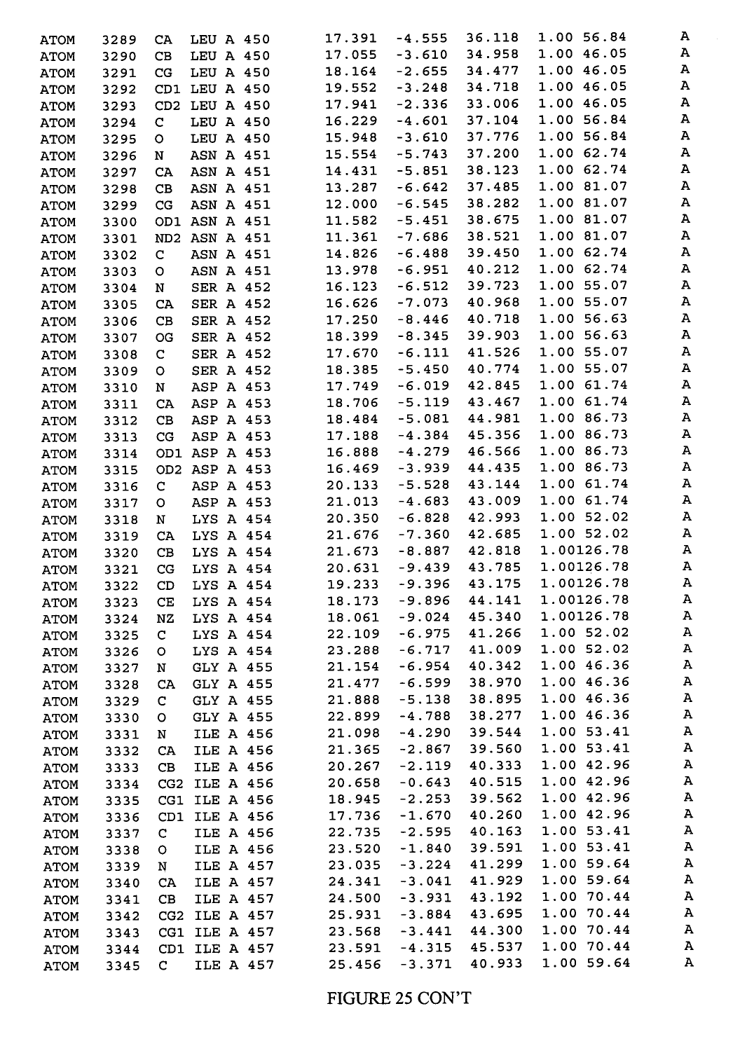

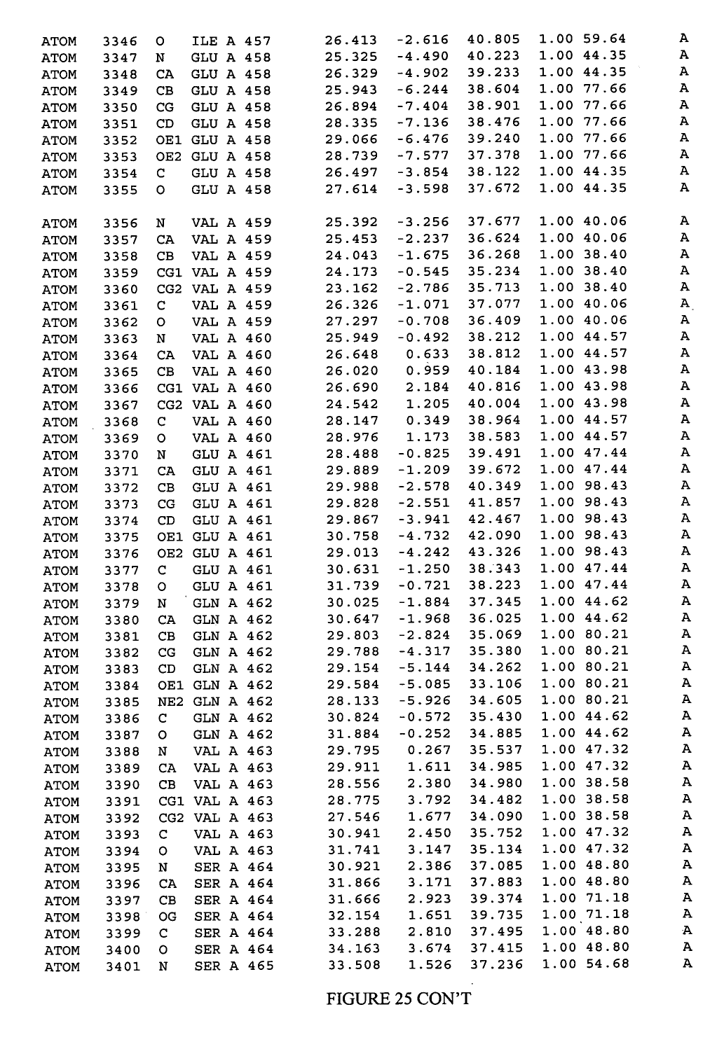

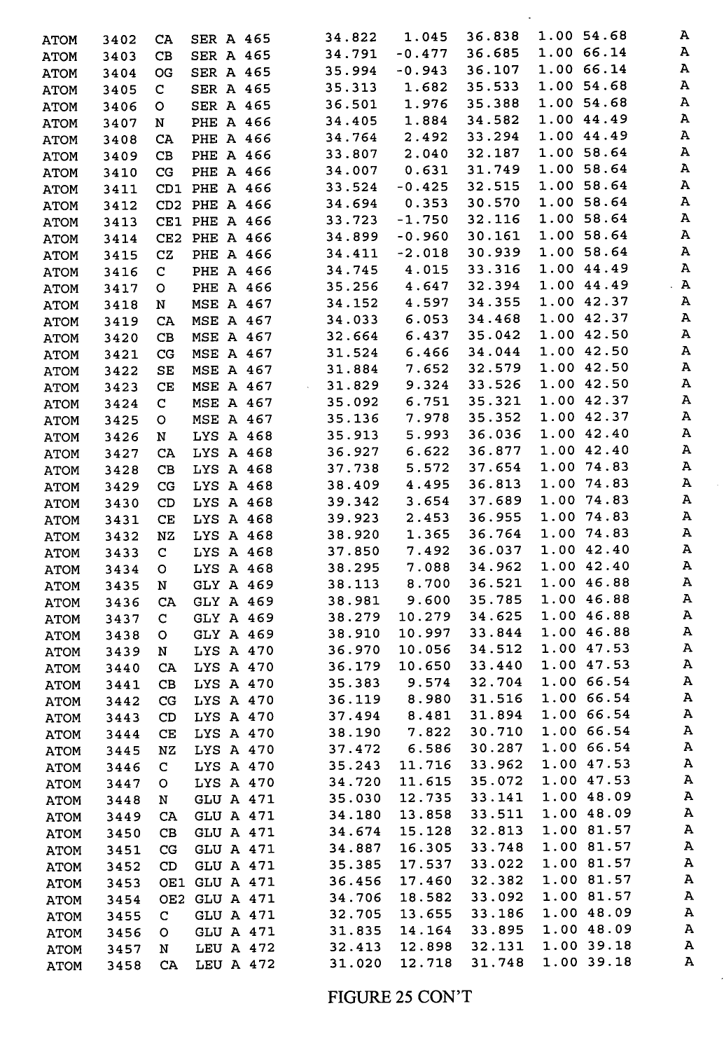

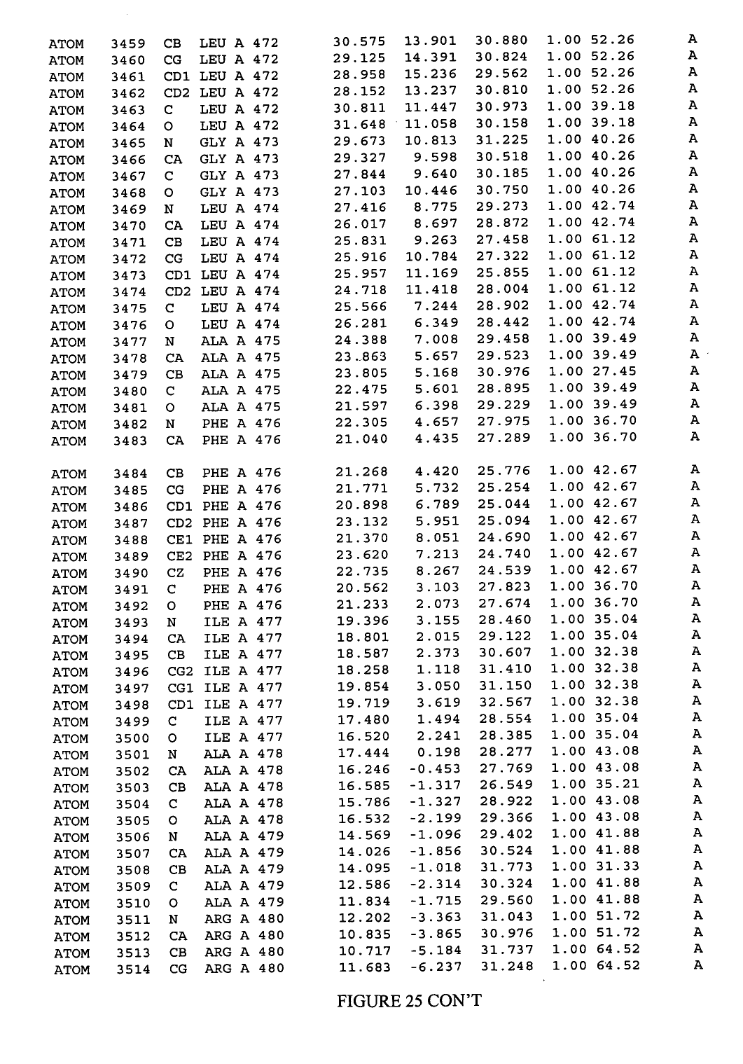

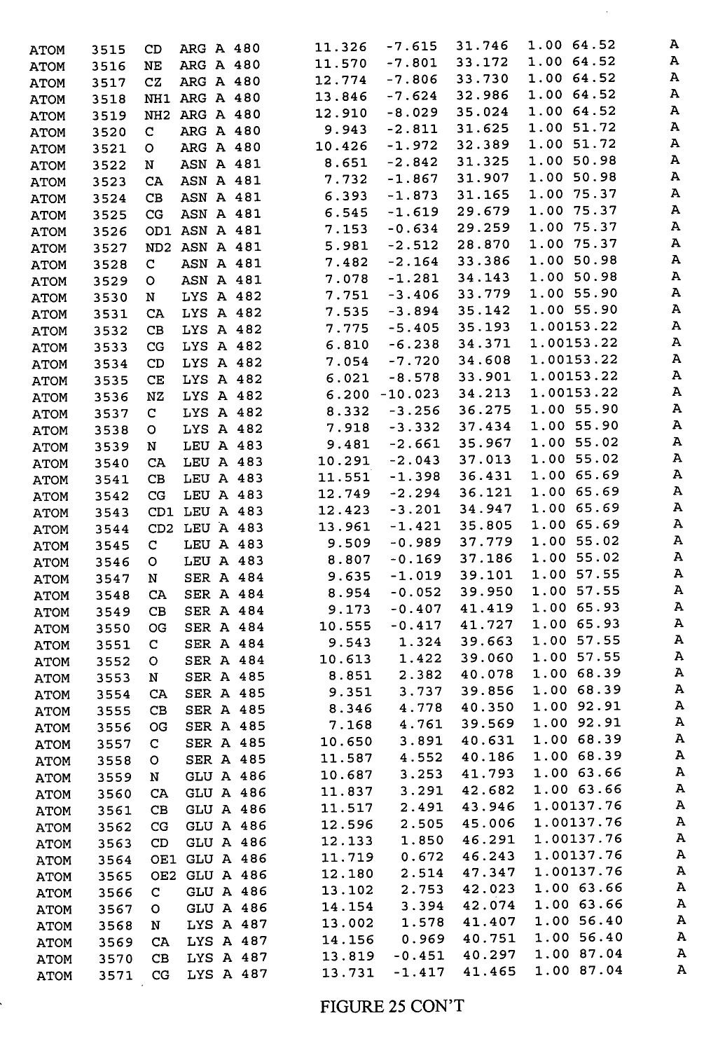

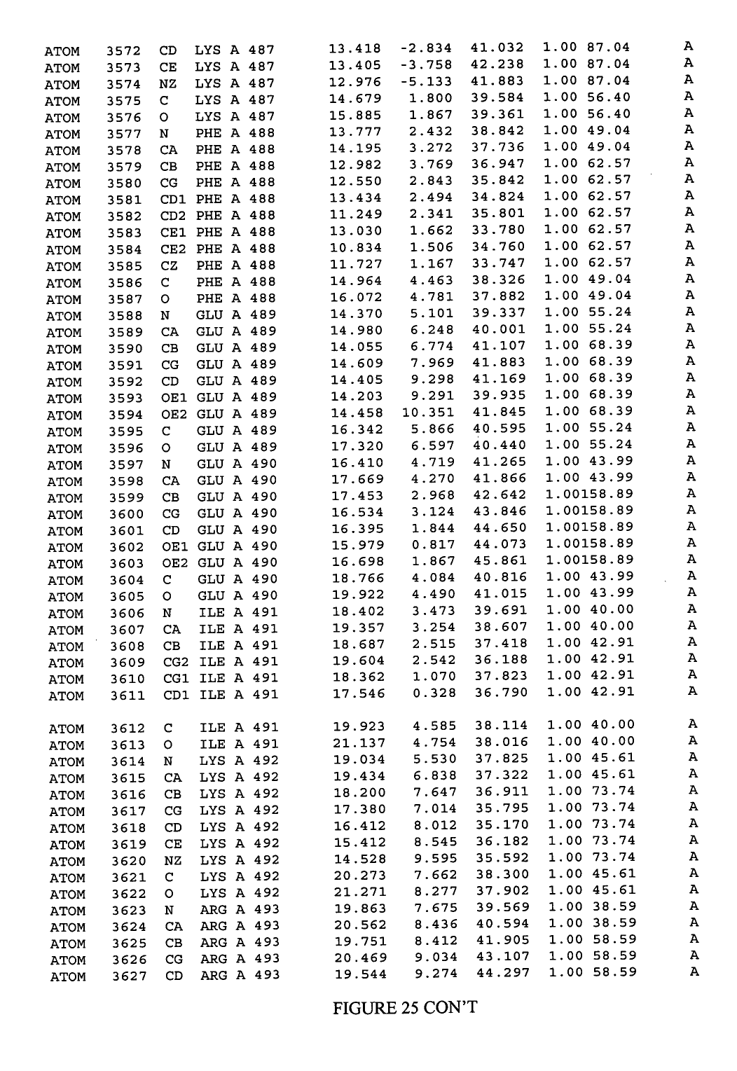

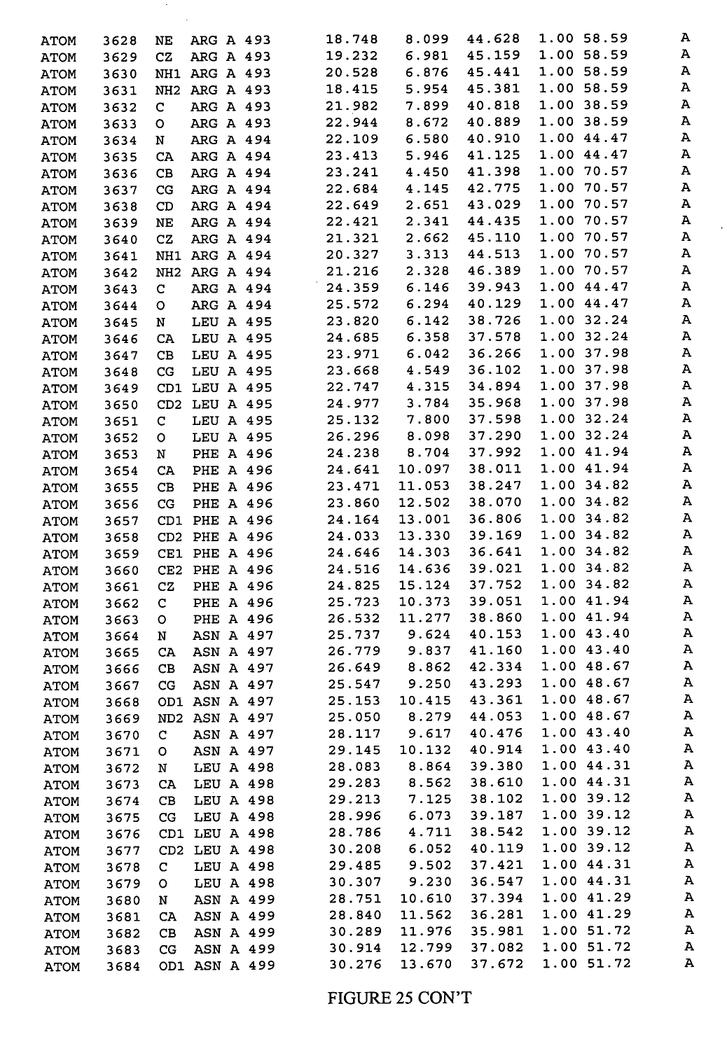

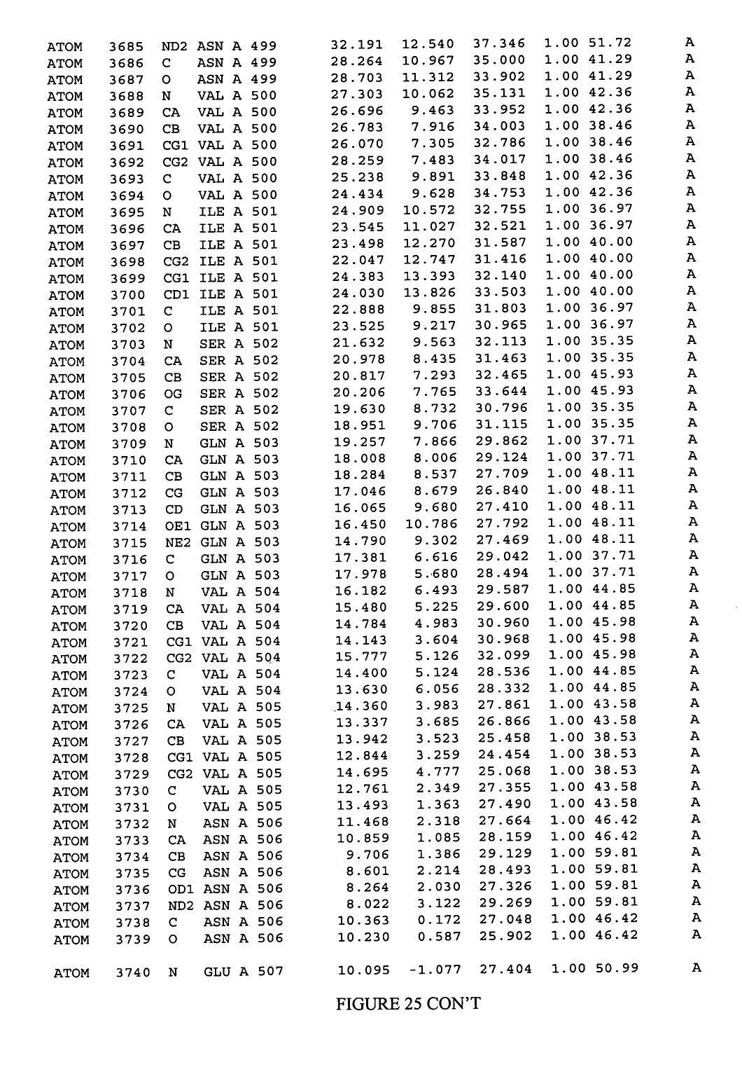

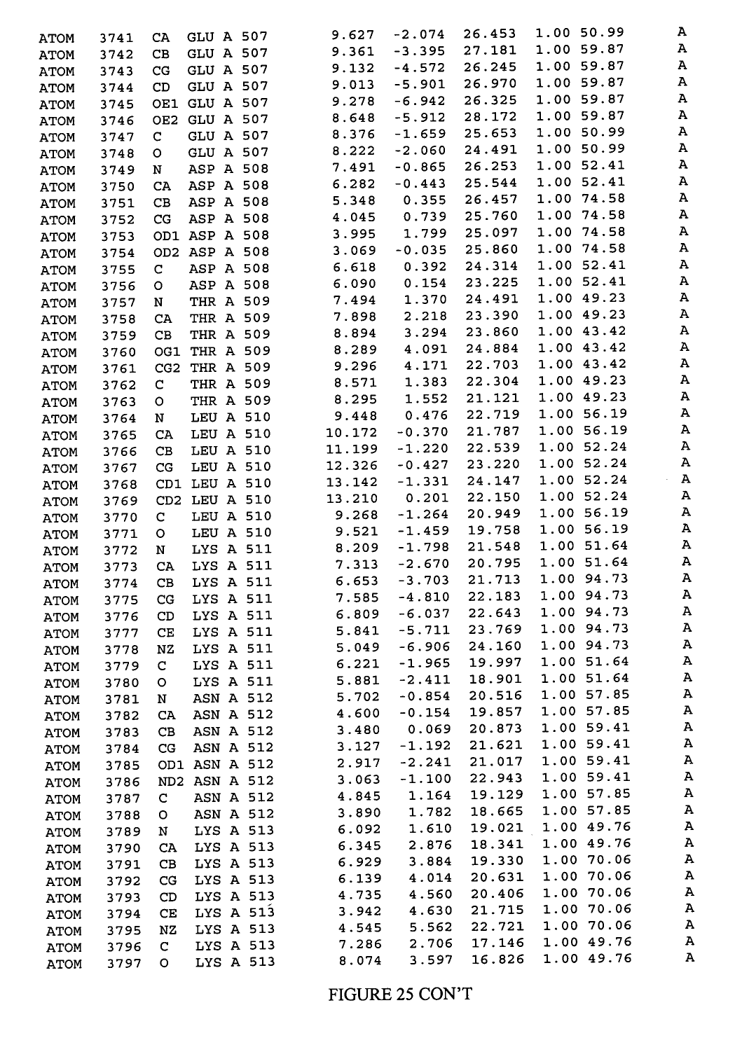

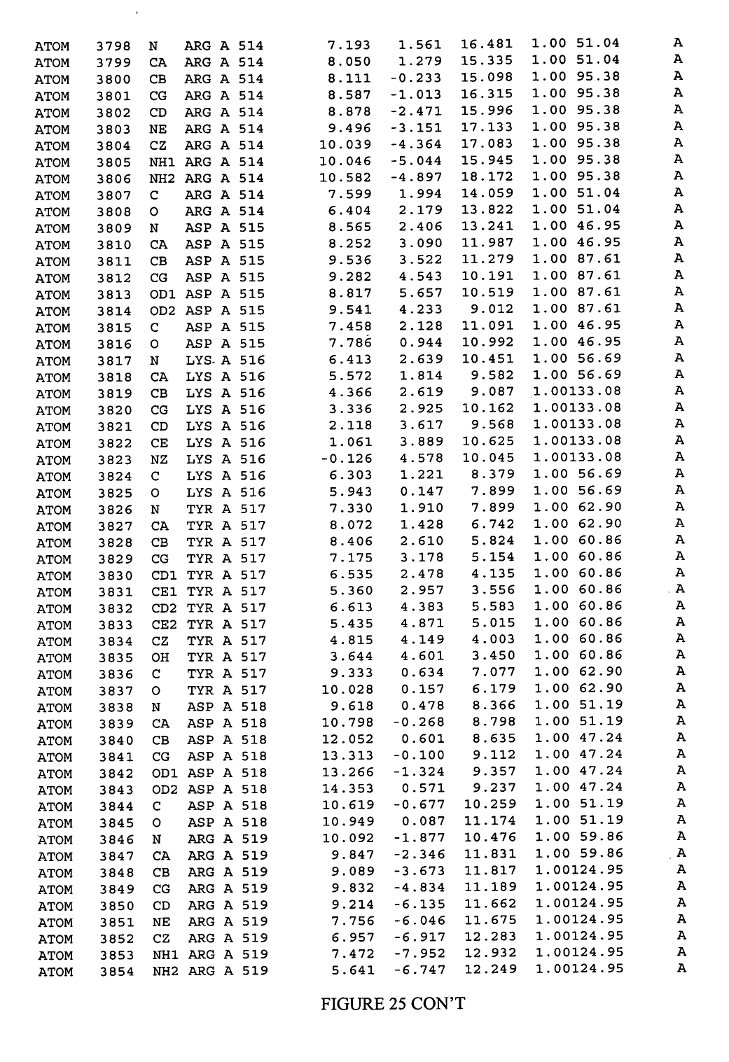

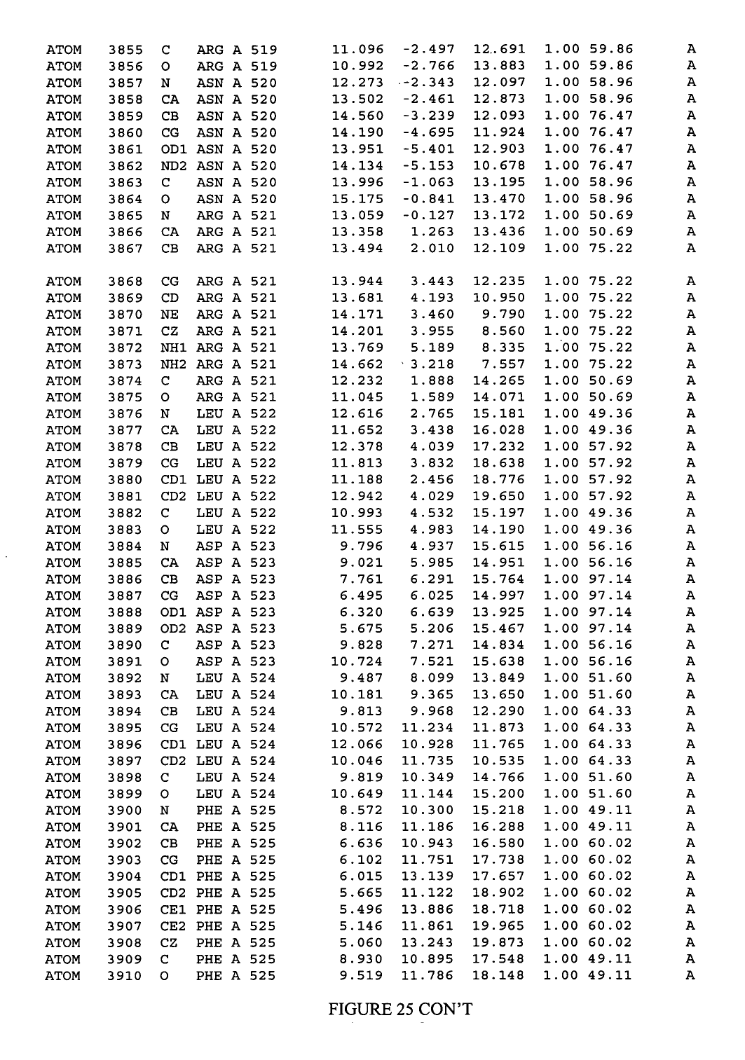

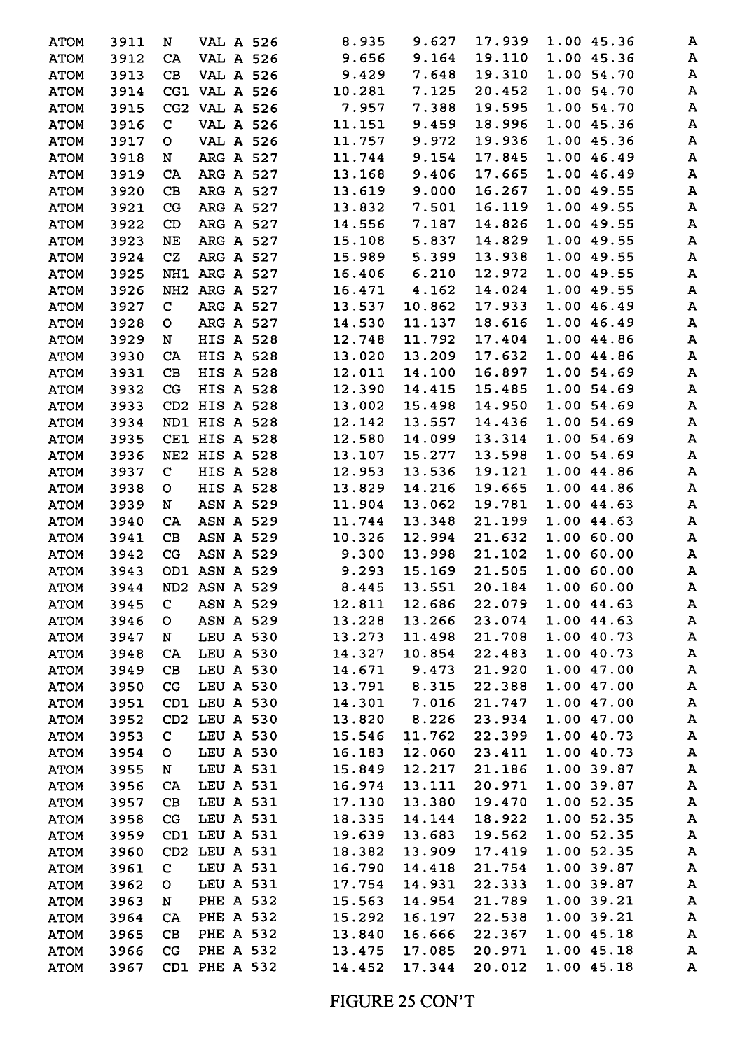

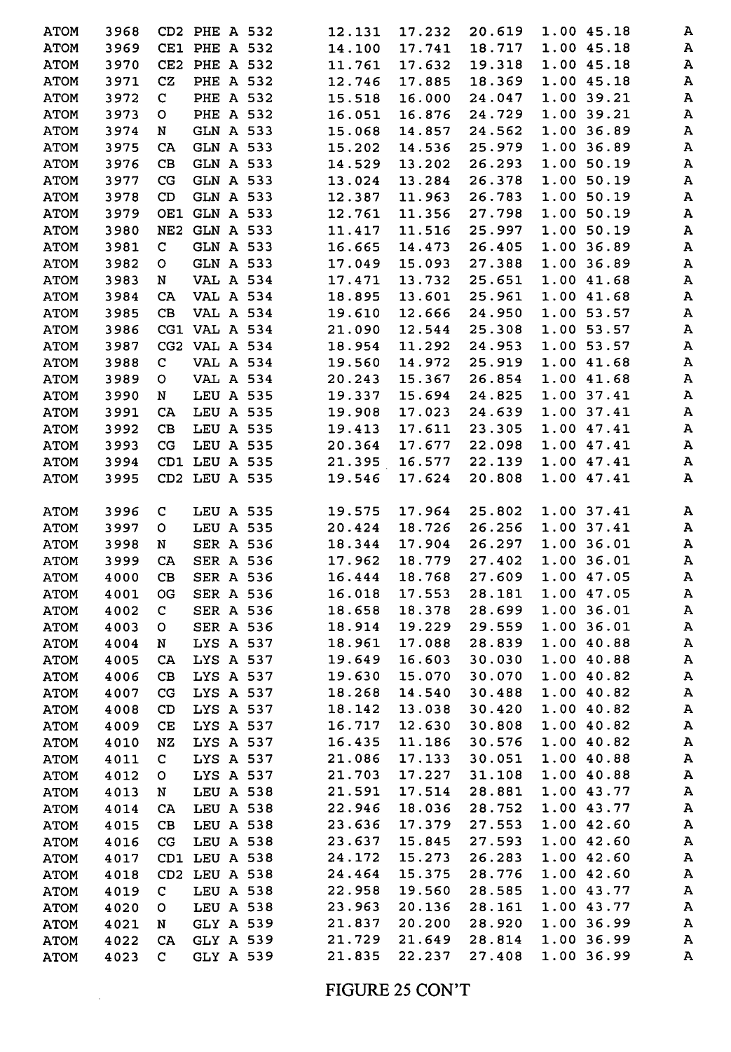

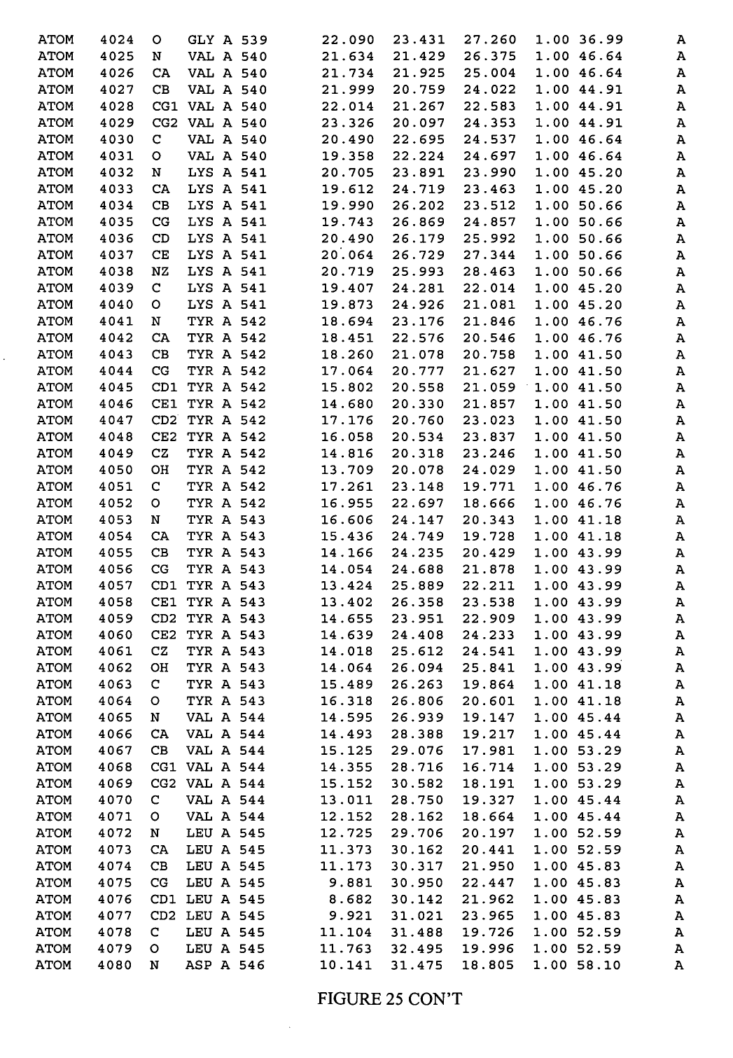

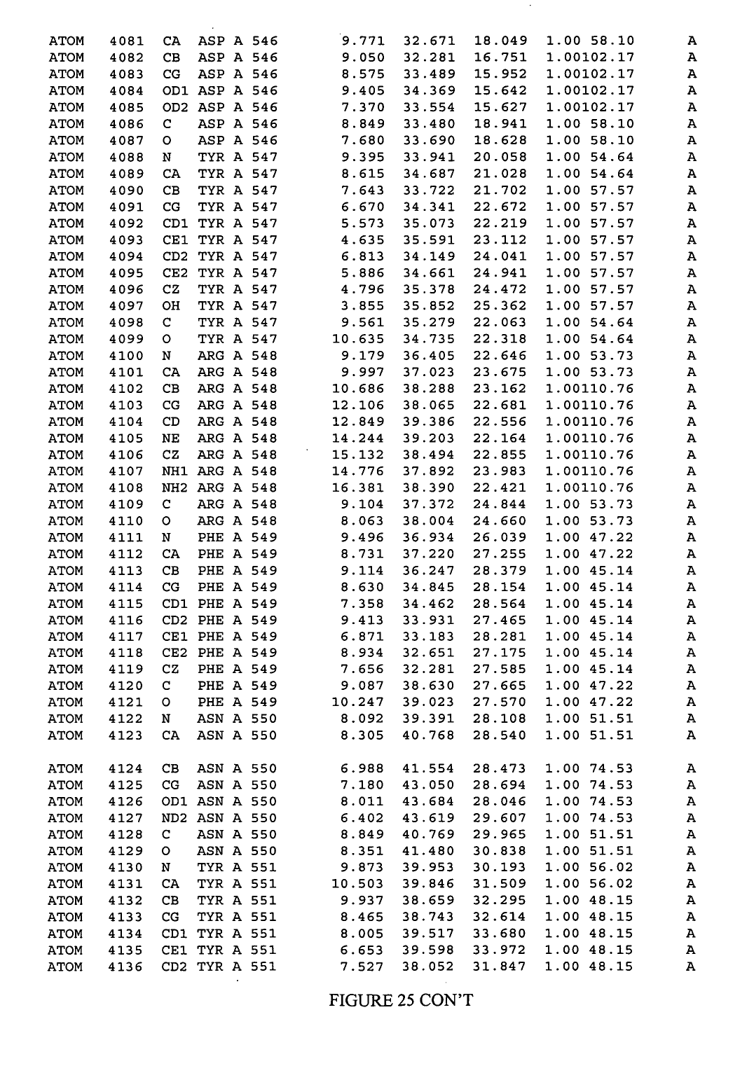

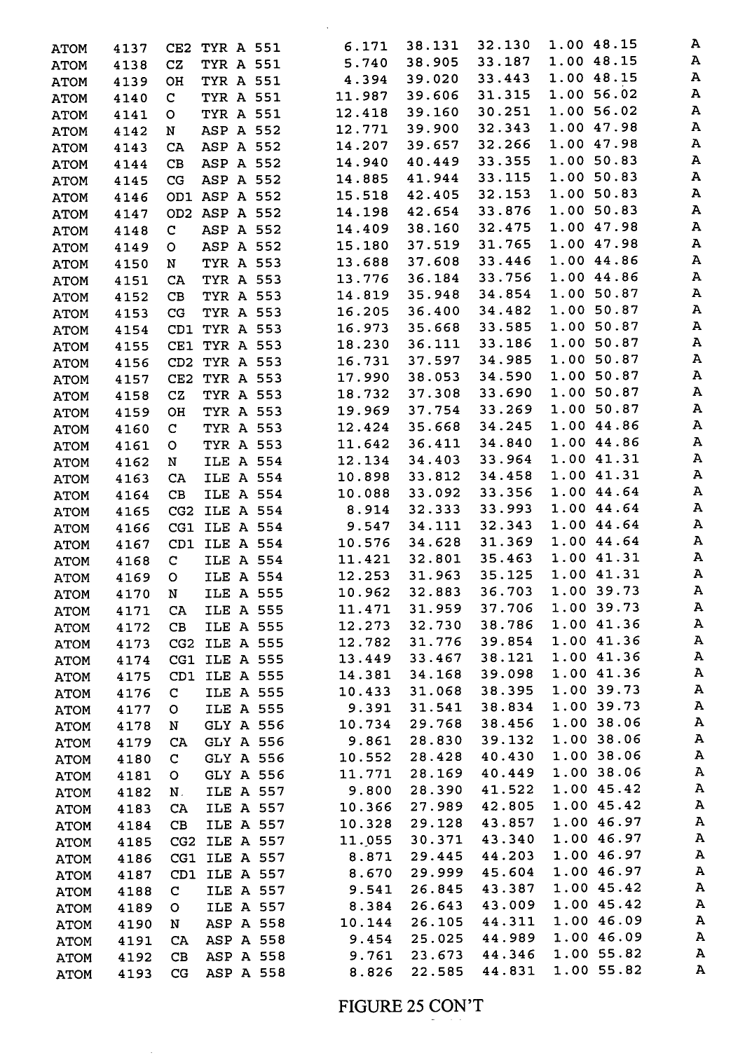

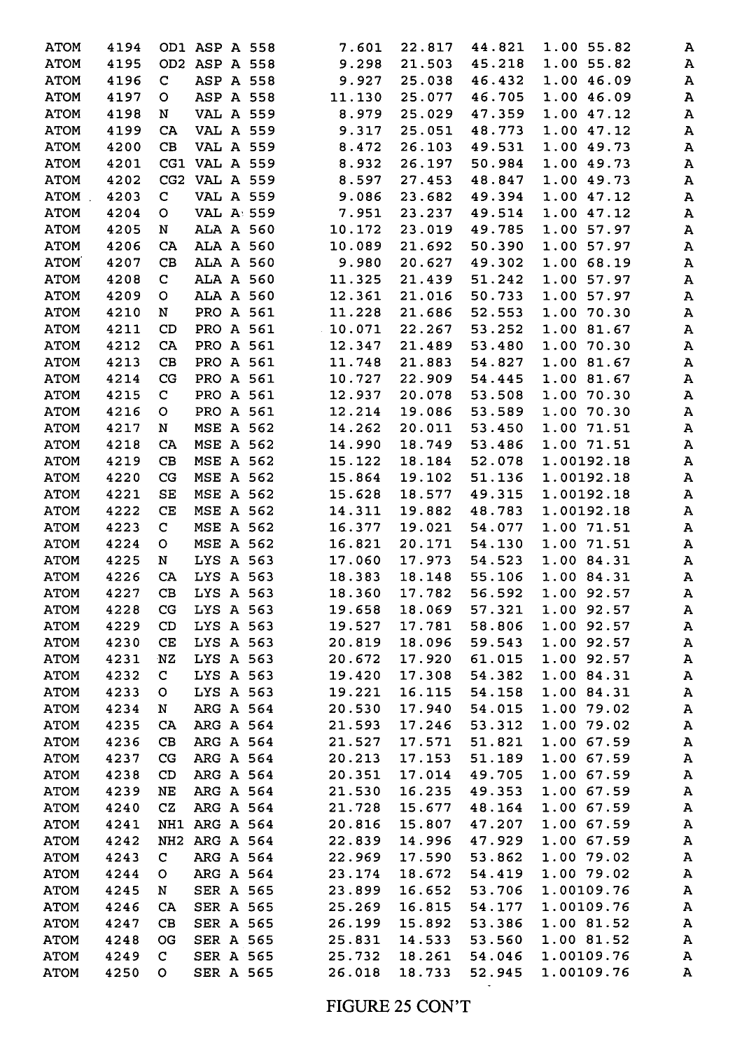

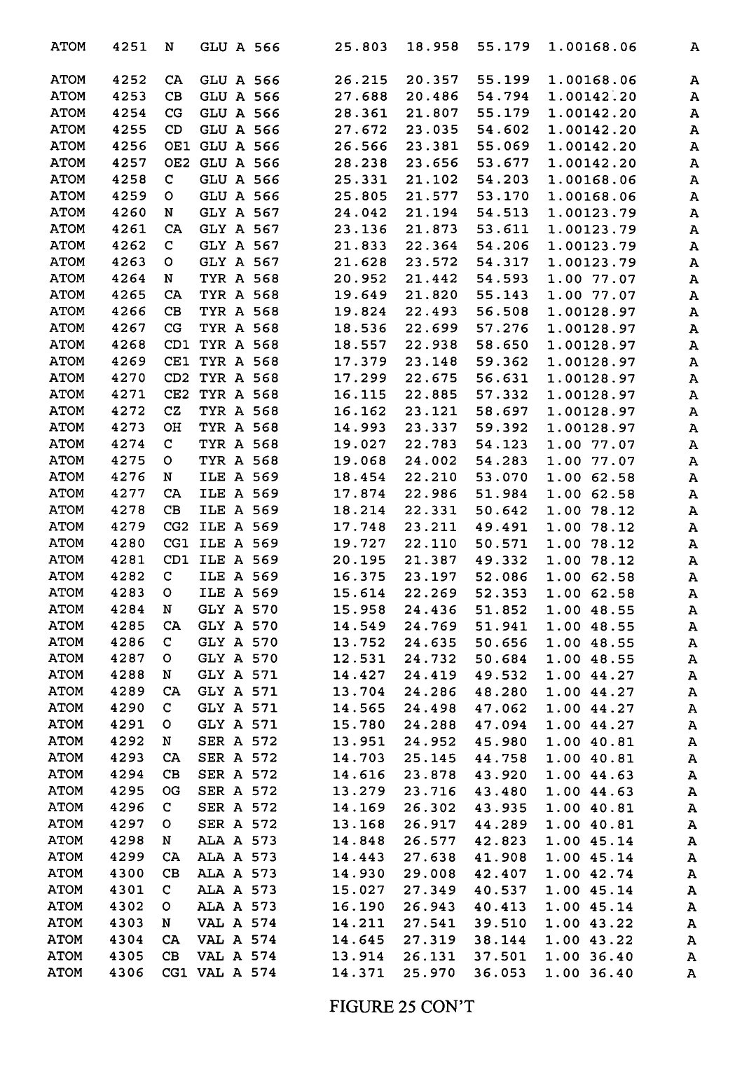

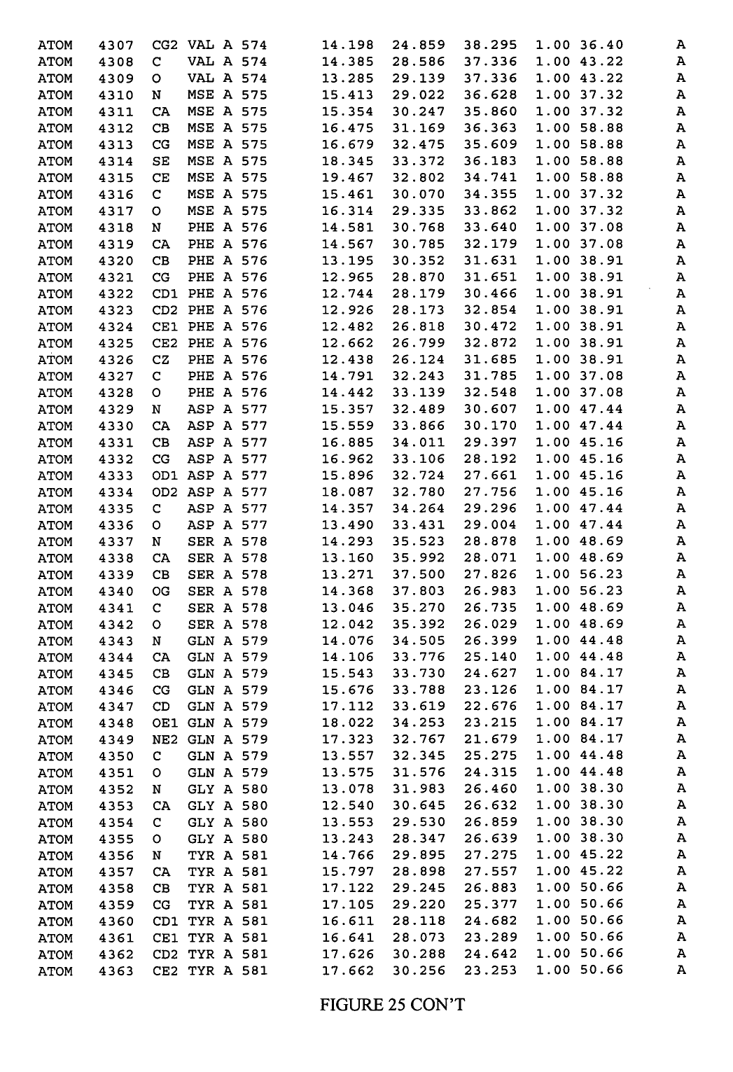

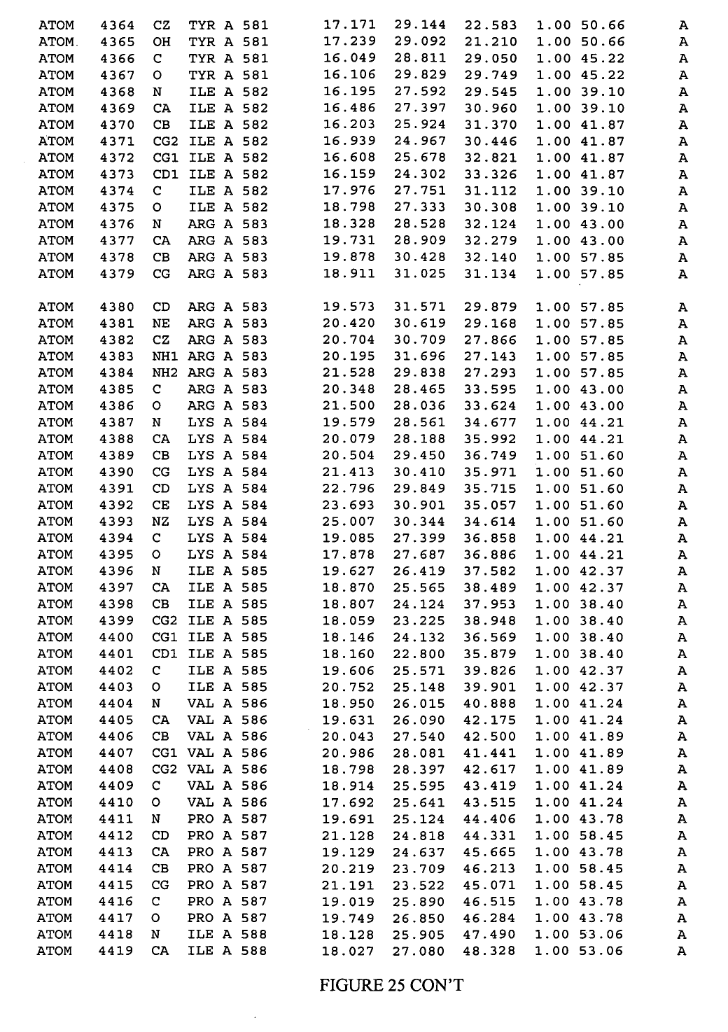

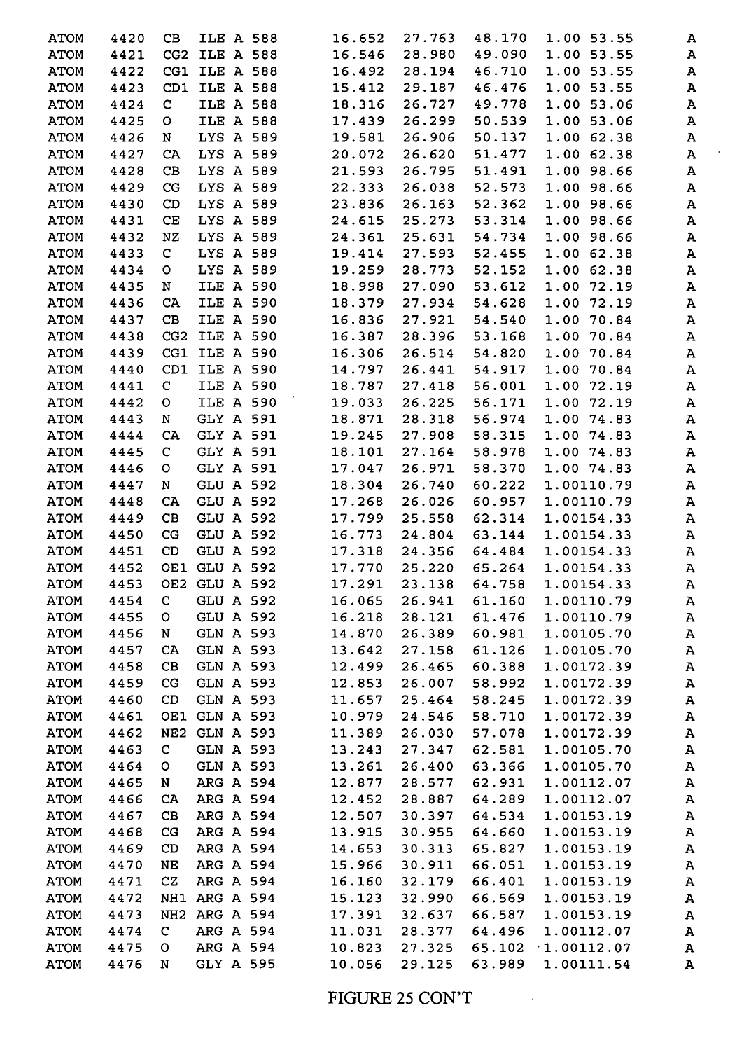

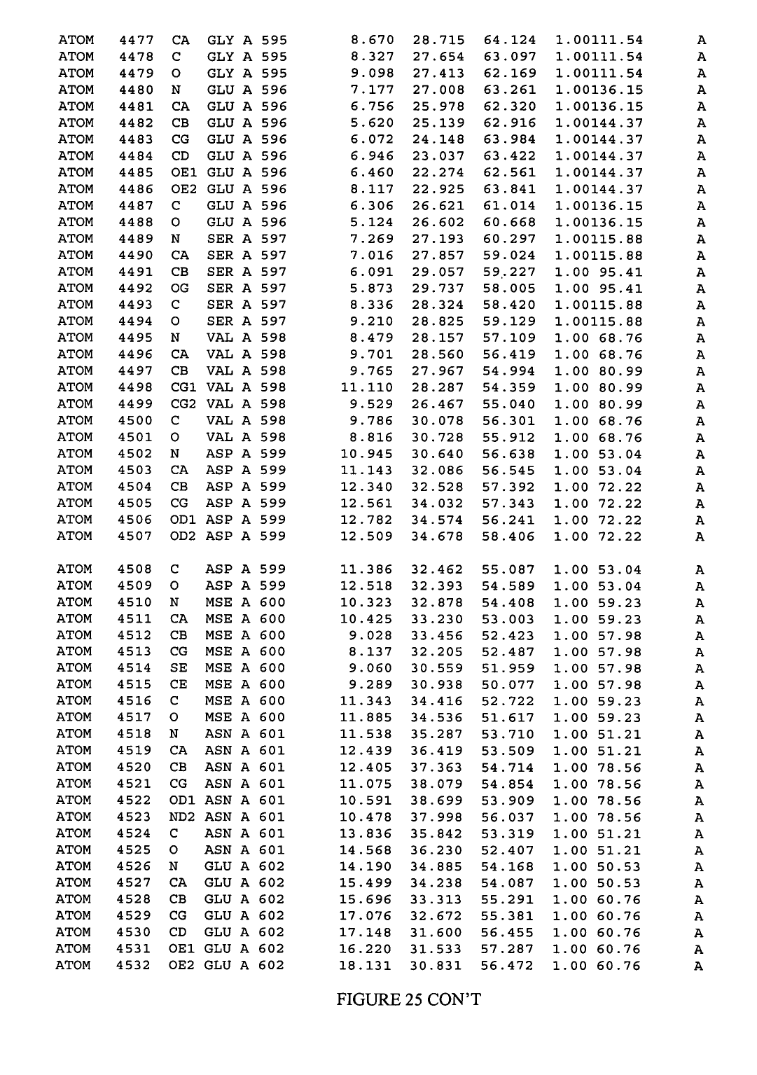

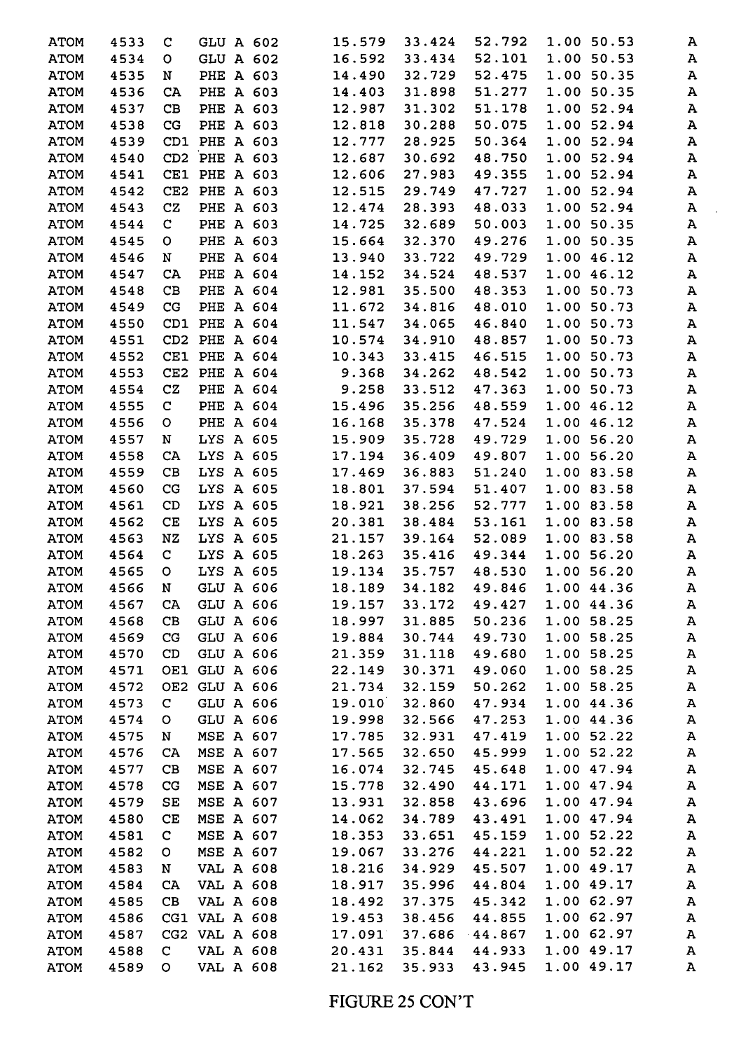

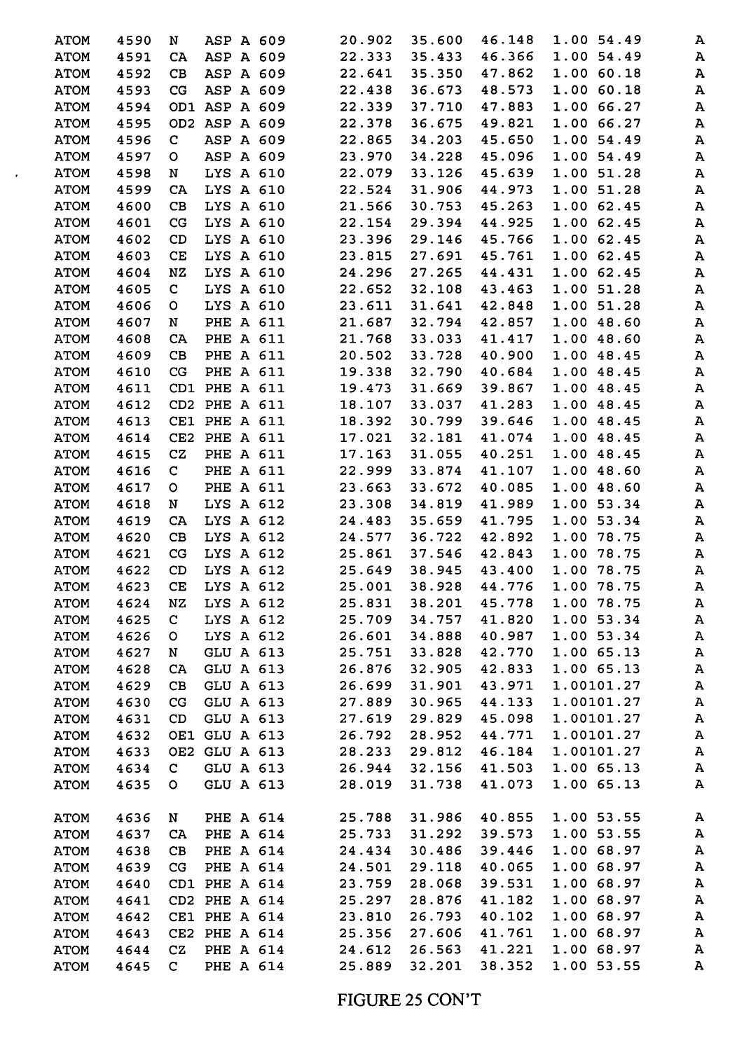

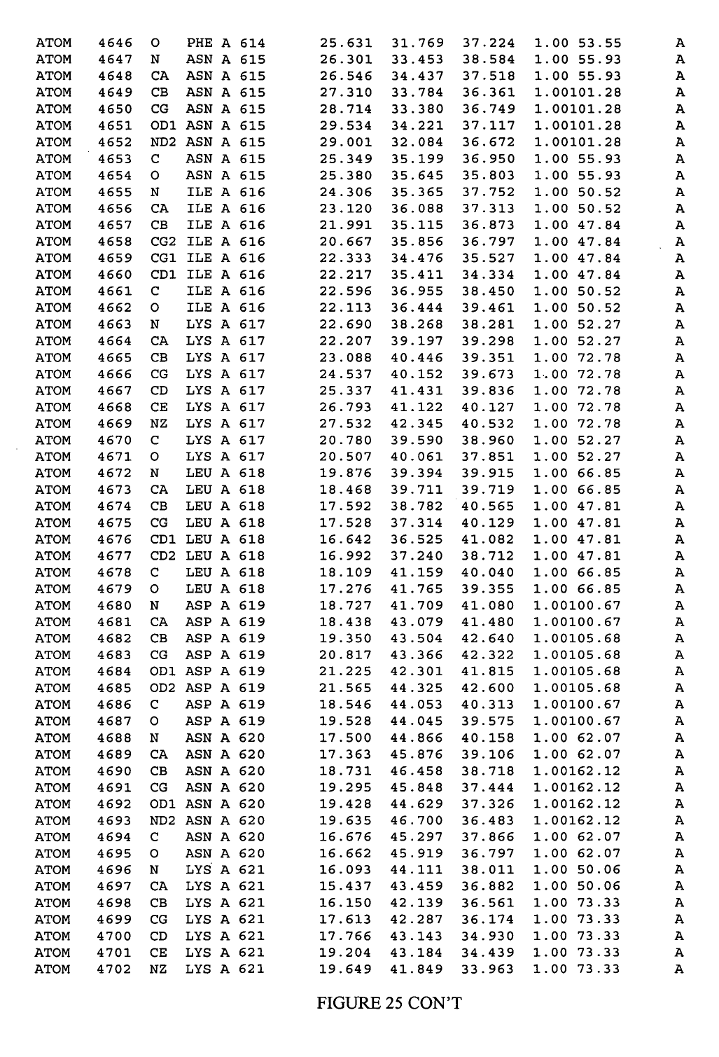

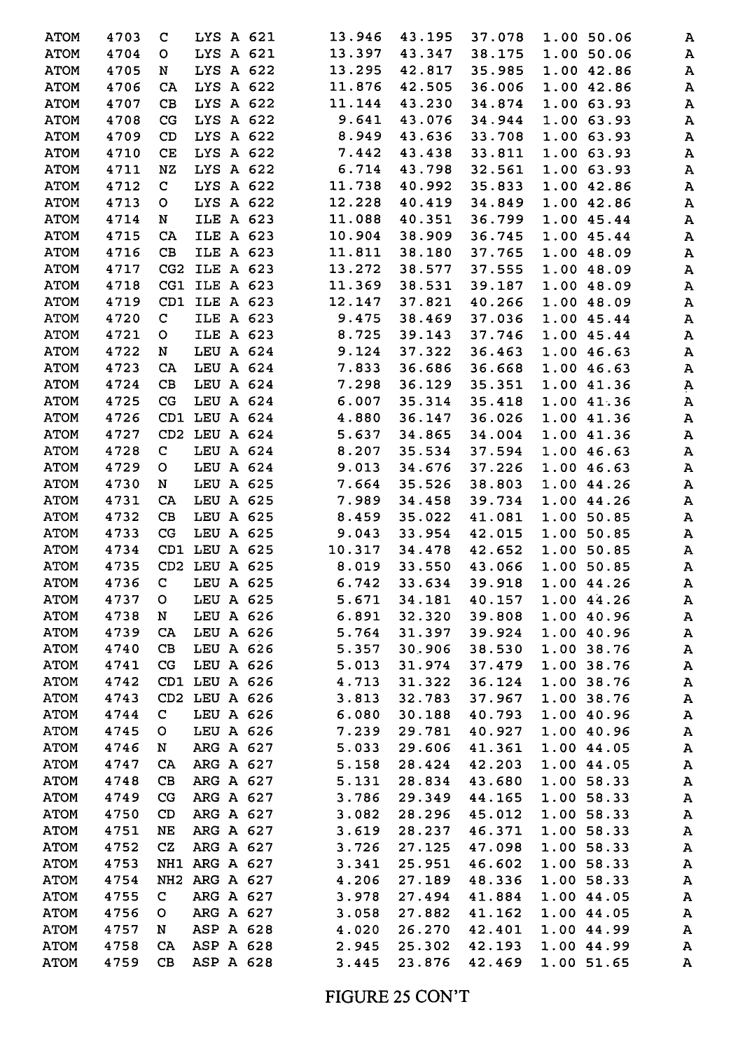

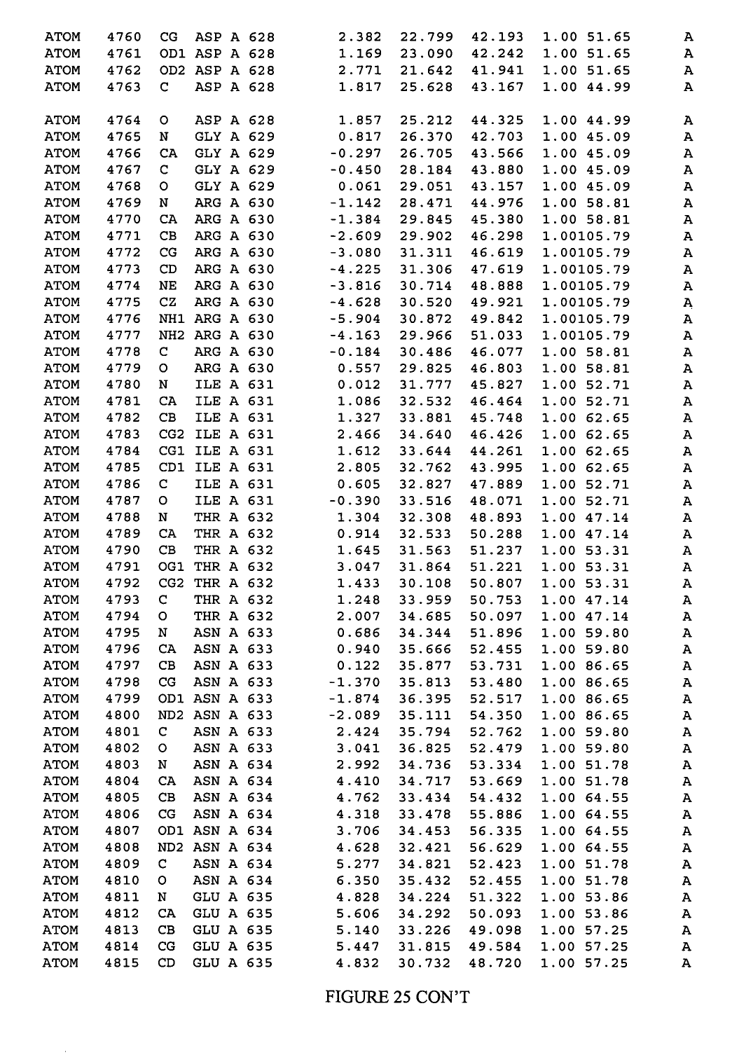

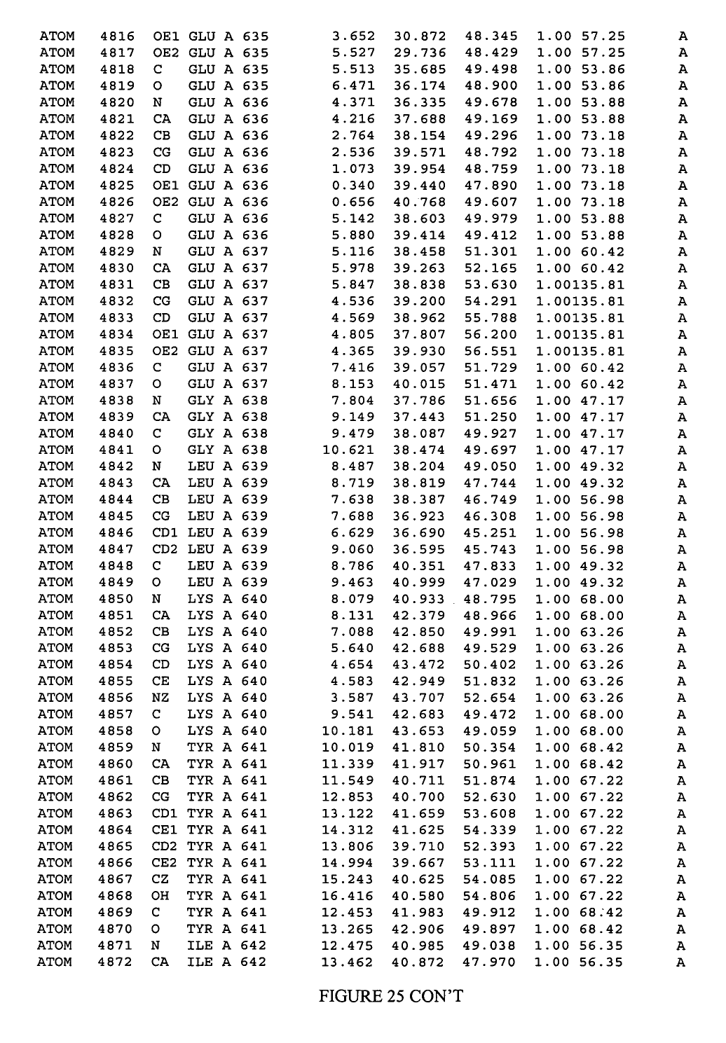

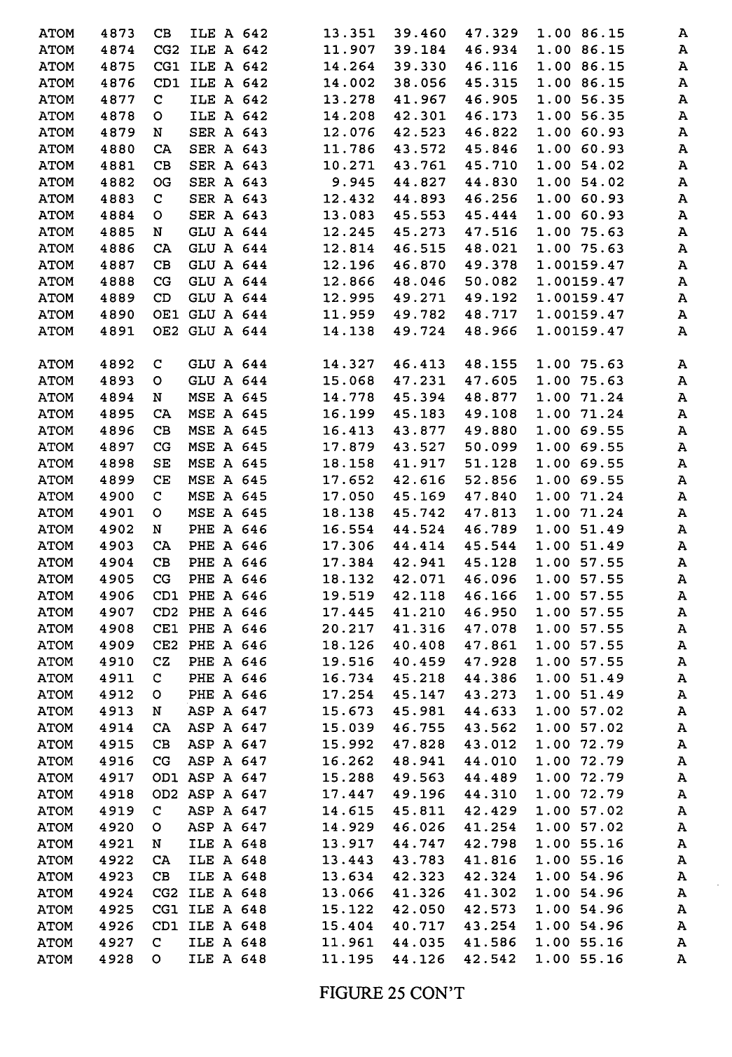

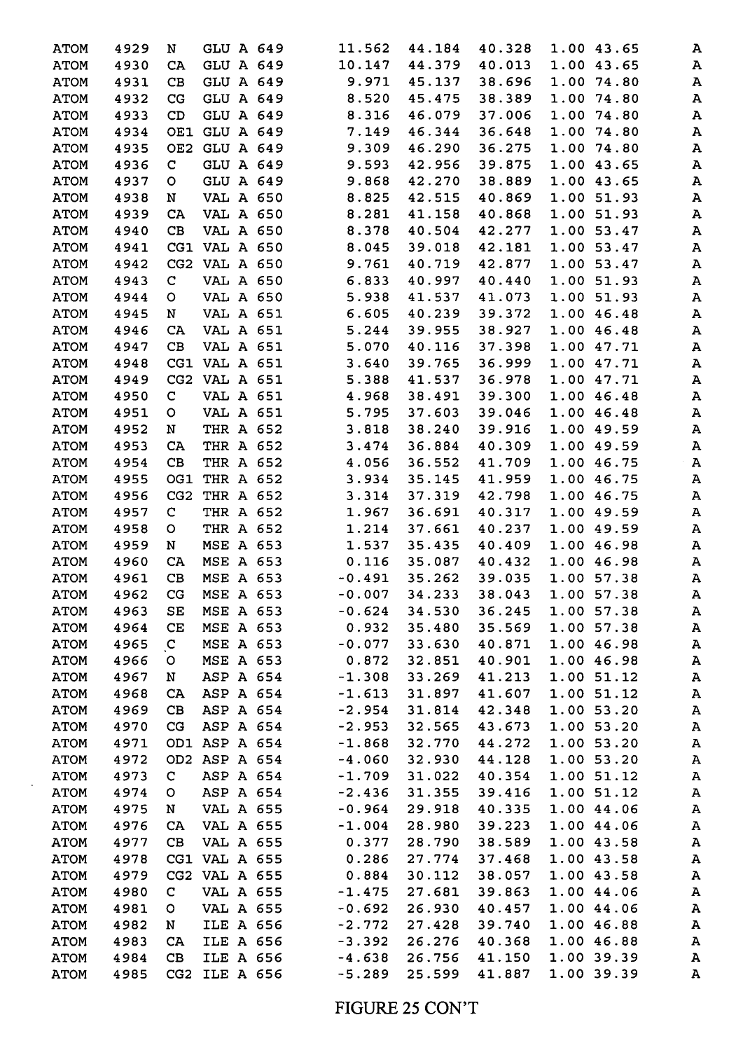

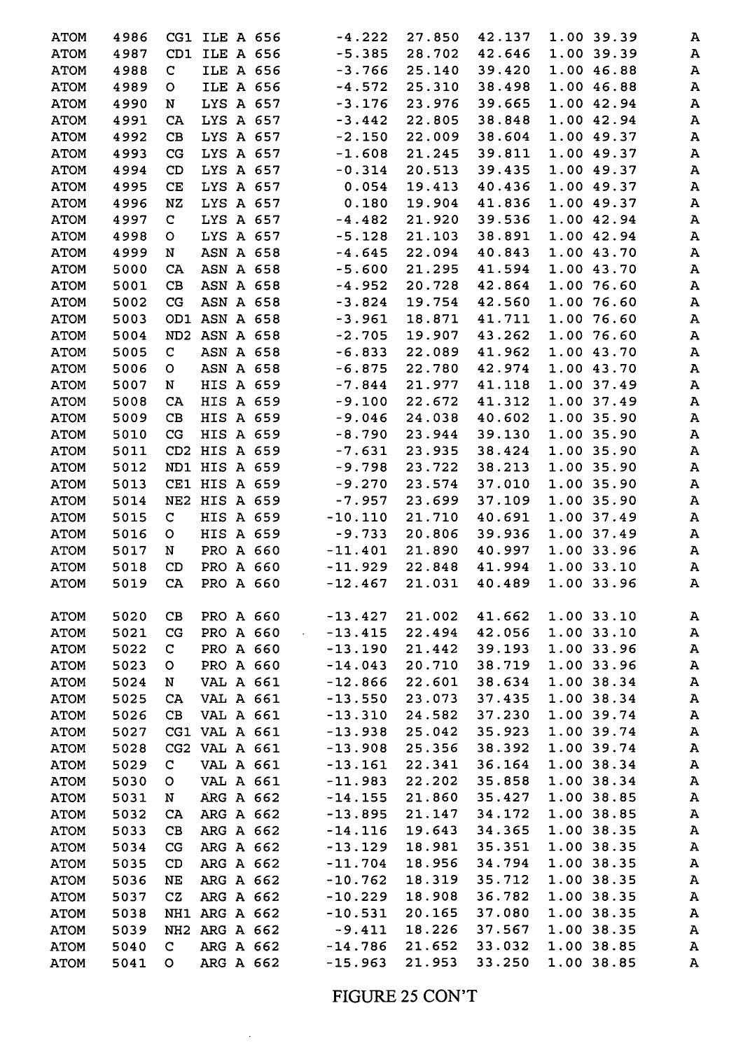

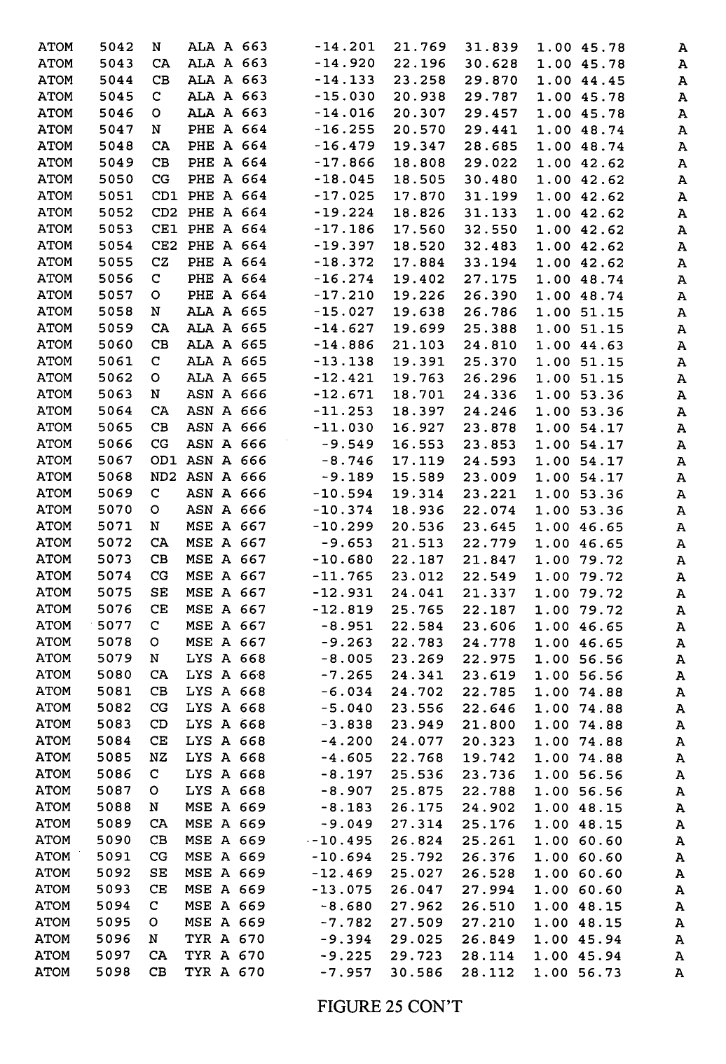

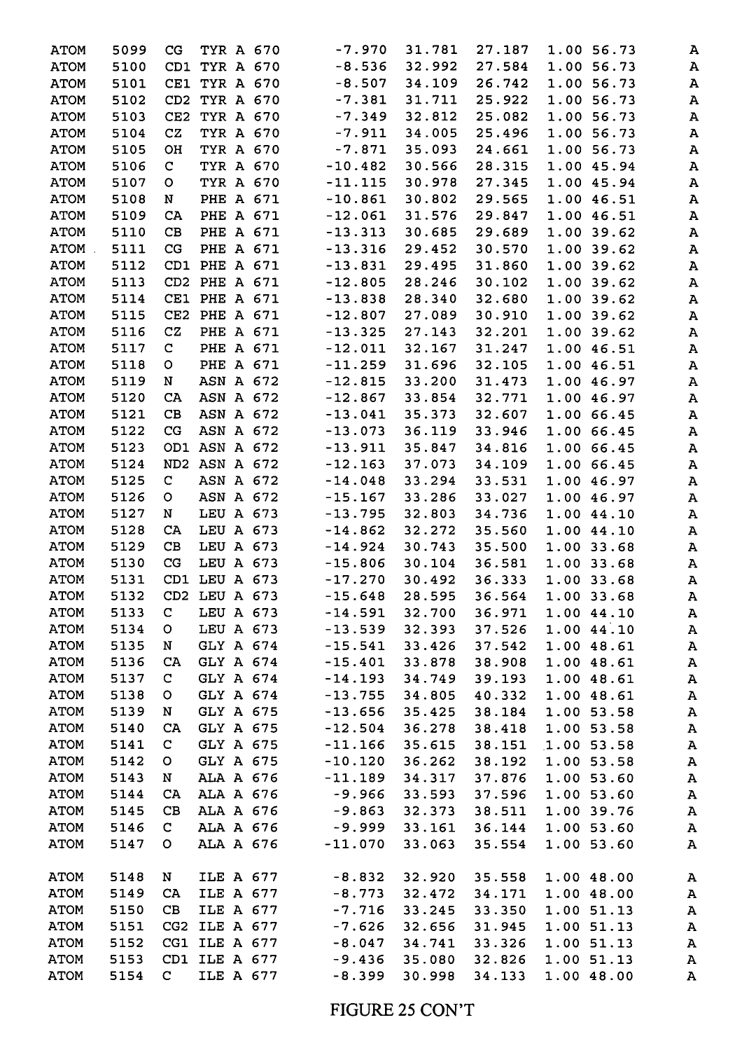

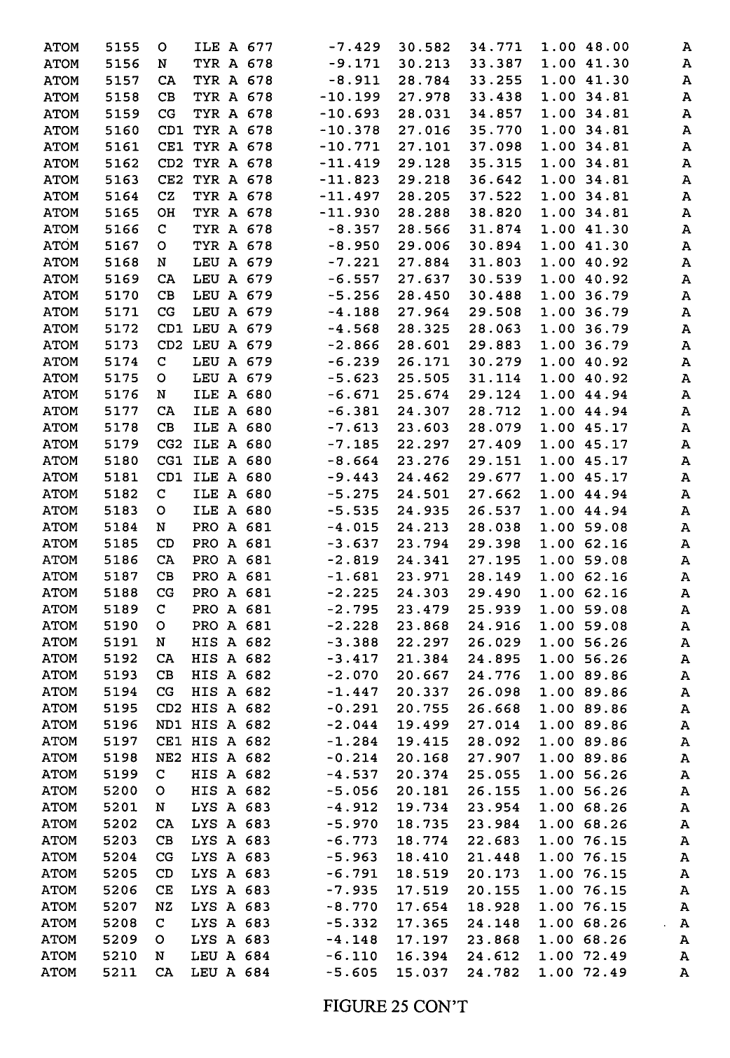

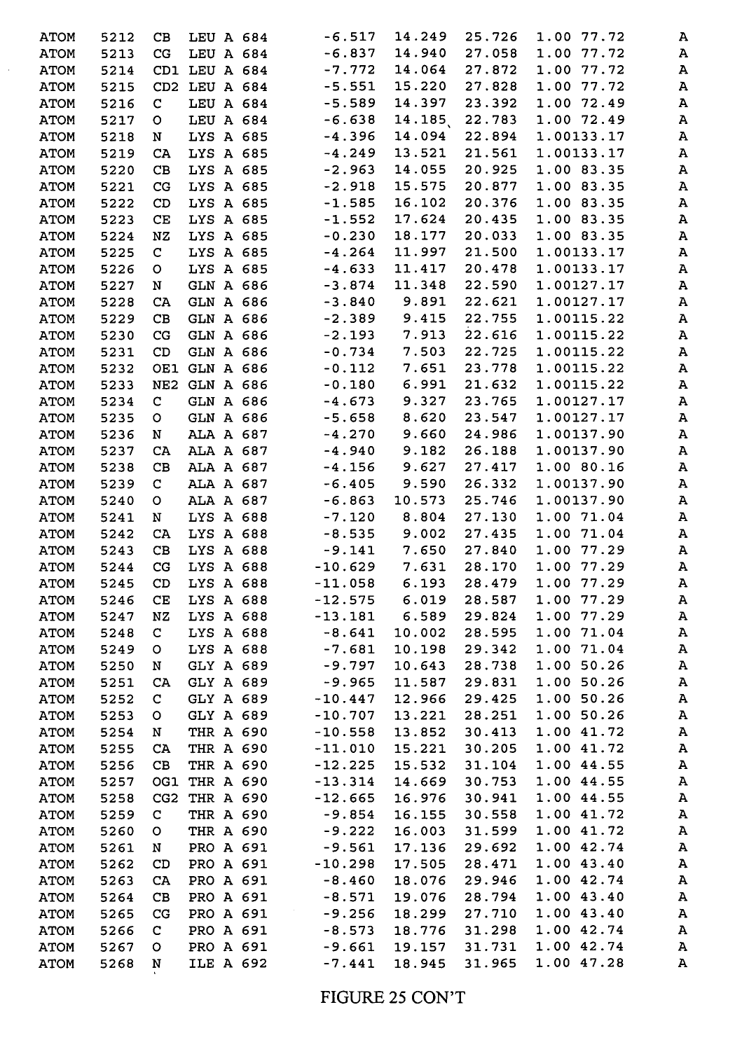

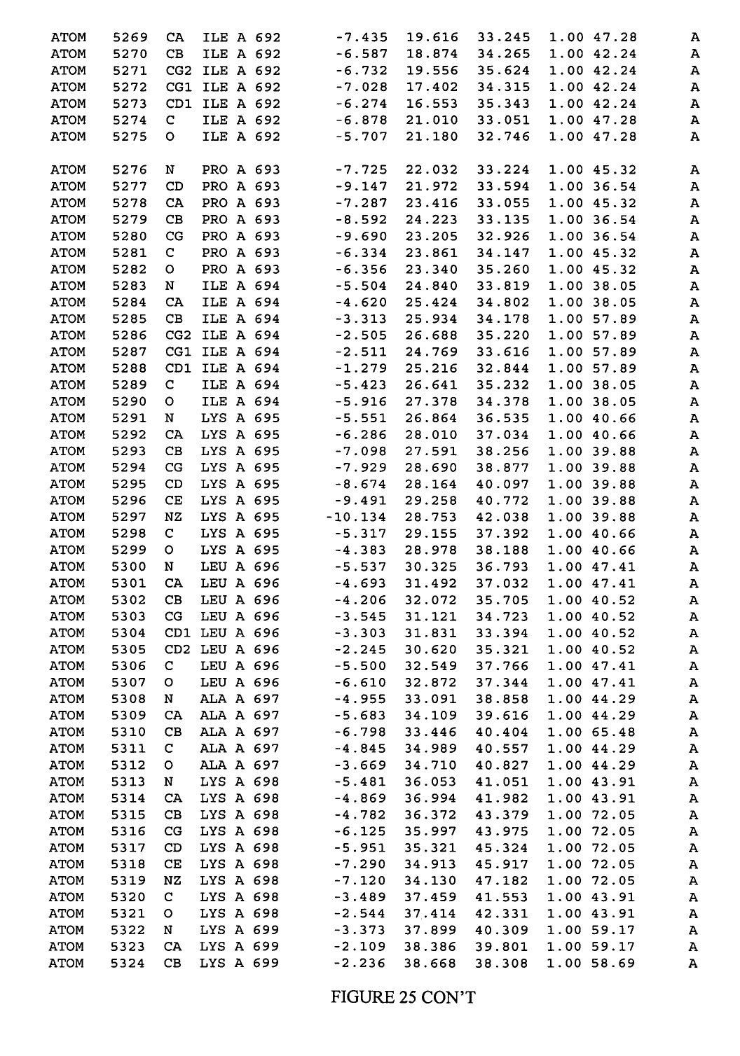

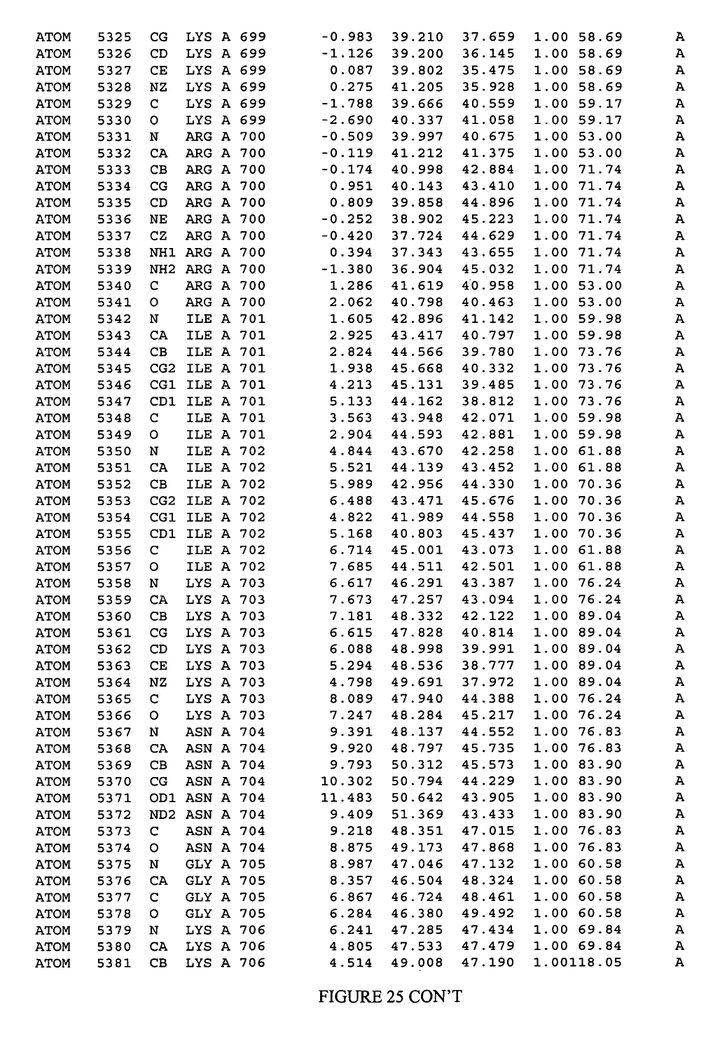

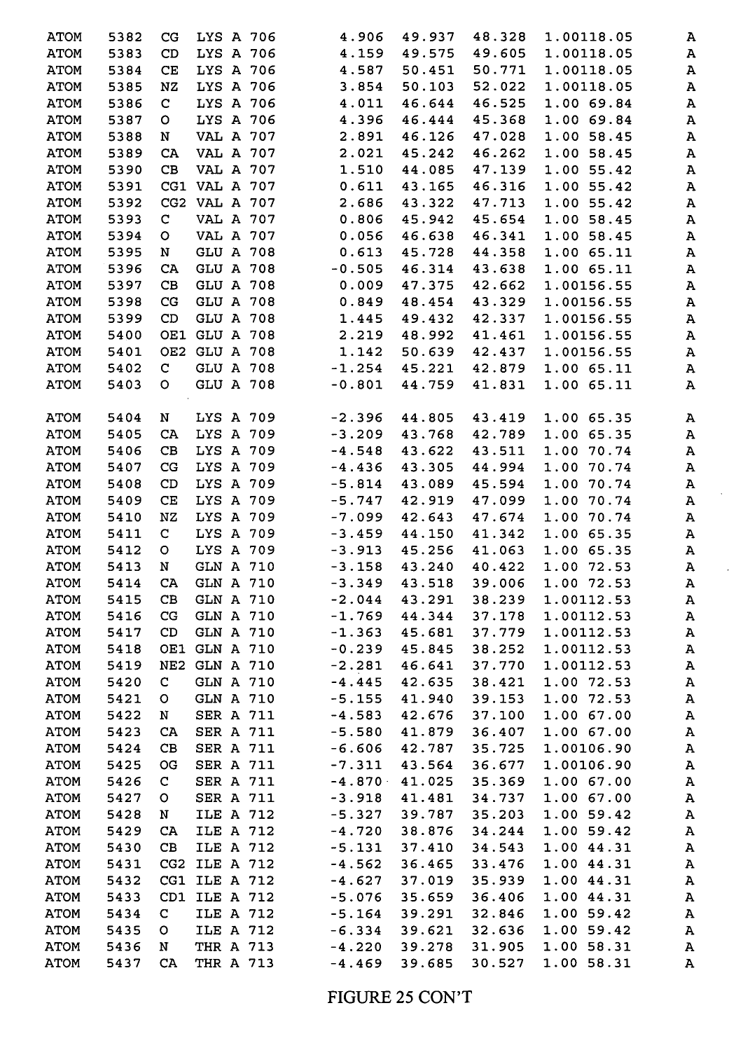

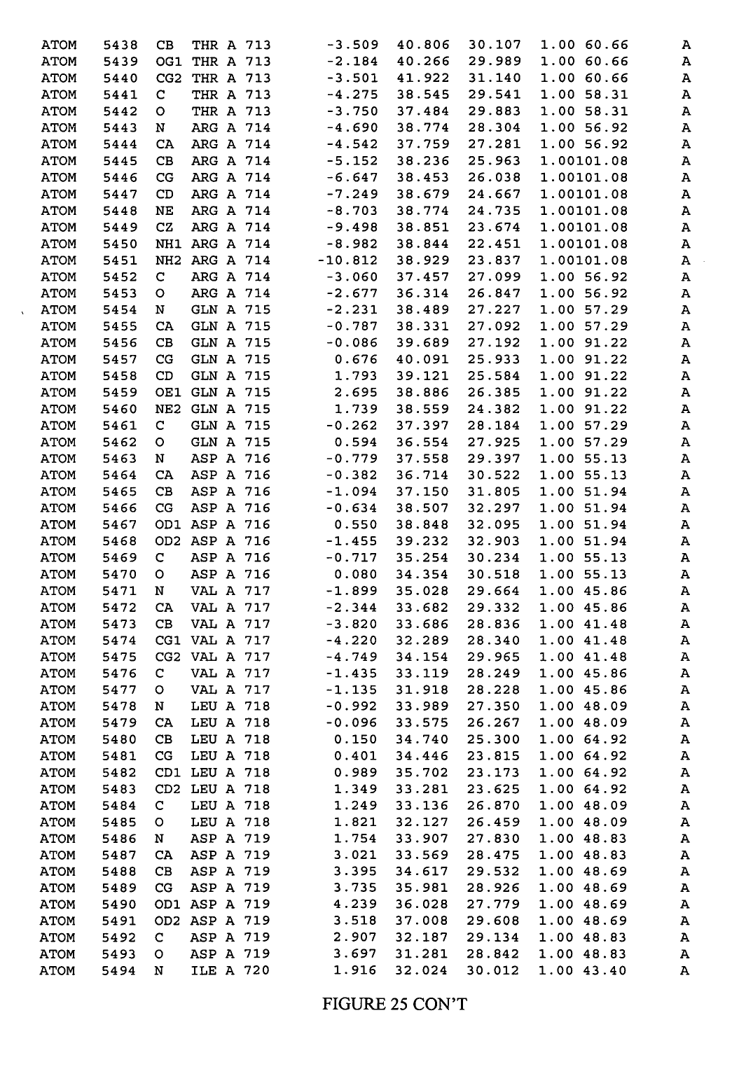

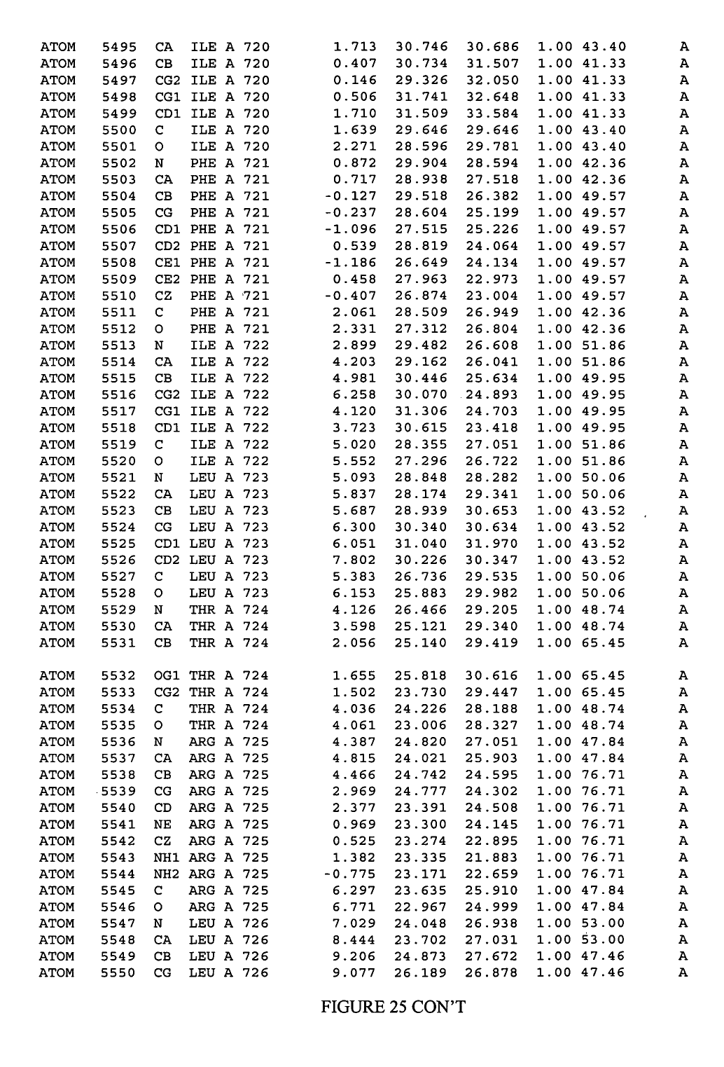

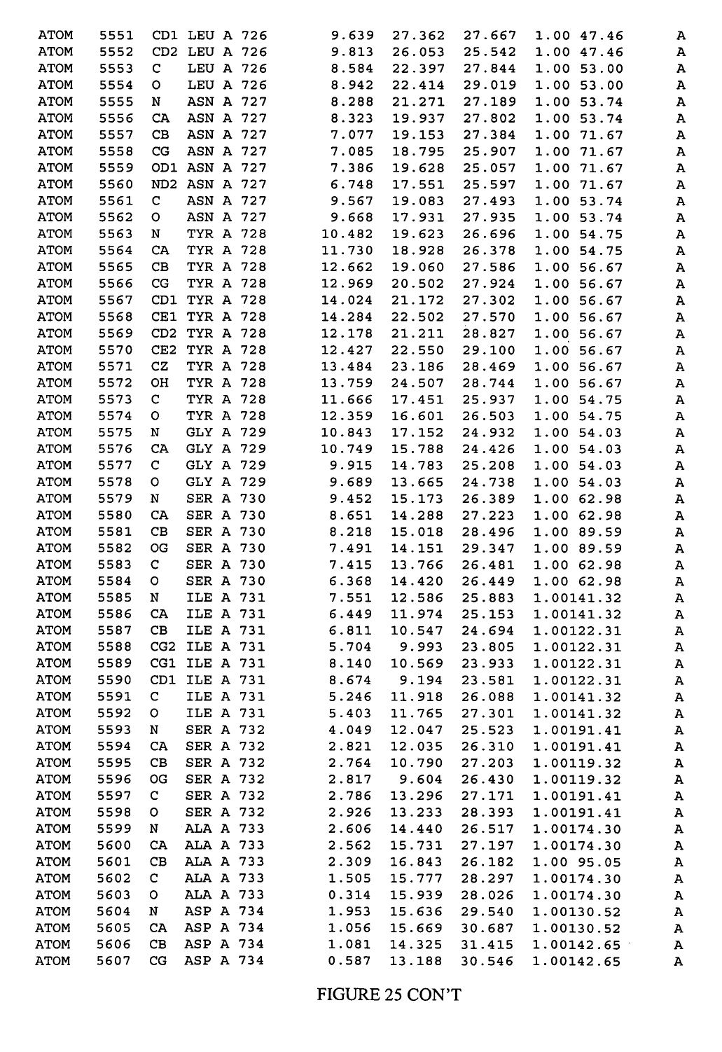

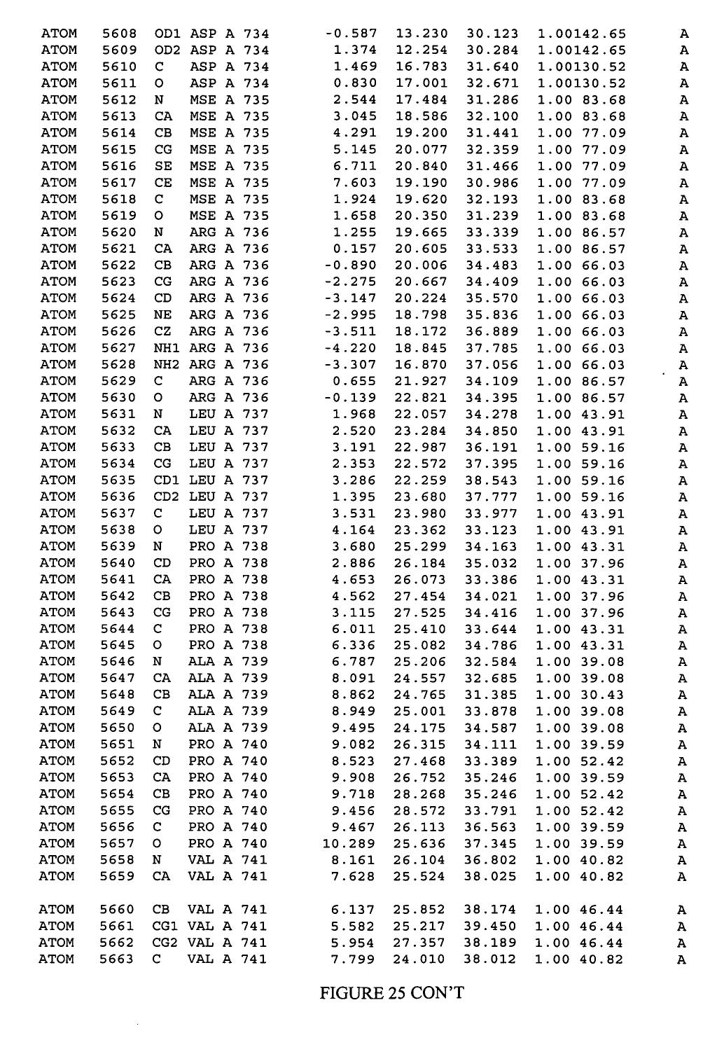

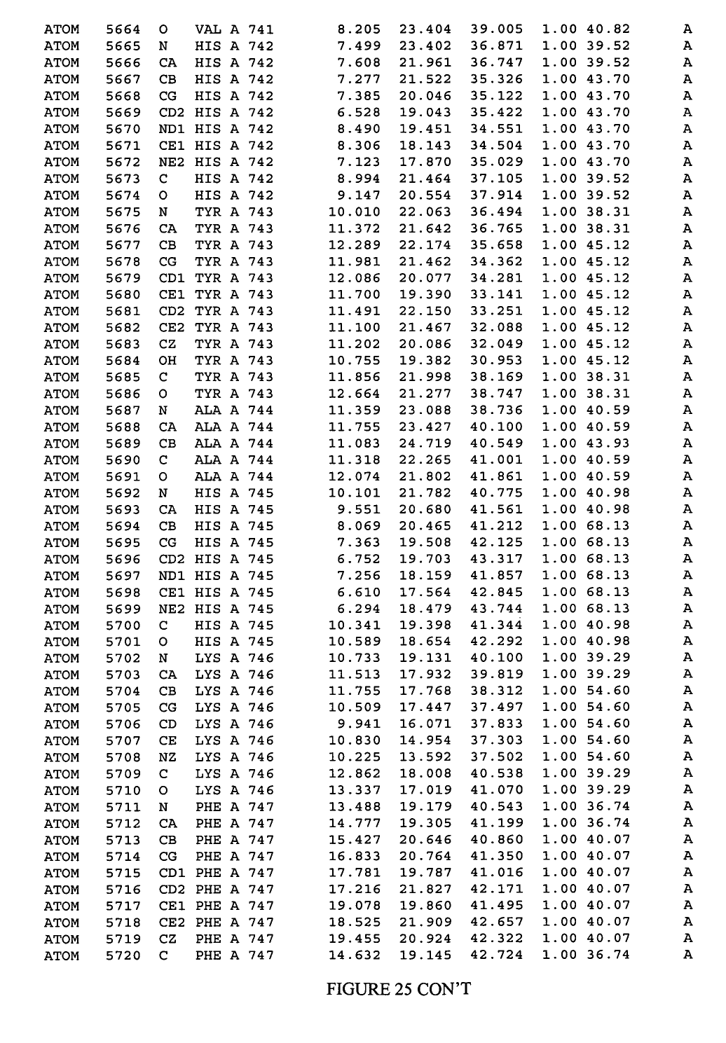

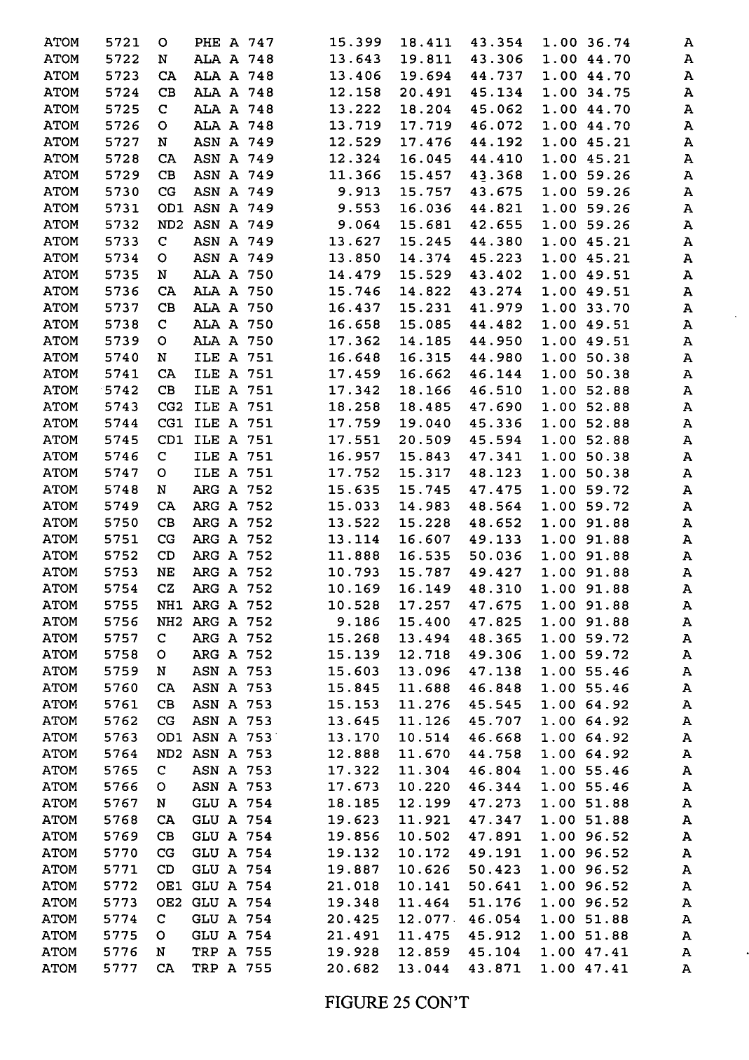

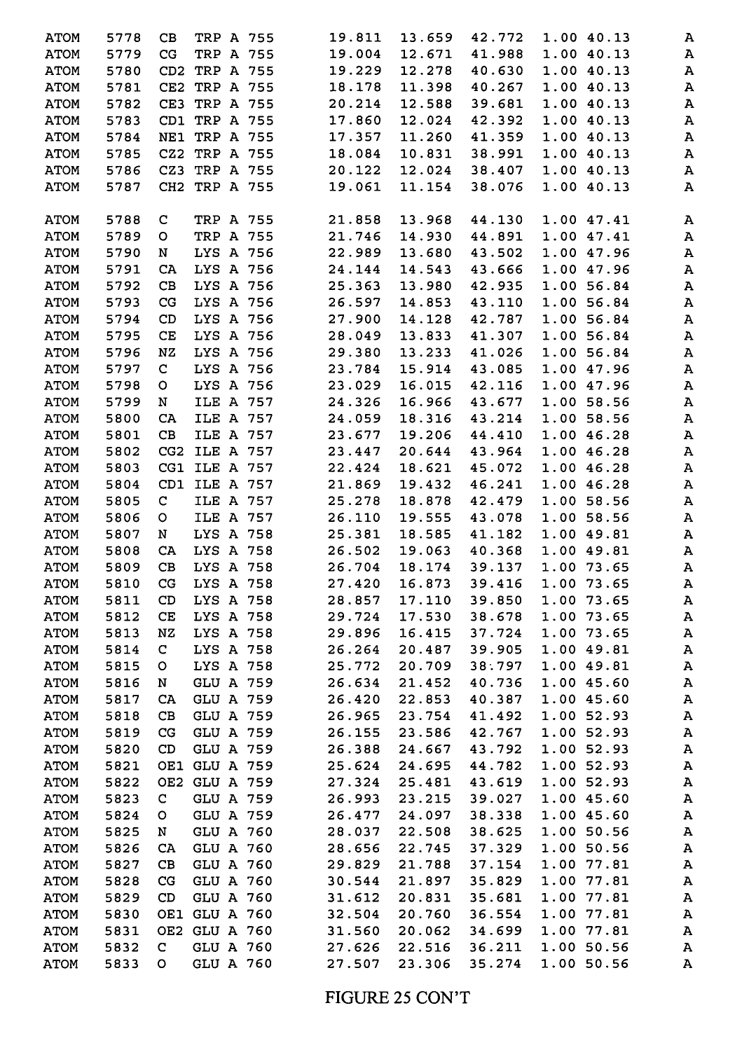

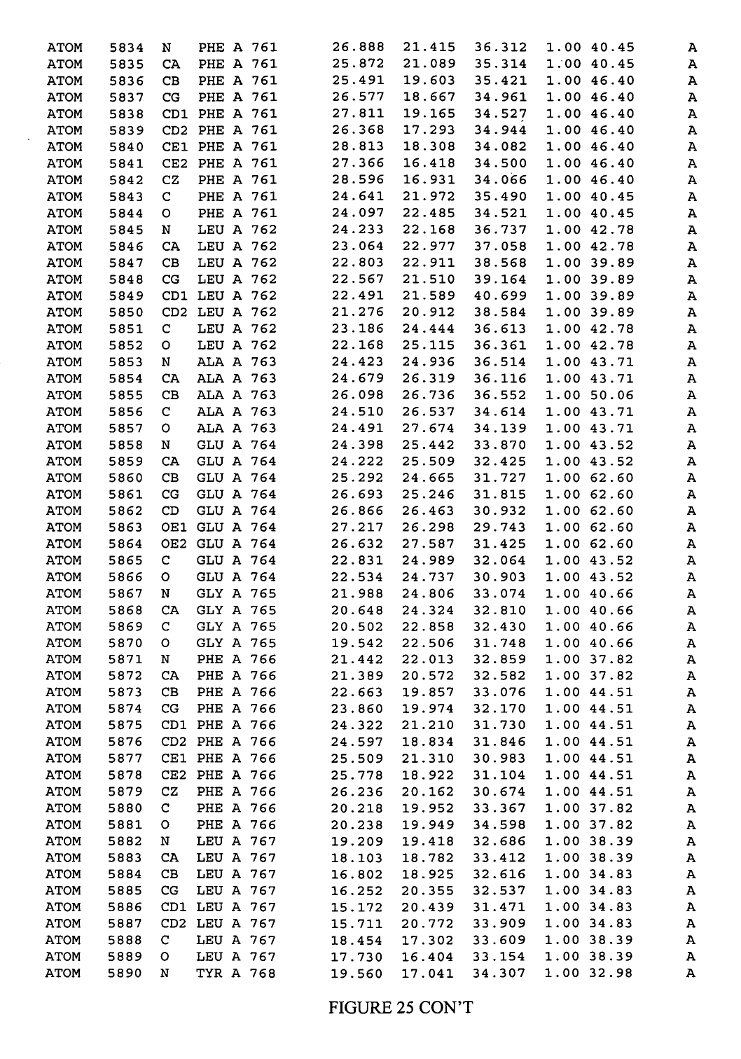

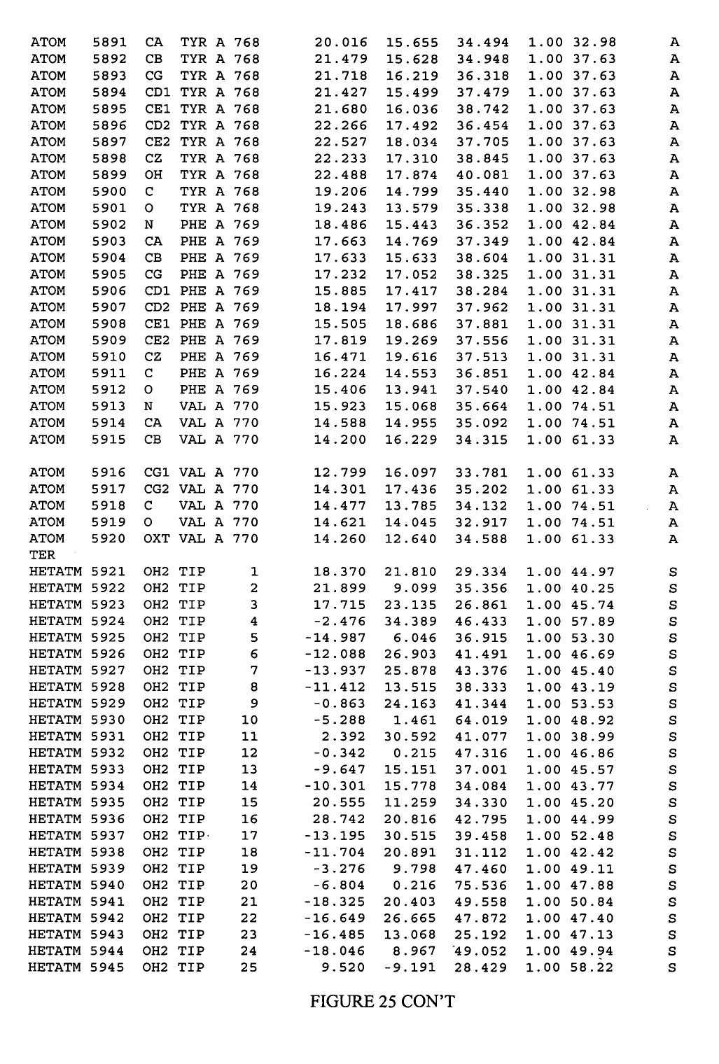

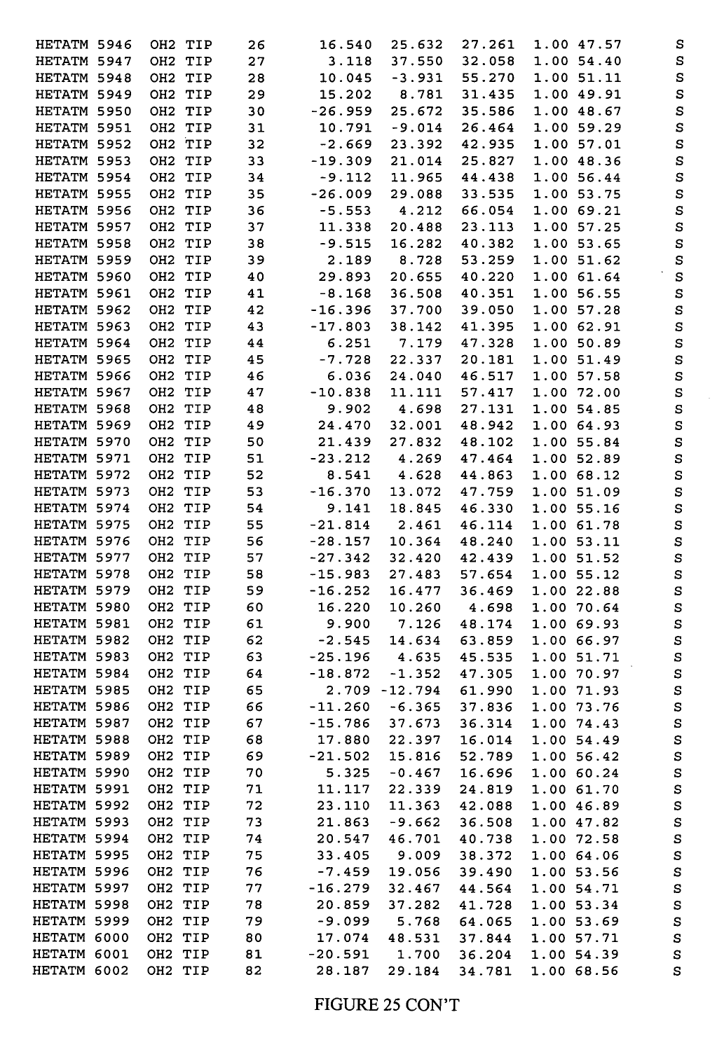

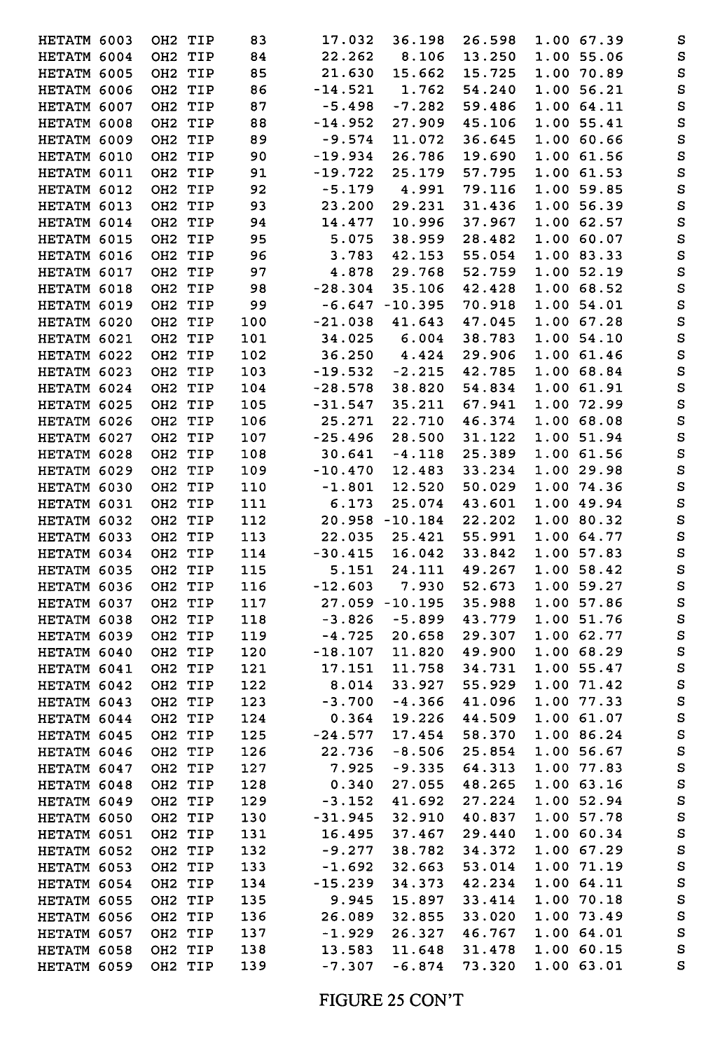

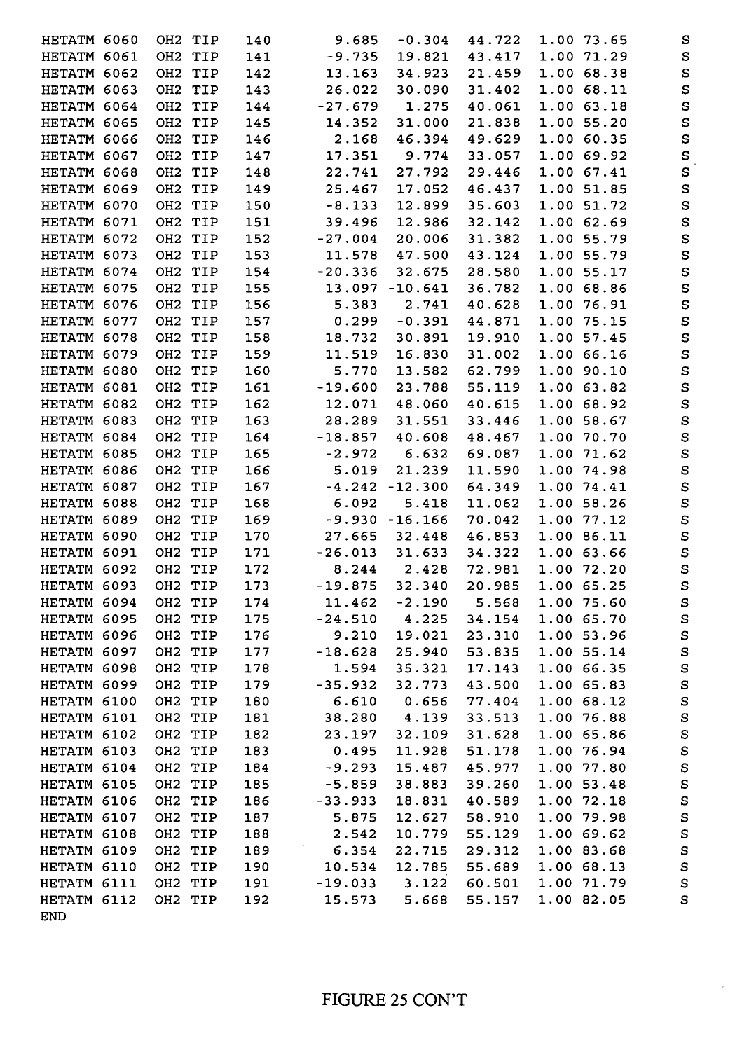

(FIG. 5B) A model for si-RNA and mRNA binding. Argonaute is shown as a ribbon representation in gray. A 3′ portion of the siRNA, shown in purple, was placed by superposition of the PAZ domain of the hAgo1-PAZ domain-RNA complex on the PAZ domain of PfAgo. The two nucleotides at the 3′-end of the siRNA are inserted in the PAZ cleft and the nucleotides 5′ to those bind along the PAZ domain. The passenger strand of the hAgo1-PAZ complex placed in a similar manner was used to model the mRNA strand, shown in light blue, by extending the RNA 2 nucleotides at the 5′-end, and from the middle of that strand along the binding groove towards the active site in PIWI. The 5′-end of the mRNA is nested between the PAZ and N-terminal domains, across the stalk. The phosphate between the 11th and 12th nucleotides from the 5′-end of the mRNA falls near the active site residues shown in red.

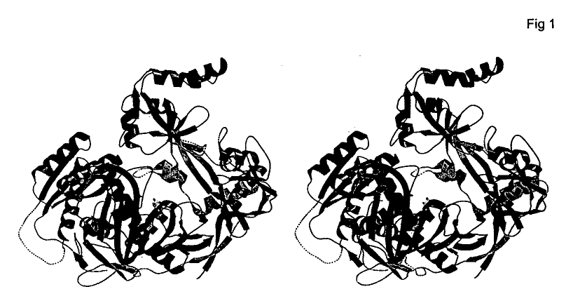

(FIG. 5C) Schematic depiction of the model for siRNA-guided mRNA cleavage. The domains are colored as in FIG. 1. The siRNA, shown in yellow, binds with its 3′-end in the PAZ cleft and the 5′ is predicted to reach the other end of the molecule and likely bind there. The mRNA is depicted in brown, comes in between the N-terminal and PAZ domains and out between the PAZ and middle domain. The active site in the PIWI domain, depicted as scissors, cleaves the mRNA opposite the middle of the siRNA guide.

FIG. 6 shows sequence alignment of the PAZ domains of PfAgo, hAgo1 and DmAgo2 based on the structural superposition of the three domains. The sequence of PfAgo-PAZ domain could not be readily aligned with PAZ domains from other species without knowledge of the structure. Invariant residues are highlighted in purple and conserved residues are highlighted in blue. The secondary structure elements for PfAgo are shown above the sequence.

FIG. 7 shows sequence alignment of the PIWI domains from Pf Argonaute and the four human Argonaute proteins. Invariant residues are highlighted in purple and conserved residues are highlighted in blue. The secondary structure elements are shown above the structure. The conserved active site carboxylate residues are marked by a red asterisk. Accession numbers are as follows: PfAgo (AAL80661), hAgo1 (NM_{—}012199), hAgo2 (NM_{—}012154), hAgo3 (NM_{—}024852) and hAgo4 (NM_{—}017629).

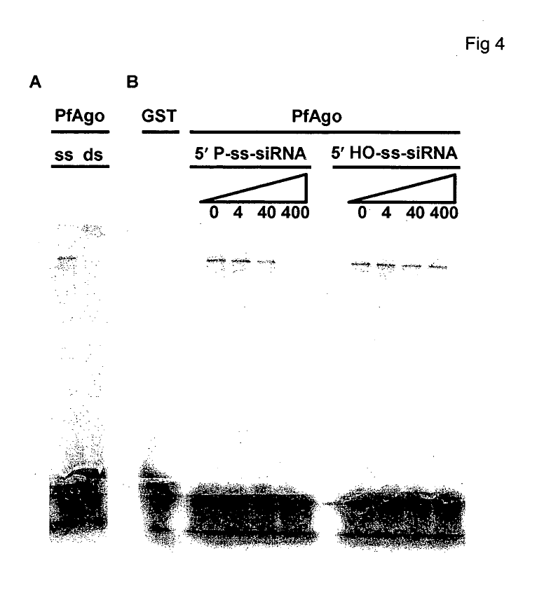

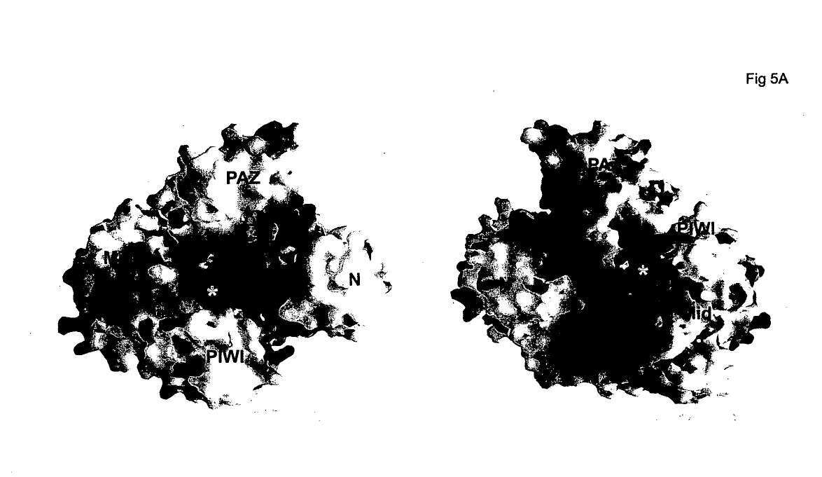

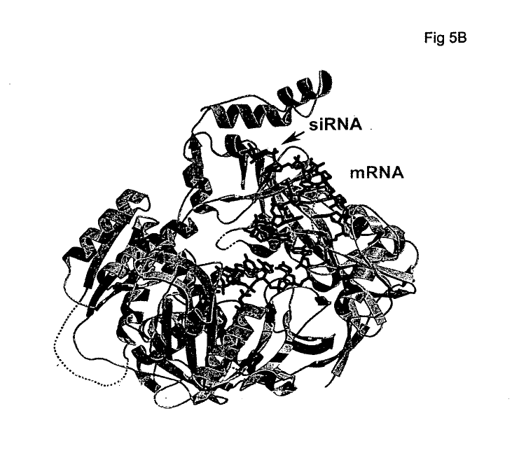

FIG. 8 shows another view of the electrostatic surface potential of PfAgo shown from the proposed exit groove of the mRNA and looking into the active site area (rotated ˜90° around y and ˜20° around x compared to the in FIG. 4A). The PIWI domain is behind the middle domain in this view.

FIG. 9 shows that only mammalian Ago2 can form cleavage-competent RISC. Panel A: The miRNA populations associated with Ago1, Ago2 and Ago3 were measured by microarray analysis as described in Methods. The heat map shows normalized log-ratio values for each dataset, with yellow representing increased relative amounts, and blue indicating decreased amounts, relative to the median. The top 25 log-ratios are shown in the expanded region. In each panel, “control” indicates parallel analysis of cells transfected with a vector control. Panel B: 293T cells were transfected with a control vector or with vectors encoding myc-tagged Ago1, Ago2 or Ago3, as indicated, along with an siRNA that targets firefly luciferase. Immunoprecipitates were tested for siRNA directed mRNA cleavage as described in Methods. Positions of 5′ and 3′ cleavage products are shown. Panel C: Immunoprecipitates as in Panel B were tested for in vivo siRNA binding by Northern blotting of Ago immunoprecipitates (see Methods). Panel D: Western blots of transfected cell lysates show similar levels of expression for each recombinant Argonaute protein.

FIG. 10 shows that Argonaute2 is essential for mouse development. Panel A: Total RNA from Wild-type or mutant embryos was tested for expression of Ago1, Ago2 or Ago3 by RT-PCR. Actin was also examined as a control. Panel B: At day E10.5, Ago2 null embryos show severe developmental delay as compared to heterozygous and wild-type littermates. These embryos also show a variety of developmental defects including swelling inside the pericardial membrane (Panel C, h=heart, indicated by the arrow) and failure to close the neural tube (Panel D, Panel E). Arrows in Panel D indicate the edges of the neural tube that has failed to close. In caudal regions where the neural tube does close, it has an abnormal appearance, being wavy as compared to wild-type embryos (Panel E, compare wt and Ago2−/−). Ago2 is expressed in most tissues of the developing embyo as measured by in situ hybridization (Panel F) or analysis of an Ago2 gene trap animal (Panel G). In Panel F, f=forebrain, b=branchial arches, h=heart and lb=limb bud, all of which are relative hot spots for Ago2 mRNA. In Panel G, the left embryo shows similar patterns when staining for the gene-trap marker, β-galactosidase, proceeds for only a short period. Longer incubation (Panel G, right) gives uniform staining throughout the embryo.

FIG. 11 shows that Argonaute2 is essential for RNAi in MEF. Panel A: RT-PCR of mRNA prepared from Wild-type or Ago2−/− MEF reveals consistent expression of Ago 1 and Ago3 but a specific lack of Ago2 expression in the null MEF. Actin mRNA serves as a control. Panel B: Wild-type and mutant MEFs were co-transfected with plasmids encoding Renilla and firefly luciferases either with or without firefly siRNA as indicated. Ratios of firefly to Renilla activity, normalized to 1 for the no-siRNA control were plotted. For each genotype, the ability of Ago1 and Ago2 to rescue suppression was tested by co-transfection with expression vectors encoding each protein as indicated. Panel C: NIH-3T3 cells, Wild-type MEF or Ago2 mutant MEF were tested as described in B (except that Renilla/firefly ratios are plotted) for their ability to suppress a reporter of repression at the level of protein synthesis. In this case, the Renilla luciferase mRNA contains multiple, imperfect binding sites for a CXCR4 siRNA. Cells were transfected with a mixture of firefly and Renilla luciferase plasmids with or without (as indicated) the siRNA.

FIG. 12 shows mapping of the requirements for assembly of cleavage-competent RISC. Ago 1, Ago2 or the indicated mutants of Ago2 were expressed as myc-tagged fusion proteins in 293T cells. In all cases, expression constructs were co-transfected with a luciferase siRNA. Western Blotting (not shown) indicated similar expression for each mutant. Immunoprecipitate containing individual proteins were tested for cleavage activity against a luciferase mRNA. Positions of 5′ and 3′ cleavage products are indicated. SiRNA binding was examined for each mutant by Northern blotting of immunoprecipitates or by staining of immunoprecipitates with Sybr Gold (Molecular Probes). Representatives for these assays are shown. In no case was a defect in interaction of mutants with siRNAs detected.

FIG. 13 shows that Argonaute2 is a candidate for Slicer. Panel A: Ago2 protein was immunoaffinity purified from transiently transfected 293T cells. The preparation contained two major proteins (Protein Gel), in addition to heavy and light chains. These were identified by mass spectrometry as Ago2 and HSP90. Immunoprecipitates were mixed (see Methods) in vitro with single- or double-stranded siRNAs or with a 21 nt DNA having the same sequence as the siRNA, as indicated. Reconstituted RISC was tested for cleavage activity with a uniformly labeled synthetic mRNA. Positions of 5′ and 3′ cleavage products are noted. Where indicated, the siRNA was not 5′ phosphorylated and in one case, ATP was not added to the reconstitution reaction. Panel B: Ago2 or Ago2 mutants (as indicated) were assembled into RISC in vivo by co-transfection with siRNAs followed by immunoaffinity purification or by in vitro reconstitution, mixing affinity purified proteins with ss-siRNAs. These were tested for activity against a complementary mRNA substrate. 5′ and 3′ cleavage products are as in Panel A. Both mutant proteins were expressed at levels similar to wild-type Ago2 and bound siRNAs as readily (Panel C, Panel D) Ago2 (H634P) and (Q633R) behave similarly in this assay.

FIG. 14 shows cleavage by Ago2-containing RISC irrespective of siRNA sequence. Ago2-containing RISCs were formed in vivo by co-transfection. Complexes were recovered by immunoprecipitation and tested for cleavage activity with a uniformly labeled, synthetic mRNA. Positions of 5′, and 3′ cleavage products expected for each reaction are indicated.

FIG. 15 shows construction of Ago2 mutant mice. The insertional disruption strategy for inactivating mouse Ago2 is shown, along with a southern blot of DNA from wild-type, heterozygous, and null embryos. Probe is indicated by asterisk. For reference, PAZ domain is encoded by exons 5-8. The insertion duplicates exons 3-6, which includes two exons of the PAZ domain, and inserts ˜10 Kb of vector sequences into the gene, creating a high probability that any truncated protein that might be generated from this allele would be non-functional. Additionally, no Ago2 mRNA was detected from these cells by RT-PCR. However, all of the coding capacity of Ago2 does still exist in the mutant genome. Therefore, although all available evidence indicates a null mutation, the possibility cannot be completely ruled out that this mutant can still synthesize a small amount of Ago2, making it a severe hypomorph rather than a null. Southern blots showing the patterns for Wild-type, heterozygous and mutant animals are shown below the disruption strategy.

FIG. 16 shows expression analysis of Ago3 in embryos. Embryonic day 9.5 embryos were collected from timed matings of Wild-type animals. These were stained for expression of Ago3 mRNA by in situ hybridization as described in Methods. Ago3 shows the same expression pattern as is seen in parallel analyses of Ago2 mRNA expression (see FIG. 10, Panel F).

FIG. 17 shows that Ago2-mutant MEF are defective for siRNA-mediated repression WT and Ago2-mutant MEF (genotypes indicated on the left) were transfected with a combination of plasmids encoding dsRed and GFP, either with or without GFP siRNAs (as indictated on the right). Microscopic examination revealed consistent co-expression of dsRed and GFP in the absence of siRNAs in both WT and mutant cells. SiRNAs eliminated co-expression of GFP in WT cells but did not alter GFP expression in Ago2−/− cells.

FIG. 18 shows that intact Ago2 is required for formation of cleavage-competent RISC. Deletions within Ago2 are indicated schematically. Plasmids encoding epitope-tagged versions of each deletion mutant were co-transfected into 293T cells with an siRNA to firefly luciferase. Wild-type Ago2 was similarly expressed as a control. RISCs were immunoaffinity purified and tested for activity against a uniformly labeled mRNA substrate. Each protein was expressed as indicated by Western blotting with a myc antiserum, but none of the deletion mutants bound siRNAs, as determined by Nothern blotting of immunoprecipitates.

FIG. 19 shows that Ago2 can be reconstituted with different siRNAs. Ago2 was immunoaffinitity purified (see FIG. 13) and reconstituted in vitro with single stranded siRNAs that target either the sense strand or the antisense strand of a firefly luciferase mRNA. Similar complexes were formed in parallel with purified Ago 1. In each case, Ago2 cleaved the complementary mRNA, whereas Ago1 complexes were inert. Positions of 5′, and 3′ cleavage products are indicated.

FIG. 20 shows that RISC is a metal-dependent nuclease. As previously shown, RISC requires a divalent metal for activity (Hannon, supra). Similarly, RISC, reconstituted in vitro with single-stranded siRNAs, depends on Mg++ for activity, as indicated by the ability to inhibit the complex with EDTA but not with EGTA (as indicated).

FIG. 21 shows that active site residues are conserved among Ago proteins. Putative active site aspartate residues in the PIWI domain were identified with reference to the structure of the P. furiosus Ago protein. These were also conserved in Ago proteins from a variety of species. Additionally, residues identified by a mutational analysis (e.g. H634) were also highly conserved.

FIG. 22 shows sequence alignment of mammalian Ago1 family members. An alignment of the protein sequences of human Argonautes 1-4 highlights a very high degree of sequence conservation. Red indicates highly conserved, blue moderately conserved residues. Residues mutated in Ago2 in this study are indicated in green and by asterisks (see below). The PAZ domain is indicated by the yellow bar and the PIWI by the orange bar (boundaries set as determined by structural data). Accession numbers for individual genes are as follows: Ago1 (NM_{—}012199), Ago2 (NM_{—}012154), Ago3(NM_{—}024852), Ago4 (NM_{—}017629).

FIG. 23 shows Table I which provides crystallographic statistics for Argonaute.

FIG. 24 shows Table 2 which provides additional crystallographic statistics for Argonaute.

FIG. 25 shows Table 3 which provides the atomic coordinates for Argonaute.

### DETAILED DESCRIPTION OF THE APPLICATION

Overview

Argonautes are often present as multiprotein families and are identified by two characteristic domains, PAZ and PIWI (21). These proteins mainly segregate into two sub-families, comprising those that are more similar to either Arabidopsis Argonaute 1 or Drosophila Piwi. The Argonaute family was first linked to RNAi through genetic studies in C. elegans, which identified Rde-1 as a gene essential for silencing (22). Subsequent placement of a Drosophila Argonaute protein in RISC (19) makes it desirable to explore the unknown roles of this protein family. Toward this end, this application provides methods and compositions related to Argonaute. These methods and compositions are based on results obtained from structural studies of Argonaute proteins, as well as biochemical, and genetic studies of a subfamily of Argonaute proteins in mammals. As used herein, the term “Argonaut” refers to a protein which (a) mediates an RNAi response and (b) has an amino acid sequence at least 50 percent identical, and more preferably at least 75, 85, 90 or 95 percent identical to SEQ ID NOs: 1-5.

Structural Studies of Argonaute

The crystal structure of Argonaute is useful for in silico screening of agents that bind to Argonaute and/or modulates its activity. The candidate agents generated from the in silico screening can be further screened in biochemical assays to select for agents that modulate the activity of Argonaute.

1. Crystallization and Structure Determination

X-ray crystallography is a method of solving the three dimensional structures of molecules. The structure of a molecule is calculated from X-ray diffraction patterns using a crystal as a diffraction grating. Three dimensional structures of protein molecules arise from crystals grown from a concentrated aqueous solution of that protein. The process of X-ray crystallography can include the following steps:

(a) synthesizing and isolating (or otherwise obtaining) a polypeptide;

(b) growing a crystal from an aqueous solution comprising the polypeptide with or without a modulator; and

(c) collecting X-ray diffraction patterns from the crystals, determining unit cell dimensions and symmetry, determining electron density, fitting the amino acid sequence of the polypeptide to the electron density, and refining the structure.

a. Production of Polypeptides

The Argonaute polypeptides described herein may be chemically synthesized in whole or part using techniques that are well-known in the art (see, e.g., Creighton (1983) Biopolymers 22(1):49-58).

Alternatively, methods which are well known to those skilled in the art can be used to construct expression vectors containing the native or mutated Argonaute polypeptide coding sequence and appropriate transcriptional/translational control signals. These methods include in vitro recombinant DNA techniques, synthetic techniques and in vivo recombination/genetic recombination. See, for example, the techniques described in Maniatis, T (1989). Molecular cloning: A laboratory Manual. Cold Spring Harbor Laboratory, New York. Cold Spring Harbor Laboratory Press; and Ausubel, F. M. et al. (1994) Current Protocols in Molecular Biology (John Wiley & Sons, Secaucus, N.J.).

A variety of host-expression vector systems may be utilized to express the Argonaute coding sequence. These include but are not limited to microorganisms such as bacteria transformed with recombinant bacteriophage DNA, plasmid DNA or cosmid DNA expression vectors containing the Argonaute domain coding sequence; yeast transformed with recombinant yeast expression vectors containing the Argonaute domain coding sequence; insect cell systems infected with recombinant virus expression vectors (e.g., baculovirus) containing the Argonaute domain coding sequence; plant cell systems infected with recombinant virus expression vectors (e.g., cauliflower mosaic virus, CaMV; tobacco mosaic virus, TMV) or transformed with recombinant plasmid expression vectors (e.g., Ti plasmid) containing the Argonaute domain coding sequence; or animal cell systems. The expression elements of these systems vary in their strength and specificities.

Depending on the host/vector system utilized, any of a number of suitable transcription and translation elements, including constitutive and inducible promoters, may be used in the expression vector. For example, when cloning in bacterial systems, inducible promoters such as pL of bacteriophage .lambda., plac, ptrp, ptac (ptrp-lac hybrid promoter) and the like may be used; when cloning in insect cell systems, promoters such as the baculovirus polyhedrin promoter may be used; when cloning in plant cell systems, promoters derived from the genome of plant cells (e.g., heat shock promoters; the promoter for the small subunit of RUBISCO; the promoter for the chlorophyll alb binding protein) or from plant viruses (e.g., the .sup.35S RNA promoter of CaMV; the coat protein promoter of TMV) may be used; when cloning in mammalian cell systems, promoters derived from the genome of mammalian cells (e.g., metallothionein promoter) or from mammalian viruses (e.g., the adenovirus late promoter; the vaccinia virus 7.5K promoter) may be used; when generating cell lines that contain multiple copies of the Argonaute domain DNA, SV40-, BPV- and EBV-based vectors may be used with an appropriate selectable marker.

Exemplary methods describing methods of DNA manipulation, vectors, various types of cells used, methods of incorporating the vectors into the cells, expression techniques, protein purification and isolation methods, and protein concentration methods are disclosed in detail in PCT publication WO 96/18738. This publication is incorporated herein by reference in its entirety, including any drawings. Those skilled in the art will appreciate that such descriptions are applicable to the present invention and can be easily adapted to it.

b. Crystal Growth

Crystals are grown from an aqueous solution containing the purified and concentrated Argonaute polypeptide by a variety of techniques. These techniques include batch, liquid, bridge, dialysis, vapor diffusion, and hanging drop methods. McPherson (1982) John Wiley, New York; McPherson (1990) Eur. J. Biochem. 189:1-23; Webber (1991) Adv. Protein Chem. 41:1-36, incorporated by reference herein in their entireties, including all figures, tables, and drawings.

The native crystals of the application are, in general, grown by adding precipitants to the concentrated solution of the polypeptide. The precipitants are added at a concentration just below that necessary to precipitate the protein. Water is removed by controlled evaporation to produce precipitating conditions, which are maintained until crystal growth ceases.

For crystals of the application, exemplary crystallization conditions are described in the Examples. Those of ordinary skill in the art will recognize that the exemplary crystallization conditions can be varied. Such variations may be used alone or in combination. In addition, other crystallizations may be found, e.g., by using crystallization screening plates to identify such other conditions.

c. X-Ray Diffraction

The diffraction data from X-ray crystallography is generally obtained as follows. When a crystal is placed in an X-ray beam, the incident X-rays interact with the electron cloud of the molecules that make up the crystal, resulting in X-ray scatter. The combination of X-ray scatter with the lattice of the crystal gives rise to nonuniformity of the scatter; areas of high intensity are called diffracted X-rays. The angle at which diffracted beams emerge from the crystal can be computed by treating diffraction as if it were reflection from sets of equivalent, parallel planes of atoms in a crystal (Bragg's Law). The most obvious sets of planes in a crystal lattice are those that are parallel to the faces of the unit cell. These and other sets of planes can be drawn through the lattice points. Each set of planes is identified by three indices, hk1. The h index gives the number of parts into which the a edge of the unit cell is cut, the k index gives the number of parts into which the b edge of the unit cell is cut, and the 1 index gives the number of parts into which the c edge of the unit cell is cut by the set of hk1 planes. Thus, for example, the 235 planes cut the a edge of each unit cell into halves, the b edge of each unit cell into thirds, and the c edge of each unit cell into fifths. Planes that are parallel to the bc face of the unit cell are the 100 planes; planes that are parallel to the ac face of the unit cell are the 010 planes; and planes that are parallel to the ab face of the unit cell are the 001 planes.

When a detector is placed in the path of the diffracted X-rays, in effect cutting into the sphere of diffraction, a series of spots, or reflections, are recorded to produce a “still” diffraction pattern. Each reflection is the result of X-rays reflecting off one set of parallel planes, and is characterized by an intensity, which is related to the distribution of molecules in the unit cell, and hk1 indices, which correspond to the parallel planes from which the beam producing that spot was reflected. If the crystal is rotated about an axis perpendicular to the X-ray beam, a large number of reflections is recorded on the detector, resulting in a diffraction pattern.

The unit cell dimensions and space group of a crystal can be determined from its diffraction pattern. First, the spacing of reflections is inversely proportional to the lengths of the edges of the unit cell. Therefore, if a diffraction pattern is recorded when the X-ray beam is perpendicular to a face of the unit cell, two of the unit cell dimensions may be deduced from the spacing of the reflections in the x and y directions of the detector, the crystal-to-detector distance, and the wavelength of the X-rays. Those of skill in the art will appreciate that, in order to obtain all three unit cell dimensions, the crystal must be rotated such that the X-ray beam is perpendicular to another face of the unit cell. Second, the angles of a unit cell can be determined by the angles between lines of spots on the diffraction pattern. Third, the absence of certain reflections and the repetitive nature of the diffraction pattern, which may be evident by visual inspection, indicate the internal symmetry, or space group, of the crystal. Therefore, a crystal may be characterized by its unit cell and space group, as well as by its diffraction pattern.

Once the dimensions of the unit cell are determined, the likely number of polypeptides in the asymmetric unit can be deduced from the size of the polypeptide, the density of the average protein, and the typical solvent content of a protein crystal, which is usually in the range of 30-70% of the unit cell volume.

The diffraction pattern is related to the three-dimensional shape of the molecule by a Fourier transform. The process of determining the solution is in essence a re-focusing of the diffracted X-rays to produce a three-dimensional image of the molecule in the crystal. Since re-focusing of X-rays cannot be done with a lens at this time, it is done via mathematical operations.

The sphere of diffraction has symmetry that depends on the internal symmetry of the crystal, which means that certain orientations of the crystal will produce the same set of reflections. Thus, a crystal with high symmetry has a more repetitive diffraction pattern, and there are fewer unique reflections that need to be recorded in order to have a complete representation of the diffraction. The goal of data collection, a dataset, is a set of consistently measured, indexed intensities for as many reflections as possible. A complete dataset is collected if at least 80%, preferably at least 90%, most preferably at least 95% of unique reflections are recorded. In one embodiment, a complete dataset is collected using one crystal. In another embodiment, a complete dataset is collected using more than one crystal of the same type.

Sources of X-rays include, but are not limited to, a rotating anode X-ray generator such as a Rigaku RU-200 or a beamline at a synchrotron light source, such as the Advanced Photon Source at Argonne National Laboratory. Suitable detectors for recording diffraction patterns include, but are not limited to, X-ray sensitive film, multiwire area detectors, image plates coated with phosphorus, and CCD cameras. Typically, the detector and the X-ray beam remain stationary, so that, in order to record diffraction from different parts of the crystal's sphere of diffraction, the crystal itself is moved via an automated system of moveable circles called a goniostat. The three dimensional (x, y, z) coordinates of Argonaute are shown in Table 3 (FIG. 25) in the standard Protein Data Bank (PDB) format. (Bemstain F. C., et al. J. Mol. Biol., 1977, 122, 535).

TABLE 3—Atomic Coordinates (FIG. 25).

Once a dataset such as the one in Table 3 (FIG. 25) is collected, the information is used to determine the three-dimensional structure of the molecule in the crystal. However, in the absence alone of a suitable molecular model, this cannot be done from a single measurement of reflection intensities because certain information, known as phase information, is lost between the three-dimensional shape of the molecule and its Fourier transform, the diffraction pattern. This phase information must be acquired by methods described below in order to perform a Fourier transform on the diffraction pattern to obtain the three-dimensional structure of the molecule in the crystal. It is the determination of phase information that in effect refocuses X-rays to produce the image of the molecule.

One method of obtaining phase information is by isomorphous replacement, in which heavy-atom derivative crystals are used. In this method, the positions of heavy atoms bound to the molecules in the heavy-atom derivative crystal are determined, and this information is then used to obtain the phase information necessary to elucidate the three-dimensional structure of a native crystal. (Blundel et al., 1976, Protein Crystallography, Academic Press).

Another method of obtaining phase information is by molecular replacement, which is a method of calculating initial phases for a new crystal of a polypeptide or polypeptide co-complex whose structure coordinates are unknown by orienting and positioning a related polypeptide whose structure coordinates are known within the unit cell of the new crystal so as to best account for the observed diffraction pattern of the new crystal. To enable this, the related molecule must have a similar three dimensional structure. Briefly, the principle behind the method of molecular replacement is as follows. A suitable search model, whose three-dimensional structure is similar to that of the unknown target, is identified first. The search model is then rotated and translated within the unit cell of the unknown. For each position of the model, a set of structure factors of the model is computed. These calculated structure factors are then compared with the measured intensities of the unknown and expressed as correlation coefficients. The solution with the highest correlation coefficient is selected as the true solution. These concepts are discussed at length in the book “The Molecular Replacement Method edited by Rossmann (1972, Int. Sci. Rev. Ser. No 13, Gordon & Breach, New York).

A third method of phase determination is multi-wavelength anomalous dispersion or MAD. In this method, X-ray diffraction data are collected at several different wavelengths from a single crystal containing at least one heavy atom with absorption edges near the energy of incoming X-ray radiation. The resonance between X-rays and electron orbitals leads to differences in X-ray scattering that permits the locations of the heavy atoms to be identified, which in turn provides phase information for a crystal of a polypeptide. A detailed discussion of MAD analysis can be found in Hendrickson, 1985, Trans. Am. Crystallogr. Assoc., 21:11; Hendrickson et al., 1990, EMBO J. 9:1665; and Hendrickson, 1991, Science 4:91.

A fourth method of determining phase information is single wavelength anomalous w dispersion or SAD. In this technique, X-ray diffraction data are collected at a single wavelength from a single native or heavy-atom derivative crystal, and phase information is extracted using anomalous scattering information from atoms such as sulfur or chlorine in the native crystal or from the heavy atoms in the heavy-atom derivative crystal. A detailed discussion of SAD analysis can be found in Brodersen et al., 2000, Acta Cryst., D56:431-441.

A fifth method of determining phase information is single isomorphous replacement with anomalous scattering or SIRAS. This technique combines isomorphous replacement and anomalous scattering techniques to provide phase information for a crystal of a polypeptide. X-ray diffraction data are collected at a single wavelength, usually from a single heavy-atom derivative crystal. Phase information obtained only from the location of the heavy atoms in a single heavy-atom derivative crystal leads to an ambiguity in the phase angle, which is resolved using anomalous scattering from the heavy atoms. Phase information is therefore extracted from both the location of the heavy atoms and from anomalous scattering of the heavy atoms. A detailed discussion of SIRAS analysis can be found in North, 1965, Acta Cryst. 18:212-216; Matthews, 1966, Acta Cryst. 20:82-86.

Once phase information is obtained, it is combined with the diffraction data to produce an electron density map, an image of the electron clouds that surround the molecules in the unit cell. The higher the resolution of the data, the more distinguishable are the features of the electron density map, e.g., amino acid side chains and the positions of carbonyl oxygen atoms in the peptide backbones, because atoms that are closer together are resolvable. A model of the macromolecule is then built into the electron density map with the aid of a computer, using as a guide all available information, such as the polypeptide sequence and the established rules of molecular structure and stereochemistry. Interpreting the electron density map is a process of finding the chemically realistic conformation that fits the map precisely.

After a model is generated, the structure is refined. Refinement is the process of minimizing the function Φ, which is the difference between observed and calculated intensity values (measured by an R-factor), and which is a function of the position, temperature factor, and occupancy of each non-hydrogen atom in the model. This usually involves alternate cycles of real space refinement, i.e., calculation of electron density maps and model building, and reciprocal space refinement, i.e., computational attempts to improve the agreement between the original intensity data and intensity data generated from each successive model. Refinement ends when the function Φ converges on a minimum wherein the model fits the electron density map and is stereochemically and conformationally reasonable. During refinement, ordered solvent molecules are added to the structure.

d. Various Representations

The atomic structure coordinates and machine readable media of the application have a variety of uses. The present invention encompasses the structure coordinates and other information, e.g., amino acid sequence, connectivity tables, vector-based representations, temperature factors, etc., used to generate the three-dimensional structures of the polypeptides for use in the software programs described below and other software programs. For example, the coordinates listed in Table 3 (FIG. 25) are useful for solving the three-dimensional crystal or solution structures of other proteins to high resolution.

Additionally, the invention encompasses machine readable media embedded with the three-dimensional structures of the models described herein, or with portions thereof. As used herein, “machine readable medium” or “computer readable medium” refers to any medium that can be read and accessed directly by a computer or scanner. Such media include, but are not limited to: magnetic storage media, such as floppy discs, hard disc storage medium and magnetic tape; optical storage media such as optical discs or CD-ROM; electrical storage media such as RAM or ROM; and hybrids of these categories such as magnetic/optical storage media. Such media further include paper on which is recorded a representation of the atomic structure coordinates, e.g., Cartesian coordinates, that can be read by a scanning device and converted into a three-dimensional structure with an Optical Character Recognition (OCR).

A variety of data storage structures are available to a skilled artisan for creating a computer readable medium having recorded thereon the atomic structure coordinates of the application or portions thereof and/or X-ray diffraction data. The choice of the data storage structure will generally be based on the means chosen to access the stored information. In addition, a variety of data processor programs and formats can be used to store the sequence and X-ray data information on a computer readable medium. Such formats include, but are not limited to, Protein Data Bank (“PDB”) format (Research Collaboratory for Structural Bioinformatics; http://www.rcsb.org/pdb/docs/format/pdbguide2.2/guide2.2_frame.html); Cambridge Crystallographic Data Centre format (http://www.ccdc.cam.ac.uk/support/csd_doc/volume3/z323.html); Structure-data (“SD”) file format (MDL Information Systems, Inc.; Dalby et al., 1992, J. Chem. Inf. Comp. Sci. 32:244-255), and line-notation, e.g., as used in SMILES (Weininger, 1988, J. Chem. Inf. Comp. Sci. 28:31-36). Methods of converting between various formats read by different computer software will be readily apparent to those of skill in the art, e.g., BABEL (v. 1.06, Walters & Stahl, © 1992, 1993, 1994; http://www.brunel.ac.uk/departments/chem/babel.htm.) All format representations of the polypeptide coordinates described herein, or portions thereof, are contemplated by the present invention. By providing computer readable medium having stored thereon the atomic coordinates of the application, one of skill in the art can routinely access the atomic coordinates of the application, or portions thereof, and related information for use in modeling and design programs, described in detail below.

While Cartesian coordinates are important and convenient representations of the three-dimensional structure of a polypeptide, those of skill in the art will readily recognize that other representations of the structure are also useful. Therefore, the three-dimensional structure of a polypeptide, as discussed herein, includes not only the Cartesian coordinate representation, but also all alternative representations of the three-dimensional distribution of atoms. For example, atomic coordinates may be represented as a Z-matrix, wherein a first atom of the protein is chosen, a second atom is placed at a defined distance from the first atom, a third atom is placed at a defined distance from the second atom so that it makes a defined angle with the first atom. Each subsequent atom is placed at a defined distance from a previously placed atom with a specified angle with respect to the third atom, and at a specified torsion angle with respect to a fourth atom. Atomic coordinates may also be represented as a Patterson function, wherein all interatomic vectors are drawn and are then placed with their tails at the origin. This representation is particularly useful for locating heavy atoms in a unit cell. In addition, atomic coordinates may be represented as a series of vectors having magnitude and direction and drawn from a chosen origin to each atom in the polypeptide structure. Furthermore, the positions of atoms in a three-dimensional structure may be represented as fractions of the unit cell (fractional coordinates), or in spherical polar coordinates.

Additional information, such as thermal parameters, which measure the motion of each atom in the structure, chain identifiers, which identify the particular chain of a multi-chain protein or protein co-complex in which an atom is located, and connectivity information, which indicates to which atoms a particular atom is bonded, is also useful for representing a three-dimensional molecular structure.

e. Structure of Argonaute

The present invention provides high-resolution three-dimensional structures and atomic structure coordinates of crystalline Argonaute as determined by X-ray crystallography. The specific methods used to obtain the structure coordinates are provided in the examples and throughout the application. The atomic structure coordinates of crystalline Argonaute are listed in Table 3 (FIG. 25).

Those having skill in the art will recognize that atomic structure coordinates as determined by X-ray crystallography are not without error. Thus, it is to be understood that any set of structure coordinates obtained for crystals of Argonaute, whether native crystals, derivative crystals or co-crystals, that have a root mean square deviation (“r.m.s.d.”) of less than or equal to about 1.5 Angstrom when superimposed, using backbone atoms (N, Cα, C and O), on the structure coordinates listed in Table 3 (FIG. 25) are considered to be identical with the structure coordinates listed in the Table 3 (FIG. 25) when at least about 50% to 100% of the backbone atoms of Argonaute are included in the superposition.

II. Crystalline Argonaute

It is to be understood that the crystalline Argonaute of the application are not limited to naturally occurring or native Argonaute. Indeed, the crystals of the application include crystals of mutants of native Argonaute. Mutants of naturally-occurring or native Argonautes are obtained by replacing at least one amino acid residue in a native Argonaute with a different amino acid residue, or by adding or deleting amino acid residues within the native polypeptide or at the N- or C-terminus of the native polypeptide, and have substantially the same three-dimensional structure as the native Argonaute from which the mutant is derived.

By having substantially the same three-dimensional structure is meant having a set of atomic structure coordinates that have a root-mean-square deviation of less than or equal to about 2 angstrom when superimposed with the atomic structure coordinates of the native Argonaute from which the mutant is derived when at least about 50% to 100% of the Ca atoms of the native Argonaute domain are included in the superposition.

Amino acid substitutions, deletions and additions which do not significantly interfere with the three-dimensional structure of the Argonaute will depend, in part, on the region of the Argonaute where the substitution, addition or deletion occurs. In highly variable regions of the molecule, non-conservative substitutions as well as conservative substitutions may be tolerated without significantly disrupting the three-dimensional, structure of the molecule. In highly conserved regions, or regions containing significant secondary structure, conservative amino acid substitutions are preferred.

Conservative amino acid substitutions are well known in the art, and include substitutions made on the basis of similarity in polarity, charge, solubility, hydrophobicity, hydrophilicity and/or the amphipathic nature of the amino acid residues involved. For example, negatively charged amino acids include aspartic acid and glutamic acid; positively charged amino acids include lysine and arginine; amino acids with uncharged polar head groups having similar hydrophilicity values include the following: leucine, isoleucine, valine; glycine, alanine; asparagine, glutamine; serine, threonine; phenylalanine, tyrosine. Other conservative amino acid substitutions are well known in the art.

For Argonaute obtained in whole or in part by chemical synthesis, the selection of amino acids available for substitution or addition is not limited to the genetically encoded amino acids. Indeed, the mutants described herein may contain non-genetically encoded amino acids. Conservative amino acid substitutions for many of the commonly known non-genetically encoded amino acids are well known in the art. Conservative substitutions for other amino acids can be determined based on their physical properties as compared to the properties of the genetically encoded amino acids.

In some instances, it may be particularly advantageous or convenient to substitute, delete and/or add amino acid residues to a native Argonaute in order to provide convenient cloning sites in cDNA encoding the polypeptide, to aid in purification of the polypeptide, and for crystallization of the polypeptide. Such substitutions, deletions and/or additions which do not substantially alter the three dimensional structure of the native Argonaute domain will be apparent to those of ordinary skill in the art.

It should be noted that the mutants contemplated herein need not all exhibit Argonaute activity. Indeed, amino acid substitutions, additions or deletions that interfere with the Argonaute activity but which do not significantly alter the three-dimensional structure of the domain are specifically contemplated by the invention. Such crystalline polypeptides, or the atomic structure coordinates obtained therefrom, can be used to identify compounds that bind to the native domain. These compounds can affect the activity of the native domain.

The co-crystals of the application generally comprise a crystalline Argonaute domain polypeptide in association with one or more compounds. The association may be covalent or non-covalent. Such compounds include, but are not limited to, cofactors, substrates, substrate analogues, modulators, allosteric effectors, etc.

Argonaute

As used herein, the term “Argonaut” refers to a protein which (a) mediates an RNAi response and (b) has an amino acid sequence at least 50 percent identical, and more preferably at least 75, 85, 90 or 95 percent identical to SEQ ID NOs.: 1-5.

Mammals contain four Argonaute1 subfamily members, Ago 1-Ago4 (nomenclature as in (Carmell et al., Genes Dev. 16, 2733 (2002)), see FIG. 22 which provides sequence alignment of human Ago1-4 proteins, corresponding to SEQ ID NOs: 1-4). Different Argonaute family members in Drosophila preferentially associate with different small RNAs, with Ago1 preferring miRNAs and Ago2 siRNAs (24). Recent studies of dmAgo1 and dmAgo2 mutants have strengthened these conclusions (25). To assess whether mammalian Ago proteins specialized in their interactions with small RNAs, Ago-associated miRNA populations were examined by microarray analysis (Example 1).

Amino Acid Sequence of Pyrococcus furiosus Argonaute Protein:

| | |
|---|---|
| MKAKVVINLVKINKKIIPDKIYVYRLFNDPEEELQKEGYSIYRLAYENVGIVIDPE | SEQ ID NO.: 5 |
| NLIIATTKELEYEGEFIPEGEISFSELRNDYQSKLVLRLLKENGIGEYELSKLLRKFRKPKT | |
| FGDYKVIPSVEMSVIKHDEDFYLVIHIIHQIQSMKTLWELVNKDPKELEEFLMTHKENL | |
| MLKDIASPLKTVYKPCFEEYTKKPKLDHNQEIVKYWYNYHIERYWNTPEAKLEFYRKF | |
| GQVDLKQPAILAKFASKIKKNKNYKIYLLPQLVVPTYNAEQLESDVAKEILEYTKLMPE | |
| ERKELLENILAEVDSDIIDKSLSEIEVEKIAQELENKIRVRDDKGNSVPISQLNVQKSQLLL | |
| WTNYSRKYPVILPYEVPEKFRKIREIPMFIILDSGLLADIQNFATNEFRELVKSMYYSLAK | |
| KYNSLAKKARSTNEIGLPFLDFRGKEKVITEDLNSDKGIIEVVEQVSSFMKGKELGLAFI | |
| AARNKLSSEKFEEIKRRLFNLNVISQVVNEDTLKNKRDKYDRNRLDLFVRHNLLFQVLS | |
| KLGVKYYVLDYRFNYDYIIGIDVAPMKRSEGYIGGSAVMFDSQGYIRKIVPIKIGEQRGE | |
| SVDMNEFFKEMVDKFKEFNIKLDNKKILLLRDGRITNNEEEGLKYISEMFDIEVVTMDVI | |
| KNHPVRAFANMKMYFNLGGAIYLIPHKLKQAKGTPIPIKLAKKRIIKNGKVEKQSITRQD | |
| VLDIFILTRLNYGSISADMRLPAPVHYAHKFANAIRNEWKIKEEFLAEGFLYFV | |

1. Overall Architecture

This application provides the structure of the full-length Argonaute from the archaebacterium Pyrococcus furiosus (PfAgo) as determined by x-ray crystallography to 2.25 Å resolution. The structure was solved by multiple anomalous dispersion (MAD) and isomorphous replacement using selenium and mercury derivatives (Table 2 shown in FIG. 24)). The N-terminal, middle, and PIWI domains form a crescent-shaped base, with the PIWI domain at the center of the crescent. The region following the N-terminus forms a “stalk” that holds the PAZ domain above the crescent and an interdomain connector cradles the molecule (FIG. 1). This architecture results in a cleft formed at the center of the crescent with the PAZ domain closing in on this cleft.

The N-terminal domain consists of a long strand at the bottom of the crescent, continuing to a region of a small four-stranded β-sheet, three α-helices and a β-hairpin, which then extends to the three-stranded antiparallel β-sheet stalk.

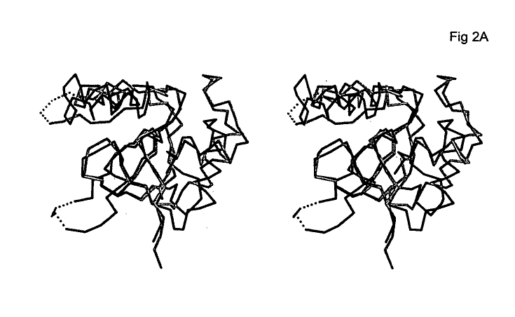

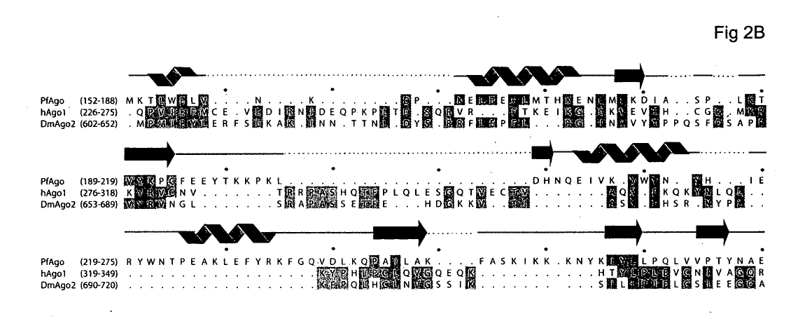

Also provided is the PAZ domain, a globular domain that adopts an OB-like β-barrel fold with an attachment on one side of the barrel and a cleft in between. This cleft was shown to be the binding site for the 2-nucleotide 3′-overhang of the siRNA (29, 32, 33) and is angled towards the crescent. The PAZ domain in PfAgo superimposes very well with the PAZ domains from Drosophila Argonaute 1 (30) and 2 (29, 31) and with the human Argonaute-1 (hAgo1) PAZ domain in complex with a “mini-siRNA” (33), though the attachment in the archael protein has two α-helices rather than an α-helix and a β-hairpin (FIGS. 2A and 2B).

The middle domain, which is located at one end of the crescent, is an α/β open sheet domain composed of a central three-stranded parallel β-sheet surrounded by α-helices. This domain is similar to the glucose-galactose-arabinose-ribose binding protein family and is most similar to Lac repressor (35). The middle domain also has small three-stranded β-sheet on the outer surface of the crescent, connecting it to the rest of the molecule.

Further provided is the PIWI domain, which is at the C-terminus of Argonaute (residues 545-770). It sits in the middle of the crescent and below the PAZ domain. The crystal structure reveals the presence of a prominent central five-stranded β-sheet flanked on both sides by α-helices at the core of the PIWI domain. A smaller β-sheet extends from the central β-sheet and attaches PIWI to the N-terminal domain and to portions of the interdomain connector.

2. Domain Structure

As mentioned above, the PAZ domain superimposes very well with all the other PAZ domains with known structures, namely, Drosophila Argonautes 1 and 2 and hAgo1 (FIG. 2A). Most of the differences lie in loop regions. The root-mean-square deviation (mmsd) between hAgo1-PAZ and the PAZ domain in this structure is approximately 1.4 Å (for 53 Ca's). Though it is now possible to align the sequence of the PAZ domain of PfAgo with PAZ domains from Argonaute proteins of higher eukaryotes (FIG. 2B) based on the structures, homologies between the archeal and eukaryotic PAZ domains was not apparent before the PfAgo structure was determined. In fact, primary sequence comparisons provided no evidence that PfAgo contained a PAZ domain. Even after attempting to align the sequences with reference to the three-dimensional structures, the sequence identity remains below 10%. The presence and location of the PIWI domain was, on the other hand, obvious from the primary sequence, and could be readily identified through BLAST searches.

The role of the PAZ domain, as shown for fly Ago-2 (29, 32) and for hAgo-1 (33) is to bind the 2-nucleotide 3′ overhang of the siRNA. Importantly, the conserved aromatic residues that fill the cleft and were shown to bind those nucleotides (29, 32, 33) are all present in the PfAgo PAZ domain. Curiously, in some cases, these side chains occupy similar positions in space even if they aren't anchored to positions on the peptide backbone corresponding to those in eukaryotic proteins. Specifically, Y212, Y216, H217 and Y190 are equivalent to Y309, Y314, H269 and Y277 of hAgo1 that were shown to bind the oxygens of the phosphate that links the two bases in the overhang. Residue Y190 of PfAgo superimposes perfectly on hAgo1-Y277 that was also shown to bind the 2′-hydroxyl of the penultimate nucleotide. Residues L263 and 1261 can assume the role of L337 and T335, which anchor the sugar ring of the terminal residue through van der Waals interactions in the hAgo1-RNA structure. There is an aromatic residue, F292 in hAgo1 that stacks against the terminal nucleotide. This position is occupied by another aromatic, W213, in PfAgo. Finally, R220 in the structure of the present application is positioned similarly to K313 that contacts the penultimate nucleotide. As for residues that were shown to bind the region of the RNA strand 5′ to the overhang, K191 is positioned as R278 in hAgo1 to bind phosphates and Y259 is equivalent to K333. Other PAZ residues, such as K252, K248, Q276 and N176 are probably used to bind that strand as well. Accordingly, the PAZ domain in PfAgo appears to have a similar function to the PAZ domains of the fly and human Argonautes and would also be capable of binding a 3′ single-stranded region of an RNA molecule.

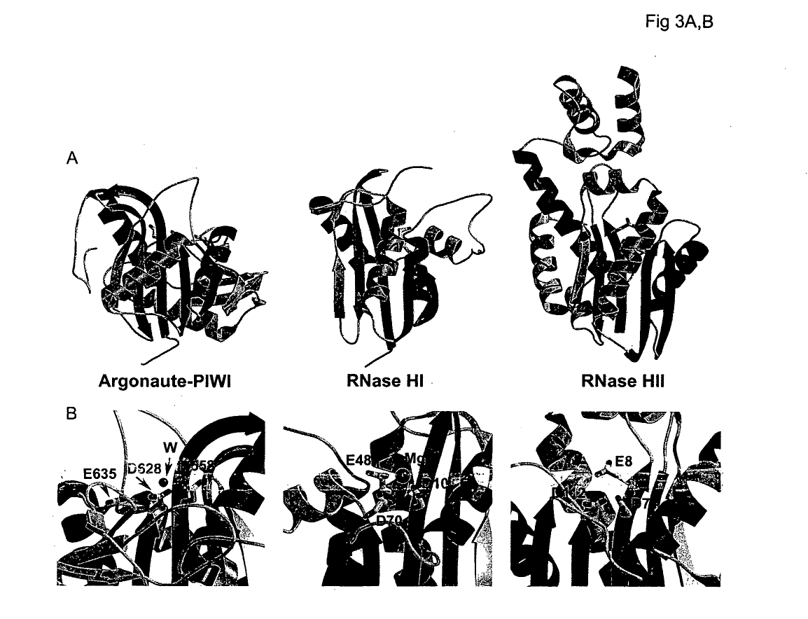

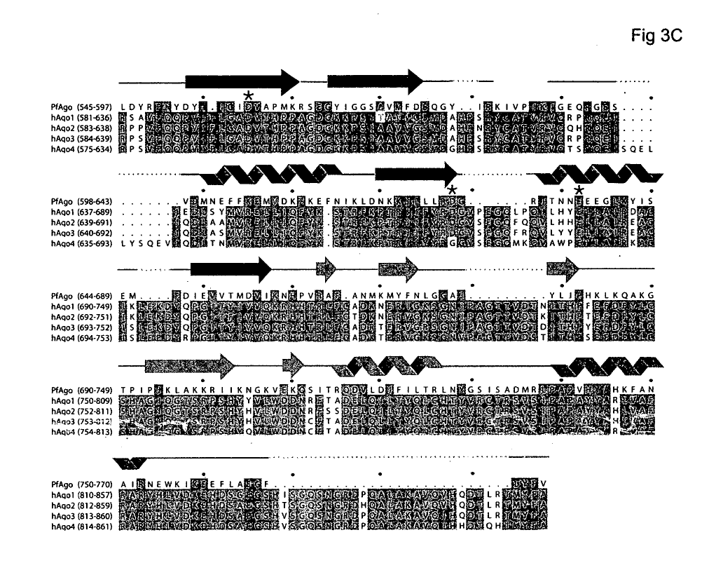

The present application also provides a PIWI domain core having a tertiary structure that belongs to the RNase H family of enzymes, which include RNase H type 1 and type 2 enzymes. This fold is also characteristic of other enzymes with nuclease or polynucleotidyl transferase activities, such as HIV and ASV integrases (36, 37), RuvC (38), a Holliday junction endonuclease, and transposases such as Mu (39) and Tn5 (40). The closest matches, however, are with RNase HII (41) and RNase H1 (42). The rmsd's between these proteins and PfAgo are of 1.9 Å and they are topologically identical (FIG. 3A). RNase H fold proteins all have a five-stranded mixed β-sheet surrounded by helices. In the RNase H enzymes as well as PIWI, there are two helices on either side of the β-sheet. On one side these are very similar, and on the other, one of the helices varies. PIWI has an insertion between the last strand and the last helix of the RNase H fold. This insertion consists of a smaller β-sheet attachment and a helix that links it to the rest of the protein. RNase HII has a cap domain that sits above the active site cleft and forms a groove for substrate binding (43). In addition, several residues from the cap domain appear to participate in substrate recognition. The positioning of the cap relative to the RNase H fold of the protein is approximately the same as the PAZ domain relative to the PIWI domain in Argonaute.

Similarity is not restricted to the protein fold. In all of these enzymes there are three highly conserved carboxylates which are essential for catalytic activity (44). Two of these carboxylate side chains are always located on the first strand, β1, which is the central strand of the β-sheet, and at the C-terminus of the fourth strand, β4, of the RNase H fold, which is adjacent to β1 (the red and green strands in FIGS. 3A and 3B). The position of the third carboxylate varies between the different RNase H fold enzymes. Remarkably, when examining a superposition between either RNase H1 or RNase HII and PIWI, two aspartate residues were located at the same positions as the invariant carboxylates of the RNase H fold (FIG. 3B). These are D558 located on the first b-strand of PIWI and D628 located at the end of the fourth strand of the PIWI domain. These aspartates are equivalent to D10 and D70 in E. coli RNase H1, D7 and D112 in Methanococcus jannaschii RNase HII, and D6 and D101 in Archaeoglobus fulgidus RNase HII. The location of the third carboxylate, a glutamate, in RNase H1 and HII is occupied by a valine in Argonaute. However, a glutamate, E635, is in close proximity to the two aspartates, and this glutamate may serve as the third active site residue. This residue is positioned on the second helix of the RNase H fold of PIWI (the blue helix in FIGS. 3A-3B). Since the position of the third carboxylate varies in these proteins, the only requirement would be for a reasonable spatial position at the active site, a criterion which E635 meets. Therefore, the active site of PfAgo is likely composed of the carboxylate triad formed by D528, D628 and E635 that make up the “DDE” motif. Interestingly, an arginine, R627, is also positioned at the center of the active site, as in the case of the IS4 family of transposases such as Tn5 which appear to have a “DDRE motif” (40, 45). The active site is thus positioned in a cleft in the middle of the crescent in the groove below the PAZ domain.

RNase H enzymes as well as other polynucleotidyl transferase enzymes require the presence of divalent metal ions for activity. However, the precise role of the metal ions remains unclear. Both one and two metal ion mechanisms have been proposed. E. coli RNase H1 is thought to work via a one-metal ion mechanism in which Mg 2+, coordinated by one carboxylate group, mediates interactions with the nucleic acid substrate. The other two carboxylates activate a water molecule that can then attack the scissile phosphate bond (46, 47). The two-metal ion mechanism was first proposed for the 3′ to 5′ exonuclease of the Klenow fragment (48, 49). In this case, one metal interacts with the substrate and stabilizes the reaction intermediate and the other activates a water molecule and positions it to attack the scissile phosphate. Indeed, only one metal is observed in the crystal structures of E. coli RNase H1 (42) and A. fulgidus RNase HII (43) while two are seen in the active site of the isolated HIV RNase H domain of reverse transcriptase (50). Though the absence of a second metal ion in a crystal structure does not preclude a two-metal ion mechanism (since the second metal may have weak binding in the absence of substrates) there are indications that RNase H1 does use a single-metal ion mechanism while HIV RNase H uses two (51). For the PIWI domain of PfAgo, a strong peak is identified in the F_{obs}-F_{calc} difference electron density map near D558, and it is assigned as a water molecule at this time. By growing crystals in the presence of divalent metal ions, this may be assigned as a metal site unambiguously. A divalent metal ion appears to be required for Argonaute activity (52, 53).

3. siRNA Binding

The role of Argonaute is presently unknown in archaebacteria. Because of its similarity to Argonautes in eukaryotes, the siRNA binding characteristics of PfAgo were examined by using crosslinking and competition assays. A single-stranded 21-mer siRNA containing an IodoU nucleotide to facilitate crosslinking gave rise to a crosslinked species, whereas a double-stranded siRNA did not (FIG. 4A). In addition, the same labeled ss-siRNA can be readily competed off with an identical unlabeled oligonucleotide. However, a similar ss-siRNA lacking the 5′-phosphate moiety was unable to compete for crosslinking, even at greater than ten-fold the concentration than that at which competition with the 5′-phosphorylated ss-siRNA was seen (FIG. 4B). Thus, there appears to be a requirement for a bona fide siRNA for binding. Preferential binding of the ss-siRNA over the ds-version is consistent with the observation that a ds-siRNA cannot be loaded in vitro to an RISC complex, though an ss-siRNA can be. Accordingly, the present application provides an RISC complex comprising an RNAi construct, e.g., an ss-siRNA. The RISC complex preferably comprises an Argonaute protein, most preferably, an Argonaute protein with the “slicer” activity, described in greater detail below.

4. “Slicer” Activity

The finding that the PIWI domain in Argonaute is an RNase H domain suggests Argonaute as the, as of yet unidentified, “Slicer” enzyme of RISC, that is, the enzyme that cleaves the mRNA. RNase H enzymes specialize in single-stranded cleavage of RNA “guided” by a DNA strand in a double-stranded RNA/DNA hybrid. In a similar manner, Argonautes may specialize in RNA cleavage, in particular mRNA, guided by the siRNA strand in a ds RNA substrate. Moreover, unlike most RNases that leave a 3′-phosphate and 5′-OH, RNase H enzymes produce products with 3′-OH and 5′ phosphate groups (54). Recently, Martinez and Tuschl, and Zamore and colleagues showed that cleavage of the mRNA by RISC produces the latter type of termini (52, 53). A dependence on Mg²⁺ for activity is another hallmark of RNase H enzymes and RISC was also shown to require Mg²⁺ for cleavage as well (52). The PAZ domain, shown to recognize and bind the 3′ ends of siRNAs, and the PIWI domain, now shown to be an RNase H domain for catalytic activity, combine the necessary features of the slicing component of the RNAi machinery. Therefore, Argonaute, the signature component of RISC, can be “Slicer” itself.

5. A Model for si-RNA-Guided mRNA Cleavage

The placement of the PAZ domain on top of the crescent formed by the N-terminal, middle and PIWI domains and cradled by the connecter region in the structure of Argonaute defines a distinct groove through the protein. The groove has a claw shape that bends around between the PAZ and N-terminal domains. A striking feature of the structure is evident when the electrostatic potential is mapped on the surface of the protein. As shown in FIG. 5A, the surface of this inner groove is completely lined with positive charges. These positive charges are of course suitable for interaction with the negatively charged phosphate backbone and with the 2′-hydroxyl moieties of an RNA molecule, implicating the groove for substrate binding. The substrate for Argonaute is a ds-RNA molecule composed of an ss-siRNA acting as a guide and the mRNA.

In order to examine possible substrate binding modes for Argonaute, the knowledge of siRNA binding to the PAZ domain using the known PAZ-RNA structure (33) and the mode of binding of RNase H substrates (43, 55-57) were combined. Since the PAZ domain of PfAgo superimposes so well with the PAZ domain of hAgo1 in the PAZ-RNA complex as shown above, the two PAZ domains were superimposed and examined for the resulting position of the RNA with respect to PfAgo. The strand that interacts with its 3′ end in the PAZ cleft was regarded as the siRNA guide. The second strand would then be regarded as the mRNA substrate strand (see FIG. 5B). The siRNA guide has its 2 nucleotides at its 3′ end inserted into the PAZ cleft. The nucleotides just 5′ to that track the top of the PAZ b-barrel making very similar, if not identical, interactions with the PAZ domain as in the crystal structure of the PAZ-RNA complex. A long loop present in the PfAgo PAZ domain would probably move up slightly to accommodate the siRNA. Upon examination of the resulting location of the passenger strand, the mRNA would be coming into the binding groove with its 5′ end between the PAZ and the N-terminal domains. The N-terminus then acts as an “mRNA grip” on that end of the molecule. It should be noted that there is another extension of the groove that lies between the N-terminal and the PIWI domains, which could accommodate a single-stranded nucleic acid.

The double-stranded RNA was further extended into the molecule along the binding groove by model building. Remarkably, the mRNA would be positioned above the active site located in the PIWI domain 9 nucleotides from the 5′-side end of the double-stranded region, or rather 11 nucleotides if the 2 nucleotides of the guide that are inserted into the PAZ domain are counted and are probably not interacting with the mRNA. In other words, the scissile bond would be predicted to be between nucleotides 11 and 12 from the 5′ end of the message or from the 3′-end of the guide. This precisely coincides with the demonstrated cleavage of mRNAs by RISC 10 nucleotides from the 5′ end of an siRNA. The remainder of the RNA would then continue along the binding groove (FIG. 5C). The interdomain connecter is also forming part of the back wall of the binding groove. As the RNA molecule would have to bend somewhat, the details of some of these interactions are not clear. However, the length of the groove appears to accommodate the length of the siRNA guide, with the 5′ end of the guide probably interacting with the other side of the groove. From studies of other RNase H enzymes, Argonaute may sense the minor groove width of the dsRNA, which is different from that of dsDNA and from the minor groove width of a RNA/DNA hybrid, and which is in accord with the inability of RISC to cut DNA substrates (53). This mode of recognition would be in addition to binding the 3′ end of the siRNA and sensing the phosphate at the 5′end, as shown in the binding experiments (FIG. 4).

The groove as observed in the crystal structure presented here, in the absence of substrate, would fit an A-RNA double helix snugly. Though a single-stranded RNA should bind fairly readily, opening the claw of the molecule somewhat might assist binding the mRNA, after which it can close down on the double stranded substrate. A hinge region may exist in the interdomain connector at residues 317-320. This hinge could lift the PAZ and the away from the crescent base. This is reasonable since a RISC loading complex appears to be required for assembling an active RISC (58, 59).

The notion that RISC “Slicer” activity, i.e. siRNA-guided mRNA cleavage, resides in Argonaute itself was tested in a mammalian system where the RNAi pathway is known to function. It appears that mammalian Argonaute proteins are distinct and that Ago2 is functional for mRNA cleavage. Based on the sequence alignment with the archael protein, D597, D669 and a third amino acid (e.g., E683) of hAgo2 correspond to D558, D628 and E635 of PfAgo to form the catalytic triad “DDE” motif. There is an insertion near E683, and E673 may also act as the third carboxylate in hAgo2. The conserved active site aspartates were mutated and the mutants lost their nuclease activity while retaining binding to the siRNA guide. Therefore, Argonaute itself functions as the Slicer enzyme in the RNAi pathway.

In siRNA-guided mRNA cleavage, once RISC is formed, it needs to identify its homologous targets, both for target cleavage and for repression at the level of protein synthesis. In the latter case, there is a presumably stable interaction that occurs between the siRNA and its target, with the target being somehow protected from cleavage. Certainly, an absence of base pairing in the region of the active site might distort the complex sufficiently to prevent catalysis.

Furthermore, several Argonaute protein family members appear to be inactive towards mRNA cleavage despite the presence of the catalytic residues. The basis for these differences may help elucidate the details of the mechanism for siRNA-guided mRNA cleavage. The situation here might be somewhat analogous to the case of the transposase Tn5 and its inhibitor, which posses a catalytic domain with a similar RNase H-like fold. Tn5 inhibitor is a truncated version of the active Tn5 transposase and retains the essential catalytic residues. However, there are major conformational differences between the two that result in domains of the proteins being in different positions relative to one another (40, 45). Similarly, mutations have been introduced into a catalytically active Ago protein, hAgo2, in the vicinity of the active site, which change residues to corresponding residues in an inactive Ago, hAgo1. These inactivate Ago2 for cleavage, indicating that there are determinants for catalysis beyond simply the catalytic triad and that relatively minor alterations in the PIWI domain can have profound effects on its activity toward RNA substrates. The common fold in the catalytic domain of Argonaute family members and transposases and integrases is also intriguing given the relationship of RNAi with control of transposition. It is worth noting that the identification of the catalytic center of RISC awaited a drive toward understanding RNAi at a structural level. Thus, it seems likely that, as in the present example, a full understanding of the underlying mechanism of RNAi will derive from a combination of detailed biochemical and structural studies of RISC.

Assays

The assays and methods described herein may used in combination or separately. For example, an in silico screening and an in vitro binding assay and/or an activity assay may be combined to identify a binding agent and/or a binding agent for a protein that also modulates activity of the protein.

I. Assays Based on the Atomic Structure Coordinates

Structural information, often in the form of atomic structure coordinates, may also be used in a variety of molecular modeling and computer-based screening applications to, for example, design variants that have altered biological properties or to computationally design, screen for and/or identify compounds that bind to the Argonaute protein or to fragments of the Argonaute protein. These compounds may modulate the activity of Argonaute protein and hence the RISC activity.

Thus, in a further aspect of the application, the data from the crystal structure of Argonaute is used to evaluate compounds for their utility as modulators of Argonuate protein. These methods comprise designing and synthesizing candidate compounds using the atomic coordinates of the three dimensional structure of such co-crystals and screening for its utility in various pharmaceutical applications.

In another embodiment, the structures are probed with a plurality of molecules to determine their ability to bind to the Argonaute protein at various sites. Such molecules may be able to modulate the activity of Argonaute protein.

In yet another embodiment, the structures can be used to computationally screen small molecule databases for chemical entities or compounds that can bind in whole, or in part, to Argonaute. In this screening, the quality of fit of such entities or compounds to the binding site may be judged either by shape complementarity or by estimated interaction energy. (Meng et al., 1992, J. Comp. Chem. 13:505-524).

The design of compounds that bind to Argonaute according to this invention generally involves consideration of two factors. First, the compound must be capable of physically and structurally associating with Argonaute. This association can be covalent or non-covalent. For example, covalent interactions may be important for designing suicide or irreversible inhibitors of a protein. Non-covalent molecular interactions important in the association of Argonaute include hydrogen bonding, ionic and other polar interactions, interactions as well as van der Waals interactions. Second, the compound must be able to assume a conformation that allows it to associate with the Argonaute protein. Although certain portions of the compound will not directly participate in this association with the protein, those portions may still influence the overall conformation of the molecule. This, in turn, may have a significant impact on potency. Such conformational requirements include the overall three-dimensional structure and orientation of the chemical group or compound in relation to all or a portion of the binding site, or the spacing between functional groups of a compound comprising several chemical groups that directly interact with the protein.

The potential modulatory or binding effect of a chemical compound on Argonaute may be analyzed prior to its actual synthesis and testing by the use of computer modeling techniques. If the theoretical structure of the given compound suggests insufficient interaction and association between it and the protein, synthesis and testing of the compound is unnecessary. However, if computer modeling indicates a strong interaction, the molecule may then be synthesized and tested for its ability to bind to the protein and inhibit its activity. In this manner, synthesis of ineffective compounds may be avoided.

A binding compound of Argonaute may be computationally evaluated and designed by means of a series of steps in which chemical groups or fragments are screened and selected for their ability to associate with the individual binding pockets or interface surfaces of each of the proteins. One skilled in the art may use one of several methods to screen chemical groups or fragments for their ability to associate with Argonaute. Docking may be accomplished using software such as QUANTA and SYBYL, followed by energy minimization and molecular dynamics with standard molecular mechanics force fields, such as CHARMM and AMBER.

Specialized computer programs may also assist in the process of selecting fragments or chemical groups. These include:

1. GRID (Goodford, 1985, J. Med. Chem. 28:849-857). GRID is available from Oxford University, Oxford, UK;

2. MCSS (Miranker & Karplus, 1991, Proteins: Structure, Function and Genetics 11:29-34). MCSS is available from Molecular Simulations, Burlington, Mass.;

3. AUTODOCK (Goodsell & Olsen, 1990, Proteins: Structure, Function, and Genetics 8:195-202). AUTODOCK is available from Scripps Research Institute, La Jolla, Calif.;

4. DOCK (Kuntz et al., 1982, J. Mol. Biol. 161:269-288). DOCK is available from University of California, San Francisco, Calif.;

5. FlexE (Clausen H, Buning C, Rarey M and Lengauer T) J. Mol. Biol. (2001) 308, 377-395. FlexE is available from Tripos, St. Louis, Mo.;

6. Glide, Glide is available from Schrodinger, Portland, Oreg.;

7. Gold, Jones et al. J. Mol. Biol. 245, 43-53, 1995;

8. QXP, McMartin C, Bohacek R S. J Comput Aided Mol Des 1997 11:333-44;

9. ICM. (http://www.molsoft.com). Available from Molsoft, San Diego, Calif.; and

10. FlexX. [Sybl, Tripos, St. Louis, Mo.

Once suitable chemical groups or fragments have been selected, they can be assembled into a single compound. Assembly may proceed by visual inspection of the relationship of the fragments to each other in the three-dimensional image displayed on a computer screen in relation to the structure coordinates of Argonaute. This would be followed by manual model building using software such as QUANTA or SYBYL.

Useful programs to aid one of skill in the art in connecting the individual chemical groups or fragments include:

1. CAVEAT (Bartlett et al., 1989, ‘CAVEAT: A Program to Facilitate the Structure-Derived Design of Biologically Active Molecules.’ In Molecular Recognition in Chemical and Biological Problems', Special Pub., Royal Chem. Soc. 78:182-196). CAVEAT is available from the University of California, Berkeley, Calif.;

2. 3D Database systems such as MACCS-3D (MDL Information Systems, San Leandro, Calif.). This area is reviewed in Martin, 1992, J. Med. Chem. 35:2145-2154); and

3. HOOK (available from Molecular Simulations, Burlington, Mass.).

Instead of proceeding to build a modulator of Argonaute in a step-wise fashion one fragment or chemical group at a time, as described above, Argonaute-binding compounds or modulators may be designed as a whole or ‘de novo’ using either an empty binding site or the surface of a protein that participates in protein/protein interactions in a co-complex, or optionally including some portion(s) of a known modulator(s). These methods include:

1. LUDI (Bohm, 1992, J. Comp. Aid. Molec. Design 6:61-78). LUDI is available from Molecular Simulations, Inc., San Diego, Calif.;

2. LEGEND (Nishibata & Itai, 1991, Tetrahedron 47:8985). LEGEND is available from Molecular Simulations, Burlington, Mass.; and

3. LeapFrog (available from Tripos, Inc., St. Louis, Mo.).

Other molecular modeling techniques may also be employed in accordance with this invention. See, e.g., Cohen et al., 1990, J. Med. Chem. 33:883-894. See also, Navia & Murcko, 1992, Current Opinions in Structural Biology 2:202-210.

Once a compound has been designed or selected by the above methods, the efficiency with which that compound may bind to Argonaute may be tested and optimized by computational evaluation. An effective modulator of Argonaute must preferably demonstrate a relatively small difference in energy between its bound and free states (i.e., it must have a small deformation energy of binding). Thus, the most efficient modulators should preferably be designed with a deformation energy of binding of not greater than about 10 kcal/mol, preferably, not greater than 7 kcal/mol. Modulators may interact with the protein in more than one conformation that is similar in overall binding energy. In those cases, the deformation energy of binding is taken to be the difference between the energy of the free compound and the average energy of the conformations observed when the modulator binds to the protein.

A compound selected or designed for binding to or inhibiting Argonaute may be further computationally optimized so that in its bound state it would preferably lack repulsive electrostatic interaction with the target protein. Such non-complementary electrostatic interactions include repulsive charge-charge, dipole-dipole and charge-dipole interactions. Specifically, the sum of all electrostatic interactions between the modulator and the protein when the modulator is bound to it preferably make a neutral or favorable contribution to the enthalpy of binding.

Specific computer software is available in the art to evaluate compound deformation energy and electrostatic interaction. Examples of programs designed for such uses include: Gaussian 92, revision C (Frisch, Gaussian, Inc., Pittsburgh, Pa. ©1992); AMBER, version 4.0 (Kollman, University of California at San Francisco, ©994); QUANTA/CHARMM (Molecular Simulations, Inc., Burlington, Mass., ©1994); and Insight II/Discover (Biosym Technologies Inc., San Diego, Calif., ©1994). These programs may be implemented, for instance, using a computer workstation, as are well-known in the art. Other hardware systems and software packages will be known to those skilled in the art.

The computer-assisted methods for designing a modulator of Argonaute activity can be de novo or based on a candidate compound. An example of a computer-assisted method for designing an modulator of Argonaute activity de novo would thus involve the steps of: (1) supplying a computer modeling application with a set of structure coordinates of a molecule or molecular complex comprising at least a portion of an Argonaute; (2) computationally building a chemical entity represented by a set of structure coordinates; and (3) determining whether the chemical entity is an modulator expected to bind to or interfere with the molecule or molecular complex, wherein binding to or interfering with the molecule or molecular complex is indicative of potential modulation of Aargonaute activity.

Once an modulator or Argonaute binding compound has been optimally selected or designed, as described above, substitutions may then be made in some of its atoms or chemical groups in order to improve or modify its binding properties. Generally, initial substitutions are conservative, i.e., the replacement group will have approximately the same size, shape, hydrophobicity and charge as the original group. One of skill in the art will understand that substitutions known in the art to alter conformation should be avoided. Such altered chemical compounds may then be analyzed for efficiency of binding to Argonaute by the same computer methods described in detail above.

An example of such a computer-assisted method for identifying an modulator of Argonaute activity would thus involve (1) supplying a computer modeling application with a set of structure coordinates of a molecule or molecular complex comprising at least a portion of an Argonaute or Argonaute-like compound, (2) supplying the computer modeling application with a set of structure coordinates of a chemical entity; and (3) determining whether the chemical entity is an modulator expected to bind to or modulate the molecule or molecular complex.

The structure coordinates of an Argonaute co-complex, or of Argonaute alone, or of portions thereof, are particularly useful to solve the structure of other co-complexes of Argonaute, of mutants, of the Argonaute co-complex further complexed to another molecule, or of the crystalline form of any other protein or protein co-complex with significant amino acid sequence homology to any functional domain of Argonaute.

One method that may be employed for this purpose is molecular replacement. In this method, the unknown co-crystal structure, whether it is another Argonaute co-complex, a mutant, a Argonaute co-complex that is further complexed to another molecule, or the crystal of some other protein or protein co-complex with significant amino acid sequence homology to any functional domain of one of the proteins in the co-complex crystal, may be determined using phase information from the present Argonaute co-complex structure coordinates. This method will provide an accurate three-dimensional structure for the unknown protein or protein co-complex in the new crystal more quickly and efficiently than attempting to determine such information ab initio.

If an unknown crystal form has the same space group as and similar cell dimensions to the known co-complex crystal form, then the phases derived from the known crystal form can be directly applied to the unknown crystal form, and in turn, an electron density map for the unknown crystal form can be calculated. Difference electron density maps can then be used to examine the differences between the unknown crystal form and the known crystal form. A difference electron density map is a subtraction of one electron density map, e.g., that derived from the known crystal form, from another electron density map, e.g., that derived from the unknown crystal form. Therefore, all similar features of the two electron density maps are eliminated in the subtraction and only the differences between the two structures remain. However, if the space groups and/or cell dimensions of the two crystal forms are different, then this approach will not work and molecular replacement must be used in order to derive phases for the unknown crystal form.

The techniques of X-ray diffraction can be employed in the study of the co-complexes of Argonaute. This information may thus be used to optimize known modulators of Argonaute and more importantly, to design and synthesize novel classes of modulators of Argonaute.

Subsets of the atomic structure coordinates can also be used in any of the above methods. Particularly useful subsets of the coordinates include, but are not limited to, coordinates of single domains, coordinates of residues lining an active site, coordinates of residues that participate in important protein-protein contacts at an interface, and Cα coordinates. For example, the coordinates of one domain of a protein that contains the active site may be used to design modulators that bind to that site, even though the protein is fully described by a larger set of atomic coordinates. Therefore, as described in detail for the specific embodiments, below, a set of atomic coordinates that define the entire polypeptide chain, although useful for many applications, do not necessarily need to be used for the methods described herein.

II. Assay for Argonaute RNase Activity

The present application provides screening methods for agents that modulate the RNase activity of the Argonaute protein. Applicants have shown that Argonaute has a RNase H domain and acts as the Slicer enzyme of RISC to cleave mRNA bound by a single-stranded siRNA. Thus, the Argonaute activity can be assayed by measuring by any standard techniques in the art for measuring RNase activity. The exemplification provides one such example.

In certain embodiments, the RNase H activity of Argonaute can be measured. For example, WO 04/59012 describes a “Molecular Beacon” Assay for measuring RNase H activity and/or other nuclease-mediated cleavage of nucleic acids. Briefly, the assay detects degradation of a nucleic acid substrate which, preferably, is an RNA substrate that is annealed to at least one region or part of an oligonucleotide probe. In preferred embodiments, the oligonucleotide probe is a DNA probe (e.g., a deoxyoligonucleotide probe), which may also be referred to in the context of this invention as the DNA “substrate” moiety. Typically, both the oligonucleotide probe and the RNA substrate will be oligonucleotide molecules that are between about 10 and about 100 nucleotides in length and may be, e.g., between about 1050 nucleotides in length, more preferably between 15-25 nucleotides length. In preferred embodiments, the oligonucleotide probe is at least 18 nucleotides in length.

Chan et al. describes a capillary electrophoretic assay to measure RNase H activity. See Anal Biochem. 2004 Aug. 15;331(2):296-302. Briefly, cleavage of a fluorescein-labeled RNA-DNA heteroduplex was monitored by capillary electrophoresis. This assay was used as a secondary assay to confirm hits from a high-throughput screening program. Since autofluorescent compounds in samples migrated differently from both substrate and product in most cases, the assay was extremely robust for assaying enzymatic inhibition of such samples, in contrast to a simple well-based approach.

The screening methods may be conducted in a high-throughput fashion using any techniques available in the art. Recently, Parniak et al. described a fluorescence-based high-throughput screening assay for inhibitors of HIV RNase H activity. See Anal Biochem 2003, 322:33-9. Briefly, the assay substrate is an 18-nucleotide 3′-fluorescein-labeled RNA annealed to a complementary 18-nucleotide 5′-Dabcyl-modified DNA. The intact duplex has an extremely low background fluorescent signal and provides up to 50-fold fluorescent signal enhancement following hydrolysis. The size and sequence of the duplex are such that HIV-1 RT-RNase H cuts the RNA strand close to the 3′ end. The fluorescein-labeled ribonucleotide fragment readily dissociates from the complementary DNA at room temperature with immediate generation of a fluorescent signal. This assay is rapid, inexpensive, and robust, providing Z′ factors of 0.8 and coefficients of variation of about 5%. The assay can be carried out both in real-time (continuous) and in “quench” modes; the latter requires only two addition steps with no washing and is thus suitable for robotic operation. Several chemical libraries totaling more than 106,000 compounds were screened with this assay in approximately 1 month.

Alternatively, McLellan et al. described a nonradioactive, 96-well plate assay designed to be used for high-throughput screening of compounds capable of inhibiting the RNase H activity of HIV-1 reverse transcriptase. See McLellan at al., Biotechniques. 2002 August;33(2):424-9. In this method, tRNA is employed as substrate that was labeled with digoxygenin-modified reporter residues. The labeled tRNA was prehybridized with a DNA oligonucleotide that contained a single biotinylated residue at its 5′-terminus to ensure its attachment to streptavidin-coated microplates. The uncleaved, immobilized DNA/tRNA substrate was detected through the use of established ELISA protocols. Incubation with purified HIV-1 reverse transcriptase initiated RNase H degradation and caused a signal reduction to negligible background levels. In contrast, the signal intensity remained unaffected when using an RNase H deficient mutant enzyme. The assay was validated using the hydrazone derivative BBNH that was previously shown to inhibit RNase H degradation below concentrations of 10 microM.

III. Reporter Gene Assay

The application also provides reporter gene assays. The reporter gene assays may be used to identify agents that modulate (e.g., increase) expression of Argonaute gene(s), e.g., by modulating Argonaute's promoter activity. For example, by operably linking an Argonaute's promoter with a reporter gene, the activity of the promoter can be monitored through monitoring/measuring the expression level of reporter gene. Many reporter gene assays have been developed and known to skilled artisans. Examples include: β-galactosidase assays; β-glucuronidase assays; B-lactamase assays (kits, β-lacatamase FRET substrates or color substrates are commercially available); CAT assays; Dual Reporter assays; GFP Assays; Luciferase Assays; SEAP Assays.

IV. Binding Assay

As described above, in silico screening or assays may be developed to identify a ligand or an inhibitor of interest, such as a ligand or an inhibitor that interacts with an Argonaute protein, e.g., a hAgo-2 protein. A ligand generally refers to a molecule (e.g., a nucleic acid molecule or a non-nucleic acid small molecule) that binds a molecule of interest (e.g., an Argonaute protein of the application). An inhibitor generally refers to a molecule that inhibits the function or activity of its target molecule, e.g., an Argonaute protein of the application.

A variety of assay formats will suffice and, in light of the present disclosure, those not expressly described herein will nevertheless be comprehended by one of ordinary skill in the art. Assay formats which approximate such conditions as formation of protein-based complexes and enzymatic activity may be generated in many different forms, and include assays based on cell-free systems, e.g., purified proteins or cell lysates, as well as cell-based assays which utilize intact cells. Simple binding assays can also be used to detect agents which bind to a protein of the application. Agents to be tested can be produced, for example, by bacteria, yeast or other organisms (e.g., natural products), produced chemically (e.g., small molecules, including peptidomimetics), or produced recombinantly. In a preferred embodiment, the test agent is a small organic molecule, e.g., other than a peptide or oligonucleotide, having a molecular weight of less than about 6,000 daltons.

In many drug screening programs which test libraries of compounds and natural extracts, high throughput assays are desirable in order to maximize the number of compounds surveyed in a given period of time. Assays of the present application which are performed in cell-free systems, such as may be developed with purified or semi-purified proteins or with lysates, are often preferred as “primary” screens in that they can be generated to permit rapid development and relatively easy detection of an alteration in a molecular target which is mediated by a test compound. Moreover, the effects of cellular toxicity and/or bioavailability of the test compound can be generally ignored in the in vitro system, the assay instead being focused primarily on the effect of the drug on the molecular target as may be manifest in the affinity of the drug to the molecular target and/or changes in enzymatic properties of the molecular target.

In certain embodiments, an Argonaute protein to be used in a binding assay is at least semi-purified proteins. By semi-purified, it is meant that the proteins utilized in the reconstituted mixture have been previously separated from other cellular or viral proteins. For instance, in contrast to cell lysates, the protein involved in the protein-based complex formation are present in the mixture to at least 50% purity relative to all other proteins in the mixture, and more preferably are present at 90-95% purity.

Assaying the protein-based complexes of the application, in the presence or absence of a candidate agent, can be accomplished in any vessel suitable for containing the reactants. Examples include microtitre plates, test tubes, and micro-centrifuge tubes.

In an exemplary binding assay, the agent or compound of interest is contacted with an Argonaute protein. Detection and quantification of the Argonaute protein-based complex (e.g., a co-complex formed by the Argonaute protein and the compound) provides a means for determining the compound's affinity for the Argonaute protein.

Protein-based complex formation may be detected by a variety of techniques, many of which are effectively described herein. For instance, formation of complexes can be quantitated using, for example, detectably labeled proteins (e.g., radiolabeled, fluorescently labeled, or enzymatically labeled), by immunoassay, or by chromatographic detection. Surface plasmon resonance systems, such as those available from Biacore International AB (Uppsala, Sweden), may also be used to detect binding interactions.

Often, it will be desirable to immobilize the protein to facilitate separation of complexes from uncomplexed forms of agents to be assayed for their binding affinity to a protein, as well as to accommodate automation of the assay. In an illustrative embodiment, a fusion protein can be provided which adds a domain that permits the protein (or a portion of the protein) to be bound to an insoluble matrix. For example, GST-Argonaute (or a portion thereof) fusion proteins can be adsorbed onto glutathione sepharose beads (Sigma Chemical, St. Louis, Mo.) or glutathione derivatized microtitre plates, which are then combined with test agents, e.g., a radio- or fluorescent-labeled agents, and incubated under conditions conducive to complex formation. Following incubation, the beads are washed to remove any unbound test agents, and the matrix bead-bound label(s) determined directly, or in the supernatant after the complexes are dissociated, e.g., when microtitre plate is used.

RNAi

The term “RNAi construct,” as used herein, comprises nucleotides that hybridize under physiological condition to a portion of a target gene and attenuates expression of the target gene. In certain embodiments, the RNAi construct, when introduced into a cell, induces a sequence-specific RNA interference process. The RNAi construct used in the present application may be single-stranded siRNAs (ssRNAs), double-stranded siRNAs (dsRNAs), which includes short “hairpin” RNAs (shRNAs). An RNAi construct used in the present application may be single-stranded siRNAs (ssRNAs), double-stranded siRNAs (dsRNAs), which include short “hairpin” RNAs (shRNAs). The RNAi construct may comprise one or more strands of polymerized ribonucleotide. It may include modifications to either the phosphate-sugar backbone or the nucleoside. For example, the phosphodiester linkages of natural RNA may be modified to include at least one of a nitrogen or sulfur heteroatom. Modifications in RNA structure may be tailored to allow specific genetic inhibition while avoiding a general panic response in some organisms which is generated by RNAi. Likewise, bases may be modified to block the activity of adenosine deaminase. The RNAi construct may be produced enzymatically or by partial/total organic synthesis, any modified ribonucleotide can be introduced by in vitro enzymatic or organic synthesis.

The RNAi construct may be directly introduced into the cell (i.e., intracellularly); or introduced extracellularly into a cavity, interstitial space, into the circulation of an organism, introduced orally, or may be introduced by bathing an organism in a solution containing RNA. Methods for oral introduction include direct mixing of RNA with food of the organism, as well as engineered approaches in which a species that is used as food is engineered to express an RNA, then fed to the organism to be affected. Physical methods of introducing nucleic acids include injection of an RNA solution directly into the cell or extracellular injection into the organism.

The double-stranded structure may be formed by a single self-complementary RNA strand (shRNA) or two complementary RNA strands. RNA duplex formation may be initiated either inside or outside the cell. The RNA may be introduced in an amount which allows delivery of at least one copy per cell. Higher doses (e.g., at least 5, 10, 100, 500 or 1000 copies per cell) of double-stranded material may yield more effective inhibition; lower doses may also be useful for specific applications. Inhibition is sequence-specific in that nucleotide sequences corresponding to the duplex region of the RNA are targeted for genetic inhibition.

RNAi constructs containing a nucleotide sequences identical to a portion, of either coding or non-coding sequence, of the target gene are preferred for inhibition. RNA sequences with insertions, deletions, and single point mutations relative to the target sequence (ds RNA similar to the target gene) have also been found to be effective for inhibition. Thus, sequence identity may be optimized by sequence comparison and alignment algorithms known in the art (see Gribskov and Devereux, Sequence Analysis Primer, Stockton Press, 1991, and references cited therein) and calculating the percent difference between the nucleotide sequences by, for example, the Smith-Waterman algorithm as implemented in the BESTFIT software program using default parameters (e.g., University of Wisconsin Genetic Computing Group). Greater than 90% sequence identity, or even 100% sequence identity, between the inhibitory RNA and the portion of the target gene is preferred. Alternatively, the duplex region of the RNA may be defined functionally as a nucleotide sequence that is capable of hybridizing with a portion of the target gene transcript (e.g., 400 mM NaCl, 40 mM PIPES pH 6.4, 1 mM EDTA, 50° C. or 70° C. hybridization for 12-16 hours; followed by washing). In certain preferred embodiments, the length of the RNAi is at least 20, 21 or 22 nucleotides in length, e.g., corresponding in size to RNA products produced by Dicer-dependent cleavage. In certain embodiments, the RNAi construct is at least 25, 50, 100, 200, 300 or 400 bases. In certain embodiments, the RNAi construct is 400-800 bases in length.

In certain embodiments, an shRNA construct is designed with about 29 bp helices. Further information on the optimization of shRNA constructs may be found, for example, in the following references: Paddison, et al. Proc Natl Acad Sci USA, 2002. 99(3): p. 1443-8; 13. Brummelkamp, et al. Science, 2002. 21: p. 21; Kawasaki, et al. Nucleic Acids Res, 2003. 31(2): p. 700-7; Lee et al. Nat Biotechnol, 2002. 20(5): p. 500-5; Miyagishi, et al. Nat Biotechnol, 2002. 20(5): p. 497-500; Paul., et al., Nat Biotechnol, 2002. 20(5): p. 505-8.

The RNAi construct may be synthesized either in vivo or in vitro. Endogenous RNA polymerase of the cell may mediate transcription in vivo, or cloned RNA polymerase can be used for transcription in vivo or in vitro. For transcription from a transgene in vivo or an expression construct, a regulatory region (e.g., promoter, enhancer, silencer, splice donor and acceptor, polyadenylation) may be used to transcribe the RNAi strand (or strands). Inhibition may be targeted by specific transcription in an organ, tissue, or cell type; stimulation of an environmental condition (e.g., infection, stress, temperature, chemical inducers); and/or engineering transcription at a developmental stage or age. The RNA strands may or may not be polyadenylated; the RNA strands may or may not be capable of being translated into a polypeptide by a cell's translational apparatus. The RNAi construct may be chemically or enzymatically synthesized by manual or automated reactions. The RNAi construct may be synthesized by a cellular RNA polymerase or a bacteriophage RNA polymerase (e.g., T3, T7, SP6). The use and production of an expression construct are known in the art (see also WO 97/32016; U.S. Pat. Nos. 5,593,874, 5,698,425, 5,712,135, 5,789,214, and 5,804,693; and the references cited therein). If synthesized chemically or by in vitro enzymatic synthesis, the RNA may be purified prior to introduction into the cell. For example, RNA can be purified from a mixture by extraction with a solvent or resin, precipitation, electrophoresis, chromatography or a combination thereof. Alternatively, the RNAi construct may be used with no or a minimum of purification to avoid losses due to sample processing. The RNAi construct may be dried for storage or dissolved in an aqueous solution. The solution may contain buffers or salts to promote annealing, and/or stabilization of the duplex strands.

Physical methods of introducing nucleic acids include injection of a solution containing the RNAi construct, bombardment by particles covered by the RNAi construct, soaking the cell or organism in a solution of the RNA, or electroporation of cell membranes in the presence of the RNAi construct. A viral construct packaged into a viral particle would accomplish both efficient introduction of an expression construct into the cell and transcription of RNAi construct encoded by the expression construct. Other methods known in the art for introducing nucleic acids to cells may be used, such as lipid-mediated carrier transport, chemical mediated transport, such as calcium phosphate, and the like. Thus the RNAi construct may be introduced along with components that perform one or more of the following activities: enhance RNA uptake by the cell, promote annealing of the duplex strands, stabilize the annealed strands, or other-wise increase inhibition of the target gene.

“Inhibition of gene expression” refers to the absence or observable decrease in the level of protein and/or mRNA product from a target gene. “Specificity” refers to the ability to inhibit the target gene without manifest effects on other genes of the cell. The consequences of inhibition can be confirmed by examination of the outward properties of the cell or organism (as presented below in the examples) or by biochemical techniques such as RNA solution hybridization, nuclease protection, Northern hybridization, reverse transcription, gene expression monitoring with a microarray, antibody binding, enzyme linked immunosorbent assay (ELISA), Western blotting, radioimmunoassay (RIA), other immunoassays, and fluorescence activated cell analysis (FACS). For RNA-mediated inhibition in a cell line or whole organism, gene expression is conveniently assayed by use of a reporter or drug resistance gene whose protein product is easily assayed. Such reporter genes include acetohydroxyacid synthase (AHAS), alkaline phosphatase (AP), beta galactosidase (LacZ), beta glucoronidase (GUS), chloramphenicol acetyltransferase (CAT), green fluorescent protein (GFP), horseradish peroxidase (HRP), luciferase (Luc), nopaline synthase (NOS), octopine synthase (OCS), and derivatives thereof. Multiple selectable markers are available that confer resistance to ampicillin, bleomycin, chloramphenicol, gentamycin, hygromycin, kanamycin, lincomycin, methotrexate, phosphinothricin, puromycin, and tetracyclin.

Depending on the assay, quantitation of the amount of gene expression allows one to determine a degree of inhibition which is greater than 10%, 33%, 50%, 90%, 95% or 99% as compared to a cell not treated according to the present application. As an example, the efficiency of inhibition may be determined by assessing the amount of gene product in the cell: mRNA may be detected with a hybridization probe having a nucleotide sequence outside the region used for the inhibitory double-stranded RNA, or translated polypeptide may be detected with an antibody raised against the polypeptide sequence of that region.

As disclosed herein, the present application is not limited to any type of target gene or nucleotide sequence. In some preferred embodiments, the target gene is an essential gene or a gene which is essential for cell viability. The following classes of possible target genes are listed for illustrative purposes: developmental genes (e.g., adhesion molecules, cyclin kinase inhibitors, Writ family members, Pax family members, Winged helix family members, Hox family members, cytokines, lymphokines and their receptors, growth/differentiation factors and their receptors, neurotransmitters and their receptors); oncogenes (e.g., ABLI, BCLI, BCL2, BCL6, CBFA2, CBL, CSFIR, ERBA, ERBB, EBRB2, ETSI, ETS1, ETV6, FGR, FOS, FYN, HCR, HRAS, JUN, KRAS, LCK, LYN, MDM2, MLL, MYB, MYC, MYCLI, MYCN, NRAS, PIM 1, PML, RET, SRC, TALI, TCL3, and YES); tumor suppressor genes (e.g., APC, BRCA1, BRCA2, MADH4, MCC, NF 1, NF2, RB 1, P53, BIM, PUMA and WTI); and enzymes (e.g., ACC synthases and oxidases, ACP desaturases and hydroxylases, ADP-glucose pyrophorylases, ATPases, alcohol dehydrogenases, amylases, amyloglucosidases, catalases, cellulases, chalcone synthases, chitinases, cyclooxygenases, decarboxylases, dextrinases, DNA and RNA polymerases, galactosidases, glucanases, glucose oxidases, granule-bound starch synthases, GTPases, helicases, hemicellulases, integrases, inulinases, invertases, isomerases, kinases, lactases, lipases, lipoxygenases, lysozymes, nopaline synthases, octopine synthases, pectinesterases, peroxidases, phosphatases, phospholipases, phosphorylases, phytases, plant growth regulator synthases, polygalacturonases, proteinases and peptidases, pullanases, recombinases, reverse transcriptases, RUBISCOs, topoisomerases, and xylanases).

The application also provides variations of the methods described herein, wherein gene expression of more than one gene is achieved. This may be achieved for example, by expressing multiple shRNAs, or by designing an shRNA to inhibit the gene expression of two or more genes which share substantial nucleotide sequence identity in a short stretch, preferably at least 90% identity over a length of 20, 22, 25, 27, or 30 nucleotides.

The compositions of the present application may be used to enhance the therapeutic effectiveness of a RNAi therapeutics. Exemplary RNAi therapeutics includes double-stranded ribonucleic acids (dsRNAs) for inhibiting the expression of a K-ras oncogene in a cell for treating pancreatic cancer, described in US20040121348, double-stranded ribonucleic acids (dsRNAs) having nucleotide sequences substantially identical to at least a part of a 3′-untranslated region (3′-UTR) of a (+) strand RNA virus useful for treating hepatitis C infection, described in US20040091457, siRNAs that down-regulate expression of neurite growth inhibitor receptor, prostaglandin D2 receptor, IkappaB kinase or protein kinase PKR genes, useful for treating cancer and inflammatory disease, described in U.S. Patent Application Publication No. 20030191077.

Furthermore, the crystal structure, the electronic representation, as well as other aspects of the application also relate to a method for identifying, designing, and/or optimizing an RNAi construct or RNAi therapeutic of the application. For example, based on the structure of the PAZ domain, particular the site that may interact with the 3′ end of a nucleic acid (e.g., an RNA or a portion of an RNAi construct), the nucleic acid sequence or structure may be designed and/or optimize to increase or decrease the nucleic acid's interaction with the PAZ domain. Similarly, based on the PIWI domain as well as the interface between the PIWI domain and the PAZ domain, an RNAi construct or RNAi therapeutic may be designed and/or optimized. An optimized RNAi therapeutic may have an improved pharmacokinetic and/or pharmacodynamic profile.

### REFERENCES

1. R. Jorgensen, Trends Biotechnol 8, 340-4. (1990).
2. D. Baulcombe, Arch Virol Suppl 15, 189-201. (1999).
3. R. F. Ketting, T. H. Haverkamp, H. G. van Luenen, R. H. Plasterk, Cell 99, 133-41 (1999).
4. I. M. Hall et al., Science 297, 2232-7. (2002).
5. T. Volpe et al., Science 22, 22 (2002).
6. G. J. Hannon, Nature 418, 244-51. (2002).
7. P. J. Paddison et al., Nature 428, 427-31 (2004).
8. A. G. Fraser et al., Nature 408, 325-30. (2000).
9. M. Boutros et al., Science 303, 832-5 (2004).
10. K. Berns et al., Nature 428,431-7 (2004).
11. A. Fire et al., Nature 391, 806-11. (1998).
12. S. M. Hammond, E. Bernstein, D. Beach, G. J. Hannon, Nature 404, 293-6 (2000).
13. P. D. Zamore, T. Tuschl, P. A. Sharp, D. P. Bartel, Cell 101, 25-33 (2000).
14. A. J. Hamilton, D. C. Baulcombe, Science 286, 950-2 (1999).
15. S. M. Elbashir, J. Martinez, A. Patkaniowska, W. Lendeckel, T. Tuschl, Embo J 20, 6877-88 (2001).
16. E. Bernstein, A. A. Caudy, S. M. Hammond, G. J. Hannon, Nature 409, 363-6. (2001).
17. S. M. Elbashir, W. Lendeckel, T. Tuschl, Genes Dev 15, 188-200 (2001).
18. A. Nykanen, B. Haley, P. D. Zamore, Cell 107, 309-21. (2001).
19. D. P. Bartel, Cell 116, 281-297 (2004).
20. Y. Lee et al., Nature 425, 415-9 (2003).
21. A. A. Caudy, M. Myers, G. J. Hannon, S. M. Hammond, Genes Dev 16, 2491-6 (2002).
22. G. Hutvagner, P. D. Zamore, Science 297, 2056-60 (2002).
23. Z. Mourelatos et al., Genes Dev 16, 720-8. (2002).
24. T. Tuschl, P. D. Zamore, R. Lehmann, D. P. Bartel, P. A. Sharp, Genes Dev 13, 3191-7 (1999).
25. D. S. Schwarz, G. Hutvagner, B. Haley, P. D. Zamore, Mol Cell 10, 537-48 (2002).
26. J. Martinez, A. Patkaniowska, H. Urlaub, R. Luhrmann, T. Tuschl, Cell 110, 563-74 (2002).
27. M. A. Carmell, Z. Xuan, M. Q. Zhang, G. J. Hannon, Genes Dev 16, 2733-42. (2002).
28. D. N. Cox et al., Genes Dev 12, 3715-27 (1998).
29. J. J. Song et al., Nat Struct Biol 10, 1026-1032 (2003).
30. K. S. Yan et al., Nature 426, 468-74 (2003).
31. A. Lingel, B. Simon, E. Izaurralde, M. Sattler, Nature 426, 465-9 (2003).
32. A. Lingel, B. Simon, E. Izaurralde, M. Sattler, Nat Struct Mol Biol 11, 576-7 (2004).
33. J. B. Ma, K. Ye, D. J. Patel, Nature 429, 318-22 (2004).
34. H. Zhang, F. A. Kolb, V. Brondani, E. Billy, W. Filipowicz, Embo J 21, 5875-85. (2002).
35. A. M. Friedman, T. O. Fischmann, T. A. Steitz, Science 268, 1721-7 (1995).
36. F. Dyda et al., Science 266, 1981-6 (1994).
37. J. Lubkowski et al., Biochemistry 38, 13512-22 (1999).
38. M. Ariyoshi et al., Cell 78, 1063-72 (1994).
39. P. Rice, K. Mizuuchi, Cell 82, 209-20 (1995).
40. D. R. Davies, I. Y. Goryshin, W. S. Reznikoff, I. Rayment, Science 289, 77-85 (2000).
41. L. Lai, H. Yokota, L. W. Hung, R. Kim, S. H. Kim, Structure Fold Des 8, 897-904 (2000).
42. K. Katayanagi, M. Okumura, K. Morikawa, Proteins 17, 337-46 (1993).
43. B. R. Chapados et al., J Mol Biol 307, 541-56 (2001).
44. W. Yang, T. A. Steitz, Structure 3, 1314 (1995).
45. D. R. Davies, L. M. Braam, W. S. Reznikoff, I. Rayment, J Biol Chem 274, 11904-13 (1999).
46. S. Kanaya, M. Ikehara, Subcell Biochem 24, 377-422 (1995).
47. M. Haruki, Y. Tsunaka, M. Morikawa, S. Iwai, S. Kanaya, Biochemistry 39, 13939-44 (2000).
48. L. S. Beese, T. A. Steitz, Embo J 10, 25-33 (1991).
49. T. A. Steitz, J. A. Steitz, Proc Natl Acad Sci USA 90, 6498-502 (1993).
50. J. F. Davies, 2nd, Z. Hostomska, Z. Hostomsky, S. R. Jordan, D. A. Matthews, Science 252, 88-95 (1991).
51. K. Klumpp et al., Nucleic Acids Res 31, 6852-9 (2003).
52. D. S. Schwarz, Y. Tomari, P. D. Zamore, Curr Biol 14, 787-91 (2004).
53. J. Martinez, T. Tuschl, Genes Dev 18, 975-80 (2004).
54. U. Wintersberger, Pharmacol Ther 48, 259-80 (1990).
55. H. Huang, R. Chopra, G. L. Verdine, S. C. Harrison, Science 282, 1669-75 (1998).
56. S. G. Sarafianos et al., Embo J 20, 1449-61 (2001).
57. M. L. Kopka, L. Lavelle, G. W. Han, H. L. Ng, R. E. Dickerson, J Mol Biol 334, 653-65 (2003).
58. Y. Tomari et al., Cell 116, 831-41 (2004).
59. J. W. Pham, J. L. Pellino, Y. S. Lee, R. W. Carthew, E. J. Sontheimer, Cell 117, 83-94 (2004).
60. R. M. Esnouf, J. Mol. Graphics 15, 132-134 (1997).
61. P. J. Kraulis, J. Appl. Cryst. 24, 946-950 (1991).
62. D. J. Bacon, W. F. Anderson, J. Molec. Graphics 6, 219-220 (1988).
63. E. A. Merritt, M. E. P. Murphy, Acta Cryst. D50, 869-873 (1994).
64. A. Nicholls, K. A. Sharp, B. Honig, Proteins—Structure, Function and Genetics 11, 281-296 (1991).

All references cited herein including the numbered references above and others throughout the application are incorporated by reference in their entirety.

### EQUIVALENTS

While this invention has been particularly shown above and in the following examples and described with references to preferred embodiments thereof, it will be understood by those skilled in the art that various changes in form and details may be made therein without departing from the scope of the invention encompassed by the appended claims.

### EXEMPLIFICATION

### Example 1

### DNA constructs and site-directed mutagenesis

cDNAs encoding full length human Ago1, Ago2, and Ago3 were generated by RT-PCR from RNAs extracted from 293T, HeLa or S2 cells. Plasmids expressing various Argonaute proteins were made by cloning the cDNAs into a pcDNA3-based myc-epitope tagging vector. Mutations were introduced by site-directed mutagenesis using the QuickChange Kit (Stratagene).

### Example 2

### Human Cell Culture and Transfection

Human 293T cells were cultured in DMEM (10% FBS) in a 37° C. incubator with 5% CO₂. Cell transfections were carried out using calcium-phosphate buffer or Mirus TransIT-LT1 transfection reagent. Luciferase GL3 siRNA duplex was purchased from Dharmacon. siRNA transfection was carried out by using Oligofectamine (Invitrogen). Procedures for immunoprecipitation and immunoblotting were described previously (Caudy et al, Genes. Dev. 16,2491(2002)). Lysis buffer contained 0.5% NP-40, 150 mM NaCl, 2 mM MgCl₂, 2 mM CaCl₂ and 20 mM Tris-HCl pH 7.5. Protease inhibitor and DTT (final 2 mM) were added immediately before lysis. The antibody to the myc tag (9E10) was purchased from Neomarker. RNAs associated with the Ago immunocomplexes were isolated using phenol-chloroform/chloroform extraction and ethanol precipitation. RNAs were stained using SYBR Gold from Molecular Probes. Small RNA Northern blotting was carried out as described previously (Caudy et al., supra).

### Example 3

### mRNA Cleavage Assays and In Vitro Reconstitution of RISC Activity

Capped and uniformly radiolabeled Luciferase mRNA target was in vitro transcribed using the Riboprobe system from Promega and was purified using PAGE as described previously. The immunoaffinity purified Ago complexes were first resuspended in 10 μl buffer containing 100 mM KCl, 2 mM MgCl₂ and 10 mM Tris pH7.5. For in vitro reconstitution of RISC activity, 4 μl of 1 μM in vitro phosphorylated (except where noted) single-stranded siRNA, duplexed siRNA or single-stranded DNA were added to the mix and incubated at 30° C. for 30 minutes. The final reaction was carried out in 20 μl which also contained 1 mM ATP, 0.2 mM GTP, 8 units of RNAsin, 0.3 μg Creatine phosphokinase and 25 mM creatine phosphate. No-ATP reactions lacked ATP, GTP and the regeneration system. After a 2 hour incubation at 30° C., RNAs were extracted using Trizol and chloroform and precipitated with isopropyl alcohol.

### Example 4

### Gene Targeting and Mice

Targeting construct was obtained by screening the lambda phage 3′ HPRT library described in (Zheng et al., Nucleic Acids Res. 27, 2354 (1999)). The resultant targeting construct, containing exons 3-6 of mAgo2, was electroporated into mouse embryonic stem (ES) cells. Targeted clones were injected into C57BL/6 blastocysts to generate chimeras, which were crossed with C57BL/6 mice. Mouse genotyping was performed by Southern blot after digestion of genomic DNA with HindIII. The probe was amplified from genomic DNA using primer sequences 5′GACAATAGTGCAGAGACTTGC3′ and 5′GGGCAGCCTGAGAATTGA3′. GenBank Accession Number for mouse Ago2 is AB081472. The Ago2 gene trap cell line RRE192 was obtained from Bay Genomics(Stryke et al., Nucleic Acids Res. 31, 278 (2003)).

### Example 5

### In Situ Hybridization

In situ hybridization was performed on whole-mount embryos essentially as described (Belo et al., Mech Dev. 68, 45 (1997)). Riboprobes for in situ hybridization were synthesized from T7-promoter containing PCR products corresponding to the 3′ UTRs of Ago2 or Ago3. The Ago2 probe was amplified from genomic DNA using the primers 5′AGCTGTGAAGGCTCTGAG3′ and 5′CAGTCCTACAGGACAAATCT3′, and the Ago3 probe was similarly constructed using primers, AGGCTGTACAGATTCACCAAGATA and CCTTTACAAGAATAGATGCACATT.

### Example 6

### MEF Culture, Transfection, and Gene Silencing Assays

Day 10.5 embryos were dissected and diced in trypsin. Mouse embryo fibroblasts (MEFs) were cultured in DMEM+10% FBS. MEFs were transfected in 24 well plates using Lipofectamine reagent according to the manufacturer's recommendations. Where indicated, each well received 2.5 picomoles of siRNA and 1 ug of plasmid DNA. Dual luciferase assays (Promega) were carried out by cotransfecting cells with plasmids containing firefly luciferase under the control of the SV40 promoter (pGL3-Control, Promega) and Renilla luciferase under the control of the SV40 early enhancer/promoter region (pSV40, Promega). Luciferase siRNA was obtained from Dharmacon (siStarter, anti-luc siRNA-1). GFP (pEGFP-C1) and dsRed (pDsRed-express-N1) plasmids were obtained from Clontech. EGFP siRNA was obtained from Dharmacon (EGFP duplex). Ago1 and Ago2 expression plasmids were as described for the IP experiments, except that proteins were fused to an HA tag rather than a myc tag. Constructs for the translational repression assay were kindly provided by P. Sharp (Doench et al., Genes Dev. 17,438 (2003)).

### Example 7

### RT-PCRs

RNA was extracted from cells and embryos using Trizol Reagent. Reverse transcription was conducted using Superscript-II RT from Invitrogen according to manufacturer's instructions. Subsequent PCR reactions were carried out using the following primers (5′-3′): mAgo1, GCATTTCAAGCAGAAATATAACCTTCA and AGACTTTGATCTCAATCCC ATTGTAG. MAgo2, GTACTTCAAGGACAGGCACAAGCTG and TGGCAATTGC TTTGTTCCTGC. MAgo3, GCTGCAGCTGAAGTACCCACA and GTACTGGAGCATA GGTGCTGGAAGTA. Mouse β-actin, CACTATTGGCAACGAGCGGT and CTTCATGGT GCTAGGAGCCA.

### Example 8

### miRNA Microarrays

RNA was recovered from immunoprecipitates with Trizol (Invitrogen) and conjugated with a Cy3 dinucleotide using T4 RNA ligase (NEB). Labeled RNA was hybridized to microarrays containing probes to 152 human mature microRNA sequences, washed, and scanned on a Genepix 400B array scanner. Log-ratios of Cy3/Cy5 values were global median center normalized for Ago-1, Ago-2, Ago-3 immunoprecipitates. For the control immunoprecipitate, data was normalized by a constant that was the average of the normalization constant for the Ago-1, Ago-2, Ago-3 datasets. Data was sorted in descending order for the Ago-2 dataset and a heat map generated using Treeview (Stanford University).

### Example 9

### miRNA Microarray Results

Ago1-, Ago2- and Ago3-associated RNAs were hybridized to microarrays that report the expression status of 152 human microRNAs. Patterns of associated RNAs were identical within experimental error in each case (FIG. 9, Panel A). Additionally, each of the tagged Ago proteins associated similarly with a co-transfected siRNA (FIG. 9, Panel C). Previous studies have used tagged siRNAs to affinity purify Argonaute-containing RISC (Martinez et al., supra). These preparations, containing mixtures of at least two mammalian Argonautes, were capable of cleaving synthetic mRNAs that were complementary to the tagged siRNA. The ability of purified complexes containing individual Argonaute proteins to catalyze similar cleavages was examined. Surprisingly, irrespective of the siRNA sequence, only Ago2-containing RISC was able to catalyze cleavage (FIG. 9, Panel B; FIG. 14). All three Ago proteins were similarly expressed and bound similar amounts of transfected siRNA (FIG. 1 Panels C and D).

These results demonstrated that mammalian Argonaute complexes are biochemically distinct, with only a single family member being competent for mRNA cleavage. To examine the possibility that Ago proteins might also be biologically specialized, the mouse Ago2 gene were disrupted by targeted insertional mutagenesis (FIG. 15; FIG. 10, Panel A) (Zheng et al., supra). Intercrosses of Ago2 heterozygous produced only wild-type and heterozygous offspring, strongly suggesting that disruption of Ago2 produced an embryonic-lethal phenotype. Ago2 deficient mice display several developmental abnormalities beginning approximately halfway through gestation. Both gene-trap and in situ hybridization data of day 9.5 embryos show broad expression of Ago2 in the embryo, with some hotspots of expression in the forebrain, heart, limb buds and branchial arches (FIG. 10, Panels F and G). The most prominent phenotype is a defect in neural tube closure (FIG. 10, Panels D and E), often accompanied by apparent mispatterning of anterior structures including the forebrain (FIG. 10, Panels C and D). Roughly half of the embryos display complete failure of neural tube closure in the head region (FIG. 10, Panel E), while all embryos display a wavy neural tube in more caudal regions. Mutant embryos also suffer from apparent cardiac failure. The hearts are enlarged, and often accompanied by pronounced swelling of the pericardial cavity (FIG. 10, Panel C). By day 10.5, mutant embryos are severely developmentally delayed compared to wildtype and heterozygous littermates (FIG. 10, Panel B). This large difference in size, like the apparent cardiac failure, may be accounted for by a general nutritional deficiency caused by yolk sac and placental defects (Conway et al., Genesis 35, 1 (2003)), as histological analysis reveals abnormalities in these tissues.

Not all Argonaute proteins are required for successful mammalian development (Deng et al., Cell 2, 819, (2002); Kuramochi-Miyagawa et al., Development 131, 839 (2004)). Ago subfamily members are expressed in overlapping patterns in humans (Sasaki et al., Genomics 82, 323 (2003)). In situ hybridization demonstrates overlapping expression patterns for Ago2 and Ago3 in mouse embryos (FIG. 10, Panel F; FIG. 16). Considered together with the essentially identical patterns of miRNA binding, the results suggest the possibility that the ability of Ago2 to assemble into catalytically active complexes might be critical for mouse development. Although most miRNAs regulate gene expression at the level of protein synthesis, recently miR196 has been demonstrated to cleave the mRNA encoding HoxB8, a developmental regulator (Yekta et al., Science 304, 594 (2004)). Evolutionary conservation of an essential cleavage-competent RISC in organisms in which miRNAs predominantly act by translational regulation raises the possibility that target cleavage by mammalian miRNAs might be more important and widespread than previously appreciated.

Numerous studies have indicated that experimentally triggered RNAi in mammalian cells proceeds through siRNA-directed mRNA cleavage since in many, but not all, cases reiterated binding sites are necessary for repression at the level of protein synthesis (see for example (Bartel, Cell 116, 281 (2004); Doench et al., supra; Kiriakidou et al., Genes Dev. 18, 1165 (2004)). If Ago2 were uniquely capable of assembling into cleavage competent complexes in mice, then embryos or cells lacking Ago2 might be resistant to experimental RNAi. To address this question, mouse embryo fibroblasts (MEF) were prepared from E10.5 embryos from Ago2 heterozygous intercrosses. RT-PCR analysis and genotyping revealed that wild-type, mutant and heterozygous MEF populations were obtained. Importantly, MEF also express other Ago proteins, including Ago1 and Ago3 (FIG. 11, Panel A). Ago2 null MEF were unable to repress gene expression in response to an siRNA (FIG. 11, Panel B; FIG. 17). This defect could be rescued by addition of a third plasmid that encoded human Ago2 but not by Ago1 (FIG. 11, Panel B). In contrast, responses were intact for a reporter of repression at the level of protein synthesis, mediated by an siRNA binding to multiple mismatched sites (Doench et al., supra) (FIG. 11, Panel C).

### Example 10

### Mapping of Determinants for Cleavage

Since Ago2 was unique in its ability form cleavage-competent complexes, determinants of this capacity were mapped. Deletion analysis indicated that an intact Ago2 was required for RISC activity (FIG. 18). Therefore, the sequence of highly conserved but cleavage-incompetent Ago proteins was used as a guide to the construction of Ago2 mutants. A series of point mutations included H634P, H634A, Q633R, Q633A, H682Y, L140W, F704Y and T744Y. While all of these mutations retain siRNA binding activity and most retain cleavage activity, changes at Q633 and H634 have a profound effect on target cleavage (FIG. 12). Both the Q633R and H634P mutations, in which residues were changed to corresponding residues in Ago1/3, abolished catalysis. Changing H634 to A also inactivated Ago2, while a similar change, Q633A, was permissive for cleavage. Thus, even relatively conservative changes can negate the ability of Ago2 to form cleavage-competent RISC.

Several possibilities could explain a lack of cleavage activity for Ago2 mutants. Such mutations could interfere with the proper folding of Ago2. However, this seems unlikely as those same residues presumably permit proper folding in closely related Argonaute proteins, and mutant Ago2 proteins retained the ability to interact with siRNAs. Alternatively, cleavage-incompetent Ago2 mutants could lose the ability to interact with the putative Slicer. Finally, Ago2 itself might be Slicer, with the conservative substitutions altering the active center of the enzyme in a way that prevents cleavage. The last possibility predicted that an active enzyme with relatively pure Ago2 protein may be reconstituted. Ago2 was immunoaffinity purified from 293T cells and attempted to reconstitute RISC in vitro. Incubation with the double-stranded siRNA produced no significant activity, whereas Ago2 could be successfully programmed with single-stranded siRNAs to cleave a complementary substrate (FIG. 13, Panel A). Formation of the active enzyme was unaffected by first washing the immunoprecipitates with up to 2.5M NaCl or IM urea. A 21 nt single stranded DNA was unable to direct cleavage (FIG. 13, Panel A). Programming could be accomplished with different siRNAs that direct activity against different substrates (FIG. 19). RISC is formed though a concerted assembly process in which the RISC-Loading Complex (RLC) acts in an ATP-dependent manner to place one strand of the small RNA into RISC (Nykanen et al., Cell 107, 309 (2001); Pham et al., Cell 117, 83 (2004); Tomari et al., Cell 116, 831 (2004)). In vitro reconstitution occurs in the absence of ATP, suggesting that Ago2 could be programmed with siRNAs without a need for the normal assembly process (FIG. 13, Panel A). However, in vitro reconstitution of RISC still required the essential characteristics of an siRNA. For example, single-stranded siRNAs that lack a 5′ phosphate group cannot reconstitute an active enzyme.

While consistent with the possibility that the catalytic activity of RISC is carried within Ago2, these results do not rule out the possibility that a putative Slicer co-purifies with Ago2. To demonstrate more conclusively that Ago2 is Slicer, the crystal structure of an Argonaute protein from an archebacterium, Pyrococcus furiosus, was analyzed. This structure revealed that the PIWI domain folds into a structure analogous to the catalytic domain of RNAseH and ASV integrase. The notion that such a domain would lie at the center of RISC cleavage is consistent with previous observations. RNAseH and integrases cleave their substrates leaving 5′ phosphate and 3′ hydroxyl groups through a metal catalyzed cleavage reaction (Chapados et al., J. Mol. Biol. 307, 541 (2001); Yang et al., Strcuture 3, 131 (1995)). Notably, previous studies have strongly indicated that the scissile phosphate in the targeted mRNA is cleaved via a metal ion in RISC to give the same phosphate polarity (Schwarz et al., Curr. Biol. 14, 787 (2004)). The in vitro data are consistent with the reconstituted RISC also requiring a divalent metal (FIG. 20). The active center of RNAseH and its relatives consists of a catalytic triad of three carboxylate groups contributed by aspartic or glutamic acid (Chapados et al., supra; Yang et al., supra). These coordinate the essential metal and activate water molecules for nucleolytic attack. Reference to the known structure of RNAseH reveals two aspartate residues in the archeal Ago protein present at the precise spatial locations predicted for formation of an RNAseH-like active site. These align with identical residues in the human Ago2 protein (FIG. 21). Therefore, to test whether the PIWI domain of Ago2 provides catalytic activity to RISC, the two conserved aspartates, D597 and D669, were changed to alanine, with the prediction that either mutation would inactivate RISC cleavage. Consistent with this hypothesis, the mutant Ago2 proteins were incapable of assembling into a cleavage-competent RISC in vitro or in vivo, despite retaining the ability to bind siRNAs (FIG. 13, Panels B-D).

Considered together, the data provide strong support for the notion that Argonaute proteins are the catalytic components of RISC. Firstly, the ability to form an active enzyme is restricted to a single mammalian family member, Ago2. This conclusion is supported both by biochemical analysis and by genetic studies in mutant MEF. Secondly, single amino acid substitutions within Ago2 that convert residues to those present in closely related proteins negate RISC cleavage. Thirdly, the structure of the P. furiosis Argonaute protein reveals provocative structural similarities between the PIWI domain and RNAseH domains, providing a hypothesis for the method by which Argonaute cleaves its substrates. This hypothesis was tested by introducing mutations in the predicted Ago2 active site.

### Example 11

### Protein Expression and Purification

The full length Argonaute gene from Pyroccocus furiosus (PfAgo) was cloned into a pSMT3 vector. PfAgo was expressed as an Smt3 fusion with an N-terminal histidine tag in BL21-RIPL cells. Smt3_Argonaute protein was purified with an NTA-agarose affinity column, and Smt3 was removed using Ulp1 protease, which cuts right after Smt3. The pSMT3 vector-Ulp1 protease system was a generous gift from Dr. Chris Lima. PfAgo was further purified with a heating step, as this protein is from a hyperthermophilic organism, anion exchange chromatography and gel filtration. Purified protein was concentrated to 12.5 mg/ml in 50 mM Tris-HCl (pH8.0) and 300 mM NaCl. Se-Met substituted protein was expressed using metabolic inhibition of methionine biosynthesis as described in (G. D. Van Duyne, R. F. Standaert, P. A. Karplus, S. L. Schreiber, J. Clardy, J Mol Biol 229, 105-24 (1993)). Se-Met incorporation was confirmed by mass spectrometry.

### Example 12

### Crystallization and Data Collection

Initial crystals were grown by vapor diffusion using the hanging-drop method in the presence of organic solvents. The quality of crystals was significantly improved by several rounds of microseeding. Selenomethionine (Se-Met) substituted protein crystals were obtained by microseeding with native crystals. Mercury-derivatized crystals were prepared by soaking native crystals in 1 mM p-chloromercuriphenylsulfonic acid for 5 hours. For cryoprotection crystals were soaked for 1 min in crystallization solution containing increasing amounts of ethylenglycol (EG) in 5% steps to a final EG concentration of 40% (v/v). Crystals diffracted to approximately 2 Å resolution. All data were collected to a resolution of 2.25A under cryogenic conditions (100 K) at beamline X25 at the National Synchrotron Light Source (NSLS) at Brookhaven National Laboratory. Data were processed with HKL2000 (http://www.hk1-xray.com) (Table 1 provided in FIG. 23).

Crystallization condition for native crystal:

1) Well solution as Water; and 2) Mixing 2 μl of 12.5 mg/ml PfAgo protein with 1 μl of water and 0.3 ul of 7% 1-butanol

Crystallization condition for Se-crystal:

1) Well solution as Water; and 2) Mixing 2 μl of 12.5 mg/ml PfAgo protein with 0.3 μl of 7% 1-butanol.

### Example 13

### Structure Determination

Phases were calculated from a three-wavelength anomalous dispersion (MAD) experiment at the selenium inflection, peak and high remote energies using a Se-Met substituted crystal at the peak energy for the mercury derivative. 17 selenium sites were located using SnB (C. M. Weeks, R. Miller, J. of Applied Crystallography 32, 120-124 (1999)) and a single Hg site was located by calculating an anomalous difference Fourier map using initial phases calculated from the selenium data. Data from all three wavelengths for the Se-Met derivative and one wavelength for the Hg derivative were used for heavy atom site refinement by the program SHARP (E. delaFortelle, G. Bricogne, Meth. Enzymol. 276, 472-494 (1997)), followed by solvent flattening. A partial model was built using the program wARP (A. Perrakis, R. Morris, V. S. Lamzin, Nature Structure Biol. 6, 458-463 (1999)). The program SIGMAA (C.C.C.P.N.4. (Acta Crystallogr. D50, 760, Daresbury, UK, 1994)) was used to combine the partial structure model with the experimental phases. Iterative model building using the program 0 (T. A. Jones, M. Kjeldgaard, Methods Enzymol. 277, 173-208 (1997)) and crystallographic refinement with the program CNS (A. T. Brünger et al., Acta Crystallogr. D54, 905-921 (1998)) lead to the final model that contains 5913 protein atoms, and 77 water molecules (Table 1 provided in FIG. 23). Several loops are disordered in the structure and were not included: L26-G38, 1253-K256, E278-V281, L347-L354, and S414-K442.

### Example 14

### UV Crosslinking

PfAgo or GST were incubated with a 21-mer 5′-32 P-labeled ssRNA with an IodoU at the 5′ end and unlabeled competitor ssRNA for 30 min at 30° C. Incubation was carried out in 10 mM Tris-HCl (pH 7.5), 2 mM MgCl₂, and 150 mM KCl. UV crosslinking was done using a Stratalinker (Stratagene) at 312 nm for 20 min at room temperature. Double-stranded RNA probes were gel purified after annealing the 5′-³²P-labeled ssRNA with an unlabeled complementary strand to form a ds-siRNA (including a 2-nucleotide 3′overhang and a 5′-phosphate group).

1. A crystalline Argonaute.

2-5. (canceled)

6. A data array comprising the atomic coordinates of an Argonaute protein as set forth in Table 3.

7. An electronic representation of a crystal structure of an Argonaute protein or a portion thereof.

8. The electronic representation of claim 7, wherein said portion is a binding site of the Argonaute protein.

9. The electronic representation of claim 7, wherein said portion is a domain of the Argonaute protein.

10. The electronic representation of claim 7, further comprising an electronic representation of an agent in a binding site of an Argonaute protein.

11. A method for obtaining the crystalline Argonaute of claim 1, comprising subjecting an Argonaute protein at 10-15 mg/ml to crystallization conditions for a time sufficient for crystal formation.

12-13. (canceled)

14. A method of identifying an agent that modulates the activity of an RNAi construct comprising:
(a) providing an isolated or recombinant Argonaute protein; and
(b) assaying the expression and/or activity of said Argonaute protein in the presence of a candidate agent,
wherein a change in the expression and/or activity of said Argonaute protein in the presence of a candidate agent is indicative of said candidate agent capable of modulating the activity of an RNAi construct.

15. A composition for targeted gene inhibition comprising an agent that modulates the RNase activity of an Argonaute protein.

16. A pharmaceutical composition comprising the composition of claim 15 and a physiologically acceptable carrier.

17. A cell line that overexpresses an Argonaute protein.

18. An assay for identifying nucleic acid sequences for conferring a particular phenotype in a cell, comprising:
(a) constructing a library of nucleic acid sequences oriented to produce double stranded RNA;
(b) introducing a dsRNA library into a culture of target cell line of claim 17;
(c) identifying members of the library which confer a particular phenotype on the cell, and identifying the sequence from the cell which is identical or homologous to the library member.

19. A nucleic acid composition comprising:
(a) a first nucleic acid comprising an RNAi construct and
(b) a second nucleic acid encoding an Argonaute protein.

20. The nucleic acid composition of claim 19, wherein the RNAi construct comprises a nucleotide sequence encoding a single-strand siRNA.

21. A pharmaceutical composition comprising the nucleic acid composition of claim 19 and a physiologically acceptable carrier.

22. A cell expressing the nucleic acid composition of claim 19.

23. A method of determining the three-dimensional structure of an Argonaute protein or a mutant, derivative, variant, analogue, homologue, sub-domain or fragment thereof comprising:
(a) aligning the amino acid sequence of the Argonaute mutant, derivative, variant, analogue, homologue, sub-domain or fragment with the amino acid sequence set forth in SEQ ID NO: 5 to match homologous regions of the amino acid sequences;
(b) modelling the structure of the matched homologous regions of said target Argonaute protein of unknown structure on the corresponding regions of the Argonaute protein structure as defined by the atomic coordinates of claim 6; and
(c) determining a conformation for the Argonaute mutant, derivative, variant, analogue, homologue, sub-domain or fragment which substantially preserves the structure of said matched homologous regions.

24. A method of identifying an agent that binds an Argonaute protein comprising:
(a) applying a 3-dimensional molecular modeling algorithm to the Argonaute atomic coordinates of claim 6 to determine the spatial coordinates of the binding pocket of the Argonaute protein; and
(b) electronically screening the stored spatial coordinates of a set of candidate agents against the spatial coordinates of the Argonaute protein binding pocket to identify agents that can bind to the Argonaute protein.

25. A computer-based method for the analysis of the interaction of a molecular structure with an Argonaute protein, comprising:
(a) providing a structure comprising a three-dimensional representation of said Argonaute protein or a portion thereof, which representation comprises all or a portion of the coordinates of claim 6;
(b) providing a molecular structure to be fitted to said Argonaute protein structure; and
(c) fitting the molecular structure to the Argonaute protein structure of (a).

26. A computer-readable storage medium encoded with the Argonaute atomic coordinates of claim 6.

27. The method of claim 14, wherein an agent that potentiates the activity of an RNAi construct is identified by assaying for an increase in the expression and/or activity of the Argonaute protein in the presence of the candidate agent.


SEQUENCE LISTING (42 sequences)

SEQ ID NO: 1 - protein, 857 aa, Homo sapiens
MEAGPSGAAAGAYLPPLQQVFQAPRRPGIGTVGKPIKLLANYFEVDIPKIDVYHYEVDIKPDKCPRRVNREVVEYMVQHFKPQIFGDRKPVYDGKKNIYTVTALPIGNERVDFEVTIPGEGKDRIFKVSIKWLAIVSWRMLHEALVSGQIPVPLESVQALDVAMRHLASMRYTPVGRSFFSPPEGYYHPLGGGREVWFGFHQSVRPAMWKMMLNIDVSATAFYKAQPVIEFMCEVLDIRNIDEQPKPLTDSQRVRFTKEIKGLKVEVTHCGQMKRKYRVCNVTRRPASHQTFPLQLESGQTVECTVAQYFKQKYNLQLKYPHLPCLQVGQEQKHTYLPLEVCNIVAGQRCIKKLTDNQTSTMIKATARSAPDRQEEISRLMKNASYNLDPYIQEFGIKVKDDMTEVTGRVLPAPILQYGGRNRAIATPNQGVWDMRGKQFYNGIEIKVWAIACFAPQKQCREEVLKNFTDQLRKISKDAGMPIQGQPCFCKYAQGADSVEPMFRHLKNTYSGLQLIIVILPGKTPVYAEVKRVGDTLLGMATQCVQVKNVVKTSPQTLSNLCLKINVKLGGINNILVPHQRSAVFQQPVIFLGADVTHPPAGDGKKPSITAVVGSMDAHPSRYCATVRVQRPRQEIIEDLSYMVRELLIQFYKSTRFKPTRIIFYRDGVPEGQLPQILHYELLAIRDACIKLEKDYQPGITYIVVQKRHHTRLFCADKNERIGKSGNIPAGTTVDTNITHPFEFDFYLCSHAGIQGTSRPSHYYVLWDDNRFTADELQILTYQLCHTYVRCTRSVSIPAPAYYARLVAFRARYHLVDKEHDSGEGSHISGQSNGRDPQALAKAVQVHQDTLRTMYFA

SEQ ID NO: 2 - protein, 859 aa, Homo sapiens
MYSGAGPALAPPAPPPPIQGYAFKPPPRPDFGTSGRTIKLQANFFEMDIPKIDIYHYELDIKPEKCPRRVNREIVEHMVQHFKTQIFGDRKPVFDGRKNLYTAMPLPIGRDKVELEVTLPGEGKDRIFKVSIKWVSCVSLQALHDALSGRLPSVPFETIQALDVVMRHLPSMRYTPVGRSFFTASEGCSNPLGGGREVWFGFHQSVRPSLWKMMLNIDVSATAFYKAQPVIEFVCEVLDFKSIEEQQKPLTDSQRVKFTKEIKGLKVEITHCGQMKRKYRVCNVTRRPASHQTFPLQQESGQTVECTVAQYFKDRHKLVLRYPHLPCLQVGQEQKHTYLPLEVCNIVAGQRCIKKLTDNQTSTMIRATARSAPDRQEEISKLMRSASFNTDPYVREFGIMVKDEMTDVTGRVLQPPSILYGGRNKAIATPVQGVWDMRNKQFHTGIEIKVWAIACFAPQRQCTEVHLKSFTEQLRKISRDAGMPIQGQPCFCKYAQGADSVEPMFRHLKNTYAGLQLVVVILPGKTPVYAEVKRVGDTVLGMATQCVQMKNVQRTTPQTLSNLCLKINVKLGGVNNILLPQGRPPVFQQPVIFLGADVTHPPAGDGKKPSIAAVVGSMDAHPNRYCATVRVQQHRQEIIQDLAAMVRELLIQFYKSTRFKPTRIIFYRDGVSEGQFQQVLHHELLAIREACIKLEKDYQPGITFIVVQKRHHTRLFCTDKNERVGKSGNIPAGTTVDTKITHPTEFDFYLCSHAGIQGTSRPSHYHVLWDDNRFSSDELQILTYQLCHTYVRCTRSVSIPAPAYYAHLVAFRARYHLVDKEHDSAEGSHTSGQSNGRDHQALAKAVQVHQDTLRTMYFA

SEQ ID NO: 3 - protein, 860 aa, Homo sapiens
MEIGSAGPAGAQPLLMVPRRPGYGTMGKPIKLLANCFQVEIPKIDVYLYEVDIKPDKCPRRVNREVVDSMVQHFKVTIFGDRRPVYDGKRSLYTANPLPVATTGVDLDVTLPGEGGKDRPFKVSIKFVSRVSWHLLHEVLTGRTLPEPLELDKPISTNPVHAVDVVLRHLPSMKYTPVGRSFFSAPEGYDHPLGGGREVWFGFHQSVRPAMWKMMLNIDVSATAFYKAQPVIQFMCEVLDIHNIDEQPRPLTDSHRVKFTKEIKGLKVEVTHCGTMRRKYRVCNVTRRPASHQTFPLQLENGQTVERTVAQYFREKYTLQLKYPHLPCLQVGQEQKHTYLPLEVCNIVAGQRCIKKLTDNQTSTMIKATARSAPDRQEEISRLVRSANYETDPFVQEFQFKVRDEMAHVTGRVLPAPMLQYGGRNRTVATPSHGVWDMRGKQFHTGVEIKMWAIACFATQRQCREEILKGFTDQLRKISKDAGMPIQGQPCFCKYAQGADSVEPMFRHLKNTYSGLQLIIVILPGKTPVYAEVKRVGDTLLGMATQCVQVKNVIKTSPQTLSNLCLKINVKLGGINNILVPHQRPSVFQQPVIFLGADVTHPPAGDGKKPSIAAVVGSMDAHPSRYCATVRVQRPRQEIIQDLASMVRELLIQFYKSTRFKPTRIIFYRDGVSEGQFRQVLYYELLAIREACISLEKDYQPGITYIVVQKRHHTRLFCADRTERVGRSGNIPAGTTVDTDITHPYEFDFYLCSHAGIQGTSRPSHYHVLWDDNCFTADELQLLTYQLCHTYVRCTRSVSIPAPAYYAHLVAFRARYHLVDKEHDSAEGSHVSGQSNGRDPQALAKAVQIHQDTLRTMYFA

SEQ ID NO: 4 - protein, 861 aa, Homo sapiens
MEALGPGPPASLFQPPRRPGLGTVGKPIRLLANHFQVQIPKIDVYHYDVDIKPEKRPRRVNREVVDTMVRHFKMQIFGDRQPGYDGKRNMYTAHPLPIGRDRVDMEVTLPGEGKDQTFKVSVQWVSVVSLQLLLEALAGHLNEVPDDSVQALDVITRHLPSMRYTPVGRSFFSPPEGYYHPLGGGREVWFGFHQSVRPAMWNMMLNIDVSATAFYRAQPIIEFMCEVLDIQNINEQTKPLTDSQRVKFTKEIRGLKVEVTHCGQMKRKYRVCNVTRRPASHQTFPLQLENGQAMECTVAQYFKQKYSLQLKYPHLPCLQVGQEQKHTYLPLEVCNIVAGQRCIKKLTDNQTSTMIKATARSAPDRQEEISRLVKSNSMVGGPDPYLKEFGIVVHNEMTELTGRVLPAPMLQYGGRNKTVATPNQGVWDMRGKQFYAGIEIKVWAVACFAPQKQCREDLLKSFTDQLRKISKDAGMPIQGQPCFCKYAQGADSVEPMFKHLKMTYVGLQLIVVILPGKTPVYAEVKRVGDTLLGMATQCVQVKNVVKTSPQTLSNLCLKINAKLGGINNVLVPHQRPSVFQQPVIFLGADVTHPPAGDGKKPSIAAVVGSMDGHPSRYCATVRVQTSRQEISQELLYSQEVIQDLTNMVRELLIQFYKSTRFKPTRIIYYRGGVSEGQMKQVAWPELIAIRKACISLEEDYRPGITYIVVQKRHHTRLFCADKTERVGKSGNVPAGTTVDSTITHPSEFDFYLCSHAGIQGTSRPSHYQVLWDDNCFTADELQLLTYQLCHTYVRCTRSVSIPAPAYYARLVAFRARYHLVDKDHDSAEGSHVSGQSNGRDPQALAKAVQIHHDTQHTMYFA

SEQ ID NO: 5 - protein, 770 aa, Pyrococcus furiosus
MKAKVVINLVKINKKIIPDKIYVYRLFNDPEEELQKEGYSIYRLAYENVGIVIDPENLIIATTKELEYEGEFIPEGEISFSELRNDYQSKLVLRLLKENGIGEYELSKLLRKFRKPKTFGDYKVIPSVEMSVIKHDEDFYLVIHIIHQIQSMKTLWELVNKDPKELEEFLMTHKENLMLKDIASPLKTVYKPCFEEYTKKPKLDHNQEIVKYWYNYHIERYWNTPEAKLEFYRKFGQVDLKQPAILAKFASKIKKNKNYKIYLLPQLVVPTYNAEQLESDVAKEILEYTKLMPEERKELLENILAEVDSDIIDKSLSEIEVEKIAQELENKIRVRDDKGNSVPISQLNVQKSQLLLWTNYSRKYPVILPYEVPEKFRKIREIPMFIILDSGLLADIQNFATNEFRELVKSMYYSLAKKYNSLAKKARSTNEIGLPFLDFRGKEKVITEDLNSDKGIIEVVEQVSSFMKGKELGLAFIAARNKLSSEKFEEIKRRLFNLNVISQVVNEDTLKNKRDKYDRNRLDLFVRHNLLFQVLSKLGVKYYVLDYRFNYDYIIGIDVAPMKRSEGYIGGSAVMFDSQGYIRKIVPIKIGEQRGESVDMNEFFKEMVDKFKEFNIKLDNKKILLLRDGRITNNEEEGLKYISEMFDIEVVTMDVIKNHPVRAFANMKMYFNLGGAIYLIPHKLKQAKGTPIPIKLAKKRIIKNGKVEKQSITRQDVLDIFILTRLNYGSISADMRLPAPVHYAHKFANAIRNEWKIKEEFLAEGFLYFV

SEQ ID NO: 6 - protein, 94 aa, Homo sapiens
GADVTHPPAGDGKKPSIAAVVGSMDAHPNRYCATVRVQQHRQKIIQDLAAMVRELLIQFYKSTRFKPTRIIFYRDGVSEGQFQQVLHHELLAIR

SEQ ID NO: 7 - protein, 94 aa, Mouse
GADVTHPPAGDGKKPSIAAVVGSMDAHPNRYCATVRVQQHRQEIIQDLAAMVRELLIQFYKSTRFKPTRIIFYRDGVSEGQFQQVLHHELLAIR

SEQ ID NO: 8 - protein, 94 aa, Rat; identical to SEQ ID NO: 7

SEQ ID NO: 9 - protein, 94 aa, Bos taurus; identical to SEQ ID NO: 7

SEQ ID NO: 10 - protein, 94 aa, Rabbit; identical to SEQ ID NO: 7

SEQ ID NO: 11 - protein, 95 aa, Drosophila melanogaster
GADVTHPPAGDNKKPSIAAVVGSMDAHPSRYAATVRVQQHRQEIIQELSSMVRELLIMFYKSTGGYKPHRIILYRDGVSEGQFPHVLQHELTAIR

SEQ ID NO: 12 - protein, 95 aa, Anopheles gambiae
GADVTHPPAGDNKKPSIAAVVGSMDAHPSRYAATVRVQQHRQEIIQELSSMVRELLIMFYKSTGGFKPHRIILYRDGVSEGQFPHVLQHELTAIR

SEQ ID NO: 13 - protein, 94 aa, Caenorhabditis briggsae
GCDITHPPAGDSRKPSIAAVVGSMDAHPSRYAATVRVQQHRQEIISDLTYMVRELLVQFYRNTRFKPARIVVYRDGVSEGQFFNVLQYELRAIR

SEQ ID NO: 14 - protein, 94 aa, Caenorhabditis elegans; identical to SEQ ID NO: 13

SEQ ID NO: 15 - protein, 94 aa, Caenorhabditis elegans
GCDITHPAAGDTRKPSIAAVVGSMDAHPSRYAATVRVQQHRQEIITDLTYMVRELLVQFYRNTRFKPARIVVYRDGVSEGQLFNVLQYELRAIR

SEQ ID NO: 16 - protein, 108 aa, Oryza sativa
GADVTHPHPGEDSSPSIAAVVASQDWPEVTKYAGLVSAQAHRQELIQDLFKVWQDPHRGTVTGGMIKELLISFKRATGQKPQRIIFYRDGVSEGQFYQVLLYELDAIR

SEQ ID NO: 17 - protein, 108 aa, Oryza sativa
GADVTHPHPGEDSSPSIAAVVASQDWPEVTKYAGLVSAQAHRQELIEDLYKIWQDPQRGTVSGGMIRELLISFKRSTGEKPQRIIFYRDGVSEGQFYQVLLYELNAIR

SEQ ID NO: 18 - protein, 108 aa, Arabidopsis
GADVTHPHPGEDSSPSIAAVVASQDWPEITKYAGLVCAQAHRQELIQDLFKEWKDPQKGVVTGGMIKELLIAFRRSTGHKPLRIIFYRDGVSEGQFYQVLLYELDAIR

SEQ ID NO: 19 - protein, 108 aa, Arabidopsis zwille
GADVTHPENGEESSPSIAAVVASQDWPEVTKYAGLVCAQAHRQELIQDLYKTWQDPVRGTVSGGMIRDLLISFRKATGQKPLRIIFYRDGVSEGQFYQVLLYELDAIR

SEQ ID NO: 20 - protein, 85 aa, Pyrococcus furiosis
GIDVAPMKRSEGYIGGSAVMFDSQGYIRKIVPIKIGEQRGESVDMNEFFKEMVDKFKEFNIKLDNKKILLLRDGRITNNEEEGLK

SEQ ID NO: 21 - DNA, 21 bp, Artificial Sequence
  chemically synthesized
gacaatagtgcagagacttgc

SEQ ID NO: 22 - DNA, 18 bp, Artificial Sequence
  chemically synthesized
gggcagcctgagaattga

SEQ ID NO: 23 - DNA, 18 bp, Artificial Sequence
  chemically synthesized
agctgtgaaggctctgag

SEQ ID NO: 24 - DNA, 20 bp, Artificial Sequence
  chemically synthesized
cagtcctacaggacaaatct

SEQ ID NO: 25 - DNA, 24 bp, Artificial Sequence
  chemically synthesized
aggctgtacagattcaccaagata

SEQ ID NO: 26 - DNA, 24 bp, Artificial Sequence
  chemically synthesized
cctttacaagaatagatgcacatt

SEQ ID NO: 27 - DNA, 27 bp, Artificial Sequence
  chemically synthesized
gcatttcaagcagaaatataaccttca

SEQ ID NO: 28 - DNA, 26 bp, Artificial Sequence
  chemically synthesized
agactttgatctcaatcccattgtag

SEQ ID NO: 29 - DNA, 25 bp, Artificial Sequence
  chemically synthesized
gtacttcaaggacaggcacaagctg

SEQ ID NO: 30 - DNA, 21 bp, Artificial Sequence
  chemically synthesized
tggcaattgctttgttcctgc

SEQ ID NO: 31 - DNA, 21 bp, Artificial Sequence
  chemically synthesized
gctgcagctgaagtacccaca

SEQ ID NO: 32 - DNA, 26 bp, Artificial Sequence
  chemically synthesized
gtactggagcataggtgctggaagta

SEQ ID NO: 33 - DNA, 20 bp, Artificial Sequence
  chemically synthesized
cactattggcaacgagcggt

SEQ ID NO: 34 - DNA, 20 bp, Artificial Sequence
  chemically synthesized
cttcatggtgctaggagcca

SEQ ID NO: 35 - protein, 123 aa, Phyrococcus furiosis
MKTLWELVNKDPKELEEFLMTHKENLMLKDIASPLKTVYKPCFEEYTKKPKLDHNQEIVKYWYNYHIERYWNTPEAKLEFYRKFGQVDLKQPAILAKFASKIKKKNYKIYLLPQLVVPTYNAE

SEQ ID NO: 36 - protein, 124 aa, Homo sapiens
QPVIEFMCEVLDIRNIDEQPKPLTDSQRVRFTKEIKGLKVEVTHCGQMKRKYRVCNVTRRPASHQTFPLQLESGQTVECTVAQYFKQKYNLQLKYPHLPCLQVGQEQKHTYLPLEVCNIVAGQR

SEQ ID NO: 37 - protein, 119 aa, Drosophila melanogaster
MPMIEYLERFSLKAKINNTTNLDYSRRFLEPFLRGINVVYTPPQSFQSAPRVYRVNGLSRAPASSETFEHDGKKVTIASYFHSRNYPLKFPQLHCLNVGSSIKSILLPIELCSIEEGQA

SEQ ID NO: 38 - protein, 226 aa, Phyrococcus furiosis
LDYRFNYDYIIGIDVAPMKRSEGYIGGSAVMFDSQGYIRKIVPIKIGEQRGESVDMNEFFKEMVDKFKEFNIKLDNKKILLLRDGRITNNEEEGLKYISEMFDIEVVTMDVIKNHPVRAFANMKMYFNLGGAIYLIPHKLKQAKGTPIPIKLAKKRIIKNGKVEKQSITRQDVLDIFILTRLNYGSISADMRLPAPVHYAHKFANAIRNEWKIKEEFLAEGFLYFV

SEQ ID NO: 39 - protein, 277 aa, Homo sapiens
RSAVFQQPVIFLGADVTHPPAGDGKKPSITAVVGSMDAHPSRYCATVRVQRPRQEIIEDLSYMVRELLIQFYKSTRFKPTRIIFYRDGVPEGQLPQILHYELLAIRDACIKLEKDYQPGITYIVVQKRHHTRLFCADKNERIGKSGNIPAGTTVDTNITHPFEFDFYLCSHAGIQGTSRPSHYYVLWDDNRFTADELQILTYQLCHTYVRCTRSVSIPAPAYYARLVAFRARYHLVDKEHDSGEGSHISGQSNGRDPQALAKAVQVHQDTLRTMYFA

SEQ ID NO: 40 - protein, 277 aa, Homo sapiens
RPPVFQQPVIFLGADVTHPPAGDGKKPSIAAVVGSMDAHPNRYCATVRVQQHRQEIIQDLAAMVRELLIQFYKSTRFKPTRIIFYRDGVSEGQFQQVLHHELLAIREACIKLEKDYQPGITFIVVQKRHHTRLFCTDKNERVGKSGNIPAGTTVDTKITHPTEFDFYLCSHAGIQGTSRPSHYHVLWDDNRFSSDELQILTYQLCHTYVRCTRSVSIPAPAYYAHLVAFRARYHLVDKEHDSAEGSHTSGQSNGRDHQALAKAVQVHQDTLRTMYFA

SEQ ID NO: 41 - protein, 277 aa, Homo sapiens
RPSVFQQPVIFLGADVTHPPAGDGKKPSIAAVVGSMDAHPSRYCATVRVQRPRQEIIQDLASMVRELLIQFYKSTRFKPTRIIFYRDGVSEGQFRQVLYYELLAIREACISLEKDYQPGITYIVVQKRHHTRLFCADRTERVGRSGNIPAGTTVDTDITHPYEFDFYLCSHAGIQGTSRPSHYHVLWDDNCFTADELQLLTYQLCHTYVRCTRSVSIPAPAYYAHLVAFRARYHLVDKEHDSAEGSHVSGQSNGRDPQALAKAVQIHQDTLRTMYFA

SEQ ID NO: 42 - protein, 287 aa, Homo sapiens
RPSVFQQPVIFLGADVTHPPAGDGKKPSIAAVVGSMDGHPSRYCATVRVQTSRQEISQELLYSQEVIQDLTNMVRELLIQFYKSTRFKPTRIIYYRGGVSEGQMKQVAWPELIAIRKACISLEEDYRPGITYIVVQKRHHTRLFCADKTERVGKSGNVPAGTTVDSTITHPSEFDFYLCSHAGIQGTSRPSHYQVLWDDNCFTADELQLLTYQLCHTYVRCTRSVSIPAPAYYARLVAFRARYHLVDKDHDSAEGSHVSGQSNGRDPQALAKAVQIHHDTQHTMYFA